(19) United States
(12) Patent Application Publication
(10) Pub. No.: US 2024/0043844 A1
(43) Pub. Date: Feb. 8, 2024

(54) FN3 Domain-siRNA Conjugates with Enzyme Replacement Therapy

(71) Applicant: ARO BIOTHERAPEUTICS COMPANY, Philadelphia, PA (US)

(72) Inventors: Swapnil KULKARNI, Philadelphia, PA (US); Russell C. ADDIS, Philadelphia, PA (US); Steven G. NADLER, Philadelphia, PA (US); Yao XIN, Philadelphia, PA (US); Zhanna DRUZINA, Philadelphia, PA (US); Karyn T. O'NEIL, Philadelphia, PA (US); Robert V. KOLAKOWSKI, Philadelphia, PA (US); Stephen J. ANDERSON, Philadelphia, PA (US)

(21) Appl. No.: 18/301,036

(22) Filed: Apr. 14, 2023

Related U.S. Application Data
(60) Provisional application No. 63/330,797, filed on Apr. 14, 2022.

(51) Int. Cl. C12N 15/113; A61P 3/00

(52) U.S. Cl. CPC C12N 15/1137; C12Y 302/0102; A61P 3/00; C12N 2310/14; C12N 2310/351; C12N 2320/31

(57) ABSTRACT

The present disclosure relates to compositions, such as siRNA molecules and FN3 domains conjugated to the same, in combination with enzyme replacement therapies, as well as methods of making and using the same.

### CROSS-REFERENCE TO RELATED APPLICATIONS

This application claims priority to and the benefit of U.S. Provisional Application No. 63/330,797, filed Apr. 14, 2022, which is hereby incorporated by reference in its entirety.

### REFERENCE TO AN ELECTRONIC SEQUENCE LISTING

This application contains a Sequence Listing which has been submitted electronically in XML format and is hereby incorporated by reference in its entirety. The Sequence Listing is named “ROO-030US_SL.xml”, was created on Sep. 19, 2023, and is 2,074,533 bytes in size.

### FIELD

The present embodiments relate to siRNA molecules that can be conjugated fibronectin type III domains (FN3) and methods of using the molecules in combination with enzyme replacement therapies.

### BACKGROUND

Therapeutic nucleic acids include, e.g., small interfering RNA (siRNA), micro RNA (miRNA), antisense oligonucleotides, ribozymes, plasmids, immune stimulating nucleic acids, antisense, antagomir, antimir, microRNA mimic, supermir, U1 adaptor, and aptamer. In the case of siRNA or miRNA, these nucleic acids can down-regulate intracellular levels of specific proteins through a process termed RNA interference (RNAi). The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models. In addition, siRNA constructs are currently being evaluated in clinical studies and have been approved for a variety of diseases.

However, two problems currently faced by siRNA constructs are, first, their susceptibility to nuclease digestion in plasma and, second, their limited ability to gain access to the intracellular compartment where they can bind the RISC (RNA-induced Silencing Complex) when administered systemically as the free siRNA or miRNA. Certain delivery systems, such as lipid nanoparticles formed from cationic lipids with other lipid components, such as cholesterol and PEG lipids, carbohydrates (such as GalNac trimers) have been used to facilitate the cellular uptake of the oligonucleotides. However, these have not been shown to be successful in efficiently and effectively delivering siRNA to its intended target in tissues other than the liver.

There remains a need for compositions and methods for delivering siRNA to its intended cellular target. Further, what is needed is a FN3 domain with optimized properties for clinical use that can specifically bind to CD71, and methods of using such molecules for novel therapeutics that enable intracellular access via receptor mediated internalization of CD71. The present embodiments fulfill these needs as well as others.

### SUMMARY

In some embodiments, a method of treating a glycogen storage disease in a subject in need thereof is provided. In some embodiments, the method comprises the administration of a composition comprising one or more FN3 domains linked to an siRNA molecule comprising a sense strand and antisense strand, as provided herein, and an enzyme replacement therapy (ERT).

In some embodiments, the glycogen storage disease is selected from the group consisting of Pompe Disease (GSD2, glucosidase alpha acid (GAA) deficiency), Cori's disease or Forbes' disease (GSD3, Glycogen debranching enzyme (AGL) deficiency), Andersen's disease (GSD4, glycogen branching enzyme (GBE1) deficiency), McArdle disease (GSD5, Muscle glycogen phosphorylase (PYGM) deficiency), Tarui's Disease (GSD7, Muscle phosphofructokinase (PFKM) deficiency), Aldolase A Deficiency (GSD12, aldolase A (ALDOA) deficiency), type II Diabetes/diabetic nephropathy, Lafora Disease, hypoxia, and adult polyglucosan body disease.

In some embodiments, the ERT comprises one or more enzymes selected from the group consisting of glucosidase alpha acid (GAA), glycogen debranching enzyme (AGL), glycogen branching enzyme (BGE1), muscle glycogen phosphorylase (PYGM), muscle phosphofructokinase (PFKM), aldolase A (ALDOA), malin, laforin, glycogen synthase (GYS2), glucose-6-phosphatase (G6PC/SLC37A4), phosphorylase kinase (PHKA2/PHKB/PHKG2/PHKA1), phosphoglycerate mutase (PGAM2), muscle lactate dehydrogenase (LDHA), glucose transporter (GLUT2), β-enolase (ENO3), and glycogenin-1 (GYG1)

In some embodiments, siRNA conjugated to FN3 domains that bind CD71 protein are provided.

In some embodiments, FN3 domains are provided that comprise the amino acid sequence of any FN3 domain provided herein. In some embodiments, the FN3 domains bind to CD71. In some embodiments, the FN3 domains specifically bind to CD71.

In some embodiments, the composition comprises two FN3 domains connected by a linker, such as a flexible linker. In some embodiments, the two FN3 domains bind to different targets. In some embodiments, a first FN3 domain binds to CD71. In some embodiments, a second FN3 domain binds to a different target that is not CD71.

In some embodiments, oligonucleotides, such as dsRNA or siRNA molecules, are provided herein. In some embodiments, the oligonucleotides have the sequences as provided herein, with or without the modifications provided herein. In some embodiments, the oligonucleotides are provided in a composition, such as a pharmaceutical composition. In some embodiments, the oligonucleotides are conjugated to a polypeptide.

In some embodiments, compositions comprising one or more FN3 domains conjugated to a siRNA molecule are provided.

In some embodiments, compositions having a formula of (X1)ₙ-(X2)_{q}-(X3)_{y}-L-X4, wherein X1 is a first FN3 domain; X2 is a second FN3 domain; X3 is a third FN3 domain or half-life extender molecule; L is a linker; and X4 is an oligonucleotide molecule, wherein n, q, and y are each independently 0 or 1, are provided.

In some embodiments, compositions having a formula of C—(X1)ₙ-(X2)_{q}-(X3)_{y}-L-X4, wherein C is a polymer or albumin binding domain (ABD); X1 is a first FN3 domain; X2 is a second FN3 domain; X3 is a third FN3 domain or half-life extender molecule; L is a linker; and X4 is an oligonucleotide molecule, wherein n, q, and y are each independently 0 or 1, are provided.

In some embodiments, compositions having a formula of (X1)ₙ-(X2)_{q}-(X3)_{y}-L-X4-C, wherein X1 is a first FN3 domain; X2 is a second FN3 domain; X3 is a third FN3 domain or half-life extender molecule; L is a linker; X4 is an oligonucleotide molecule; and C is a polymer or albumin binding domain (ABD), wherein n, q, and y are each independently 0 or 1, are provided.

In some embodiments, compositions having a formula of X4-L-(X1)ₙ-(X2)_{q}-(X3)_{y}, wherein X1 is a first FN3 domain; X2 is a second FN3 domain; X3 is a third FN3 domain or half-life extender molecule; L is a linker; and X4 is an oligonucleotide molecule, wherein n, q, and y are each independently 0 or 1, are provided.

In some embodiments, compositions having a formula of C—X4-L-(X1)ₙ-(X2)_{q}-(X3)_{y}, wherein C is a polymer or albumin binding domain (ABD); X1 is a first FN3 domain; X2 is a second FN3 domain; X3 is a third FN3 domain or half-life extender molecule; L is a linker; and X4 is an oligonucleotide molecule, wherein n, q, and y are each independently 0 or 1, are provided.

In some embodiments, compositions having a formula of X4-L-(X1)ₙ-(X2)_{q}-(X3)_{y}-C, wherein X1 is a first FN3 domain; X2 is a second FN3 domain; X3 is a third FN3 domain or half-life extender molecule; L is a linker; X4 is an oligonucleotide molecule; and C is a polymer or albumin binding domain (ABD), wherein n, q, and y are each independently 0 or 1, are provided.

In some embodiments, compositions having a formula of C—(X1)ₙ-(X2)_{q}[L-X4]-(X3)_{y}, wherein X1 is a first FN3 domain; X2 is a second FN3 domain; X3 is a third FN3 domain or half-life extender molecule; L is a linker; X4 is an oligonucleotide molecule; and C is a polymer or albumin binding domain (ABD), wherein n, q, and y are each independently 0 or 1, are provided.

In some embodiments, compositions having a formula of (X1)ₙ-(X2)_{q}[L-X4]-(X3)_{y}-C, wherein X1 is a first FN3 domain; X2 is a second FN3 domain; X3 is a third FN3 domain or half-life extender molecule; L is a linker; X4 is an oligonucleotide molecule; and C is a polymer or albumin binding domain (ABD), wherein n, q, and y are each independently 0 or 1, are provided.

In some embodiments, pharmaceutical compositions comprising one or more of the compositions provided herein are provided.

In some embodiments, uses of a composition as provided herein or of any of in the preparation of a pharmaceutical composition or medicament for treating a condition as provided for herein are provided.

In some embodiments, methods of selectively reducing GYS1 mRNA and protein in skeletal muscle and administering an enzyme as provided herein are provided. In certain embodiments, GYS1 mRNA and protein is not reduced or not reduced significantly in the liver and/or the kidney.

In some embodiments, isolated polynucleotides encoding the FN3 domains described herein are provided.

In some embodiments, vectors comprising the polynucleotides described herein are provided.

In some embodiments, host cells comprising the vectors described herein are provided.

In some embodiments, methods of producing the FN3 domains are provided. In some embodiments, the method comprises culturing a host cell comprising a vector encoding or expressing the FN3 domain. In some embodiments, the method further comprises purifying the FN3 domain. In some embodiments, the FN3 domain binds CD71.

In some embodiments, pharmaceutical compositions comprising a FN3 domain that binds to CD71 linked to an oligonucleotide molecule and a pharmaceutically acceptable carrier are provided. In some embodiments, kits comprising one or more of the FN3 domains with or without the oligonucleotide molecules are provided.

### DESCRIPTION OF THE DRAWINGS

FIG. 1 is a flow chart representing the properties assessed and considered for siRNA screening.

FIG. 2 is a graph of an RNA sequencing experiment which identifies the changes in the transcriptome after cells are transfected with siRNA pair HHH, wherein the arrow identifies the significant decrease in GYS1 transcript.

FIG. 3 provides the results of a target binding assay using over 6,000 receptors in the proteome array, wherein the data demonstrates that CD71 is the exclusive binding target of the FN3 domain.

FIG. 4A demonstrates the knockdown of GYS1 mRNA in mouse gastrocnemius muscle using 3 difference FN3 domain-siRNA conjugates compared with vehicle alone. FIG. 4B demonstrates the knockdown of GYS1 protein in mouse gastrocnemius muscle using 3 difference FN3 domain-siRNA conjugates compared with vehicle alone.

FIG. 5 demonstrates the GYS1 knockdown is highly specific for skeletal muscle using 3 different FN3 domain-siRNA conjugates compared with a siRNA to a different target (AHA-1).

### DETAILED DESCRIPTION OF THE DISCLOSURE

As used in this specification and the appended claims, the singular forms “a,” “an,” and “the” include plural referents unless the content clearly dictates otherwise. Thus, for example, reference to “a cell” includes a combination of two or more cells, and the like.

“Fibronectin type III (FN3) domain” (FN3 domain) refers to a domain occurring frequently in proteins including fibronectins, tenascin, intracellular cytoskeletal proteins, cytokine receptors and prokaryotic enzymes (Bork and Doolittle, Proc Nat Acad Sci. USA, 1992, 89:8990-8994; Meinke et al., J Bacteriol., 1993, 175:1910-1918; Watanabe et al., J Biol Chem., 1990, 265:15659-15665). Exemplary FN3 domains are the 15 different FN3 domains present in human tenascin C, the 15 different FN3 domains present in human fibronectin (FN), and non-natural synthetic FN3 domains as described for example in U.S. Pat. No. 8,278,419. Individual FN3 domains are referred to by domain number and protein name, e.g., the 3^{rd} FN3 domain of tenascin (TN3), or the 10^{th} FN3 domain of fibronectin (FN10). As used throughout, “centyrin” also refers to a FN3 domain. Further, FN3 domains as described herein are not antibodies as they do not have the structure of a variable heavy (V_{H}) and/or light (V_{L}) chain.

As used herein, “autoimmune disease” refers to disease conditions and states wherein the immune response of an individual is directed against the individual's own constituents, resulting in an undesirable and often debilitating condition. As used herein, “autoimmune disease” is intended to further include autoimmune conditions, syndromes, and the like. Autoimmune diseases include, but are not limited to, Addison's disease, allergy, allergic rhinitis, ankylosing spondylitis, asthma, atherosclerosis, autoimmune diseases of the ear, autoimmune diseases of the eye, autoimmune atrophic gastritis, autoimmune hepatitis, autoimmune hymolytic anemia, autoimmune parotitis, autoimmune uveitis, celiac disease, primary biliary cirrhosis, benign lymphocytic aniitis, COPD, colitis, coronary heart disease, Crohn's disease, diabetes (Type I), depression, diabetes, including Type 1 and/or Type 2 diabetes, epididymitis, glomerulonephritis, Goodpasture's syndrome, Graves' disease, Guillain-Barre syndrome, Hashimoto's disease, hemolytic anemia, idiopathic thrombocytopenic purpura, inflammatory bowel disease (IBD), immune response to recombinant drug products, e.g., factor VII in hemophilia, juvenile idiopathic arthritis, systemic lupus erythematosus, lupus nephritis, male infertility, mixed connective tissue disease, multiple sclerosis, myasthenia gravis, oncology, osteoarthritis, pain, primary myxedema, pemphigus, pernicious anemia, polymyositis, psoriasis, psoriatic arthritis, reactive arthritis, rheumatic fever, rheumatoid arthritis, sarcoidosis, scleroderma, Sjogren's syndrome, spondyloarthropathies, sympathetic ophthalmia, T-cell lymphoma, T-cell acute lymphoblastic leukemia, testicular antiocentric T-cell lymphoma, thyroiditis, transplant rejection, ulcerative colitis, autoimmune uveitis, and vasculitis. Autoimmune diseases include, but are not limited to, conditions in which the tissue affected is the primary target, and in some cases, the secondary target. Such conditions include, but are not limited to, AIDS, atopic allergy, bronchial asthma, eczema, leprosy, schizophrenia, inherited depression, transplantation of tissues and organs, chronic fatigue syndrome, Alzheimer's disease, Parkinson's disease, myocardial infarction, stroke, autism, epilepsy, Arthus's phenomenon, anaphylaxis, and alcohol and drug addiction.

As used herein, “capture agent” refers to substances that bind to a particular type of cell and enable the isolation of that cell from other cells. Exemplary capture agents are magnetic beads, ferrofluids, encapsulating reagents, molecules that bind the particular cell type and the like.

As used herein, “sample” refers to a collection of similar fluids, cells, or tissues isolated from a subject, as well as fluids, cells, or tissues present within a subject. Exemplary samples are tissue biopsies, fine needle aspirations, surgically resected tissue, organ cultures, cell cultures and biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium and lavage fluids and the like.

“Substituting” or “substituted” or “mutating” or “mutated” refers to altering, deleting, or inserting one or more amino acids or nucleotides in a polypeptide or polynucleotide sequence to generate a variant of that sequence.

“Variant” refers to a polypeptide or a polynucleotide that differs from a reference polypeptide or a reference polynucleotide by one or more modifications, for example, substitutions, insertions or deletions.

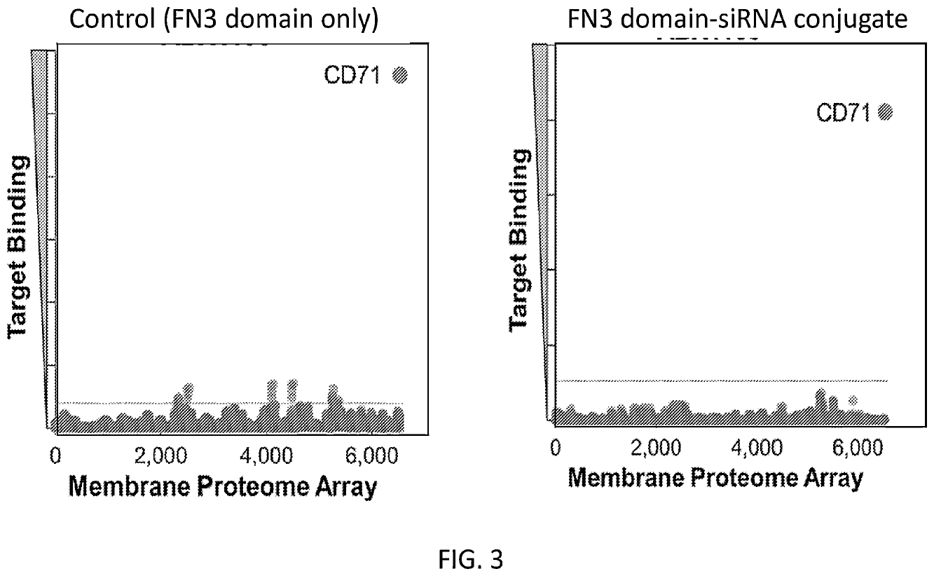

“Specifically binds” or “specific binding” refers to the ability of a FN3 domain to bind to its target, such as CD71, with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Alternatively, “specific binding” refers to the ability of a FN3 domain to bind to its target (e.g. CD71) at least 5-fold above a negative control in standard solution ELISA assay. Specific binding can also be demonstrated using the proteome array as described herein and shown in FIG. 3. In some embodiments, a negative control is an FN3 domain that does not bind CD71. In some embodiments, an FN3 domain that specifically binds CD71 may have cross-reactivity to other related antigens, for example, to the same predetermined antigen from other species (homologs), such as Macaca fascicularis (cynomolgous monkey, cyno) or Pan troglodytes (chimpanzee).

“Library” refers to a collection of variants. The library may be composed of polypeptide or polynucleotide variants.

“Stability” refers to the ability of a molecule to maintain a folded state under physiological conditions such that it retains at least one of its normal functional activities, for example, binding to a predetermined antigen such as CD71.

“CD71” refers to human CD71 protein having the amino acid sequence of SEQ ID NOs: 2 or 5. In some embodiments, SEQ ID NO: 2 is the full length human CD71 protein. In some embodiments, SEQ ID NO: 5 is the extracellular domain of human CD71.

“Tencon” refers to the synthetic fibronectin type III (FN3) domain having the consensus sequence shown in SEQ ID NO: 1 (LPAPKNLVVSEVTEDSLRLSWTAPDAAFDSFLIQYQESEKVGEAINLTVPGSERSYDLTG LKPGTEYTVSIYGVKGGHRSNPLSAEFTT) and described in U.S. Pat. Publ. No. 2010/0216708, which is hereby incorporated by reference in its entirety.

A “cancer cell” or a “tumor cell” refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific markers levels, invasiveness, tumor growth or suppression in suitable animal hosts such as nude mice, and the like, in vitro, in vivo, and ex vivo (Freshney, Culture of Animal Cells: A Manual of Basic Technique (3rd ed. 1994)).

A “dendritic cell” refers to a type of antigen-presenting cell (APC) that forms an important role in the adaptive immune system. The main function of dendritic cells is to present antigens to T lymphocytes, and to secrete cytokines that may further modulate the immune response directly or indirectly. Dendritic cells have the capacity to induce a primary immune response in the inactive or resting naïve T lymphocytes.

An “immune cell” refers to the cells of the immune system categorized as lymphocytes (T-cells, B-cells and NK cells), neutrophils, or monocytes/macrophages. These are all types of white blood cells.

Myocytes are cells making up various muscular tissues, categorized as skeletal, smooth, and cardiac muscle cells. Skeletal muscle cells are multi-nucleated and striated. Smooth muscle cells are single-nucleated and not striated. Cardiac muscle cells are single-nucleated and striated, and are found only in the heart. As used herein, a “muscle cell” refers to skeletal muscle cells and smooth muscle cells, and does not refer to cardiac muscle cells. As used herein, a “heart cell” refers to cardiac muscle cells only.

“Vector” refers to a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

“Expression vector” refers to a vector that can be utilized in a biological system or in a reconstituted biological system to direct the translation of a polypeptide encoded by a polynucleotide sequence present in the expression vector.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than about 50 amino acids may be referred to as “peptides”.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Subject” includes any human or nonhuman animal. “Nonhuman animal” includes all vertebrates, e.g., mammals and non-mammals, such as nonhuman primates, sheep, dogs, cats, horses, cows chickens, amphibians, reptiles, etc. Except when noted, the terms “patient” or “subject” are used interchangeably.

“Isolated” refers to a homogenous population of molecules (such as synthetic polynucleotides or a polypeptide such as FN3 domains) which have been substantially separated and/or purified away from other components of the system the molecules are produced in, such as a recombinant cell, as well as a protein that has been subjected to at least one purification or isolation step. “Isolated FN3 domain” refers to an FN3 domain that is substantially free of other cellular material and/or chemicals and encompasses FN3 domains that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Enzyme replacement therapy” or “ERT” refers to the use of a pharmaceutical composition that is administered to a subject that comprises one or more enzymes to the subject. In some embodiments, the enzyme is being administered to supplement or replace an enzyme that is missing or deficient in some aspect in the subject. Typically, the enzyme or enzymes involved in the therapy are administered to a subject by intravenous infusion of a pharmaceutical composition comprising the enzyme or enzymes. As described herein, the enzyme replacement therapy can be administered along with a composition comprising an FN3 domain which can be linked to an additional therapeutic, such as an oligonucleotide (e.g., siRNA, mRNA, cDNA, antisense oligonucleotide, and the like). In some embodiments, the administration of the enzyme replacement therapy can be before, during, or after the administration of the composition comprising an FN3 domain linked to the additional therapeutic as provided herein.

In some embodiments, a composition comprising a polypeptide, such as a polypeptide comprising a FN3 domain, which can be linked to an oligonucleotide molecule are provided. The oligonucleotide molecule can be, for example, a siRNA molecule, antisense oligonucleotide, mRNA, and the like.

Accordingly, in some embodiments, the siRNA is a double-stranded RNAi (dsRNA) agent capable of inhibiting the expression of a target gene. The dsRNA agent comprises a sense strand (passenger strand) and an antisense strand (guide strand). In some embodiments, each strand of the dsRNA agent can range from 12-40 nucleotides in length. For example, each strand can be from 14-40 nucleotides in length, 17-37 nucleotides in length, 25-37 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

In some embodiments, the sense strand and antisense strand typically form a duplex dsRNA. The duplex region of a dsRNA agent may be from 12-40 nucleotide pairs in length. For example, the duplex region can be from 14-40 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length.

In some embodiments, the dsRNA comprises one or more overhang regions and/or capping groups of dsRNA agent at the 3′-end, or 5′-end or both ends of a strand. The overhang can be 1-10 nucleotides in length, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or it can be another sequence. The first and second strands can also be joined, e.g., by additional bases to form a hairpin, or by other non-base linkers.

In some embodiments, the nucleotides in the overhang region of the dsRNA agent can each independently be a modified or unmodified nucleotide including, but not limited to, 2′-sugar modified, such as 2-F, 2′-Omethyl, 2′-O-(2-methoxyethyl), 2′-O-(2-methoxyethyl), 2′-O-(2-methoxyethyl), and any combinations thereof. For example, TT (UU) can be an overhang sequence for either end on either strand. The overhang can form a mismatch with the target mRNA or it can be complementary to the gene sequences being targeted or it can be another sequence.

The 5′- or 3′-overhangs at the sense strand, antisense strand or both strands of the dsRNA agent may be phosphorylated. In some embodiments, the overhang region contains two nucleotides having a phosphorothioate, phosphorodithioate, phosphonate, phosphoramidate, or mesyl phosphoramidate between the two nucleotides, where the two nucleotides can be the same or different. In one embodiment, the overhang is present at the 3′-end of the sense strand, antisense strand or both strands. In one embodiment, this 3′-overhang is present in the antisense strand. In one embodiment, this 3′-overhang is present in the sense strand.

The dsRNA agent may comprise only a single overhang, which can strengthen the interference activity of the dsRNA, without affecting its overall stability. For example, the single-stranded overhang is located at the 3′-terminal end of the sense strand or, alternatively, at the 3′-terminal end of the antisense strand. The dsRNA may also have a blunt end, located at the 5′-end of the antisense strand (or the 3′-end of the sense strand) or vice versa. Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC. For example, the single overhang comprises at least two, three, four, five, six, seven, eight, nine, or ten nucleotides in length.

In some embodiments, the dsRNA agent may also have two blunt ends, at both ends of the dsRNA duplex.

In some embodiments, every nucleotide in the sense strand and antisense strand of the dsRNA agent may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2 hydroxyl on the ribose sugar; wholesale replacement of the phosphate moiety with “dephospho” linkers; modification or replacement of a naturally occurring base; and replacement or modification of the ribose-phosphate backbone.

In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate, phosphorodithoate, phosphonate, phosphoramidate, or mesyl phosphoramidate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In one embodiment, each residue of the sense strand and antisense strand is independently modified with 2′-O-methyl or 2′-fluoro.

In some embodiments, at least two different modifications are typically present on the sense strand and antisense strand. Those two modifications may be the 2′-deoxy, 2′-O-methyl or 2′-fluoro modifications, acyclic nucleotides or others.

In one embodiment, the sense strand and antisense strand each comprises two differently modified nucleotides selected from 2′-fluoro, 2′-O-methyl or 2′-deoxy.

The dsRNA agent may further comprise at least one phosphorothioate, phosphorodithoate, phosphonate, phosphoramidate, mesyl phosphoramidate, or methylphosphonate internucleotide linkage. The phosphorothioate, phosphorodithoate, phosphonate, phosphoramidate, mesyl phosphoramidate, or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA agent comprises the phosphorothioate, phosphorodithoate, phosphonate, phosphoramidate, mesyl phosphoramidate, or methylphosphonate internucleotide linkage modification in the overhang region. For example, the overhang region comprises two nucleotides having a phosphorothioate, phosphorodithoate, phosphonate, phosphoramidate, mesyl phosphoramidate, or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate, phosphorodithoate, phosphonate, phosphoramidate, mesyl phosphoramidate, or methylphosphonate internucleotide linkage, and optionally, there may be additional phosphorothioate, phosphorodithoate, phosphonate, phosphoramidate, mesyl phosphoramidate, or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. In some embodiments, these terminal three nucleotides may be at the 3-end of the antisense strand.

In some embodiments, the dsRNA composition is linked by a modified base or nucleoside analogue as described in U.S. Pat. No. 7,427,672, which is incorporated herein by reference. In some embodiments, the modified base or nucleoside analogue is referred to as the linker or L in formulas described herein.

In some embodiments, the modified base or nucleoside analogue has the structure as shown in Chemical Formula I and a salt thereof:

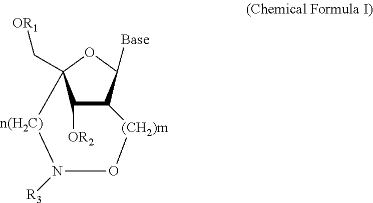

where Base represents an aromatic heterocyclic group or aromatic hydrocarbon ring group optionally having a substituent, R₁ and R₂ are identical or different, and each represent a hydrogen atom, a protective group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected with a protective group for nucleic acid synthesis, or —P(R₄)R₅ where R₄ and R₅ are identical or different, and each represent a hydroxyl group, a hydroxyl group protected with a protective group for nucleic acid synthesis, a mercapto group, a mercapto group protected with a protective group for nucleic acid synthesis, an amino group, an alkoxy group having 1 to 5 carbon atoms, an alkylthio group having 1 to 5 carbon atoms, a cyanoalkoxy group having 1 to 6 carbon atoms, or an amino group substituted by an alky group having 1 to 5 carbon atoms, R₃ represents a hydrogen atom, an alkyl group, an alkenyl group, a cycloalkyl group, an aryl group, an aralkyl group, an acyl group, a sulfonyl group, or a functional molecule unit substituent, and m denotes an integer of 0 to 2, and n denotes an integer of 0 to 3. In some embodiments, m and n are 0.

In some embodiments, the modified base or nucleoside analogue has the structure as shown in Chemical Formula I and salts thereof, wherein R₁ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the modified base or nucleoside analogue has the structure as shown in Chemical Formula I and salts thereof, wherein R₁ is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the modified base or nucleoside analogue has the structure as shown in Chemical Formula I and salts thereof, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the modified base or nucleoside analogue has the structure as shown in Chemical Formula I and salts thereof, wherein R₂ is a hydrogen atom, an acetyl group, a benzoyl group, a methanesulfonyl group, a p-toluenesulfonyl group, a benzyl group, a p-methoxybenzyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the modified base or nucleoside analogue has the structure as shown in Chemical Formula I and salts thereof, wherein R₃ is a hydrogen atom, a phenoxyacetyl group, an alkyl group having 1 to 5 carbon atoms, an alkenyl group having 1 to 5 carbon atoms, an aryl group having 6 to 14 carbon atoms, a methyl group substituted by one to three aryl groups, a lower aliphatic or aromatic sulfonyl group such as a methanesulfonyl group or a p-toluenesulfonyl group, an aliphatic acyl group having 1 to 5 carbon atoms such as an acetyl group, or an aromatic acyl group such as a benzoyl group.

In some embodiments, the modified base or nucleoside analogue has the structure as shown in Chemical Formula I and salts thereof, wherein the functional molecule unit substituent as R₃ is a fluorescent or chemiluminescent labeling molecule, a nucleic acid incision activity functional group, or an intracellular or nuclear transfer signal peptide.

In some embodiments, the modified base or nucleoside analogue has the structure as shown in Chemical Formula I and salts thereof, wherein Base is a purin-9-yl group, a 2-oxopyrimidin-1-yl group, or a purin-9-yl group or a 2-oxopyrimidin-1-yl group having a substituent selected from the following α group: α group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the modified base or nucleoside analogue has the structure as shown in Chemical Formula I and salts thereof, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the modified base or nucleoside analogue has the structure as shown in Chemical Formula I and salts thereof, wherein m is 0, and n is 1.

In some embodiments, the modified base or nucleoside analogue is a DNA oligonucleotide or RNA oligonucleotide analogue, containing one or two or more of one or more types of unit structures of nucleoside analogues having the structure as shown in Chemical Formula II, or a pharmacologically acceptable salt thereof, provided that a form of linking between respective nucleosides in the oligonucleotide analogue may contain one or two or more phosphorothioate bonds [—OP(O)(S⁻)O—], phosphorodithioate bonds [—O₂PS₂—], phosphonate bonds [—PO(OH)₂—], phosphoramidate bonds [—O═P(OH)₂—], or mesyl phosphoramidate bonds [—OP(O)(N)(SO₂)(CH₃)O—] aside from a phosphodiester bond [—OP(O₂⁻)O—] identical with that in a natural nucleic acid, and if two or more of one or more types of these structures are contained, Base may be identical or different between these structures:

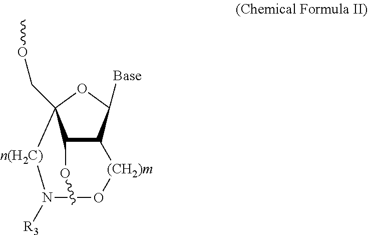

where Base represents an aromatic heterocyclic group or aromatic hydrocarbon ring group optionally having a substituent, R₃ represents a hydrogen atom, an alkyl group, an alkenyl group, a cycloalkyl group, an aryl group, an aralkyl group, an acyl group, a sulfonyl group, a silyl group, or a functional molecule unit substituent, and m denotes an integer of 0 to 2, and n denotes an integer of 0 to 3. In some embodiments, m and n are 0.

In some embodiments, the oligonucleotide analogue or the pharmacologically acceptable salt thereof has the structure as shown in Chemical Formula II, wherein R₁ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the oligonucleotide analogue or the pharmacologically acceptable salt thereof has the structure as shown in Chemical Formula II, wherein R₁ is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the oligonucleotide analogue or the pharmacologically acceptable salt thereof has the structure as shown in Chemical Formula II, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the oligonucleotide analogue or the pharmacologically acceptable salt thereof has the structure as shown in Chemical Formula II, wherein R₂ is a hydrogen atom, an acetyl group, a benzoyl group, a benzyl group, a p-methoxybenzyl group, a methanesulfonyl group, a p-toluenesulfonyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the oligonucleotide analogue or the pharmacologically acceptable salt thereof has the structure as shown in Chemical Formula II, wherein R₃ is a hydrogen atom, a phenoxyacetyl group, an alkyl group having 1 to 5 carbon atoms, an alkenyl group having 1 to 5 carbon atoms, an aryl group having 6 to 14 carbon atoms, a methyl group substituted by one to three aryl groups, a lower aliphatic or aromatic sulfonyl group such as a methanesulfonyl group or a p-toluenesulfonyl group, an aliphatic acyl group having 1 to 5 carbon atoms such as an acetyl group, or an aromatic acyl group such as a benzoyl group.

In some embodiments, the oligonucleotide analogue or the pharmacologically acceptable salt thereof has the structure as shown in Chemical Formula II, wherein the functional molecule unit substituent as R₃ is a fluorescent or chemiluminescent labeling molecule, a nucleic acid incision activity functional group, or an intracellular or nuclear transfer signal peptide.

In some embodiments, the oligonucleotide analogue or the pharmacologically acceptable salt thereof has the structure as shown in Chemical Formula II, wherein Base is a purin-9-yl group, a 2-oxopyrimidin-1-yl group, or a purin-9-yl group or a 2-oxopyrimidin-1-yl group having a substituent selected from the following a group: a group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the oligonucleotide analogue or the pharmacologically acceptable salt thereof has the structure as shown in Chemical Formula II, wherein Base is 6-aminopurin-9-yl (i.e. adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl group having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the oligonucleotide analogue or the pharmacologically acceptable salt thereof has the structure as shown in Chemical Formula II, wherein m is 0, and n is 1.

In some embodiments, compositions described herein further comprise a polymer (polymer moiety C). In some instances, the polymer is a natural or synthetic polymer, consisting of long chains of branched or unbranched monomers, and/or cross-linked network of monomers in two or three dimensions. In some instances, the polymer includes a polysaccharide, lignin, rubber, or polyalkylen oxide (e.g., polyethylene glycol). In some instances, the at least one polymer includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane, as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the polymer comprises polyalkylene oxide. In some instances, the polymer comprises PEG. In some instances, the polymer comprises polyethylene imide (PEI) or hydroxy ethyl starch (HES).

In some instances, C is a PEG moiety. In some instances, the PEG moiety is conjugated at the 5′ terminus of the oligonucleotide molecule while the binding moiety is conjugated at the 3′ terminus of the oligonucleotide molecule. In some instances, the PEG moiety is conjugated at the 3′ terminus of the oligonucleotide molecule while the binding moiety is conjugated at the 5′ terminus of the oligonucleotide molecule. In some instances, the PEG moiety is conjugated to an internal site of the oligonucleotide molecule. In some instances, the PEG moiety, the binding moiety, or a combination thereof, are conjugated to an internal site of the oligonucleotide molecule. In some instances, the conjugation is a direct conjugation. In some instances, the conjugation is via native ligation.

In some embodiments, the polyalkylene oxide (e.g., PEG) is a polydisperse or monodisperse compound. In some instances, polydisperse material comprises disperse distribution of different molecular weights of the material, characterized by mean weight (weight average) size and dispersity. In some instances, the monodisperse PEG comprises one size of molecules. In some embodiments, C is poly- or monodispersed polyalkylene oxide (e.g., PEG) and the indicated molecular weight represents an average of the molecular weight of the polyalkylene oxide (e.g., PEG) molecules.

In some embodiments, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, C is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some instances, the molecular weight of C is about 200 Da. In some instances, the molecular weight of C is about 300 Da. In some instances, the molecular weight of C is about 400 Da. In some instances, the molecular weight of C is about 500 Da. In some instances, the molecular weight of C is about 600 Da. In some instances, the molecular weight of C is about 700 Da. In some instances, the molecular weight of C is about 800 Da. In some instances, the molecular weight of C is about 900 Da. In some instances, the molecular weight of C is about 1000 Da. In some instances, the molecular weight of C is about 1100 Da. In some instances, the molecular weight of C is about 1200 Da. In some instances, the molecular weight of C is about 1300 Da. In some instances, the molecular weight of C is about 1400 Da. In some instances, the molecular weight of C is about 1450 Da. In some instances, the molecular weight of C is about 1500 Da. In some instances, the molecular weight of C is about 1600 Da. In some instances, the molecular weight of C is about 1700 Da. In some instances, the molecular weight of C is about 1800 Da. In some instances, the molecular weight of C is about 1900 Da. In some instances, the molecular weight of C is about 2000 Da. In some instances, the molecular weight of C is about 2100 Da. In some instances, the molecular weight of C is about 2200 Da. In some instances, the molecular weight of C is about 2300 Da. In some instances, the molecular weight of C is about 2400 Da. In some instances, the molecular weight of C is about 2500 Da. In some instances, the molecular weight of C is about 2600 Da. In some instances, the molecular weight of C is about 2700 Da. In some instances, the molecular weight of C is about 2800 Da. In some instances, the molecular weight of C is about 2900 Da. In some instances, the molecular weight of C is about 3000 Da. In some instances, the molecular weight of C is about 3250 Da. In some instances, the molecular weight of C is about 3350 Da. In some instances, the molecular weight of C is about 3500 Da. In some instances, the molecular weight of C is about 3750 Da. In some instances, the molecular weight of C is about 4000 Da. In some instances, the molecular weight of C is about 4250 Da. In some instances, the molecular weight of C is about 4500 Da. In some instances, the molecular weight of C is about 4600 Da. In some instances, the molecular weight of C is about 4750 Da. In some instances, the molecular weight of C is about 5000 Da. In some instances, the molecular weight of C is about 5500 Da. In some instances, the molecular weight of C is about 6000 Da. In some instances, the molecular weight of C is about 6500 Da. In some instances, the molecular weight of C is about 7000 Da. In some instances, the molecular weight of C is about 7500 Da. In some instances, the molecular weight of C is about 8000 Da. In some instances, the molecular weight of C is about 10,000 Da. In some instances, the molecular weight of C is about 12,000 Da. In some instances, the molecular weight of C is about 20,000 Da. In some instances, the molecular weight of C is about 35,000 Da. In some instances, the molecular weight of C is about 40,000 Da. In some instances, the molecular weight of C is about 50,000 Da. In some instances, the molecular weight of C is about 60,000 Da. In some instances, the molecular weight of C is about 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some instances, a dPEG comprises about 3 or more repeating ethylene oxide units. In some instances, a dPEG comprises about 4 or more repeating ethylene oxide units. In some instances, a dPEG comprises about 5 or more repeating ethylene oxide units. In some instances, a dPEG comprises about 6 or more repeating ethylene oxide units. In some instances, a dPEG comprises about 7 or more repeating ethylene oxide units. In some instances, a dPEG comprises about 8 or more repeating ethylene oxide units. In some instances, a dPEG comprises about 9 or more repeating ethylene oxide units. In some instances, a dPEG comprises about 10 or more repeating ethylene oxide units. In some instances, a dPEG comprises about 11 or more repeating ethylene oxide units. In some instances, a dPEG comprises about 12 or more repeating ethylene oxide units. In some instances, a dPEG comprises about 13 or more repeating ethylene oxide units. In some instances, a dPEG comprises about 14 or more repeating ethylene oxide units. In some instances, a dPEG comprises about 15 or more repeating ethylene oxide units. In some instances, a dPEG comprises about 16 or more repeating ethylene oxide units. In some instances, a dPEG comprises about 17 or more repeating ethylene oxide units. In some instances, a dPEG comprises about 18 or more repeating ethylene oxide units. In some instances, a dPEG comprises about 19 or more repeating ethylene oxide units. In some instances, a dPEG comprises about 20 or more repeating ethylene oxide units. In some instances, a dPEG comprises about 22 or more repeating ethylene oxide units. In some instances, a dPEG comprises about 24 or more repeating ethylene oxide units. In some instances, a dPEG comprises about 26 or more repeating ethylene oxide units. In some instances, a dPEG comprises about 28 or more repeating ethylene oxide units. In some instances, a dPEG comprises about 30 or more repeating ethylene oxide units. In some instances, a dPEG comprises about 35 or more repeating ethylene oxide units. In some instances, a dPEG comprises about 40 or more repeating ethylene oxide units. In some instances, a dPEG comprises about 42 or more repeating ethylene oxide units. In some instances, a dPEG comprises about 48 or more repeating ethylene oxide units. In some instances, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, C is an albumin binding domain. In certain aspects, the albumin binding domain specifically binds to serum albumin, e.g., human serum albumin (HSA), to prolong the half-life of the domain or of another therapeutic to which the albumin binding domain is associated or linked with. In some embodiments, the human serum albumin binding domain comprises an initiator methionine (Met) linked to the N-terminus of the molecule. In some embodiments, the human serum albumin binding domain comprises a cysteine (Cys) linked to a C-terminus or the N-terminus of the domain. The addition of the N-terminal Met and/or the C-terminal Cys may facilitate expression and/or conjugation to another molecule, which can be another half-life extending molecule, such as PEG, a Fc region, and the like.

In some embodiments, the albumin binding domain comprises the amino acid sequence of SEQ ID NOs: 101, 102, 103, 104, 105, 106, 107, 108, 109, 110, 111, 112, 113, 114, 115, 116, 117, 118, or 119, provided in Table 1, below. In some embodiments, the albumin binding domain (protein) is isolated. In some embodiments, the albumin binding domain comprises an amino acid sequence that is at least, or is, 85%, 86%, 87%, 88%, 89%, 90%, 91%, 92%, 93%, 94%, 95%, 96%, 97%, 98%, or 99% identical to the amino acid sequence of SEQ ID NO: 101, 102, 103, 104, 105, 106, 107, 108, 109, 110, 111, 112, 113, 114, 115, 116, 117, 118, or 119. In some embodiments, the albumin binding domain comprises an amino acid sequence that is at least, or is, 85%, 86%, 87%, 88%, 89%, 90%, 901%, 92%, 93%, 94%, 95%, 96%, 97%, 98%, or 99% identical to the amino acid sequence of SEQ ID NO: 101, 102, 103, 104, 105, 106, 107, 108, 109, 110, 111, 112, 113, 114, 115, 116, 117, 118, or 119 provided that the protein has a substitution that corresponds to position 10 of SEQ ID NO: 101, 102, 103, 104, 105, 106, 107, 108, 109, 110, 111, 112, 113, 114, 115, 116, 117, 118, or 119. In some embodiments, the substitution is A10V. In some embodiments, the substitution is A10G, A10L, A10I, A10T, or A10S. In some embodiments, the substitution at position 10 is any naturally occurring amino acid. In some embodiments, the isolated albumin binding domain comprises an amino acid sequence that has 1, 2, 3, 4, 5, 6, 7, 8, 9, 10, 11, 12, 13, or 14 substitutions when compared to the amino acid sequence of SEQ ID NOs: 101, 102, 103, 104, 105, 106, 107, 108, 109, 110, 111, 112, 113, 114, 115, 116, 117, 118, or 119. In some embodiments, the substitution is at a position that corresponds to position 10 of SEQ ID NOs: 101, 102, 103, 104, 105, 106, 107, 108, 109, 110, 111, 112, 113, 114, 115, 116, 117, 118, or 119. In some embodiments, FN3 domains provided comprise a cysteine residue in at least one residue position corresponding to residue positions 6, 11, 22, 25, 26, 52, 53, 61, 88 or positions 6, 8, 10, 11, 14, 15, 16, 20, 30, 34, 38, 40, 41, 45, 47, 48, 53, 54, 59, 60, 62, 64, 70, 88, 89, 90, 91, or 93 of SEQ ID NO: 101, 102, 103, 104, 105, 106, 107, 108, 109, 110, 111, 112, 113, 114, 115, 116, 117, 118, or 119, or at a C-terminus. Although the positions are listed in a series, each position can also be chosen individually. In some embodiments, the cysteine is at a position that corresponds to position 6, 53, or 88. In some embodiments, additional examples of albumin binding domains can be found in U.S. Pat. No. 10,925,932, which is hereby incorporated by reference.

TABLE 1

| SEQ | |
|---|---|
| ID | |
| NO: | SEQUENCE |
| 101 | MLPAPKNLVASRVTEDSARLSWTAP |
| | DAAFDSFNIAYWEPGIGGEAIWLRV |
| | PGSERSYDLTGLKPGTEYKVWIHGV |
| | KGGASSPPLIARFTT |
| 102 | MLPAPKNLVVSRVTEDSARLSWTAP |
| | DAAFDSFNIAYWEPGIGGEAIWLRV |
| | PGSERSYDLTGLKPGTEYKVWIHGV |
| | KGGASSPPLIARFTT |
| 103 | MLPAPKNLVVSRVTEDSARLSWTAP |
| | DAAFDSFAIAYWEPGIGGEAIWLRV |
| | PGSERSYDLTGLKPGTEYKVWIHGV |
| | KGGASSPPLIARFTT |
| 104 | MLPAPKNLVVSRVTEDSARLSWTAP |
| | DAAFDSFNISYWEPGIGGEAIWLRV |
| | PGSERSYDLTGLKPGTEYKVWIHGV |
| | KGGASSPPLIARFTT |
| 105 | MLPAPKNLVVSRVTEDSARLSWTAP |
| | DAAFDSFNIAYAEPGIGGEAIWLRV |
| | PGSRSYDLTGLKPGTEYKVWIHGVK |
| | GGASSPPLIARFTT |
| 106 | MLPAPKNLVVSRVTEDSARLSWTAP |
| | DAAFDSFNIAYWEAGIGGEAIWLRV |
| | PGSERSYDLTGLKPGTEYKVWIHGV |
| | KGGASSPPLIARFTT |
| 107 | MLPAPKNLVVSRVTEDSARLSWTAP |
| | DAAFDSFNIAYWEPAIGGEAIWLRV |
| | PGSERSYDLTGLKPGTEYKVWIHGV |
| | KGGASSPPLIARFTT |
| 108 | MLPAPKNLVVSRVTEDSARLSWTAP |
| | DAAFDSFNIAYWEPGAGGEAIWLRV |
| | PGSERSYDLTGLKPGTEYKVWIHGV |
| | KGGASSPPLIARFTT |
| 109 | MLPAPKNLVVSRVTEDSARLSWTAP |
| | DAAFDSFNIAYWEPGIAGEAIWLRV |
| | PGSERSYDLTGLKPGTEYKVWIHGV |
| | KGGASSPPLIARFTT |
| 110 | MLPAPKNLVVSRVTEDSARLSWTAP |
| | DAAFDSFNIAYWEPGIGGEAIALRV |
| | PGSERSYDLTGLKPGTEYKVWIHGV |
| | KGGASSPPLIARFTT |
| 111 | MLPAPKNLVVSRVTEDSARLSWTAP |
| | DAAFDSFNIAYWEPGIGGEAIWLAV |
| | PGSERSYDLTGLKPGTEYKVWIHGV |
| | KGGASSPPLIARFTT |
| 112 | MLPAPKNLVVSRVTEDSARLSWTAP |
| | DAAFDSFNIAYWEPGIGGEAIWLRV |
| | PGSERSYDLTGLKPGTEYAVWIHGV |
| | KGGASSPPLIARFTT |
| 113 | MLPAPKNLVVSRVTEDSARLSWTAP |
| | DAAFDSFNIAYWEPGIGGEAIWLRV |
| | PGSERSYDLTGLKPGTEYKVAIHGV |
| | KGGASSPPLIARFTT |
| 114 | MLPAPKNLVVSRVTEDSARLSWTAP |
| | DAAFDSFNIAYWEPGIGGEAIWLRV |
| | PGSERSYDLTGLKPGTEYKVWIAGV |
| | KGGASSPPLIARFTT |
| 115 | MLPAPKNLVVSRVTEDSARLSWTAP |
| | DAAFDSFNIAYWEPGIGGEAIWLRV |
| | PGSERSYDLTGLKPGTEYKVWIHGV |
| | KGGSSSPPLIARFTT |
| 116 | MLPAPKNLVVSRVTEDSARLSWTAP |
| | DAAFDSFNIAYWEPGIGGEAIWLRV |
| | PGSERSYDLTGLKPGTEYKVWIHGV |
| | KGGAASPPLIARFTT |
| 117 | MLPAPKNLVVSRVTEDSARLSWTAP |
| | DAAFDSFNIAYWEPGIGGEAIWLRV |
| | PGSERSYDLTGLKPGTEYKVWIHGV |
| | KGGASSAPLIARFTT |
| 118 | MLPAPKNLVVSRVTEDSARLSWTAP |
| | DAAFDSFNIAYWEPGIGGEAIWLRV |
| | PGSERSYDLTGLKPGTEYKVWIHGV |
| | KGGASSPPLAARFTT |
| 119 | MLPAPKNLVVSRVTEDSARLSWTAP |
| | DAAFDSFNIAYWEPGIGGEAIWLRV |
| | PGSERSYDLTGLKPGTEYKVWIHGV |
| | KGGASSPPLIAAFTT |

In some embodiments, the dsRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation, A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), 5′-end mesyl phosphoramidate (5′MsPA), or 5′-deoxy-5′-C-malonyl. When the 5′-end phosphorus-containing group is 5′-end vinylphosphonate (5′-VP), the 5′-VP can be either 5′-E-VP isomer, such as trans-vinylphosphate or cis-vinylphosphate, or mixtures thereof. Representative structures of these modifications can be found in, for example, U.S. Pat. No. 10,233,448, which is hereby incorporated by reference in its entirety.

In some embodiments, nucleotide analogues or synthetic nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, methyl, ethyl, n-propyl, iso-propyl, n-butyl, iso-butyl, tert-butyl, C₁-C₁₀ chain lengths both linear and branched. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazine or hydroxylamino) group, an isocyanate or cyanate group, or a sulfur containing group (e.g., sulfoxide, sulfone, sulfide, and disulfide). In some instances, the alkyl moiety further comprises additional hetero atom such as O, S, N, Se and each of these hetero atoms can be further substituted with alky groups as described above. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. Exemplary chemical structures of a 2′-O-methyl modification of an adenosine molecule and 2′O-methoxyethyl modification of an uridine are illustrated below.

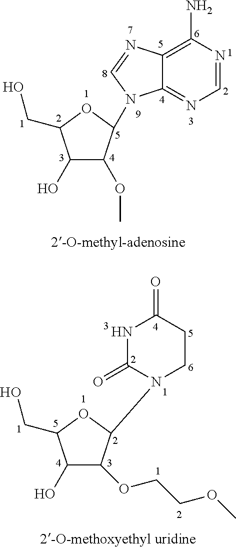 [SMILES: COC1C(O)C(CO)OC1n1cnc2c(N)ncnc21 ; COCCOC1C(O)C(CO)OC1N1CCC(=O)NC1=O]

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

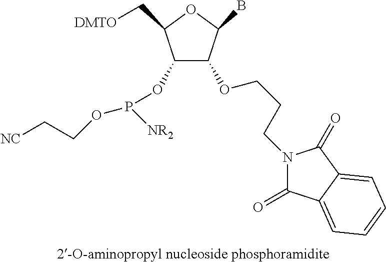

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

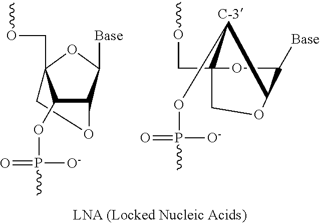

In some instances, the modification at the 2′ hydroxyl group comprises ethylene nucleic acids (ENA) such as for example 2′-4′-ethylene-bridged nucleic acid, which locks the sugar conformation into a C3′-endo sugar puckering conformation. ENA are part of the bridged nucleic acids class of modified nucleic acids that also comprises LNA. Exemplary chemical structures of the ENA and bridged nucleic acids are illustrated below.

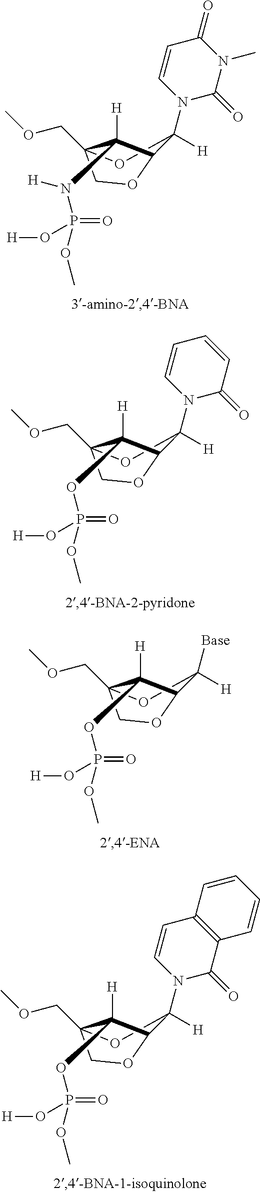

In some embodiments, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some embodiments, nucleotide analogues comprise modified bases such as, but not limited to, 5-propynyluridine, 5-propynylcytidine, 6-methyladenine, 6-methylguanine, N,N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O-and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, nucleotide analogues further comprise morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. In some instances, the five-member ribose ring is substituted with a six-member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

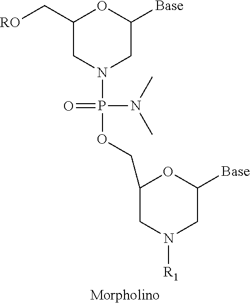

In some embodiments, peptide nucleic acid (PNA) does not contain sugar ring or phosphate linkage and the bases are attached and appropriately spaced by oligoglycine-like molecules, therefore, eliminating a backbone charge.

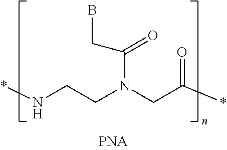

In some embodiments, one or more modifications optionally occur at the internucleotide linkage. In some instances, modified internucleotide linkage include, but is not limited to, phosphorothioates, mesyl phosphoramidate, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. Mesyl phosphoramidate antisense oligonucleotides (MsPA ASO) are antisense oligonucleotides comprising a mesyl phosphoramidate linkage.

In some instances, the modification is a methyl or thiol modification such as methylphosphonate, mesyl phosphoramidate, or thiolphosphonate modification. In some instances, a modified nucleotide includes, but is not limited to, 2′-fluoro N3-P5′-phosphoramidites.

In some instances, a modified nucleotide includes, but is not limited to, hexitol nucleic acid (or 1′,5′-anhydrohexitol nucleic acids (HNA)).

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, the oligonucleotide molecule comprises one or more of the synthetic nucleotide analogues described herein. In some instances, the oligonucleotide molecule comprises 1, 2, 3, 4, 5, 6, 7, 8, 9, 10, 11, 12, 13, 14, 15, 16, 17, 18, 20, 25, or more of the synthetic nucleotide analogues described herein. In some embodiments, the synthetic nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide molecule comprises 1, 2, 3, 4, 5, 6, 7, 8, 9, 10, 11, 12, 13, 14, 15, 16, 17, 18, 20, 25, or more of the synthetic nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the oligonucleotide molecule comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the oligonucleotide molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the oligonucleotide molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification. In some instances, the oligonucleotide molecule comprises 100% modification

In some cases, the oligonucleotide molecule comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification.

In some cases, the oligonucleotide molecule comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification.

In some instances, the oligonucleotide molecule comprises at least one of: from about 10% to about 70% modification, from about 20% to about 70% modification, from about 30% to about 70% modification, from about 40% to about 70% modification, from about 50% to about 70% modification, and from about 60% to about 70% modification.

In some instances, the oligonucleotide molecule comprises at least one of: from about 10% to about 60% modification, from about 20% to about 60% modification, from about 30% to about 60% modification, from about 40% to about 60% modification, and from about 50% to about 60% modification.

In some cases, the oligonucleotide molecule comprises at least one of: from about 10% to about 50% modification, from about 20% to about 50% modification, from about 30% to about 50% modification, and from about 40% to about 50% modification.

In some cases, the oligonucleotide molecule comprises at least one of: from about 10% to about 40% modification, from about 20% to about 40% modification, and from about 30% to about 40% modification.

In some cases, the oligonucleotide molecule comprises at least one of: from about 10% to about 30% modification, and from about 20% to about 30% modification.

In some cases, the oligonucleotide molecule comprises from about 10% to about 20% modification.

In some cases, the oligonucleotide molecule comprises from about 15% to about 90%, from about 20% to about 80%, from about 30% to about 70%, or from about 40% to about 60% modifications.

In additional cases, the oligonucleotide molecule comprises at least about 15%, 20%, 30%, 40%, 50%, 60%, 70%, 80%, 90%, 95%, or 99% modification.

In some embodiments, the oligonucleotide molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 modifications.

In some instances, the oligonucleotide molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 modified nucleotides.

In some instances, from about 5 to about 100% of the oligonucleotide molecule comprise the synthetic nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95% or 100% of the oligonucleotide molecule comprise the synthetic nucleotide analogues described herein. In some instances, about 5% of the oligonucleotide molecule comprises the synthetic nucleotide analogues described herein. In some instances, about 10% of the oligonucleotide molecule comprises the synthetic nucleotide analogues described herein. In some instances, about 15% of the oligonucleotide molecule comprises the synthetic nucleotide analogues described herein. In some instances, about 20% of the oligonucleotide molecule comprises the synthetic nucleotide analogues described herein. In some instances, about 25% of the oligonucleotide molecule comprises the synthetic nucleotide analogues described herein. In some instances, about 30% of the oligonucleotide molecule comprises the synthetic nucleotide analogues described herein. In some instances, about 35% of the oligonucleotide molecule comprises the synthetic nucleotide analogues described herein. In some instances, about 40% of the oligonucleotide molecule comprises the synthetic nucleotide analogues described herein. In some instances, about 45% of the oligonucleotide molecule comprises the synthetic nucleotide analogues described herein. In some instances, about 50% of the oligonucleotide molecule comprises the synthetic nucleotide analogues described herein. In some instances, about 55% of the oligonucleotide molecule comprises the synthetic nucleotide analogues described herein. In some instances, about 60% of the oligonucleotide molecule comprises the synthetic nucleotide analogues described herein. In some instances, about 65% of the oligonucleotide molecule comprises the synthetic nucleotide analogues described herein. In some instances, about 70% of the oligonucleotide molecule comprises the synthetic nucleotide analogues described herein. In some instances, about 75% of the oligonucleotide molecule comprises the synthetic nucleotide analogues described herein. In some instances, about 80% of the oligonucleotide molecule comprises the synthetic nucleotide analogues described herein. In some instances, about 85% of the oligonucleotide molecule comprises the synthetic nucleotide analogues described herein. In some instances, about 90% of the oligonucleotide molecule comprises the synthetic nucleotide analogues described herein. In some instances, about 95% of the oligonucleotide molecule comprises the synthetic nucleotide analogues described herein. In some instances, about 96% of the oligonucleotide molecule comprises the synthetic nucleotide analogues described herein. In some instances, about 97% of the oligonucleotide molecule comprises the synthetic nucleotide analogues described herein. In some instances, about 98% of the oligonucleotide molecule comprises the synthetic nucleotide analogues described herein. In some instances, about 99% of the oligonucleotide molecule comprises the synthetic nucleotide analogues described herein. In some instances, about 100% of the oligonucleotide molecule comprises the synthetic nucleotide analogues described herein. In some embodiments, the synthetic nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the oligonucleotide molecule comprises from about 1 to about 25 modifications, in which the modification comprises synthetic nucleotide analogues described herein. In some embodiments, the oligonucleotide molecule comprises about 1 modification in which the modification comprises a synthetic nucleotide analogue described herein. In some embodiments, the oligonucleotide molecule comprises about 2 modifications in which the modifications comprise a synthetic nucleotide analogue described herein. In some embodiments, the oligonucleotide molecule comprises about 3 modifications in which the modifications comprise a synthetic nucleotide analogue described herein. In some embodiments, the oligonucleotide molecule comprises about 4 modifications in which the modifications comprise a synthetic nucleotide analogue described herein. In some embodiments, the oligonucleotide molecule comprises about 5 modifications in which the modifications comprise a synthetic nucleotide analogue described herein. In some embodiments, the oligonucleotide molecule comprises about 6 modifications in which the modifications comprise a synthetic nucleotide analogue described herein. In some embodiments, the oligonucleotide molecule comprises about 7 modifications in which the modifications comprise a synthetic nucleotide analogue described herein. In some embodiments, the oligonucleotide molecule comprises about 8 modifications in which the modifications comprise a synthetic nucleotide analogue described herein. In some embodiments, the oligonucleotide molecule comprises about 9 modifications in which the modifications comprise a synthetic nucleotide analogue described herein. In some embodiments, the oligonucleotide molecule comprises about 10 modifications in which the modifications comprise a synthetic nucleotide analogue described herein. In some embodiments, the oligonucleotide molecule comprises about 11 modifications in which the modifications comprise a synthetic nucleotide analogue described herein. In some embodiments, the oligonucleotide molecule comprises about 12 modifications in which the modifications comprise a synthetic nucleotide analogue described herein. In some embodiments, the oligonucleotide molecule comprises about 13 modifications in which the modifications comprise a synthetic nucleotide analogue described herein. In some embodiments, the oligonucleotide molecule comprises about 14 modifications in which the modifications comprise a synthetic nucleotide analogue described herein. In some embodiments, the oligonucleotide molecule comprises about 15 modifications in which the modifications comprise a synthetic nucleotide analogue described herein. In some embodiments, the oligonucleotide molecule comprises about 16 modifications in which the modifications comprise a synthetic nucleotide analogue described herein. In some embodiments, the oligonucleotide molecule comprises about 17 modifications in which the modifications comprise a synthetic nucleotide analogue described herein. In some embodiments, the oligonucleotide molecule comprises about 18 modifications in which the modifications comprise a synthetic nucleotide analogue described herein. In some embodiments, the oligonucleotide molecule comprises about 19 modifications in which the modifications comprise a synthetic nucleotide analogue described herein. In some embodiments, the oligonucleotide molecule comprises about 20 modifications in which the modifications comprise a synthetic nucleotide analogue described herein. In some embodiments, the oligonucleotide molecule comprises about 21 modifications in which the modifications comprise a synthetic nucleotide analogue described herein. In some embodiments, the oligonucleotide molecule comprises about 22 modifications in which the modifications comprise a synthetic nucleotide analogue described herein. In some embodiments, the oligonucleotide molecule comprises about 23 modifications in which the modifications comprise a synthetic nucleotide analogue described herein. In some embodiments, the oligonucleotide molecule comprises about 24 modifications in which the modifications comprise a synthetic nucleotide analogue described herein. In some embodiments, the oligonucleotide molecule comprises about 25 modifications in which the modifications comprise a synthetic nucleotide analogue described herein. In some embodiments, the oligonucleotide molecule comprises about 26 modifications in which the modifications comprise a synthetic nucleotide analogue described herein. In some embodiments, the oligonucleotide molecule comprises about 27 modifications in which the modifications comprise a synthetic nucleotide analogue described herein. In some embodiments, the oligonucleotide molecule comprises about 28 modifications in which the modifications comprise a synthetic nucleotide analogue described herein. In some embodiments, the oligonucleotide molecule comprises about 29 modifications in which the modifications comprise a synthetic nucleotide analogue described herein. In some embodiments, the oligonucleotide molecule comprises about 30 modifications in which the modifications comprise a synthetic nucleotide analogue described herein. In some embodiments, the oligonucleotide molecule comprises about 31 modifications in which the modifications comprise a synthetic nucleotide analogue described herein. In some embodiments, the oligonucleotide molecule comprises about 32 modifications in which the modifications comprise a synthetic nucleotide analogue described herein. In some embodiments, the oligonucleotide molecule comprises about 33 modifications in which the modifications comprise a synthetic nucleotide analogue described herein. In some embodiments, the oligonucleotide molecule comprises about 34 modifications in which the modifications comprise a synthetic nucleotide analogue described herein. In some embodiments, the oligonucleotide molecule comprises about 35 modifications in which the modifications comprise a synthetic nucleotide analogue described herein. In some embodiments, the oligonucleotide molecule comprises about 36 modifications in which the modifications comprise a synthetic nucleotide analogue described herein. In some embodiments, the oligonucleotide molecule comprises about 37 modifications in which the modifications comprise a synthetic nucleotide analogue described herein. In some embodiments, the oligonucleotide molecule comprises about 38 modifications in which the modifications comprise a synthetic nucleotide analogue described herein. In some embodiments, the oligonucleotide molecule comprises about 39 modifications in which the modifications comprise a synthetic nucleotide analogue described herein. In some embodiments, the oligonucleotide molecule comprises about 40 modifications in which the modifications comprise a synthetic nucleotide analogue described herein.

In some embodiments, an oligonucleotide molecule is assembled from two separate polynucleotides wherein one polynucleotide comprises the sense strand and the second polynucleotide comprises the antisense strand of the oligonucleotide molecule. In other embodiments, the sense strand is connected to the antisense strand via a linker molecule, which in some instances is a polynucleotide linker or a non-nucleotide linker.

In some embodiments, an oligonucleotide molecule comprises a sense strand and antisense strand, wherein pyrimidine nucleotides in the sense strand comprises 2′-O-methylpyrimidine nucleotides and purine nucleotides in the sense strand comprise 2′-deoxy purine nucleotides. In some embodiments, an oligonucleotide molecule comprises a sense strand and antisense strand, wherein pyrimidine nucleotides present in the sense strand comprise 2′-deoxy-2′-fluoro pyrimidine nucleotides and wherein purine nucleotides present in the sense strand comprise 2′-deoxy purine nucleotides.

In some embodiments, an oligonucleotide molecule comprises a sense strand and antisense strand, wherein the pyrimidine nucleotides when present in said antisense strand are 2′-deoxy-2′-fluoro pyrimidine nucleotides and the purine nucleotides when present in said antisense strand are 2′-O-methyl purine nucleotides.

In some embodiments, an oligonucleotide molecule comprises a sense strand and antisense strand, wherein the pyrimidine nucleotides when present in said antisense strand are 2′-deoxy-2′-fluoro pyrimidine nucleotides and wherein the purine nucleotides when present in said antisense strand comprise 2′-deoxy-purine nucleotides.

In some embodiments, an oligonucleotide molecule comprises a sense strand and antisense strand, and at least one of sense strand and antisense strands has a plurality of (e.g., two or more, three or more, four or more, five or more, six or more, seven or more, eight or more, etc) 2′-O-methyl or 2′-deoxy-2′-fluoro modified nucleotides. In some embodiments, at least two, three, four, five, six, or seven out of the a plurality of 2′-O-methyl or 2′-deoxy-2′-fluoro modified nucleotides are consecutive nucleotides. In some embodiments, consecutive 2′-O-methyl or 2′-deoxy-2′-fluoro modified nucleotides are located at the 5′-end of the sense strand and/or the antisense strand. In some embodiments, consecutive 2′-O-methyl or 2′-deoxy-2′-fluoro modified nucleotides are located at the 3′-end of the sense strand and/or the antisense strand. In some embodiments, the sense strand of oligonucleotide molecule includes at least four, at least five, at least six consecutive 2′-O-methyl modified nucleotides at its 5′ end and/or 3′-end, or both. Optionally, in such embodiments, the sense strand of oligonucleotide molecule includes at least one, at least two, at least three, at least four 2′-deoxy-2′-fluoro modified nucleotides at the 3′ end of the at least four, at least five, at least six consecutive 2′-O-methyl modified nucleotides at the polynucleotides' 5′ end, or at the 5′ end of the at least four, at least five, at least six consecutive 2′-O-methyl modified nucleotides at polynucleotides' 3′ end. Also optionally, such at least two, at least three, at least four 2′-deoxy-2′-fluoro modified nucleotides are consecutive nucleotides.

In some embodiments, an oligonucleotide molecule comprises a sense strand and antisense strand, and at least one of sense strand and antisense strand has 2′-O-methyl modified nucleotide located at the 5′-end of the sense strand and/or the antisense strand. In some embodiments, at least one of sense strand and antisense strands has 2′-O-methyl modified nucleotide located at the 3′-end of the sense strand and/or the antisense strand. In some embodiments, the 2′-O-methyl modified nucleotide located at the 5′-end of the sense strand and/or the antisense strand is a purine nucleotide. In some embodiments, the 2′-O-methyl modified nucleotide located at the 5′-end of the sense strand and/or the antisense strand is a pyrimidine nucleotide.

In some embodiments, an oligonucleotide molecule comprises a sense strand and antisense strand, and one of sense strand and antisense strand has at least two consecutive 2′-deoxy-2′-fluoro modified nucleotides located at the 5′-end, while another strand has at least two consecutive 2′-O-methyl modified nucleotides located at the 5′-end. In some embodiments, where the strand has at least two consecutive 2′-deoxy-2′-fluoro modified nucleotides located at the 5′-end, the strand also includes at least two, at least three consecutive 2′-O-methyl modified nucleotides at the 3′end of the at least two consecutive 2′-deoxy-2′-fluoro modified nucleotides. In some embodiments, one of sense strand and antisense strand has at least two, at least three, at least four, at least five, at least six, or at least seven consecutive 2′-O-methyl modified nucleotides that are linked to a 2′-deoxy-2′-fluoro modified nucleotide on its 5′-end and/or 3′-end. In some embodiments, one of sense strand and antisense strand has at least four, at least five nucleotides that have alternating 2′-O-methyl modified nucleotide and 2′-deoxy-2′-fluoro modified nucleotide.

In some embodiments, the oligonucleotide molecule, such as an siRNA, has the formula as illustrated in Formula I:

N₁N₂N₃N₄N₅NGN₇N₈N₉N₁₀N₁₁N₁₂N₁₃N₁₄
N₁₅N₁₆₁₇N₁₈N₁₉
Sense-strand (SS)
N₂₁N₂₀N₁₉N₁₈N₁₇N₁₆N₁₅N₁₄N₁₃N₁₂N₁N₁₀
N₉N₈N₇N₆GN₅N₄N₃N₂N₁
Antisense-strand (AS),

wherein each nucleotide represented by N, is independently, A, U, C, or G or a modified nucleotide base, such as those provided for herein. The N₁ nucleotides of the sense strand and the antisense strand represent the 5′ end of the respective strands. For clarity, although Formula I utilizes N₁, N₂, N₃, etc. in both the sense and the antisense strand, the nucleotide bases do not need to be the same and are not intended to be the same. The siRNA that is illustrated in Formula I would be complementary to a target sequence.

For example, in some embodiments, the sense strand comprises a 2′O-methyl modified nucleotide with a phosphorothioate (PS) modified backbone at N₁ and N₂, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, N₁₈, and N₁₉.

In some embodiments, the antisense strand comprises a vinylphosphonate moiety attached to N₁, a 2′fluoro-modified nucleotide with a phosphorothioate (PS) modified backbone at N₂, a 2′O-methyl modified nucleotide at N₃, N₄, N₅, N₆, N₇, N₈, N₉, N₁₀, N₁₁, N₁₂, N₁₃, N₁₅, N₁₆, N₁₇, N₁₈, and N₁₉, a 2′fluoro-modified nucleotide at N₁₄, and a 2′O-methyl modified nucleotide with a phosphorothioate (PS) modified backbone at N₂₀ and N₂₁.

In some embodiments, an oligonucleotide molecule comprises a sense strand and antisense strand, wherein the sense strand includes a terminal cap moiety at the 5′-end, the 3′-end, or both of the 5′ and 3′ ends of the sense strand. In other embodiments, the terminal cap moiety is an inverted deoxy abasic moiety.

In some embodiments, an oligonucleotide molecule comprises a sense strand and an antisense strand, wherein the antisense strand comprises a glyceryl modification at the 3′ end of the antisense strand.

In some embodiments, an oligonucleotide molecule comprises a sense strand and an antisense strand, in which the sense strand comprises one or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and in which the antisense strand comprises about 1 to about 10 or more, specifically about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate, phosphorodithioate, phosphonate, phosphoramidate, or mesyl phosphoramidate internucleotide linkages and/or a terminal cap molecule at the 3′-end, the 5′-end, or both of the 3′- and 5′-ends, being present in the same or different strand.

In some embodiments, an oligonucleotide molecule comprises a sense strand and an antisense strand, in which the sense strand comprises about 1 to about 25, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10, or more) 2′-deoxy, 2′-O-methyl, 2′-deoxy-2′-fluoro, and/or one or more (e.g., about 1, 2, 3, 4, 5, 6, 7, 8, 9, 10, or more) universal base modified nucleotides, and optionally a terminal cap molecule at the 3-end, the 5′-end, or both of the 3′- and 5′-ends of the sense strand; and in which the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages and/or a terminal cap molecule at the 3′-end, the 5′-end, or both of the 3′- and 5′-ends, being present in the same or different strand.

In some embodiments, an oligonucleotide molecule comprises a sense strand and an antisense strand, in which the antisense strand comprises one or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or about one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides at the 3′-end, the 5′-end, or both of the 3′- and 5′-ends of the sense strand and/or antisense strand, and optionally a terminal cap molecule at the 3′-end, the 5′-end, or both of the 3′- and 5′-ends of the sense strand. In some embodiments, the antisense strand comprises about 1 to about 10 or more, specifically about 1, 2, 3, 4, 5, 6, 7, 8, 9, 10 or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, 11, 12, 13, 14, 15, 16, 17, 18, 19, 20, or more pyrimidine nucleotides of the sense and/or antisense strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate, phosphorodithioate, phosphonate, phosphoramidate, or mesyl phosphoramidate internucleotide linkages and/or a terminal cap molecule at the 3′-end, the 5′-end, or both of the 3′ and 5′-ends, being present in the same or different strand.

In some embodiments, an oligonucleotide molecule comprises a sense strand and an antisense strand, in which the antisense strand comprises about 1 to about 25 or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10 or more pyrimidine nucleotides of the sense and/or antisense strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate, phosphorodithioate, phosphonate, phosphoramidate, or mesyl phosphoramidate internucleotide linkages and/or a terminal cap molecule at the 3′-end, the 5′-end, or both of the 3′- and 5′-ends, being present in the same or different strand.

In some embodiments, an oligonucleotide molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate, phosphorodithioate, phosphonate, phosphoramidate, or mesyl phosphoramidate internucleotide linkages in each strand of the oligonucleotide molecule. In some embodiments, an oligonucleotide molecule comprises a sense strand and an antisense strand, and the antisense strand comprises a phosphate backbone modification at the 3′ end of the antisense strand. Alternatively and/or additionally, an oligonucleotide molecule comprises a sense strand and an antisense strand, and the sense strand comprises a phosphate backbone modification at the 5′ end of the antisense strand. In some instances, the phosphate backbone modification is a phosphorothioate. In some instances, the phosphate backbone modification is a phosphorodithioate. In some instances, the phosphate backbone modification is a phosphonate. In some instances, the phosphate backbone modification is a phosphoramidate. In some instances, the phosphate backbone modification is a mesyl phosphoramidate. In some embodiments, the sense or antisense strand has three consecutive nucleosides that are coupled via two phosphorothioate backbone. In some embodiments, the sense or antisense strand has three consecutive nucleosides that are coupled via two phosphorodithioate backbone. In some embodiments, the sense or antisense strand has three consecutive nucleosides that are coupled via two phosphonate backbone. In some embodiments, the sense or antisense strand has three consecutive nucleosides that are coupled via two phosphoramidate backbone. In some embodiments, the sense or antisense strand has three consecutive nucleosides that are coupled via two mesyl phosphoramidate backbone.

In another embodiment, an oligonucleotide molecule described herein comprises 2′-5′ internucleotide linkages. In some instances, the 2′-5′ internucleotide linkage(s) is at the 3′-end, the 5′-end, or both of the 3′- and 5′-ends of one or both sequence strands. In addition instances, the 2′-5′ internucleotide linkage(s) is present at various other positions within one or both sequence strands, for example, about 1, 2, 3, 4, 5, 6, 7, 8, 9, 10, or more including every internucleotide linkage of a pyrimidine nucleotide in one or both strands of the oligonucleotide molecule comprise a 2′-5′ internucleotide linkage, or about 1, 2, 3, 4, 5, 6, 7, 8, 9, 10, or more including every internucleotide linkage of a purine nucleotide in one or both strands of the oligonucleotide molecule comprise a 2′-5′ internucleotide linkage.

In some embodiments, an oligonucleotide molecule is a single stranded molecule that mediates RNAi activity in a cell or reconstituted in vitro system, wherein the oligonucleotide molecule comprises a single stranded polynucleotide having complementarity to a target nucleic acid sequence, and wherein one or more pyrimidine nucleotides present in the oligonucleotide molecule are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the oligonucleotide molecule are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the oligonucleotide molecule optionally further comprising about 1 to about 4 (e.g., about 1, 2, 3, or 4) terminal 2′-deoxynucleotides at the 3′-end of the oligonucleotide molecule, wherein the terminal nucleotides further comprise one or more (e.g., 1, 2, 3, or 4) phosphorothioate or mesyl phosphoramidate internucleotide linkages, and wherein the oligonucleotide molecule optionally further comprises a terminal phosphate group, such as a 5′-terminal phosphate group.

In some cases, one or more of the synthetic nucleotide analogues described herein are resistant toward nucleases such as, for example, ribonuclease such as RNase H, deoxyribonuclease such as DNase, or exonuclease such as 5′-3′ exonuclease and 3′-5′ exonuclease when compared to natural polynucleic acid molecules and endonucleases. In some instances, synthetic nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, 2′-O-methyl modified oligonucleotide molecule is nuclease resistance (e.g., RNase H, DNase, 5′-3′ exonuclease or 3′-5′ exonuclease resistance). In some instances, 2′O-methoxyethyl (2′-O-MOE) modified oligonucleotide molecule is nuclease resistance (e.g., RNase H, DNase, 5′-3′ exonuclease or 3′-5′ exonuclease resistance). In some instances, 2′-O-aminopropyl modified oligonucleotide molecule is nuclease resistance (e.g., RNase H, DNase, 5′-3′ exonuclease or 3′-5′ exonuclease resistance). In some instances, 2′-deoxy modified oligonucleotide molecule is nuclease resistance (e.g., RNase H, DNase, 5′-3′ exonuclease or 3′-5′ exonuclease resistance). In some instances, 2′-deoxy-2′-fluoro modified oligonucleotide molecule is nuclease resistance (e.g., RNase H, DNase, 5′-3′ exonuclease or 3′-5′ exonuclease resistance). In some instances, 2′-O-aminopropyl (2′-O-AP) modified oligonucleotide molecule is nuclease resistance (e.g., RNase H, DNase, 5′-3′ exonuclease or 3′-5′ exonuclease resistance). In some instances, 2′-O-dimethylaminoethyl (2′-O-DMAOE) modified oligonucleotide molecule is nuclease resistance (e.g., RNase H, DNase, 5′-3′ exonuclease or 3′-5′ exonuclease resistance). In some instances, 2′-O-dimethylaminopropyl (2′-O-DMAP) modified oligonucleotide molecule is nuclease resistance (e.g., RNase H, DNase, 5′-3′ exonuclease or 3′-5′ exonuclease resistance). In some instances, 2′-O-dimethylaminoethyloxyethyl (2′-O-DMAEOE) modified oligonucleotide molecule is nuclease resistance (e.g., RNase H, DNase, 5′-3′ exonuclease or 3′-5′ exonuclease resistance). In some instances, 2′-O—N-methylacetamido (2′-O-NMA) modified oligonucleotide molecule is nuclease resistance (e.g., RNase H, DNase, 5′-3′ exonuclease or 3′-5′ exonuclease resistance). In some instances, LNA modified oligonucleotide molecule is nuclease resistance (e.g., RNase H, DNase, 5′-3′ exonuclease or 3′-5′ exonuclease resistance). In some instances, ENA modified oligonucleotide molecule is nuclease resistance (e.g., RNase H, DNase, 5′-3′ exonuclease or 3′-5′ exonuclease resistance). In some instances, HNA modified oligonucleotide molecule is nuclease resistance (e.g., RNase H, DNase, 5′-3′ exonuclease or 3′-5′ exonuclease resistance). In some instances, morpholinos is nuclease resistance (e.g., RNase H, DNase, 5′-3′ exonuclease or 3′-5′ exonuclease resistance). In some instances, PNA modified oligonucleotide molecule is resistant to nucleases (e.g., RNase H, DNase, 5′-3′ exonuclease or 3′-5′ exonuclease resistance). In some instances, methylphosphonate nucleotides modified oligonucleotide molecule is nuclease resistance (e.g., RNase H, DNase, 5′-3′ exonuclease or 3′-5′ exonuclease resistance). In some instances, thiolphosphonate nucleotides modified oligonucleotide molecule is nuclease resistance (e.g., RNase H, DNase, 5′-3′ exonuclease or 3′-5′ exonuclease resistance). In some instances, oligonucleotide molecule comprising 2′-fluoro N3-P5′-phosphoramidites is nuclease resistance (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

In some embodiments, one or more of the synthetic nucleotide analogues described herein have increased binding affinity toward their mRNA target relative to an equivalent natural polynucleic acid molecule. The one or more of the synthetic nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, 2′-O-methyl modified oligonucleotide molecule has increased binding affinity toward their mRNA target relative to an equivalent natural polynucleic acid molecule. In some instances, 2′-O-methoxyethyl (2′-O-MOE) modified oligonucleotide molecule has increased binding affinity toward their mRNA target relative to an equivalent natural polynucleic acid molecule. In some instances, 2′-O-aminopropyl modified oligonucleotide molecule has increased binding affinity toward their mRNA target relative to an equivalent natural polynucleic acid molecule. In some instances, 2′-deoxy modified oligonucleotide molecule has increased binding affinity toward their mRNA target relative to an equivalent natural polynucleic acid molecule. In some instances, 2′-deoxy-2′-fluoro modified oligonucleotide molecule has increased binding affinity toward their mRNA target relative to an equivalent natural polynucleic acid molecule. In some instances, 2′-O-aminopropyl (2′-O-AP) modified oligonucleotide molecule has increased binding affinity toward their mRNA target relative to an equivalent natural polynucleic acid molecule. In some instances, 2′-O-dimethylaminoethyl (2′-O-DMAOE) modified oligonucleotide molecule has increased binding affinity toward their mRNA target relative to an equivalent natural polynucleic acid molecule. In some instances, 2′-O-dimethylaminopropyl (2′-O-DMAP) modified oligonucleotide molecule has increased binding affinity toward their mRNA target relative to an equivalent natural polynucleic acid molecule. In some instances, 2′-O-dimethylaminoethyloxyethyl (2′-O-DMAEOE) modified oligonucleotide molecule has increased binding affinity toward their mRNA target relative to an equivalent natural polynucleic acid molecule. In some instances, 2′-O—N-methylacetamido (2′-O-NMA) modified oligonucleotide molecule has increased binding affinity toward their mRNA target relative to an equivalent natural polynucleic acid molecule. In some instances, LNA modified oligonucleotide molecule has increased binding affinity toward its mRNA target relative to an equivalent natural polynucleic acid molecule. In some instances, ENA modified oligonucleotide molecule has increased binding affinity toward its mRNA target relative to an equivalent natural polynucleic acid molecule. In some instances, PNA modified oligonucleotide molecule has increased binding affinity toward its mRNA target relative to an equivalent natural polynucleic acid molecule. In some instances, HNA modified oligonucleotide molecule has increased binding affinity toward its mRNA target relative to an equivalent natural polynucleic acid molecule. In some instances, morpholino modified oligonucleotide molecule has increased binding affinity toward its mRNA target relative to an equivalent natural polynucleic acid molecule. In some instances, methylphosphonate nucleotides modified oligonucleotide molecule has increased binding affinity toward its mRNA target relative to an equivalent natural polynucleic acid molecule. In some instances, thiolphosphonate nucleotides modified oligonucleotide molecule has increased binding affinity toward its mRNA target relative to an equivalent natural polynucleic acid molecule. In some instances, oligonucleotide molecule comprising 2′-fluoro N3-P5′-phosphoramidites has increased binding affinity toward its mRNA target relative to an equivalent natural polynucleic acid molecule. In some cases, the increased affinity is illustrated with a lower Kd, a higher melt temperature (Tm), or a combination thereof.

In some embodiments, an oligonucleotide molecule described herein is a chirally pure (or stereo pure) polynucleic acid molecule, or a polynucleic acid molecule comprising a single enantiomer. In some instances, the oligonucleotide molecule comprises L-nucleotide. In some instances, the oligonucleotide molecule comprises D-nucleotides. In some instance, an oligonucleotide molecule composition comprises less than 30%, 25%, 20%, 15%, 10%, 5%, 4%, 3%, 2%, 1%, or less of its mirror enantiomer. In some cases, an oligonucleotide molecule composition comprises less than 30%, 25%, 20%, 15%, 10%, 5%, 4%, 3%, 2%, 1%, or less of a racemic mixture.

In some embodiments, an oligonucleotide molecule described herein is further modified to include an aptamer conjugating moiety. In some instances, the aptamer conjugating moiety is a DNA aptamer conjugating moiety. In some instances, the aptamer conjugating moiety is Alphamer, which comprises an aptamer portion that recognizes a specific cell-surface target and a portion that presents a specific epitopes for attaching to circulating antibodies.

In additional embodiments, an oligonucleotide molecule described herein is modified to increase its stability. In some embodiment, the oligonucleotide molecule is RNA (e.g., siRNA). In some instances, the oligonucleotide molecule is modified by one or more of the modifications described above to increase its stability. In some cases, the oligonucleotide molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the oligonucleotide molecule is modified by 2′-O-methyl and/or 2′-O-methoxyethyl ribose. In some cases, the oligonucleotide molecule also includes morpholinos, PNAs, HNA, methylphosphonate nucleotides, thiolphosphonate nucleotides, and/or 2′-fluoro N3-P5′-phosphoramidites to increase its stability. In some instances, the oligonucleotide molecule is a chirally pure (or stereo pure) oligonucleotide molecule. In some instances, the chirally pure (orstereo pure) oligonucleotide molecule is modified to increase its stability. Suitable modifications to the RNA to increase stability for delivery will be apparent to the skilled person.

In some embodiments, the oligonucleotide molecule comprises 2′ modifications. In some embodiments, the nucleotides of the oligonucleotide molecule at positions 3, 7, 8, 9, 12, and 17 from the 5′ end of the sense strand are not modified with a 2′O-methyl modification. In some embodiments, the nucleotides of the oligonucleotide molecule at positions 3, 7, 8, 9, 12, and 17 from the 5′ end of the sense strand are modified with a 2′fluoro modification. In some embodiments, the nucleotides of the oligonucleotide molecule at positions 2 and 14 from the 5′ end of the anti-sense strand are not modified with a 2′O-methyl modification. In some embodiments, the nucleotides of the oligonucleotide molecule at positions 2 and 14 from the 5′ end of the anti-sense strand are modified with a 2′fluoro modification. In some embodiments, any of the nucleotides may further comprise a 5′-phosphorothioate group modification. In some embodiments, the nucleotides of the oligonucleotide molecule at positions 1 and 2 from the 5′ end of the sense strand are modified with a 5′-phosphorothioate group modification. In some embodiments, the nucleotides of the oligonucleotide molecule at positions 1, 2, 20, and 21 from the 5′ end of the antisense strand are modified with a 5′-phosphorothioate group modification. In some embodiments, the 5′ end of the sense or antisense strand of the oligonucleotide molecule may further comprise a vinylphosphonate modification. In some embodiments, the nucleotide of the oligonucleotide molecule at position 1 from the 5′ end of the antisense strand is modified with a vinylphosphonate modification.

In some instances, the oligonucleotide molecule is a double stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. In some instances, the oligonucleotide molecule is assembled from two separate polynucleotides, where one strand is the sense strand and the other is the antisense strand, wherein the antisense and sense strands are self-complementary (e.g., each strand comprises nucleotide sequence that is complementary to nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the oligonucleotide molecule is assembled from a single oligonucleotide, where the self-complementary sense and antisense regions of the oligonucleotide molecule are linked by means of a nucleic acid based or non-nucleic acid-based linker(s).

In some cases, the oligonucleotide molecule is a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. In other cases, the oligonucleotide molecule is a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide is processed either in vivo or in vitro to generate an active oligonucleotide molecule capable of mediating RNAi. In additional cases, the oligonucleotide molecule also comprises a single stranded polynucleotide having nucleotide sequence complementary to nucleotide sequence in a target nucleic acid molecule or a portion thereof (for example, where such oligonucleotide molecule does not require the presence within the oligonucleotide molecule of nucleotide sequence corresponding to the target nucleic acid sequence or a portion thereof), wherein the single stranded polynucleotide further comprises a terminal phosphate group, such as a 5′-phosphate, or 5′,3′-diphosphate.

In some instances, an asymmetric hairpin is a linear oligonucleotide molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin oligonucleotide molecule comprises an antisense region having length sufficient to mediate RNAi in a cell or in vitro system (e.g., about 19 to about 22 nucleotides) and a loop region comprising about 4 to about 8 nucleotides, and a sense region having about 3 to about 18 nucleotides that are complementary to the antisense region. In some cases, the asymmetric hairpin oligonucleotide molecule also comprises a 5′-terminal phosphate group that is chemically modified. In additional cases, the loop portion of the asymmetric hairpin oligonucleotide molecule comprises nucleotides, non-nucleotides, linker molecules, or conjugate molecules.

In some embodiments, an asymmetric duplex is an oligonucleotide molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex oligonucleotide molecule comprises an antisense region having length sufficient to mediate RNAi in a cell or in vitro system (e.g., about 19 to about 22 nucleotides) and a sense region having about 3 to about 19 nucleotides that are complementary to the antisense region.

In some cases, a universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art.

In some embodiments, the dsRNA agents are 5′ phosphorylated or include a phosphoryl analog at the 5′ prime terminus. 5′-phosphate modifications include those which are compatible with RISC mediated gene silencing. Suitable modifications include: 5′-monophosphate ((HO₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)2(O)P—S-5′); phosphorodithioate [—O₂PS₂—]; phosphonate [—PO(OH)₂—]; phosphoramidate [—O═P(OH)₂—]; mesyl phosphoramidate (CH₃)(SO₂)(N)P(O)₂—O-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH2-), 5′-alkyletherphosphonates (R=alkylether=methoxymethyl (MeOCH2-), ethoxymethyl, etc., e.g. RP(OH)(O)—O-5′-). In some embodiments, the modification can in placed in the antisense strand of a dsRNA agent.

In some embodiments, the sequence of the oligonucleotide molecule is at least 40%, 50%, 55%, 60%, 65%, 70%, 75%, 80%, 85%, 90%, 95%, 98%, 99%, or 99.5% complementary to a target sequence of GYS1. In some embodiments, the target sequence of GYS1 is a nucleic acid sequence of about 10-50 base pair length, about 15-50 base pair length, 15-40 base pair length, 15-30 base pair length, or 15-25 base pair length sequences in GYS1, in which the first nucleotide of the target sequence starts at any nucleotide in GYS1 mRNA transcript in the coding region, or in the 5′ or 3′-untranslated region (UTR). For example, the first nucleotide of the target sequence can be selected so that it starts at the nucleic acid location (nal, number starting from the 5′-end of the full length of GYS1 mRNA, e.g., the 5′-end first nucleotide is nal.1) 1, nal 2, nal 3, nal 4, nal 5, nal 6, nal 7, nal 8, nal 9, nal 10, nal 11, nal 12, nal 13, nal 14, nal 15, nal 15, nal 16, nal 17, or any other nucleic acid location in the coding or noncoding regions (5′ or 3′-untranslated region) of GYS1 mRNA. In some embodiments, the first nucleotide of the target sequence can be selected so that it starts at a location within, or between, nal 10-nal 15, nal 10-nal 20, nal 50-nal 60, nal 55-nal 65, nal 75-nal 85, nal 95-nal 105, nal 135-nal 145, nal 155-nal 165, nal 225-nal 235, nal 265-nal 275, nal 275-nal 245, nal 245-nal 255, nal 285-nal 335, nal 335-nal 345, nal 385-nal 395, nal 515-nal 525, nal 665-nal 675, nal 675-nal 685, nal 695-nal 705, nal 705-nal 715, nal 875-nal 885, nal 885-nal 895, nal 895-nal 905, nal 1035-nal 1045, nal 1045-nal 1055, nal 1125-nal 1135, nal 1135-nal 1145, nal 1145-nal 1155, nal 1155-nal 1165, nal 1125-nal 1135, nal 1155-nal 1165, nal 1225-nal 1235, nal 1235-nal 1245, nal 1275-nal 1245, nal 1245-nal 1255, nal 1265-nal 1275, nal 1125-nal 1135, nal 1155-nal 1165, nal 1225-nal 1235, nal 1235-nal 1245, nal 1275-nal 1245, nal 1245-nal 1255, nal 1265-nal 1275, nal 1275-nal 1285, nal 1335-nal 1345, nal 1345-nal 1355, nal 1525-nal 1535, nal 1535-nal 1545, nal 1605-nal 1615, nal 1615-c.1625, nal 1625-nal 1635, nal 1635-1735, nal 1735-1835, nal 1835-1935, nal. 1836-1856, nal 1935-2000, nal 2000-2100, nal 2100-2200, nal 2200-2260, nal 2260-2400, nal 2400-2500, nal 2500-2600, nal 2600-2700, nal 2700-2800, nal 2800-2500, nal 2500-2600, nal 2600-2700, nal 2700-2800, nal 2800-2860, etc. In some embodiments, the sequence of GYS1 mRNA is provided as NCBI Reference Sequence: NM_002103.

In some embodiments, the antisense strand of the dsRNA agent is 100% complementary to a target RNA to hybridize thereto and inhibits its expression through RNA interference. The target RNA can be any RNA expressed in a cell. In another embodiment, the cell is a tumor cell, a liver cell, a muscle cell, an immune cell, a dendritic cell, a heart cell, or a cell of the central nervous system. In another embodiment, the antisense strand of the dsRNA agent is at least 99%, at least 98%, at least 97%, at least 96%, 95%, at least 90%, at least 85%, at least 80%, at least 75%, at least 70%, at least 65%, at least 60%, at least 55%, or at least 50% complementary to a target RNA. In some embodiments, the target RNA is GYS1 RNA. In some embodiments, the siRNA molecule is a siRNA that reduces the expression of GYS1. In some embodiments, the siRNA molecule is a siRNA that reduces the expression of GYS and does not reduce the expression of other RNAs by more than 50% in an assay described herein at a concentration of no more than 200 nm as described herein.

The siRNA can be targeted against any gene or RNA (e.g. mRNA) transcript of interest.

Other modifications and patterns of modifications can be found in, for example, U.S. Pat. No. 10,233,448, which is hereby incorporated by reference.

Other modifications and patterns of modifications can be found in, for example, Anderson et al. (Nucleic Acids Research, 2021, 49 (16), 9026-9041), which is hereby incorporated by reference.

Other modifications and patterns of modifications can be found in, for example, International Patent Application Publication No. WO2021/030778, which is hereby incorporated by reference.

Other modifications and patterns of modifications can be found in, for example, International Patent Application Publication No. WO2021/030763, which is hereby incorporated by reference.

In some embodiments, the siRNA is linked to a protein, such as a FN3 domain. The siRNA can be linked to multiple FN3 domains that bind to the same target protein or different target proteins. In some embodiments, the linker is attached to the sense strand, which is used to facilitate the linkage of the sense strand to the FN3 domain.

In some embodiments, compositions are provided herein having a formula of (X1)ₙ-(X2)_{q}-(X3)_{y}-L-X4, wherein X1 is a first FN3 domain, X2 is a second FN3 domain, X3 is a third FN3 domain or half-life extender molecule, L is a linker, and X4 is a nucleic acid molecule, such as, but not limited to a siRNA molecule, wherein n, q, and y are each independently 0 or 1. In some embodiments, X1, X2, and X3 bind to different target proteins. In some embodiments, y is 0. In some embodiments, n is 1, q is 0, and y is 0. In some embodiments, n is 1, q is 1, and y is 0. In some embodiments, n is 1, q is 1, and y is 1. In some embodiments, the third FN3 domain increases the half-life of the molecule as a whole as compared to a molecule without X3. In some embodiments, the half-life extending moiety is a FN3 domain that binds to albumin. Examples of such FN3 domains include, but are not limited to, those described in U.S. Patent Application Publication No. 20170348397 and U.S. Pat. No. 9,156,887, which is hereby incorporated by reference in its entirety. The FN3 domains may incorporate other subunits for example via covalent interaction. In some embodiments, the FN3 domains further comprise a half-life extending moiety. Exemplary half-life extending moieties are albumin, albumin variants, albumin-binding proteins and/or domains, transferrin and fragments and analogues thereof, and Fe regions. Amino acid sequences of the human Fe regions are well known, and include IgG1, IgG2, IgG3, IgG4, IgM, IgA and IgE Fc regions. In some embodiments, the FN3 domains may incorporate a second FN3 domain that binds to a molecule that extends the half-life of the entire molecule, such as, but not limited to, any of the half-life extending moieties described herein. In some embodiments, the second FN3 domain binds to albumin, albumin variants, albumin-binding proteins and/or domains, and fragments and analogues thereof.

In some embodiments, compositions are provided herein having a formula of (X1)-(X2)-L-(X4), wherein X1 is a first FN3 domain, X2 is a second FN3 domain, L is a linker, and X4 is a nucleic acid molecule. In some embodiments, X4 is a siRNA molecule. In some embodiments, X1 is a FN3 domain that binds to one of CD71. In some embodiments, X2 is a FN3 domain that binds to one of CD71. In some embodiments X1 and X2 do not bind to the same target protein. In some embodiments, X1 and X2 bind to the same target protein, but at different binding sites on the protein. In some embodiments, X1 and X2 bind to the same target protein. In some embodiments, X1 and X2 are FN3 domains that bind to CD71. In some embodiments, the composition does not comprise (e.g. is free of) a compound or protein that binds to ASGPR.

In some embodiments, compositions are provided herein having a formula of C—(X1)ₙ-(X2)_{q}[L-X4]-(X3)_{y}, wherein X1 is a first FN3 domain; X2 is a second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions are provided herein having a formula of (X1)ₙ-(X2)_{q}[L-X4]-(X3)_{y}-C, wherein X1 is a first FN3 domain; X2 is a second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions are provided herein having a formula of C—(X1)ₙ-(X2)_{q}[L-X4]L-(X3)_{y}, wherein X1 is a first FN3 domain; X2 is a second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions are provided herein having a formula of (X1)ₙ-(X2)_{q}[L-X4]L-(X3)_{y}-C, wherein X1 is a first FN3 domain; X2 is a second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions or complexes are provided having a formula of A₁-B₁, wherein A₁ has a formula of C-L₁-Xₛ and B₁ has a formula of X_{AS}-L₂-F₁, wherein:
C is a polymer, such as PEG;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
wherein X_{S} and X_{AS} form a double stranded oligonucleotide molecule to form the composition/complex.

In some embodiments, C can be a molecule that extends the half-life of the molecule. Examples of such moieties are described herein. In some embodiments, C can also be Endoporter, INF-7, TAT, polyarginine, polylysine, or an amphipathic peptide. These moieties can be used in place of or in addition to other half-life extending moieties provided for herein. In some embodiments, C can be a molecule that delivers the complex into the cell, the endosome, or the ER; said molecules are selected from those peptides listed in Table 2, below.

TABLE 2

| SEQ | | |
|---|---|---|
| ID | | |
| NO: | Name | Sequence |
| 1043 | TAT | RKKRRQRRR |
| 1044 | Penetratin | RQIKIWFQNRRMKWKK |
| 1045 | Transportan | GWTLNSAGYLLGKINKALAALAKKIL |
| 1046 | MAP | KLALKLALKALKAALKLA |
| 1047 | Pep-1 | KETWWETWWTEWSQPKKKRKV |
| 1048 | KDEL | KDEL |
| 1049 | GALA | WEAALAEALAELAEHLAEALAEAL |
| | | EALAA |
| 1050 | HA2 | GDIMGEWGNEIFGAIAGFLGC |
| 1051 | Aurine 1.2 | GLFDIIKKIAESF |
| 1052 | MPG | GALFLGWLGAAGSTMGAPKSKRKV |
| 1053 | TP-10 | AGYLLGKINLKALAALAKKIL |
| 1054 | EB-1 | LIRLWSHLIHIWFQNRRLKWKKK |
| 1055 | HA2-Penetratin | GLFGAIAGFIENGWEGMIDGRQIK |
| | | IWFQNRRMKWKK |
| 1056 | Endosomolytic | FFKKLAHALHLLALLALHLAHALKKA |
| 1057 | Endosomolytic | LFEAIEGFIENGWGMIDGWYG |
| 1058 | Endosomolytic | LFEAIEGFIENGWEGMIDGWYGRKK |
| | | RRQRRR |
| 1059 | Endosomolytic | IGAVLKVLTTGLPALISWIKRKRQQ |
| 1060 | ER Targeting | MKLAVTLTLVTLALSSSSASA |
| 1061 | ER Targeting | RLIEDICLPRWGCLWEDDKDEL |
| 1062 | ER Targeting | MIRTLLLSTLVAGALSK |
| 1063 | ER Targeting | ILSSLTVTQLLRRLHQWIK |
| 1064 | ER Targeting | MIRTLLLSTLVAGALSKDEL |

In some embodiments, compositions or complexes are provided having a formula of A₁-B₁, wherein A₁ has a formula of Xₛ and B₁ has a formula of X_{AS}-L₂-F₁.

In some embodiments, compositions or complexes are provided having a formula of A₁-B₁, wherein A1 has a formula of C-L₁-Xₛ and B₁ has a formula of X_{AS}.

In some embodiments, the sense strand is a sense strand as provided for herein.

In some embodiments, the antisense strand is an antisense strand as provided for herein.

In some embodiments, the sense and antisense strand form a double stranded siRNA molecule that targets GYS1. In some embodiments, the double stranded oligonucleotide is about 21-23 nucleotides base pairs in length. In certain embodiments, C is optional.

In some embodiments, compositions or complexes are provided having a formula of A₁-B₁, wherein A₁ has a formula of F₁-L₁-Xₛ and B₁ has a formula of X_{AS}-L₂-C, wherein:
F₁ is a polypeptide comprising at least one FN3 domain;
L₁ and L₂ are each, independently, a linker;
C is a polymer, such as PEG;
X_{S} is a 5′ to 3′ oligonucleotide sense strand of a double stranded siRNA molecule;
X_{AS} is a 3′ to 5′ oligonucleotide antisense strand of a double stranded siRNA molecule;
   wherein X_{S} and X_{AS} form a double stranded oligonucleotide molecule to form the composition/complex. In certain embodiments, C is optional.

In some embodiments, compositions or complexes are provided having a formula of A₁-B₁, wherein A₁ has a formula of Xₛ and B₁ has a formula of X_{AS}-L₂-C.

In some embodiments, compositions or complexes are provided having a formula of A₁-B₁, wherein A₁ has a formula of F₁-L₁-Xₛ and B₁ has a formula of X_{AS}.

In some embodiments, C is a natural or synthetic polymer, consisting of long chains of branched or unbranched monomers, and/or cross-linked network of monomers in two or three dimensions. In some instances, the polymer includes a polysaccharide, lignin, rubber, or polyalkylen oxide, which can be for example, polyethylene glycol. In some instances, the at least one polymer includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene-B-Bterephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer comprises polyalkylene oxide. In some instances, the polymer comprises PEG. In some instances, the polymer comprises polyethylene imide (PEI) or hydroxy ethyl starch (HES).

In some embodiments, the polyalkylene oxide (e.g., PEG) is a polydisperse or monodisperse compound. In some instances, polydisperse material comprises disperse distribution of different molecular weight of the material, characterized by mean weight (weight average) size and dispersity. In some instances, the monodisperse PEG comprises one size of molecules. In some embodiments, C is poly- or monodispersed polyalkylene oxide (e.g., PEG) and the indicated molecular weight represents an average of the molecular weight of the polyalkylene oxide, e.g., PEG, molecules.

In some embodiments, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, C is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some instances, the molecular weight of C is about 200 Da. In some instances, the molecular weight of C is about 300 Da. In some instances, the molecular weight of C is about 400 Da. In some instances, the molecular weight of C is about 500 Da. In some instances, the molecular weight of C is about 600 Da. In some instances, the molecular weight of C is about 700 Da. In some instances, the molecular weight of C is about 800 Da. In some instances, the molecular weight of C is about 900 Da. In some instances, the molecular weight of C is about 1000 Da. In some instances, the molecular weight of C is about 1100 Da. In some instances, the molecular weight of C is about 1200 Da. In some instances, the molecular weight of C is about 1300 Da. In some instances, the molecular weight of C is about 1400 Da. In some instances, the molecular weight of C is about 1450 Da. In some instances, the molecular weight of C is about 1500 Da. In some instances, the molecular weight of C is about 1600 Da. In some instances, the molecular weight of C is about 1700 Da. In some instances, the molecular weight of C is about 1800 Da. In some instances, the molecular weight of C is about 1900 Da. In some instances, the molecular weight of C is about 2000 Da. In some instances, the molecular weight of C is about 2100 Da. In some instances, the molecular weight of C is about 2200 Da. In some instances, the molecular weight of C is about 2300 Da. In some instances, the molecular weight of C is about 2400 Da. In some instances, the molecular weight of C is about 2500 Da. In some instances, the molecular weight of C is about 2600 Da. In some instances, the molecular weight of C is about 2700 Da. In some instances, the molecular weight of C is about 2800 Da. In some instances, the molecular weight of C is about 2900 Da. In some instances, the molecular weight of C is about 3000 Da. In some instances, the molecular weight of C is about 3250 Da. In some instances, the molecular weight of C is about 3350 Da. In some instances, the molecular weight of C is about 3500 Da. In some instances, the molecular weight of C is about 3750 Da. In some instances, the molecular weight of C is about 4000 Da. In some instances, the molecular weight of C is about 4250 Da. In some instances, the molecular weight of C is about 4500 Da. In some instances, the molecular weight of C is about 4600 Da. In some instances, the molecular weight of C is about 4750 Da. In some instances, the molecular weight of C is about 5000 Da. In some instances, the molecular weight of C is about 5500 Da. In some instances, the molecular weight of C is about 6000 Da. In some instances, the molecular weight of C is about 6500 Da. In some instances, the molecular weight of C is about 7000 Da. In some instances, the molecular weight of C is about 7500 Da. In some instances, the molecular weight of C is about 8000 Da. In some instances, the molecular weight of C is about 10,000 Da. In some instances, the molecular weight of C is about 12,000 Da. In some instances, the molecular weight of C is about 20,000 Da. In some instances, the molecular weight of C is about 35,000 Da. In some instances, the molecular weight of C is about 40,000 Da. In some instances, the molecular weight of C is about 50,000 Da. In some instances, the molecular weight of C is about 60,000 Da. In some instances, the molecular weight of C is about 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some instances, a dPEG comprises about 3 or more repeating ethylene oxide units. In some instances, a dPEG comprises about 4 or more repeating ethylene oxide units. In some instances, a dPEG comprises about 5 or more repeating ethylene oxide units. In some instances, a dPEG comprises about 6 or more repeating ethylene oxide units. In some instances, a dPEG comprises about 7 or more repeating ethylene oxide units. In some instances, a dPEG comprises about 8 or more repeating ethylene oxide units. In some instances, a dPEG comprises about 9 or more repeating ethylene oxide units. In some instances, a dPEG comprises about 10 or more repeating ethylene oxide units. In some instances, a dPEG comprises about 11 or more repeating ethylene oxide units. In some instances, a dPEG comprises about 12 or more repeating ethylene oxide units. In some instances, a dPEG comprises about 13 or more repeating ethylene oxide units. In some instances, a dPEG comprises about 14 or more repeating ethylene oxide units. In some instances, a dPEG comprises about 15 or more repeating ethylene oxide units. In some instances, a dPEG comprises about 16 or more repeating ethylene oxide units. In some instances, a dPEG comprises about 17 or more repeating ethylene oxide units. In some instances, a dPEG comprises about 18 or more repeating ethylene oxide units. In some instances, a dPEG comprises about 19 or more repeating ethylene oxide units. In some instances, a dPEG comprises about 20 or more repeating ethylene oxide units. In some instances, a dPEG comprises about 22 or more repeating ethylene oxide units. In some instances, a dPEG comprises about 24 or more repeating ethylene oxide units. In some instances, a dPEG comprises about 26 or more repeating ethylene oxide units. In some instances, a dPEG comprises about 28 or more repeating ethylene oxide units. In some instances, a dPEG comprises about 30 or more repeating ethylene oxide units. In some instances, a dPEG comprises about 35 or more repeating ethylene oxide units. In some instances, a dPEG comprises about 40 or more repeating ethylene oxide units. In some instances, a dPEG comprises about 42 or more repeating ethylene oxide units. In some instances, a dPEG comprises about 48 or more repeating ethylene oxide units. In some instances, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, L₁ is any linker that can be used to link the polymer C to the sense strand X_{S} or to link the polypeptide of F₁ to the sense strand X_{S}. In some embodiments, L₁ has a formula of:

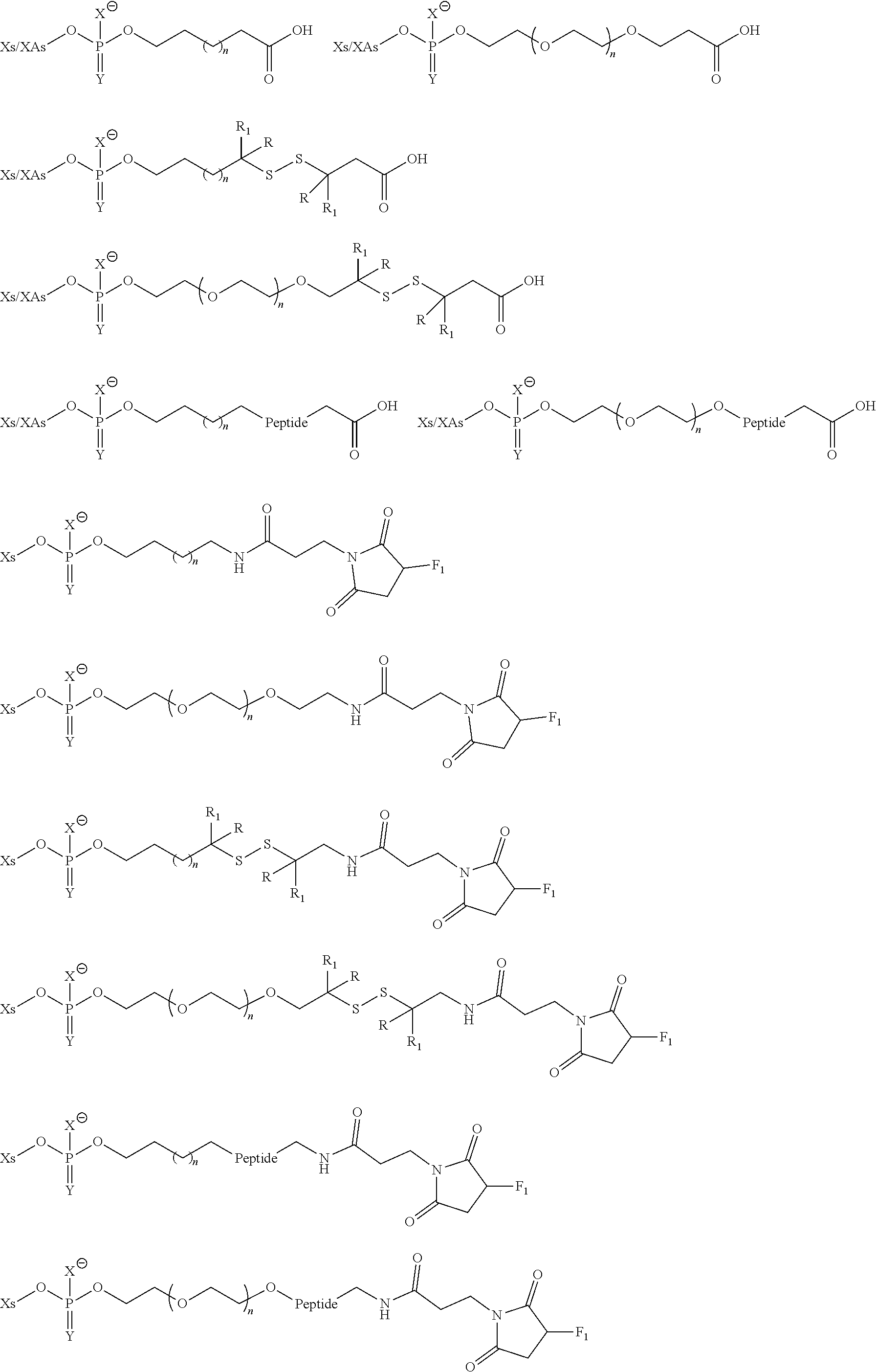

wherein X_{S}, X_{AS}, and F₁ are as defined above.

In some embodiments, n=0-20. In some embodiments, R and R1 are independently methyl. In some embodiments, R and R1 are independently present or both are absent. In some embodiments, X and Y are independently S. In some embodiments, X and Y are independently present or absent. In some embodiments, Peptide is an enzymatically cleavable peptide, such as, but not limited to, Val-Cit, Val-Ala etc.

In some embodiments, L₂ is any linker that can be used to link the polypeptide of F1 to the antisense strand X_{AS} or to link the polymer C to the antisense strand X_{AS}.

In some embodiments, L₂ has a formula of in the complex of:

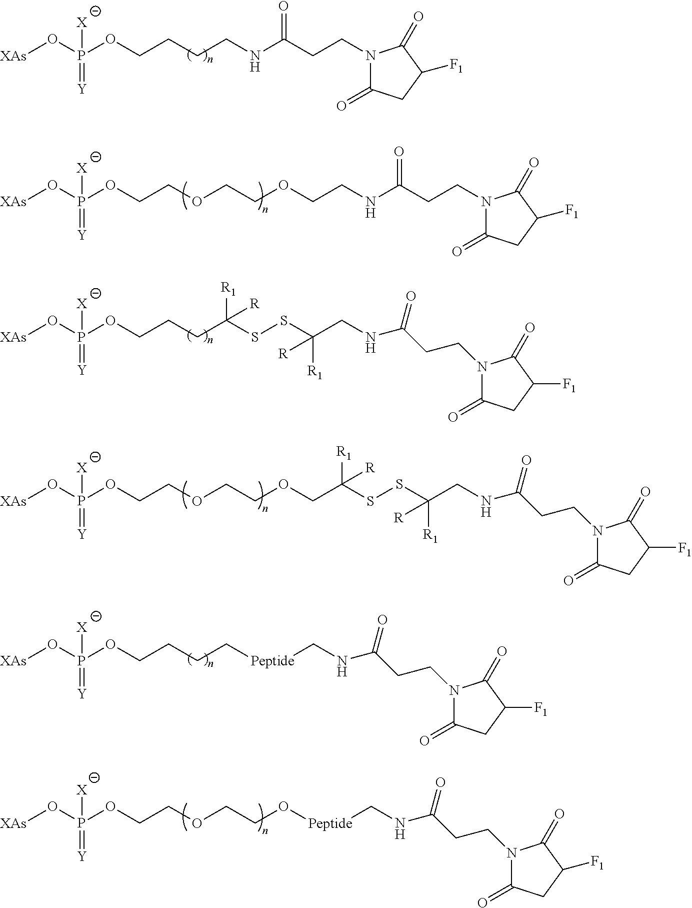

wherein X_{AS} and F₁ are as defined above.

In some embodiments, n=0-20. In some embodiments, R and R1 are independently methyl. In some embodiments, R and R1 are independently present or both are absent. In some embodiments, X and Y are independently S. In some embodiments, X and Y are independently present or absent. In some embodiments, Peptide is an enzymatically cleavable peptide, such as, but not limited to, Val-Cit, Val-Ala, etc.

In some embodiments, the linker is covalently attached to F1 through a cysteine residue present on F1, which can be illustrated as follows:

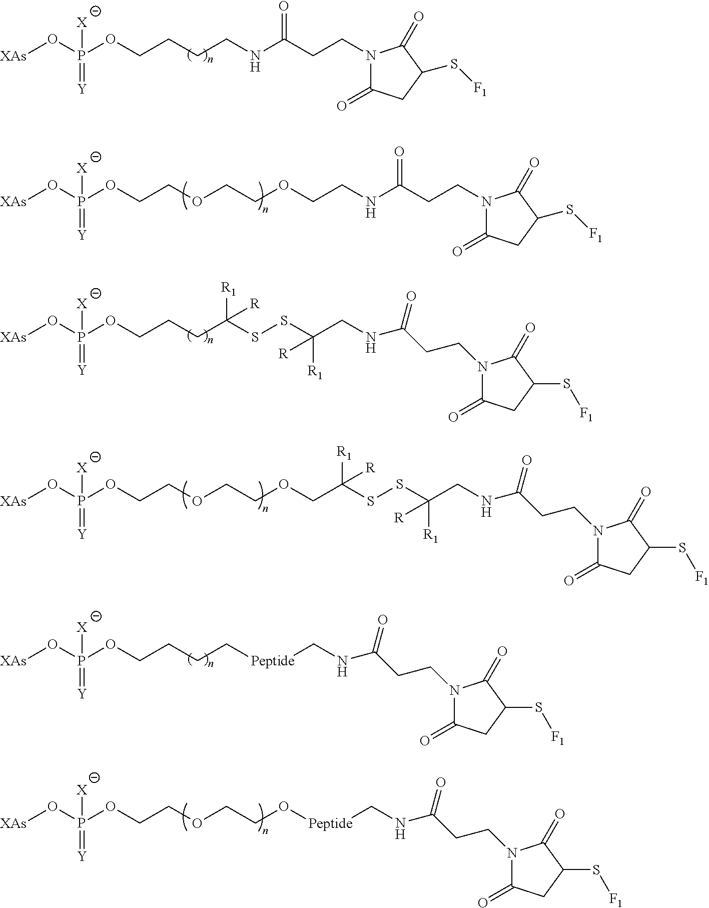

In some embodiments, A1-B1 has a formula of:

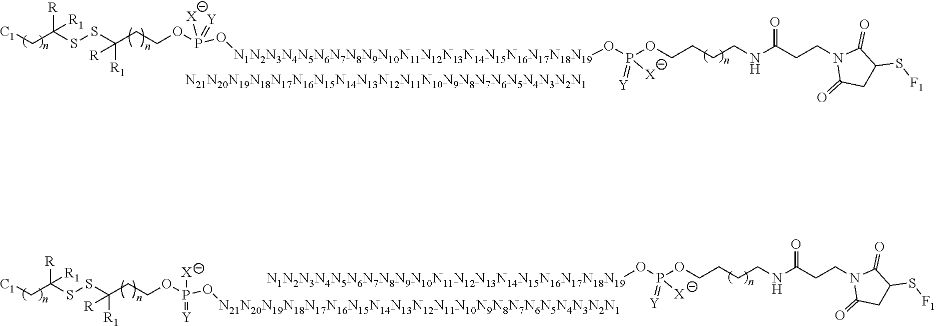

wherein C is the polymer, such as PEG, Endoporter, INF-7, TAT, polyarginine, polylysine, an amphipathic peptide, or peptides listed in Table 2 as provided for herein, X_{S} is a 5′ to 3′ oligonucleotide sense strand of a double stranded siRNA molecule; X_{AS} is a 3′ to 5′ oligonucleotide antisense strand of a double stranded siRNA molecule; and F₁ is a polypeptide comprising at least one FN3 domain, wherein X_{S} and X_{AS} form a double stranded siRNA molecule. The sense and antisense strands are represented by the “N” notations, wherein each nucleotide represented by N, is independently, A, U, C, or G or a modified nucelobase, such as those provided for herein. The N₁ nucleotides of the sense strand and the antisense strand represent the 5′ end of the respective strands. For clarity, although Formula I utilizes N₁, N₂, N₃, etc. in both the sense and the antisense strand, the nucleotide bases do not need to be the same and are not intended to be the same. The siRNA that is illustrated in Formula I would be complementary to a target sequence.

For example, in some embodiments, the sense strand comprises a 2′O-methyl modified nucleotide with a phosphorothioate (PS) modified backbone at N₁ and N2, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, N₁₈, and N₁₉.

In some embodiments, the antisense strand comprises a vinylphosphonate moiety attached to N₁, a 2′fluoro-modified nucleotide with a phosphorothioate (PS) modified backbone at N₂, a 2′O-methyl modified nucleotide at N₃, N₄, N₅, N₆, N₇, N₈, N₉, N₁₀, N₁₁, N₁₂, N₁₃, N₁₅, N₁₆, N₁₇, N₁₈, and N₁₉, a 2′fluoro-modified nucleotide at N₁₄, and a 2′O-methyl modified nucleotide with a phosphorothioate (PS) modified backbone at N₂₀ and N₂₁.

In some embodiments, a compound having a formula of:

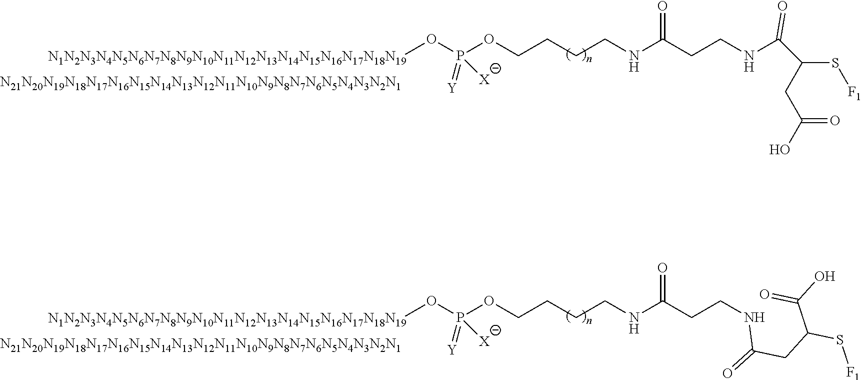

wherein F₁ is a polypeptide comprising at least one FN3 domain and is conjugated to a linker, L₁, L₁ is linked to X_{S}, wherein X_{S} is a 5′ to 3′ oligonucleotide sense strand of a double stranded siRNA molecule and X_{AS} is a 3′ to 5′ oligonucleotide antisense strand of a double stranded siRNA molecule; and wherein X_{S} and X_{AS} form a double stranded siRNA molecule. The linker illustrated above, is a non-limiting example, and other types of linkers can be used.

In some embodiments, F₁ comprises polypeptide having a formula of (X₁)ₙ—(X₂)_{q}—(X₃)_{y}, wherein X1 is a first FN3 domain; X₂ is a second FN3 domain; X₃ is a third FN3 domain or half-life extender molecule; wherein n, q, and y are each independently 0 or 1, provided that at least one of n, q, and y is 1. In some embodiments, n, q, and y are each 1. In some embodiments, n and q are 1 and y is 0. In some embodiments n and y are 1 and q is 0.

In some embodiment X1 is a CD71 FN3 binding domain, such as one provided herein. In some embodiments, X₂ is a CD71 FN3 binding domain. In some embodiments, X1 and X₂ are different CD71 FN3 binding domains. In some embodiments, the binding domains are the same. In some embodiments, X₃ is a FN3 domain that binds to human serum albumin. In some embodiments, X₃ is a Fc domain without effector function that extends the half-life of a protein. In some embodiments, X1 is a first CD71 binding domain, X₂ is a second CD71 binding domain, and X₃ is a FN3 albumin binding domain. Examples of such polypeptides are provided herein and below. In some embodiments, compositions are provided herein having a formula of C—(X₁)ₙ—(X₂)_{q}—(X₃)_{y}-L-X₄, wherein C is a polymer, such as PEG, Endoporter, INF-7, TAT, polyarginine, polylysine, an amphipathic peptide, or peptides provided in Table 2; X₁ is a first FN3 domain; X₂ is a second FN3 domain; X₃ is a third FN3 domain or half-life extender molecule; L is a linker; and X₄ is a nucleic acid molecule, wherein n, q, and y are each independently 0 or 1.

In some embodiments, compositions are provided herein having a formula of (X1)ₙ-(X2)_{q}-(X3)_{y}-L-X4-C, wherein X1 is a first FN3 domain; X2 is a second FN3 domain; X3 is a third FN3 domain or half-life extender molecule; L is a linker; X4 is a nucleic acid molecule; and C is a polymer, wherein n, q, and y are each independently 0 or 1.

In some embodiments, compositions are provided herein having a formula of X4-L-(X1)ₙ-(X2)_{q}-(X3)_{y}, wherein X1 is a first FN3 domain; X2 is a second FN3 domain; X3 is a third FN3 domain or half-life extender molecule; L is a linker; and X4 is a nucleic acid molecule, wherein n, q, and y are each independently 0 or 1.

In some embodiments, compositions are provided herein having a formula of C—X4-L-(X1)ₙ-(X2)_{q}-(X3)_{y}, wherein C is a polymer; X1 is a first FN3 domain; X2 is a second FN3 domain; X3 is a third FN3 domain or half-life extender molecule; L is a linker; and X4 is a nucleic acid molecule, wherein n, q, and y are each independently 0 or 1.

In some embodiments, compositions are provided herein having a formula of X4-L-(X1)ₙ-(X2)_{q}-(X3)_{y}-C, wherein X1 is a first FN3 domain; X2 is a second FN3 domain; X3 is a third FN3 domain or half-life extender molecule; L is a linker; X4 is a nucleic acid molecule; and C is a polymer, wherein n, q, and y are each independently 0 or 1.

In some embodiments, the GYS1 siRNA pair may follow the sequence: sense strand (5′-3′) nsnsnnnnNfNfNfnnnnnnnnsnsa and antisense strand (5′-3′) UfsNfsnnnNfnnnnnnnNfnNfnnnsusu, wherein (n) is 2′-O-Me (methyl), (Nf) is 2′-F (fluoro), (s) is phosphorothioate backbone modification. Each nucleotide in both sense and antisense strands are modified independently or in combination at ribosugar and nucleobase positions.

In some embodiments, the siRNA molecule comprises a sequence pair from Tables 3A or 3B.

TABLE 3A
siRNA Sense and Anti-sense Sequences

| SİRNA | SEQ ID | | SEQ ID | |
|---|---|---|---|---|
| Pair | NO: | Sense Strand 5′-3′ | NO: | Antisense Strand 5′-3′ |
| A | 10 | [fC][*mU][*fG][mG][fG][mA][fG] | 11 | [mU][*fU][*mC][fG][mA][fA][fU] |
| | | [fG][fA][mU][fG][mA][mA][mU][f | | [fU][mC][fA][mU][mC][mC][fU][m |
| | | U][mC][fG][mA][mA][T] | | C][fC][mC][fA][mG][*fU][*mU] |
| B | 12 | [fC][*mA][*fA][mG][fG][mU][fG] | 13 | [mU][*fU][*mA][fG][mA][fU] |
| | | [fG][fG][mU][fG][mG][mC][mA][f | | [fG][fC][mC][fA][mC][mC][mC] |
| | | U][mC][fU][mA][mA][T] | | [fA][mC][fC][mU][fU][mG][*fU] |
| | | | | [*mU] |
| C | 14 | [fU][*mC][*fC][mG][fC][mA][fG] | 15 | [mU][*fA][*mA][fU][mC][fA][fU] |
| | | [fC][fC][mU][fG][mG][mA][mU][f | | [fC][mC][fA][mG][mG][mC][fU][m |
| | | G][mA][fU][mU][mA][T] | | G][fC][mG][fG][mA][*fU][*mU] |
| D | 16 | [mG][*mC][*fG][mA][mC][mU][fG] | 17 | [fU][*fG][*mU][mU][mG][mC][mU] |
| | | [fC][fC][mU][mG][fU][mA][mG] | | [mA][mC][mA][mG][mG][mC][fA][m |
| | | [mC][mA][fA][mC][mA][idT] | | G][mU][mC][mG][mC][*mU][*mU] |
| E | 18 | [mU][*mU][*fU][mA][mU][mG][fG] | 19 | [fU][*fA][*mG][mU][mC][mC][mA] |
| | | [fG][fC][mA][mU][fC][mU][mG][m | | [mG][mA][mU][mG][mC][mC][fC][m |
| | | G][mA][fC][mU][mA][idT] | | A][mU][mA][mA][mA][*mU][*mU] |
| F | 20 | [mG][*mC][*fG][mC][mG][mG][fA] | 21 | [fU][*fG][*mA][mA][mA][mU][mU] |
| | | [fC][fC][mA][mA][fC][mA][mA][mU] | | [mG][mU][mU][mG][mG][mU][fC][m |
| | | [mU][fU][mC][mA][idT] | | C][mG][mC][mG][mC][*mU][*mU] |
| G | 22 | [mG][*mG][*fA][mC][mC][mA][fA] | 23 | [fU][*fC][*mG][mU][mU][mG][mA] |
| | | [fC][fA][mA][mU][fU][mU][mC][m | | [mA][mA][mU][mU][mG][mU][fU][m |
| | | A][mA][fC][mG][mA][idT] | | G][mG][mU][mC][mC][*mU][*mU] |
| H | 24 | [mA][*mC][*fC][mA][mA][mC][fA] | 25 | [fU][*fC][*mA][mC][mG][mU][mU] |
| | | [fA][fU][mU][mU][fC][mA][mA][m | | [mG][mA][mA][mA][mU][mU][fG][m |
| | | C][mG][fU][mG][mA][idT] | | U][mU][mG][mG][mU][*mU][*mU] |
| I | 26 | [mG][*mC][*fG][mC][mA][mA][fA] | 27 | [fU][*fU][*mC][mC][mC][mA][mA] |
| | | [fC][fA][mG][mC][fU][mU][mU][m | | [mA][mG][mC][mU][mG][mU][fU][m |
| | | G][mG][fG][mA][mA][idT] | | U][mG][mC][mG][mC][*mU][*mU] |
| J | 28 | [mA][*mG][*fA][mG][mC][mC][fA] | 29 | [fU][*fC][*mG][mU][mU][mG][mC] |
| | | [fU][fC][mU][mU][fU][mG][mC][m | | [mA][mA][mA][mG][mA][mU][fG][m |
| | | A][mA][fC][mG][mA][idT] | | G][mC][mU][mC][mU][*mU][*mU] |
| K | 30 | [mG][*mA][*fG][mC][mC][mA][fU] | 31 | [fU][*fG][*mC][mG][mU][mU][mG] |
| | | [fC][fU][mU][mU][fG][mC][mA][m | | [mC][mA][mA][mA][mG][mA][fU][m |
| | | A][mC][fG][mC][mA][idT] | | G][mG][mC][mU][mC][*mU][*mU] |
| L | 32 | [mA][*mG][*fC][mC][mA][mU][fC] | 33 | [fU][*fU][*mG][mC][mG][mU][mU] |
| | | [fU][fU][mU][mG][fC][mA][mA][m | | [mG][mC][mA][mA][mA][mG][fA][m |
| | | C][mG][fC][mA][mA][idT] | | U][mG][mG][mC][mU][*mU][*mU] |
| M | 34 | [mC][*mC][*fA][mU][mC][mU][fU] | 35 | [fU][*fG][*mC][mU][mG][mC][mG] |
| | | [fU][fG][mC][mA][fA][mC][mG][m | | [mU][mU][mG][mC][mA][mA][fA][m |
| | | C][mA][fG][mC][mA][idT] | | G][mA][mU][mG][mG][*mU][*mU] |
| N | 36 | [mA][*mC][*fG][mC][mA][mG][fC] | 37 | [fU][*fG][*mA][mA][mA][mG][mA] |
| | | [fG][fG][mC][mA][fG][mU][mC][m | | [mC][mU][mG][mC][mC][mG][fC][m |
| | | U][mU][fU][mC][mA][idT] | | U][mG][mC][mG][mU][*mU][*mU] |
| O | 38 | [mU][*mG][*fA][mC][mC][mA][fC] | 39 | [fU][*fU][*mU][mC][mG][mG][mC] |
| | | [fC][fA][mU][mC][fC][mG][mC][m | | [mG][mG][mA][mU][mG][mG][fU][m |
| | | C][mG][fA][mA][mA][idT] | | G][mG][mU][mC][mA][*mU][*mU] |
| P | 40 | [mC][*mG][*fA][mA][mU][mC][fG] | 41 | [fU][*fA][*mU][mU][mG][mA][mA] |
| | | [fG][fC][mC][mU][fC][mU][mU][m | | [mG][mA][mG][mG][mC][mC][fG][m |
| | | C][mA][fA][mU][mA][idT] | | A][mU][mU][mC][mG][*mU][*mU] |
| Q | 42 | [mC][*mU][*fU][mC][mA][mA][fU] | 43 | [fU][*fU][*mC][mG][mG][mC][mA] |
| | | [fA][fG][mC][mA][fG][mU][mG][mC] | | [mC][mU][mG][mC][mU][mA][fU][m |
| | | [mC][fG][mA][mA][idT] | | U][mG][mA][mA][mG][*mU][*mU] |
| R | 44 | [mC][*mA][*fG][mU][mA][mU][fC] | 45 | [fU][*fA][*mG][mA][mU][mU][mG] |
| | | [fU][fC][mC][mA][fC][mC][mA][m | | [G][mU][mG][mG][mA][mG][fA][mU |
| | | A][mU][fC][mU][mA][idT] | | ][mA][mC][mU][mG][*mU][*mU] |
| S | 46 | [mC][*mC][*fA][mC][mC][mA][A][ | 47 | [fU][*fA][*mG][mC][mC][mG][mG] |
| | | fU][fC][mU][mC][fU][mC][mC][mG | | [mA][mG][mA][mG][mA][mU][fU][m |
| | | ][mG][fC][mU][mA][idT] | | G][mG][mU][mG][mG][*mU][*mU] |
| T | 48 | [mC][*mA][*fC][mC][mA][mA][fU] | 49 | [fU][*fA][*mA][mG][mC][mC][mG] |
| | | [fC][fU][mC][mU][fC][mC][mG][m | | [mG][mA][mG][mA][mG][mA][fU][m |
| | | G][mC][fU][mU][mA][idT] | | U][mG][mG][mU][mG][*mU][*mU] |
| U | 50 | [mC][*mC][*fC][mC][mU][mC][mA] | 51 | [fU][*fA][*mU][mA][mC][mC][mG] |
| | | [fG][fC][mU][mU][fA][mC][mG][m | | [mU][mA][mA][mG][mC][mU][fG][m |
| | | G][mU][fA][mU][mA][idT] | | A][mG][mG][mG][mG][*mU][*mU] |
| V | 52 | [mC][*mC][*fC][mU][mC][mA][fG] | 53 | [fU][*fG][*mA][mU][mA][mC][mC] |
| | | [fC][fU][mU][mA][fC][mG][mG][m | | [mG][mU][mA][mA][mG][mC][fU][m |
| | | U][mA][fU][mC][mA][idT] | | G][mA][mG][mG][mG][*mU][*mU] |
| W | 54 | [mU][*mU][*fA][mC][mG][mG][fU] | 55 | [fU][*fA][*mG][mA][mA][mU][mG] |
| | | [fA][fU][mC][mU][fA][mC][mA][m | | [mU][mA][mG][mA][mU][mA][fC][m |
| | | U][mU][fC][mU][mA][idT] | | C][mG][mU][mA][mA][*mU][*mU] |
| X | 56 | [mG][*mG][*fA][mA][mC][mC][fG] | 57 | [fU][*fG][*mG][mC][mG][mC][mU] |
| | | [fC][fA][mC][mG][fG][mA][mG][m | | [mC][mC][mG][mU][mG][mC][fG][m |
| | | C][mG][fC][mC][mA][idT] | | G][mU][mU][mC][mC][*mU][*mU] |
| Y | 58 | [mA][*mC][*fG][mG][mA][mG][fC] | 59 | [fU][*fG][*mU][mC][mG][mG][mA] |
| | | [fG][fC][mC][mU][fC][mU][mC][m | | [mG][mA][mG][mG][mC][mG][fC][m |
| | | C][mG][fA][mC][mA][idT] | | U][mC][mC][mG][mU][*mU][*mU] |
| Z | 60 | [mA][*mG][*fC][mA][mA][mG][fC] | 61 | [fU][*fC][*mA][mC][mA][mG][mA] |
| | | [fG][fC][mA][mA][fC][mU][mC][m | | [mG][mU][mU][mG][mC][mG][fC][m |
| | | U][mG][fU][mG][mA][idT] | | U][mU][mG][mC][mU][*mU][*mU] |
| AA | 62 | [mC][*mA][*fG][mA][mU][mG][fG] | 63 | [fU][*fG][*mA][mA][mA][mU][mG] |
| | | [fU][fC][mC][mU][fC][mC][mA][m | | [mG][mA][mG][mG][mA][mC][fC][m |
| | | U][mU][fU][mC][mA][idT] | | A][mU][mC][mU][mG][*mU][*mU] |
| BB | 64 | [mU][*mC][*fU][mA][mG][mA][fU] | 65 | [fU][*fU][*mA][mA][mG][mG][mU] |
| | | [fC][fU][mG][fA][mA][mC][mC] | | [mU][mC][mC][mA][mG][mA][fU][m |
| | | [mU][fU][mA][mA][idT] | | C][mU][mA][mG][mA][*mU][*mU] |
| CC | 66 | [mC][*mC][*fC][mA][mC][mA][fU] | 67 | [fU][*fC][*mC][mU][mC][mG][mA] |
| | | [fU][fU][mA][mC][fC][mU][mC][m | | [mG][mG][mU][mA][mA][mA][fU][m |
| | | G][mA][fG][mG][mA][idT] | | G][mU][mG][mG][mG][*mU][*mU] |
| DD | 68 | [mA][*mC][*fC][mG][mC][mU][fU] | 69 | [fU][*fA][*mA][mC][mU][mG][mC] |
| | | [fG][fG][mU][mU][fU][mG][mC][m | | [mA][mA][mA][mC][mC][mA][fA][m |
| | | A][mG][fU][mU][mA][idT] | | G][mC][mG][mG][mU][*mU][*mU] |
| EE | 600 | [fC][*mU][*fG][mG][fG][mA][fG] | 601 | [mU][*fU][*mC][fG][mA][fA][fU] |
| | | [fG][fA][mU][fG][mA][mA][mU][f | | [fU][mC][fA][mU][mC][mC][fU][m |
| | | U][mC][fG][mG][mA][T] | | C][fC][mC][fA][mG][*fU][*mU] |
| FF | 602 | [fC][*mU][*fG][mG][fG][mA][fG] | 603 | [mU][*fU][*mC][fG][mA][fA][fU] |
| | | [fG][fA][mU][fG][mA][mA][mU][f | | [fU][mC][fA][mU][mC][mC][fU] |
| | | U][mA][fG][mG][mA][T] | | [mc][mC][fA][mG][*fU][*mU] |
| GG | 604 | [fC][*mU][*fG][mG][fG][mA][fG] | 605 | [mU][*fU][*mC][fG][mA][fA][fU] |
| | | [fG][fA][mU][fG][mA][mA][mU][f | | [fU][mC][fA][mU][mC][mC][fU][m |
| | | G][mA][fG][mG][mA][T] | | C][fC][mC][fA][mG][*fU][*mU] |
| HH | 606 | [fC][*mU][*fG][mG][fG][mA][fG] | 607 | [fU][*fU][*fC][G][fA][fA][fU][ |
| | | [fG][fA][mU][fG][mA][mA][mU][f | | fU][fC][fA][fU][{C][fC][fU][fC |
| | | U][mC][fG][mA][mA][T] | | ][{C][{C][{A][fG][*fU][*fU] |
| II | 608 | [fC][*mU][*fG][mG][fG][mA][fG] | 609 | [mU][*fU][*fC][fG][fA][fA][fU] |
| | | [fG][fA][mU][fG][mA][mA][mU][f | | [fU][mC][fA][mU][fC][fC][fU][f |
| | | U][mC][fG][mA][mA][T] | | C][fC][fC][fA][fG][*fU][*mU] |
| JJ | 610 | [fC][*mU][*fG][mG][fG][mA][fG] | 611 | [mU][*fU][*mC][fG][mA][fA][fU] |
| | | [G][fA][mU][fG][mA][mA][*fU][i | | [fU][mC][fA][mU][mC][mC][fU][m |
| | | dT] | | C][fC][mC][fA][*mG][*fU][*mU] |
| KK | 612 | [fC][*mA][*fA][mG][fG][mU][fG] | 613 | [mU][*fU][*mA][fG][mA][fU][fG] |
| | | [fG][fG][mU][fG][mG][mC][mA][f | | [fC][mC][fA][mC][mC][mC][fA][m |
| | | U][mC][fU][mA][mA][idT] | | C][fC][mU][fU][mG][*fU][*mU] |
| LL | 614 | [fC][*mA][*fA][mG][fG][mU][G][ | 615 | [mU][*fU][*mA][fG][mA][fU][fG] |
| | | fG][fG][mU][fG][mG][mC][mA][fU] | | [fC][mC][fA][mC][mC][mC][fA][m |
| | | [mC][fU][mA][mG][idT] | | C][fC][mU][fU][mG][*fU][*mU] |
| MM | 616 | [fC][*mA][*fA][mG][fG][mU][fG] | 617 | [mU][*fU][*mA][fG][mA][fU][fG] |
| | | [fG][fG][mU][fG][mG][mC][mA][ | | [fC][mC][fA][mC][mC][mC][fA][m |
| | | fU][mC][fU][mG][mA][idT] | | C][fC][mU][fU][mG][*fU][*mU] |
| NN | 618 | [fC][*mA][*fA][mG][fG][mU][fG] | 619 | [mU][*fU][*mA][fG][mA][fU][fG] |
| | | [fG][fG][mU][fG][mG][mC][mA][f | | [fC][mC][fA][mC][mC][mC][fA][m |
| | | U][mC][fG][mA][mA][idT] | | C][fC][mU][fU][mG][*fU][*mU] |
| OO | 620 | [fC][*mA][*fA][mG][fG][mU][fG] | 621 | [mU][*fU][*mA][fG][mA][fU][fG] |
| | | [fG][fG][mU][fG][mG][mC][mA][f | | [fC][mC][fA][mC][mC][mC][fA][m |
| | | U][mA][fU][mA][mA][idT] | | C][fC][mU][fU][mG][*fU][*mU] |
| PP | 622 | [fC][*mA][*fA][mG][fG][mU][fG] | 623 | [mU][*fU][*mA][fG][mA][fU][fG] |
| | | [fG][fG][mU][fG][mG][mC][mA][f | | [fC][mC][fA][mC][mC][mC][fA][m |
| | | G][mC][fU][mA][mA][idT] | | C][fC][mU][fU][mG][*fU][*mU] |
| QQ | 624 | [fC][*mA][*fA][mG][fG][mU][fG] | 625 | [mU][*fU][*mA][fG][mA][fU][fG] |
| | | [fG][fG][mU][fG][mG][mC][mA][f | | [fC][mC][fA][mC][mC][mC][fA][m |
| | | G][mC][fU][mA][mG][idT] | | C][fC][mU][fU][mG][*fU][*mU] |
| RR | 626 | [mG][*mA][*fG][mC][mC][mA][fU] | 627 | [mU][*fG][*mC][mG][mU][mU][mG] |
| | | [fC][fU][mU][mU][fG][mC][mA][m | | [mC][mA][mA][mA][mG][mA][fU][m |
| | | A][mC][fG][mC][mA][idT] | | G][mG][mC][mU][mC][*mU][*mU] |
| SS | 628 | [mG][*mA][*fG][mC][mC][mA][fU] | 629 | [mU][*fG][*mC][mG][mU][mU][mG] |
| | | [fC][fU][mU][mU][fG][mC][mA][m | | [mC][mA][mA][mA][mG][mA][fU][m |
| | | A][mC][fG][mC][mG][idT] | | G][mG][mC][mU][mC][*mU][*mU] |
| TT | 630 | [mG][*mA][*fG][mC][mC][mA][fU] | 631 | [mU][*fG][*mC][mG][mU][mU][mG] |
| | | [fC][fU][mU][mU][fG][mC][mA][m | | [mC][mA][mA][mA][mG][mA][fU][m |
| | | A][mC][fG][mA][mA][idT] | | G][mG][mC][mU][mC][*mU][*mU] |
| UU | 632 | [mG][*mA][*fG][mC][mC][mA][fU] | 633 | [mU][*fG][*mC][mG][mU][mU][mG] |
| | | [fC][fU][mU][mU][fG][mC][mA][m | | [mC][mA][mA][mA][mG][mA][fU][m |
| | | A][mC][fA][mC][mA][idT] | | G][mG][mC][mU][mC][*mU][*mU] |
| VV | 634 | [mG][*mA][*fG][mC][mC][mA][fU] | 635 | [mU][*fG][*mC][mG][mU][mU][mG] |
| | | [fC][fU][mU][mU][fG][mC][mA][m | | [mC][mA][mA][mA][mG][mA][fU][m |
| | | A][mA][fG][mC][mA][idT] | | G][mG][mC][mU][mC][*mU][*mU] |
| WW | 636 | [mG][*mA][*fG][mC][mC][mA][fU][f | 637 | [mU][*fG][*mC][mG][mU][mU][mG] |
| | | C][fU][mU][mU][fG][mC][mA][mG][ | | [mC][mA][mA][mA][mG][mA][fU][m |
| | | mC][fG][mC][mA][idT] | | G][mG][mC][mU][mC][*mU][*mU] |
| XX | 638 | [mG][*mA][*fG][mC][mC][mA][fU] | 639 | [mU][*fG][*mC][mG][mU][mU][mG] |
| | | [fC][fU][mU][mU][fG][mC][mA][m | | [mC][mA][mA][mA][mG][mA][fU][m |
| | | G][mC][fG][mC][mG][idT] | | G][mG][mC][mU][mC][*mU][*mU] |
| YY | 640 | [mU][*mU][*fA][mC][mG][mG][fU] | 641 | [mU][*fA][*mG][mA][mA][mU][mG][mU |
| | | [fA][fU][mC][mU][fA][mC][mA][m | | ][mA][mG][mA][mU][mA][fC][mC][mG][ |
| | | U][mU][fC][mU][mA][idT] | | mU][mA][mA][*mU][*mU] |
| ZZ | 642 | [mU][*mU][*fA][mC][mG][mG][fU] | 643 | [mU][*fA][*mG][mA][mA][mU][mG][mU |
| | | [fA][fU][mC][mU][fA][mC][mA][m | | ][mA][mG][mA][mU][mA][fC][mC][mG][ |
| | | U][mU][fC][mU][mG][idT] | | mU][mA][mA][*mU][*mU] |
| AAA | 644 | [mU][*mU][*fA][mC][mG][mG][fU] | 645 | [mU][*fA][*mG][mA][mA][mU][mG][mU |
| | | [fA][fU][mC][mU][fA][mC][mA][m | | ][mA][mG][mA][mU][mA][fC][mC][mG][ |
| | | U][mU][fC][mG][mA][idT] | | mU][mA][mA][*mU][*mU] |
| BBB | 646 | [mU][*mU][*fA][mC][mG][mG][fU] | 647 | [mU][*fA][*mG][mA][mA][mU][mG] |
| | | [fA][fU][mC][mU][fA][mC][mA][m | | [mU][mA][mG][mA][mU][mA][fC][m |
| | | U][mU][fG][mU][mA][idT] | | C][mG][mU][mA][mA][*mU][*mU] |
| CCC | 648 | [mU][*mU][*fA][mC][mG][mG][fU] | 649 | [mU][*fA][*mG][mA][mA][mU][mG][mU |
| | | [fA][fU][mC][mU][fA][mC][mA][m | | ][mA][mG][mA][mU][mA][fC][mC][mG][ |
| | | U][mG][fC][mU][mA][idT] | | mU][mA][mA][*mU][*mU] |
| DDD | 650 | [mU][*mU][*fA][mC][mG][mG][fU] | 651 | [mU][*fA][*mG][mA][mA][mU][mG][mU |
| | | [fA][fU][mC][mU][fA][mC][mA][m | | ][mA][mG][mA][mU][mA][fC][mC][mG][ |
| | | U][mG][fC][mU][mA][idT] | | mU][mA][mA][*mU][*mU] |
| EEE | 652 | [mU][*mU][*fA][mC][mG][mG][fU] | 653 | [mU][*fA][*mG][mA][mA][mU][mG] |
| | | [fA][fU][mC][mU][fA][mC][mA][m | | [mU][mA][mG][mA][mU][mA][fC][m |
| | | G][mU][fC][mU][mA][idT] | | C][mG][mU][mA][mA][*mU][*mU] |
| FFF | 654 | [mU][*mU][*fA][mC][mG][mG][fU] | 655 | [mU][*fA][*mG][mA][mA][mU][mG] |
| | | [fA][fU][mC][mU][fA][mC][mA][m | | [mU][mA][mG][mA][mU][mA][fC][m |
| | | G][mU][fC][mU][mG][idT] | | C][mG][mU][mA][mA][*mU][*mU] |
| GGG | 656 | [fC][*mA][*fA][mG][fG][mU][fG] | 657 | [mU][*fU][*mA][fG][mA][fU][fG] |
| | | [fG][fG][mU][fG][mG][mC][mA][f | | [fC][mC][fA][mC][mC][mC][fA][m |
| | | U][mC][fU][mA][mG][idT] | | C][fC][mU][fU][mG][*fU][*mU] |
| HHH | 704 | [mU][*mU][*fA][mC][mG][mG][fU] | 705 | [mU][*fA][*mG][mA][mA][mU][mG] |
| | | [fA][fU][mC][mU][fA][mC][mA][m | | [mU][mA][mG][mA][mU][mA][fC][m |
| | | U][mU][fC][mU][mA] | | C][mG][mU][mA][mA][*mU][*mU] |

Abbreviations Key: (n/N=any nucleotide) mN=2′-O-methyl residues, fN=2′-F residues, *=phosphorothioate and (idT)=inverted Dt, (VP) 2′-O methyl vinlyl phosphonate uridine. The brackets indicate the individual bases.

TABLE 3B

| SEQ | | SEQ | |
|---|---|---|---|
| ID | | ID | |
| NO: | Sense Strand (5′-3′) | NO: | Antisense Strand (5′-3′) |
| 801 | caggggugcg gucuugcaa | 861 | uugcaagacc gcaccccugu u |
| 802 | ggugcggucu ugcaauagg | 862 | ccuauugcaa gaccgcaccu u |
| 803 | gugcggucuu gcaauagga | 863 | uccuauugca agaccgcacu u |
| 804 | agccaugccu uuaaaccgc | 864 | gcgguuuaaa ggcauggcuu u |
| 805 | augccuuuaa accgcacuu | 865 | aagugcgguu uaaaggcauu u |
| 806 | ugggaggaug aauucgacc | 866 | ggucgaauuc auccucccau u |
| 807 | aacgcagugc ucuucgaag | 867 | cuucgaagag cacugcguuu u |
| 808 | ggacgaaugg ggcgacaac | 868 | guugucgccc cauucguccu u |
| 809 | ggcgacugcc uguagcaac | 869 | guugcuacag gcagucgccu u |
| 810 | cacgcugcug gggcgcuac | 870 | guagcgcccc agcagcgugu u |
| 811 | ugcgcucacg ucuucacua | 871 | uagugaagac gugagcgcau u |
| 812 | ccuuauacuu cuuuaucgc | 872 | gcgauaaaga aguauaaggu u |
| 813 | cuuuaucgcc ggccgcuau | 873 | auagcggccg gcgauaaagu u |
| 814 | ucgccggccg cuaugaguu | 874 | aacucauagc ggccggcgau u |
| 815 | gccgcuauga guucuccaa | 875 | uuggagaacu cauagcggcu u |
| 816 | ccgcuaugag uucuccaac | 876 | guuggagaac ucauagcggu u |
| 817 | ggcucggcuc aacuaucug | 877 | cagauaguug agccgagccu u |
| 818 | gcucggcuca acuaucugc | 878 | gcagauaguu gagccgagcu u |
| 819 | ucggcucaac uaucugcuc | 879 | gagcagauag uugagccgau u |
| 820 | ccagcgcgga ccaacaauu | 880 | aauuguuggu ccgcgcuggu u |
| 821 | agcgcggacc aacaauuuc | 881 | gaaauuguug guccgcgcuu u |
| 822 | ucgggaggaa gcuuuauga | 882 | ucauaaagcu uccucccgau u |
| 823 | acgcageggc agucuuucc | 883 | ggaaagacug ccgcugcguu u |
| 824 | gaccaccauc cgccgaauc | 884 | gauucggcgg augguggucu u |
| 825 | ccgaaucggc cucuucaau | 885 | auugaagagg ccgauucggu u |
| 826 | aaucggccuc uucaauagc | 886 | gcuauugaag aggccgauuu u |
| 827 | caauagcagu gccgacagg | 887 | ccugucggca cugcuauugu u |
| 828 | aggaguuugu ccguggcug | 888 | cagccacgga caaacuccuu u |
| 829 | cggcugagug cacgguuau | 889 | auaaccgugc acucagccgu u |
| 830 | ugggaauccc caguaucuc | 890 | gagauacugg ggauucccau u |
| 831 | cagaccccuc agcuuacgg | 891 | ccguaagcug aggggucugu u |
| 832 | ccccucagcu uacgguauc | 892 | gauaccguaa gcugaggggu u |
| 833 | cucagcuuac gguaucuac | 893 | guagauaccg uaagcugagu u |
| 834 | gcuuacggua ucuacauuc | 894 | gaauguagau accguaagcu u |
| 835 | ccggcggcag cguaucauc | 895 | gaugauacgc ugccgccggu u |
| 836 | agcguaucau ccagcggaa | 896 | uuccgcugga ugauacgcuu u |
| 837 | gcguaucauc cagcggaac | 897 | guuccgcugg augauacgcu u |
| 838 | uaccuaggcc gguacuaua | 898 | uauaguaccg gccuagguau u |
| 839 | cuaggccggu acuauaugu | 899 | acauauagua ccggccuagu u |
| 840 | aggccgguac uauaugucu | 900 | agacauauag uaccggccuu u |
| 841 | ggccgguacu auaugucug | 901 | cagacauaua guaccggccu u |
| 842 | gccgguacua uaugucugc | 902 | gcagacauau aguaccggcu u |
| 843 | cgguacuaua ugucugcgc | 903 | gcgcagacau auaguaccgu u |
| 844 | gguacuauau gucugcgcg | 904 | cgcgcagaca uauaguaccu u |
| 845 | cuauaugucu gcgcgccac | 905 | guggcgcgca gacauauagu u |
| 846 | uaugucugcg cgccacaug | 906 | cauguggcgc gcagacauau u |
| 847 | augucugcgc gccacaugg | 907 | ccauguggcg cgcagacauu u |
| 848 | ucgcccucgc ugucacgac | 908 | gucgugacag cgagggcgau u |
| 849 | cggcgagcgc uacgaugag | 909 | cucaucguag cgcucgccgu u |
| 850 | caaggaccgg cgcaacauc | 910 | gauguugcgc cgguccuugu u |
| 851 | acauccgugc accagagug | 911 | cacucuggug cacggauguu u |
| 852 | cugggcgagg agcguaacu | 912 | aguuacgcuc cucgcccagu u |
| 853 | gggcgaggag cguaacuaa | 913 | uuaguuacgc uccucgcccu u |
| 854 | gcgaggagcg uaacuaagu | 914 | acuuaguuac gcuccucgcu u |
| 855 | gaggagcgua acuaagucc | 915 | ggacuuaguu acgcuccucu u |
| 856 | aguccgccaa acacuccac | 916 | guggaguguu uggcggacuu u |
| 857 | ggcgaucaag uccagagcc | 917 | ggcucuggac uugaucgccu u |
| 858 | cccuaaccug gcuuauucc | 918 | ggaauaagcc agguuagggu u |
| 859 | ugugaaacca cuagguucu | 919 | agaaccuagu gguuucacau u |
| 860 | accacuaggu ucuaggucc | 920 | ggaccuagaa ccuagugguu u |
| 921 | caggggugcg gucuugcaa | 981 | uugcaagacc gcaccccugu u |
| 922 | ggugcggucu ugcaauaga | 982 | ucuauugcaa gaccgcaccu u |
| 923 | gugcggucuu gcaauagga | 983 | uccuauugca agaccgcacu u |
| 924 | agccaugccu uuaaaccga | 984 | ucgguuuaaa ggcauggcuu u |
| 925 | augccuuuaa accgcacua | 985 | uagugcgguu uaaaggcauu u |
| 926 | ugggaggaug aauucgaca | 986 | ugucgaauuc auccucccau u |
| 927 | aacgcagugc ucuucgaaa | 987 | uuucgaagag cacugcguuu u |
| 928 | ggacgaaugg ggcgacaaa | 988 | uuugucgccc cauucguccu u |
| 929 | ggcgacugcc uguagcaaa | 989 | uuugcuacag gcagucgccu u |
| 930 | cacgcugcug gggcgcuaa | 990 | uuagcgcccc agcagcgugu u |
| 931 | ugcgcucacg ucuucacua | 991 | uagugaagac gugagcgcau u |
| 932 | ccuuauacuu cuuuaucga | 992 | ucgauaaaga aguauaaggu u |
| 933 | cuuuaucgcc ggccgcuaa | 993 | uuagcggccg gcgauaaagu u |
| 934 | ucgccggccg cuaugagua | 994 | uacucauagc ggccggcgau u |
| 935 | gccgcuauga guucuccaa | 995 | uuggagaacu cauagcggcu u |
| 936 | ccgcuaugag uucuccaaa | 996 | uuuggagaac ucauagcggu u |
| 937 | ggcucggcuc aacuaucua | 997 | uagauaguug agccgagccu u |
| 938 | gcucggcuca acuaucuga | 998 | ucagauaguu gagccgagcu u |
| 939 | ucggcucaac uaucugcua | 999 | uagcagauag uugagccgau u |
| 940 | ccagcgcgga ccaacaaua | 1000 | uauuguuggu ccgcgcuggu u |
| 941 | agcgcggacc aacaauuua | 1001 | uaaauuguug guccgcgcuu u |
| 942 | ucgggaggaa gcuuuauga | 1002 | ucauaaagcu uccucccgau u |
| 943 | acgcagcggc agucuuuca | 1003 | ugaaagacug ccgcugcguu u |
| 944 | gaccaccauc cgccgaaua | 1004 | uauucggcgg augguggucu u |
| 945 | ccgaaucggc cucuucaaa | 1005 | uuugaagagg ccgauucggu u |
| 946 | aaucggccuc uucaauaga | 1006 | ucuauugaag aggccgauuu u |
| 947 | caauagcagu gccgacaga | 1007 | ucugucggca cugcuauugu u |
| 948 | aggaguuugu ccguggcua | 1008 | uagccacgga caaacuccuu u |
| 949 | cggcugagug cacgguuaa | 1009 | uuaaccgugc acucagccgu u |
| 950 | ugggaauccc caguaucua | 1010 | uagauacugg ggauucccau u |
| 951 | cagaccccuc agcuuacga | 1011 | ucguaagcug aggggucugu u |
| 952 | ccccucagcu uacgguaua | 1012 | uauaccguaa gcugaggggu u |
| 953 | cucagcuuac gguaucuaa | 1013 | uuagauaccg uaagcugagu u |
| 954 | gcuuacggua ucuacauua | 1014 | uaauguagau accguaagcu u |
| 955 | ccggcggcag cguaucaua | 1015 | uaugauacgc ugccgccggu u |
| 956 | agcguaucau ccagcggaa | 1016 | uuccgcugga ugauacgcuu u |
| 957 | gcguaucauc cagcggaaa | 1017 | uuuccgcugg augauacgcu u |
| 958 | uaccuaggcc gguacuaua | 1018 | uauaguaccg gccuagguau u |
| 959 | cuaggccggu acuauauga | 1019 | ucauauagua ccggccuagu u |
| 960 | aggccgguac uauauguca | 1020 | ugacauauag uaccggccuu u |
| 961 | ggccgguacu auaugucua | 1021 | uagacauaua guaccggccu u |
| 962 | gccgguacua uaugucuga | 1022 | ucagacauau aguaccggcu u |
| 963 | cgguacuaua ugucugcga | 1023 | ucgcagacau auaguaccgu u |
| 964 | gguacuauau gucugcgca | 1024 | ugcgcagaca uauaguaccu u |
| 965 | cuauaugucu gcgcgccaa | 1025 | uuggcgcgca gacauauagu u |
| 966 | uaugucugcg cgccacaua | 1026 | uauguggcgc gcagacauau u |
| 967 | augucugcgc gccacauga | 1027 | ucauguggcg cgcagacauu u |
| 968 | ucgcccucgc ugucacgaa | 1028 | uucgugacag cgagggcgau u |
| 969 | cggcgagcgc uacgaugaa | 1029 | uucaucguag cgcucgccgu u |
| 970 | caaggaccgg cgcaacaua | 1030 | uauguugcgc cgguccuugu u |
| 971 | acauccgugc accagagua | 1031 | uacucuggug cacggauguu u |
| 972 | cugggcgagg agcguaaca | 1032 | uguuacgcuc cucgcccagu u |
| 973 | gggcgaggag cguaacuaa | 1033 | uuaguuacgc uccucgcccu u |
| 974 | gcgaggagcg uaacuaaga | 1034 | ucuuaguuac gcuccucgcu u |
| 975 | gaggagcgua acuaaguca | 1035 | ugacuuaguu acgcuccucu u |
| 976 | aguccgccaa acacuccaa | 1036 | uuggaguguu uggcggacuu u |
| 977 | ggcgaucaag uccagagca | 1037 | ugcucuggac uugaucgccu u |
| 978 | cccuaaccug gcuuauuca | 1038 | ugaauaagcc agguuagggu u |
| 979 | ugugaaacca cuagguuca | 1039 | ugaaccuagu gguuucacau u |
| 980 | accacuaggu ucuagguca | 1040 | ugaccuagaa ccuagugguu u |
| 1041 | uggacuucaaccuagacaa | 1042 | uugucuagguugaaguccauu |
| 783 | cugggaggaugaauucgaa | 784 | uucgaauucauccucccaguu |
| 785 | gggugacaacuacuaucua | 786 | uagauaguaguugucacccuu |
| 787 | cugggaggaugaauucgaa | 788 | uucgaauucauccucccaguu |
| 789 | caagguggguggcaucuaa | 790 | uuagaugccacccaccuuguu |
| 791 | uccgcagccuggaugauua | 792 | uaaucauccaggcugcggauu |
| 1065 | uuacgguaucuacauucua | 1066 | uagaauguagauaccguaauu |

In some embodiments, the polynucleotides illustrated above include those that do not include a 2′-O methyl vinlyl phosphonate uridine as the 5′ nucleotide on the antisense strand of the siRNA.

In some embodiments, a polynucleotide is as provided for herein. In some embodiments, the polynucleotide comprises a first strand and a second strand to for a portion that comprises a duplex. In some embodiments, the polynucleotide comprises a sense strand and an antisense strand. In some embodiments, comprises the sequences as illustrated in Tables 3A or 3B. In some embodiments, comprises the sequences as illustrated in Tables 3A or 3B but without the base modifications. In some embodiments, a pharmaceutical composition comprises a siRNA pair as provided herein. In some embodiments, the siRNA pair is not conjugated to a FN3 domain.

In some embodiments, an oligonucleotide molecule described herein is constructed using chemical synthesis and/or enzymatic ligation reactions using procedures known in the art. For example, an oligonucleotide molecule is chemically synthesized using naturally occurring nucleotides or variously modified nucleotides designed to increase the biological stability of the molecules or to increase the physical stability of the duplex formed between the oligonucleotide molecule and target nucleic acids. Alternatively, the oligonucleotide molecule is produced biologically using an expression vector into which a oligonucleotide molecule has been subcloned in an antisense orientation (i.e., RNA transcribed from the inserted oligonucleotide molecule will be of an antisense orientation to a target polynucleic acid molecule of interest).

In some embodiments, an oligonucleotide molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, an oligonucleotide molecule is also assembled from two distinct nucleic acid strands or fragments wherein one fragment includes the sense region and the second fragment includes the antisense region of the molecule.

In some instances, while chemical modification of the oligonucleotide molecule internucleotide linkages with phosphorothioate, phosphorodithioate, phosphonate, phosphoramidate, or mesyl phosphoramidate, linkages improves stability. Excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

As described herein, in some embodiments, the nucleic acid molecules can be modified to include a linker at the 5′ end of the of the sense strand of the dsRNA. In some embodiments, the nucleic acid molecules can be modified to include a vinyl phosphonate or modified vinyl phosphonate at the 5′ end of the of the anti-sense strand of the dsRNA. In some embodiments, the nucleic acid molecules can be modified to include a linker at the 3′ end of the of the sense strand of the dsRNA. In some embodiments, the nucleic acid molecules can be modified to include a vinyl phosphonate at the 3′ end of the of the anti-sense strand of the dsRNA. The linker can be used to link the dsRNA to the FN3 domain. The linker can covalently attach, for example, to a cysteine residue on the FN3 domain that is there naturally or that has been substituted as described herein, and for example, in U.S. Pat. No. 10,196,446, which is hereby incorporated by reference in its entirety. Non-limiting examples of such modified strands of the dsRNA are illustrated in Table 4, below.

TABLE 4
Pairs with Linker and/or Vinyl Phosphonate

| | SEQ | | SEQ | | |
|---|---|---|---|---|---|
| siRNA | ID | | ID | | |
| Pair | NO: | Sense Strand 5′-3′ | NO: | Antisense Strand 5′-3′ | Linker |
| III | 70 | [mU][*mC][*fU][mC][fG][mU][fG] | 71 | [VP][*fU][*mU][fC][mA][fU][mU][fA] | Mal- |
| | | [mG][fC][mC][fU][mU][fA][mA][fU] | | [mA][fG][mG][fC][mC][fA][mC][fG] | C₂H₄C |
| | | [mG][fA][mA]-L | | [mA][fG][mA][*mU][*mU] | (O)(NH)- |
| | | | | | (CH₂)₆- |
| JJJ | 72 | [fC][*mU][*fG][mG][fG][mA][fG][fG] | 73 | [VP][*fU][*mC][mG][mA][mA][mU] | Mal- |
| | | [fA][mU][fG][mA][mA][mU][fU][mC] | | [mU][mC][mA][mU][mC][mC][fU] | C₂H₄C |
| | | [fG[mA][mA]-L | | [mC][mC][mC][mA][mG][*mU][*mU] | (O)(NH)- |
| | | | | | (CH₂)₆- |
| KKK | 74 | [fC][*mU][*fG][mG][fG][mA][fG][fG] | 75 | [VP][*fU][*mA][[G][mA][fU][fG][fC] | Mal- |
| | | [fA][mU][fG][mA][mA][mU][fU][mC] | | [mC][fA][mC][mC][mC][fA][mC][fC] | C₂H₄C |
| | | [fG[mA][mA]-L | | [mU][fU][mG][*fU][*mU] | (O)(NH)- |
| | | | | | (CH₂)₆- |
| LLL | 76 | [mG][*mC][*fG][mC][mG][mG][fA] | 77 | [VP][*fG][*mA][mA][mA][mU][mU] | Mal- |
| | | [fC][fC][mA][mA][fC][mA][mA][mU] | | [mG][mU][mU][mG][mG][mU][fC] | C₂H₄C |
| | | [mU][fU][mC][mA]-L | | [mC][mG][mC][mG][mC][*mU][*mU] | (O)(NH)- |
| | | | | | (CH₂)₆- |
| MMM | 78 | [mG][*mA][*fG][mC][mC][mA][fU] | 79 | [VP][*fG][*mC][mG][mU][mU][mG] | Mal- |
| | | [fC][fU][mU][mU][fG][mC][mA][mA] | | [mC][mA][mA][mA][mG][mA][fU] | C₂H₄C |
| | | [mC][fG][mC][mA]-L | | [mG][mG][mC][mU][mC][*mU][*mU] | (O)(NH)- |
| | | | | | (CH₂)₆- |
| NNN | 80 | [mA][*mG][*fC][mC][mA][mU][fC] | 81 | [VP][*fU][*mG][mC][mG][mU][mU] | Mal- |
| | | U[f][fU][mU][mG][fC][mA][mA][mC] | | [mG][mC][mA][mA][mA][mG][fA] | C₂H₄C |
| | | [mG][fC][mA][mA]-L | | [mU][mG][mG][mC][mU][*mU][*mU] | (O)(NH)- |
| | | | | | (CH₂)₆- |
| OOO | 82 | [mU][*mU][*fA][mC][mG][mG][fU] | 83 | [VP][*fA][*mG][mA][mA][mU][mG] | Mal- |
| | | [fA][fU][mC][mU][fA][mC][mA][mU] | | [mU][mA][mG][mA][mU][mA][fC] | C₂H₄C |
| | | [mU][fC][mU][mA]-L | | [mC][mG][mU][mA][mA][*mU][*mU] | (O)(NH)- |
| | | | | | (CH₂)₆- |
| PPP | 84 | [mC][*mC][*fC][mA][mC][mA][fU] | 85 | [VP][*fC][*mC][mU][mC][mG][mA] | Mal- |
| | | [fU][fU][mA][mC][fC][mU][mC][mG] | | [mG][mG][mU][mA][mA][mA][fU] | C₂H₄C |
| | | [mA][fG][mG][mA]-L | | [mG][mU][mG][mG][mG][*mU][*mU] | (O)(NH)- |
| | | | | | (CH₂)₆- |
| QQQ | 86 | [mU][*mC][*fU][mC][fG][mU][fG] | 87 | [VP][*fU][*mU][fC][mA][fU][mU] | Mal- |
| | | [mG][fC][mC][fU][mU][fA][mA][fU] | | [fA][mA][fG][mG][fC][mC][fA][mC] | C₂H₄C |
| | | [mG][mG][mA]-L | | [fG][mA][fG][mA][*mU][*mU] | (O)(NH)- |
| | | | | | (CH₂)₆- |
| RRR | 658 | [fC][*mA][*fA][mG][fG][mU][fG][fG] | 659 | [VP][mU][*fU][*mA][fG][mA][fU][fG] | Mal- |
| | | [fG][mU][fG][mG][mC][mA][fU][mC] | | [fC][mC][fA][mC][mC][mC][fA][mC] | C₂H₄C |
| | | [fU][mA][mA][idT]-L | | [fC][mU][fU][mG][*fU][*mU] | (O)(NH)- |
| | | | | | (CH₂)₆- |
| SSS | 660 | [fC][*mA][*fA][mG][fG][mU][[G][[G] | 661 | [VP][mU][*fU][*mA][fG][mA][fU][fG] | Mal- |
| | | [fG][mU][fG][mG][mC][mA][fU][mC] | | [fC][mC][fA][mC][mC][mC][fA][mC] | C₂H₄C |
| | | [fU][mA][mG][idT]-L | | [fC][mU][fU][mG][*fU][*mU] | (O)(NH)- |
| | | | | | (CH₂)₆- |
| TTT | 662 | [fC][*mA][*fA][mG][fG][mU][fG][fG] | 663 | [VP][mU][*fU][*mA][fG][mA][fU][fG] | Mal- |
| | | [fG][mU][fG][mG][mC][mA][fU][mC] | | [fC][mC][fA][mC][mC][mC][fA][mC] | C₂H₄C |
| | | [fU][mG][mA][idT]-L | | [fC][mU][fU][mG][*fU][*mU] | (O)(NH)- |
| | | | | | (CH₂)₆- |
| UUU | 664 | [fC][*mA][*fA][mG][fG][mU][fG][fG] | 665 | [VP][mU][*fU][*mA][fG][mA][fU][fG] | Mal- |
| | | [fG][mU][fG][mG][mC][mA][fU][mC] | | [fC][mC][fA][mC][mC][mC][fA][mC] | C₂H₄C |
| | | [fG][mA][mA][idT]-L | | [fC][mU][fU][mG][*fU][*mU] | (O)(NH)- |
| | | | | | (CH₂)₆- |
| VVV | 666 | [fC][*mA][*fA][mG][fG][mU][fG][fG] | 667 | [VP][mU][*fU][*mA][fG][mA][fU][fG] | Mal- |
| | | [fG][mU][fG][mG][mC][mA][fU][mA] | | [fC][mC][fA][mC][mC][mC][fA][mC] | C₂H₄C |
| | | [fU][mA][mA][idT]-L | | [fC][mU][fU][mG][*fU][*mU] | (O)(NH)- |
| | | | | | (CH₂)₆- |
| WWW | 668 | [fC][*mA][*fA][mG][fG][mU][fG][fG] | 669 | [VP][mU][*fU][*mA][fG][mA][fU][fG] | Mal- |
| | | [fG][mU][fG][mG][mC][mA][fG][mC] | | [fC][mC][fA][mC][mC][mC][fA][mC] | C₂H₄C |
| | | [fU][mA][mA][idT]-L | | [fC][mU][fU][mG][*fU][*mU] | (O)(NH)- |
| | | | | | (CH₂)₆- |
| XXX | 670 | [fC][*mA][*fA][mG][fG][mU][fG][G] | 671 | [VP][mU][*fU][*mA][fG][mA][fU][fG] | Mal- |
| | | [fG][mU][fG][mG][mC][mA][fG][mC | | [fC][mC][fA][mC][mC][mC][fA][mC] | C₂H₄C |
| | | ][fU][mA][mG][idT]-L | | [fC][mU][fU][mG][*fU][*mU] | (O)(NH)- |
| | | | | | (CH₂)₆- |
| YYY | 672 | [mG][*mA][*fG][mC][mC][mA][fU] | 673 | [VP][mU][*fG][*mC][mG][mU][mU] | Mal- |
| | | [fC][fU][mU][mU][fG][mC][mA][mA] | | [mG][mC][mA][mA][mA][mG][mA] | C₂H₄C |
| | | [mC][fG][mC][mA][idT]-L | | [fU][mG][mG][mC][mU][mC][*mU] | (O)(NH)- |
| | | | | [*mU] | (CH₂)₆- |
| ZZZ | 674 | [mG][*mA][*fG][mC][mC][mA][fU] | 675 | [VP][mU][*fG][*mC][mG][mU][mU] | Mal- |
| | | [fC][fU][mU][mU][fG][mC][mA][mA] | | [mG][mC][mA][mA][mA][mG][mA] | C₂H₄C |
| | | [mC][fG][mC][mG][idT]-L | | [fU][mG][mG][mC][mU][mC][*mU] | (O)(NH)- |
| | | | | [*mU] | (CH₂)₆- |
| AAAA | 676 | [mG][*mA][*fG][mC][mC][mA][fU] | 677 | [VP][mU][*fG][*mC][mG][mU][mU] | Mal- |
| | | [fC][fU][mU][mU][fG][mC][mA][mA] | | [mG][mC][mA][mA][mA][mG][mA] | C₂H₄C |
| | | m[C][fG][mA][mA][idT]-L | | [fU][mG][mG][mC][mU][mC][*mU] | (O)(NH)- |
| | | | | [*mU] | (CH₂)₆- |
| BBBB | 678 | [mG][*mA][*fG][mC][mC][mA][fU] | 679 | [VP][mU][*fG][*mC][mG][mU][mU] | Mal- |
| | | [fC][fU][mU][mU][fG][mC][mA][mA] | | [mG][mC][mA][mA][mA][mG][mA] | C₂H₄C |
| | | [mC][fA][mC][mA][idT]-L | | [fU][mG][mG][mC][mU][mC][*mU] | (O)(NH)- |
| | | | | [*mU] | (CH₂)₆- |
| CCCC | 680 | [mG][*mA][*fG][mC][mC][mA][fU] | 681 | [VP][mU][*fG][*mC][mG][mU][mU] | Mal- |
| | | [fC][fU][mU][mU][fG][mC][mA][mA] | | [mG][mC][mA][mA][mA][mG][mA] | C₂H₄C |
| | | [mA][fG][mC][mA][idT]-L | | [fU][mG][mG][mC][mU][mC][*mU] | (O)(NH)- |
| | | | | [*mU] | (CH₂)₆- |
| DDDD | 682 | [mG][*mA][*fG][mC][mC][mA][fU] | 683 | [VP][mU][*fG][*mC][mG][mU][mU] | Mal- |
| | | [fC][fU][mU][mU][fG][mC][mA][mG] | | [mG][mC][mA][mA][mA][mG][mA] | C₂H₄C |
| | | [mC][fG][mC][mA][idT]-L | | [fU][mG][mG][mC][mU][mC][*mU] | (O)(NH)- |
| | | | | [*mU] | (CH₂)₆- |
| EEEE | 684 | [mG][*mA][*fG][mC][mC][mA][fU] | 685 | [VP][mU][*fG][*mC][mG][mU][mU] | Mal- |
| | | [fC][fU][mU][mU][fG][mC][mA][mG] | | [mG][mC][mA][mA][mA][mG][mA] | C₂H₄C |
| | | [mC][fG][mC][mG][idT]-L | | [fU][mG][mG][mC][mU][mC][*mU] | (O)(NH)- |
| | | | | [*mU] | (CH₂)₆- |
| FFFF | 686 | [mU][*mU][*fA][mC][mG][mG][fU] | 687 | [VP][mU][*fA][*mG][mA][mA][mU] | Mal- |
| | | [fA][fU][mC][mU][fA][mC][mA][mU] | | [mG][mU][mA][mG][mA][mU][mA] | C₂H₄C |
| | | [mU][fC][mU][mA][idT]-L | | [fC][mC][mG][mU][mA][mA][*mU] | (O)(NH)- |
| | | | | [*mU] | (CH₂)₆- |
| GGGG | 688 | [mU][*mU][*fA][mC][mG][mG][fU] | 689 | [VP][mU][*fA][*mG][mA][mA][mU] | Mal- |
| | | [fA][fU][mC][mU][fA][mC][mA][mU] | | [mG][mU][mA][mG][mA][mU][mA] | C₂H₄C |
| | | [mU][fC][mU][mG][idT]-L | | [fC][mC][mG][mU][mA][mA][*mU] | (O)(NH)- |
| | | | | [*mU] | (CH₂)₆- |
| HHHH | 690 | [mU][*mU][*fA][mC][mG][mG][fU] | 691 | [VP][mU][*fA][*mG][mA][mA][mU] | Mal- |
| | | [fA][fU][mC][mU][fA][mC][mA][mU] | | [mG][mU][mA][mG][mA][mU][mA] | C₂H₄C |
| | | [mU][fC][mG][mA][idT]-L | | [fC][mC][mG][mU][mA][mA][*mU] | (O)(NH)- |
| | | | | [*mU] | (CH₂)₆- |
| IIII | 692 | [mU][*mU][*fA][mC][mG][mG][fU] | 693 | [VP][mU][*fA][*mG][mA][mA][mU] | Mal- |
| | | [fA][fU][mC][mU][fA][mC][mA][mU] | | [mG][mU][mA][mG][mA][mU][mA] | C₂H₄C |
| | | [mU][fG][mU][mA][idT]-L | | [fC][mC][mG][mU][mA][mA][*mU] | (O)(NH)- |
| | | | | [*mU] | (CH₂)₆- |
| JJJJ | 694 | [mU][*mU][*fA][mC][mG][mG][fU] | 695 | [VP][mU][*fA][*mG][mA][mA][mU] | Mal- |
| | | [fA][fU][mC][mU][fA][mC][mA][mU] | | [mG][mU][mA][mG][mA][mU][mA] | C₂H₄C |
| | | [mG][fC][mU][mA][idT]-L | | [fC][mC][mG][mU][mA][mA][*mU] | (O)(NH)- |
| | | | | [*mU] | (CH₂)₆- |
| KKKK | 696 | [mU][*mU][*fA][mC][mG][mG][fU] | 697 | [VP][mU][*fA][*mG][mA][mA][mU] | Mal- |
| | | [fA][fU][mC][mU][fA][mC][mA][mU] | | [mG][mU][mA][mG][mA][mU][mA] | C₂H₄C |
| | | [mG][fC][mU][mA][idT]-L | | [fC][mC][mG][mU][mA][mA][*mU] | (O)(NH)- |
| | | | | [*mU] | (CH₂)₆- |
| LLLL | 698 | [mU][*mU][*fA][mC][mG][mG][fU] | 699 | [VP][mU][*fA][*mG][mA][mA][mU] | Mal- |
| | | [fA][fU][mC][mU][fA][mC][mA][mG] | | [mG][mU][mA][mG][mA][mU][mA] | C₂H₄C |
| | | [mU][fC][mU][mA][idT]-L | | [fC][mC][mG][mU][mA][mA][*mU] | (O)(NH)- |
| | | | | [*mU] | (CH₂)₆- |
| MMMM | 700 | [mU][*mU][*fA][mC][mG][mG][fU] | 701 | [VP][mU][*fA][*mG][mA][mA][mU] | Mal- |
| | | [fA][fU][mC][mU][fA][mC][mA][mG] | | [mG][mU][mA][mG][mA][mU][mA] | C₂H₄C |
| | | [mU][fC][mU][mG][idT]-L | | [fC][mC][mG][mU][mA][mA][*mU] | (O)(NH)- |
| | | | | [*mU] | (CH₂)₆- |
| NNNN | 702 | [fC][*mA][*fA][mG][fG][mU][G][fG] | 703 | [VP][mU][*fU][*mA][fG][mA][fU][fG] | Mal- |
| | | [fG][mU][fG][mG][mC][mA][fU][mC] | | [fC][mC][fA][mC][mC][mC][fA][mC] | C₂H₄C |
| | | [fU][mA][mG][idT]-L | | [fC][mU][fU][mG][*fU][*mU] | (O)(NH)- |
| | | | | | (CH₂)₆- |
| OOOO | 706 | [mU][*mU][*fA][mC][mG][mG][fU] | 707 | [VP][mU][*fA][*mG][mA][mA][mU] | Mal- |
| | | [fA][fU][C][mU][fA][mC][mA][mU] | | [mG][mU][mA][mG][mA][mU][mA] | C₂H₄C |
| | | [mU][fC][mU][mA]-L | | [fC][mC][mG][mU][mA][mA][*mU] | (O)(NH)- |
| | | | | [*mU] | (CH₂)₆- |
| PPPP | 711 | [mU][*mG][*fG][mA][mC][mU][fU] | 712 | [VP][mU][*fU][*mG][mU][mC][mU] | Mal- |
| | | [fC][fA][mA][mC][fC][mU][mA][mG] | | [mA][mG][mG][mU][mU][mG][mA] | C₂H₄C |
| | | [mA][fC][mA][mA]-L | | [fA][mG][mU][mC][mC][mA][*mU] | (O)(NH)- |
| | | | | [*mU] | (CH₂)₆- |
| QQQQ | 793 | [mG][*mG][*fG][mU][mG][mA][fC] | 794 | [VP][mU][fA][*mG][*mA][mU][mA] | Mal- |
| | | [fA][fA][mC][mU][fA][mC][mU][mA] | | [mG][mU][mA][mG][mU][mU][mG] | C₂H₄C |
| | | [mU][fC][mU][mA] | | [fU][mC][mA][mC][mC][mC][*mU] | (O)(NH)- |
| | | | | [*mU] | (CH₂)₆- |
| RRRR | 795 | [fC][*mU][*fG][mG][fG][mA][fG] | 796 | [VP][mU][*fU][*mC][fG][mA][fA] | Mal- |
| | | [mG][fA][mU][fG][mA][fA][mU][fU] | | [mU][fU][mC][fA][mU][fC][mC][fU] | C₂H₄C |
| | | [mC][fG][mA][fA] | | [mC][[C][mC][fA][mG][*fU][*mU] | (O)(NH)- |
| | | | | | (CH₂)₆- |
| SSSS | 797 | [fC][*mA][*fA][mG][fG][mU][fG] | 798 | [VP][mU][*fU][*mA][fG][mA][fU] | Mal- |
| | | [mG][fG][mU][fG][mG][fC][mA][fU] | | [mG][fC][mC][fA][mC][fC][mC][fA] | C₂H₄C |
| | | [mC][fU][mA][[A] | | [mC][fC][mU][fU][mG][*fU][*mU] | (O)(NH)- |
| | | | | | (CH₂)₆- |
| TTTT | 799 | [fU][*mC][*fC][mG][fC][mA][fG] | 800 | [VP]mU][*fA][*mA][fU][mC][fA][mU] | Mal- |
| | | [mC][fC][mU][fG][mG][fA][mU][fG] | | [fC][mC][fA][mG][fG][mC][fU][mG] | C₂H₄C |
| | | [mA][fU][mU][fA] | | [fC][mG][fG][mA][*fU][*mU] | (O)(NH)- |
| | | | | | (CH₂)₆- |
| UUUU | 70 | [mU][*mC][*fU][mC][fG][mU][fG] | 1067 | [VP][fU][*mU][*fC][mA][fU][mU][fA] | Mal- |
| | | [mG][fC][mC][fU][mU][fA][mA][fU] | | [mA][fG][mG][fC][mC][fA][mC][fG] | C2H4C( |
| | | [mG][fA][mA]-L | | [mA][fG][mA][*mU][*mU] | O)(NH)- |
| | | | | | (CH2)6- |
| VVVV | 72 | [fC][*mU][*fG][mG][fG][mA][fG][fG] | 1068 | [VP][fU][*mC][*mG][mA][mA][mU] | Mal- |
| | | [fA][mU][fG][mA][mA][mU][fU][mC] | | [mU][mC][mA][mU][mC][mC][fU] | C₂H₄C |
| | | [fG[mA][mA]-L | | [mC][mC][mC][mA][mG][*mU][*mU] | (O)(NH)- |
| | | | | | (CH₂)₆- |
| WWWW | 74 | [fC][*mA][*fA][mG][fG][mU][fG][fG] | 1069 | [VP][fU][*mA][*fG][mA][fU][fG] | Mal- |
| | | [fG][mU][fG][mG][mC][mA][fU][mC | | [fC][mC][fA][mC][mC][mC][fA][mC] | C₂H₄C |
| | | ][fU][mA][mA]-L | | [fC][mU][fU][mG][*fU][*mU] | (O)(NH)- |
| | | | | | (CH₂)₆- |
| XXXX | 76 | [mG][*mC][*fG][mC][mG][mG][fA] | 1070 | [VP][fG][*mA][*mA][mA][mU][mU] | Mal- |
| | | [fC][fC][mA][mA][fC][mA][mA][mU] | | [mG][mU][mU][mG][mG][mU][fC] | C₂H₄C |
| | | [mU][fU][mC][mA]-L | | [mC][mG][mC][mG][mC][*mU][*mU] | (O)(NH)- |
| | | | | | (CH₂)₆- |
| YYYY | 78 | [mG][*mA][*fG][mC][mC][mA][fU] | 1071 | [VP][fG][*mC][*mG][mU][mU][mG] | Mal- |
| | | [fC][fU][mU][mU][fG][mC][mA][mA] | | [mC][mA][mA][mA][mG][mA][fU] | C₂H₄C |
| | | [mC][fG][mC][mA]-L | | [mG][mG][mC][mU][mC][*mU][*mU] | (O)(NH)- |
| | | | | | (CH₂)₆- |
| ZZZZ | 80 | [mA][*mG][*fC][mC][mA][mU][fC] | 1072 | [VP][fU][*mG][*mC][mG][mU][mU] | Mal- |
| | | [fU][fU][mU][mG][fC][mA][mA][mC] | | [mG][mC][mA][mA][mA][mG][fA] | C₂H₄C |
| | | [mG][fC][mA][mA]-L | | [mU][mG][mG][mC][mU][*mU][*mU] | (O)(NH)- |
| | | | | | (CH₂)₆- |
| AAAAA | 82 | [mU][*mU][*fA][mC][mG][mG][fU] | 1073 | [VP][fA][*mG][*mA][mA][mU][mG] | Mal- |
| | | [fA][fU][mC][mU][fA][mC][mA][mU] | | [mU][mA][mG][mA][mU][mA][fC] | C₂H₄C |
| | | [mU][fC][mU][mA]-L | | [mC][mG][mU][mA][mA][*mU][*mU] | (O)(NH)- |
| | | | | | (CH₂)₆- |
| BBBBB | 84 | [mC][*mC][*fC][mA][mC][mA][fU] | 1074 | [VP][fC][*mC][*mU][mC][mG][mA] | Mal- |
| | | [fU][fU][mA][mC][fC][mU][mC][mG] | | [mG][mG][mU][mA][mA][mA][fU] | C₂H₄C |
| | | [mA][fG][mG][mA]-L | | [mG][mU][mG][mG][mG][*mU][*mU] | (O)(NH)- |
| | | | | | (CH₂)₆- |
| CCCCC | 86 | [mU][*mC][*fU][mC][fG][mU][fG] | 1075 | [VP][fU][*mU][*fC][mA][fU][mU][fA] | Mal- |
| | | [mG][fC][mC][fU][mU][fA][mA][fU] | | [mA][fG][mG][fC][mC][fA][mC][fG] | C₂H₄C |
| | | [mG][mG][mA]-L | | [mA][fG][m][*mU][*mU] | (O)(NH)- |
| | | | | | (CH₂)₆- |

In some embodiments, the siRNA pairs of A to PPPP provided for above comprise a linker at the 3′ end of the sense strand. In some embodiments, the siRNA pairs of A to PPPP provided for above comprise a vinyl phosphonate at the 5′ end of the sense strand.

Abbreviations Key: (n/N=any nucleotide) mN=2′-O-methyl residues, fN=2′-F residues, *=phosphorothioate, (idT)=inverted Dt, (VP) 2′-O methyl vinlyl phosphonate uridine, BMPS=propyl maleimide,

Structure of the linkers (L) are as follows in Table 5, below.

TABLE 5
Representative examples of Linkers (L)

| Linker Structure | Linker Name |
|---|---|
| 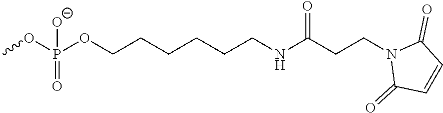 | Mal-C₂H₄C(O) (NH)—(CH₂)₆ |
| 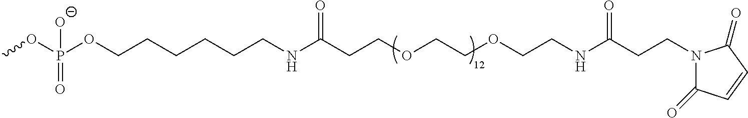 | Mal-(PEG)₁₂(NH)(CH₂)₆ |
| 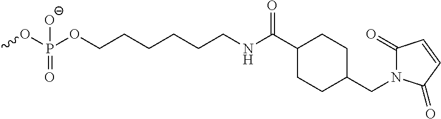 | Mal-NH—(CH₂)₆ or Aminohexyl linker-(CH₂)₆— |
| 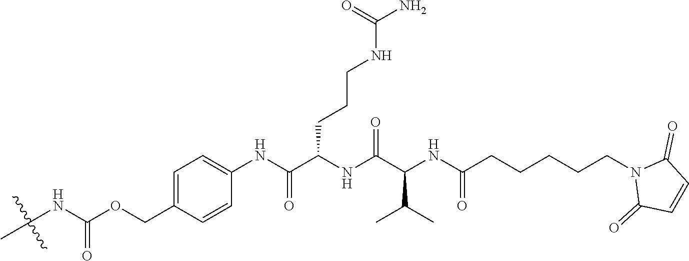 [SMILES: *NC(=O)OCc1ccc(NC(=O)[C@H](CCCNC(N)=O)NC(=O)[C@@H](NC(=O)CCCCCN2C(=O)C=CC2=O)C(C)C)cc1] | Val-Cit-PABA |

Other linkers can also be used, such as, linkers formed with click chemistry, amide coupling, reductive amination, oxime, enzymatic couplings such as transglutaminase and sortage conjugations. The linkers provided here are exemplary in nature and other linkers made with other such methods can also be used.

When connected to the siRNA, the structures, L-(X4) can be represented by the following formulas:

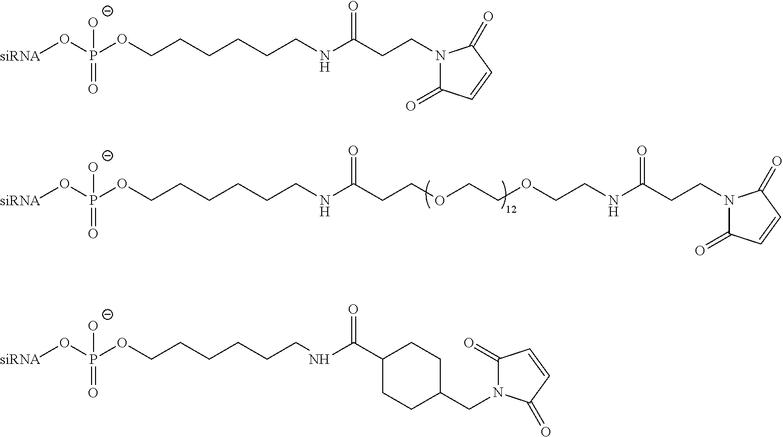

Although certain siRNA sequences are illustrated herein with certain modified nucleobases, the sequences without such modifications are also provided herein. That is, the sequence can comprise the sequences illustrated in the tables provided herein without any modifications. The unmodified siRNA sequences can still comprise, in some embodiments, a linker at the 5′ end of the of the sense strand of the dsRNA. In some embodiments, the nucleic acid molecules can be modified to include a vinyl phosphonate at the 5′ end of the of the anti-sense strand of the dsRNA. In some embodiments, the nucleic acid molecules can be modified to include a linker at the 3′ end of the of the sense strand of the dsRNA. In some embodiments, the nucleic acid molecules can be modified to include a vinyl phosphonate at the 3′ end of the of the anti-sense strand of the dsRNA. The linker can be as provided herein.

In some embodiments, the FN3 proteins comprise a polypeptide comprising a polypeptide that binds CD71 are provided. In some embodiments, the polypeptide comprises a FN3 domain that binds to CD71. In some embodiments, the polypeptide comprises a sequence of SEQ ID NOs: 273, 288-291, 301-310, 312-572, 592-599, or 708-710 are provided. In some embodiments, the polypeptide that binds CD71 comprises a sequence of SEQ ID NOs: 301-301, 310, 312-572, 592-599, or 708-710. The sequence of CD71 protein that the polypeptides can bind to can be, for example, SEQ ID Nos: 2 or 3. In some embodiments, the FN3 domain that binds to CD71 specifically binds to CD71.

In some embodiments, the FN3 domain that binds CD71 is based on Tencon sequence of SEQ ID NO: 1 or Tencon 27 sequence of SEQ ID NO: 4 (LPAPKNLVVSRVTEDSARLSWTAPDAAFDSFLIQYQESEKVGEAIVLTVPGSERSYDLT GLKPGTEYTVSIYGVKGGHRSNPLSAIFTT), optionally having substitutions at residues positions 11, 14, 17, 37, 46, 73, or 86 (residue numbering corresponding to SEQ ID NO:4).

In some embodiments, an isolated FN3 domain that binds CD71 comprises the amino acid sequence of SEQ ID NOs: 273, 288-291, 301-310, 312-572, 592-599, or 708-710.

In some embodiments, proteins comprising a polypeptide comprising an amino acid sequence of SEQ ID NO: 273. SEQ ID NO: 273 is a consensus sequence based on the sequences of SEQ ID NO: 288, SEQ ID NO: 289, SEQ ID NO: 290, and SEQ ID NO: 291. The sequence of SEQ ID NO: 273 is MLPAPKNLVVSRVTEDSARLSWTAPDAAFDSFX₁IX₂YX₃EX₄X5X₆X7GEAIX₈LX₉VPGSE RSYDLTGLKPGTEYX₁₀VX₁₁IX₁₂X₁₃VKGGX₁₄X₁₅SX₁₆PLX₁₇AX₁₈FTT wherein X₈, X₉, X₁₇, and X₁₈ are each, independently, any amino acid other than methionine or proline, and
X₁ is selected from D, F, Y, or H,
X₂ is selected from Y, G, A, or V,
X₃ is selected from I, T, L, A, or H,
X₄ is selected from S, Y or P,
X₅ is selected from Y, G, Q, or R,
X₆ is selected from G or P,
X₇ is selected from A, Y, P, D, or S,
X₁₀ is selected from W, N, S, or E,
X₁₁ is selected from L, Y, or G,
X₁₂ is selected from D, Q, H, or V,
X₁₃ is selected from G or S,
X₁₄ is selected from R, G, F, L, or D,
X₁₅ is selected from W, S, P, or L, and
X₁₆ is selected from T, V, M, or S.

In some embodiments:
X₁ is selected from D, F, Y, or H,
X₂ is selected from G, A, or V,
X₃ is selected from T, L, A, or H,
X₄ is selected from Y or P,
X₅ is selected from G, Q, or R,
X₆ is selected from G or P,
X₇ is selected from Y, P, D, or S,
X₁₀ is selected from W, N, S, or E,
X₁₁ is selected from L, Y, or G,
X₁₂ is selected from Q, H, or V,
X₁₃ is selected from G or S,
X₁₄ is selected from G, F, L, or D,
X₁₅ is selected from S, P, or L, and
X₁₆ is selected from V, M, or S.

In some embodiments, X1, X2, X₃, X₄, X₅, X₆, X₇, X₁₀, X₁₁, X₁₂, X₁₃, X₁₄, X₁₅, and X₁₆ are as shown in the sequence of SEQ ID NO: 288. In some embodiments, X1, X₂, X₃, X₄, X₅, X₆, X₇, X₁₀, X₁₁, X₁₂, X₁₃, X₁₄, X₁₅, and X₁₆ are as shown in the sequence of SEQ ID NO: 289. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 290. In some embodiments, X1, X₂, X₃, X₄, X₅, X₆, X₇, X₁₀, X₁₁, X₁₂, X₁₃, X₁₄, X₁₅, and X₁₆ are as shown in the sequence of SEQ ID NO: 291.

In some embodiments, X₈, X₉, X₁₇, and X₁₈ is, independently, alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, not alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, alanine. In some embodiments, X₈, X₉, X₁₇, and X₁₈ is, independently, arginine. In some embodiments, X₈, X₉, X₁₇, and X₁₈ is, independently asparagine. In some embodiments, X₈, X₉, X₁₇, and X₁₈ is, independently, aspartic acid. In some embodiments, X₈, X₉, X₁₇, and X₁₈ is, independently, cysteine. In some embodiments, X₈, X₉, X₁₇, and X₁₈ is, independently, glutamine. In some embodiments, X₈, X₉, X₁₇, and X₁₈ is, independently, glutamic acid. In some embodiments, X₈, X₉, X₁₇, and X₁₈ is, independently, glycine. In some embodiments, X₈, X₉, X₁₇, and X₁₈ is, independently, histidine. In some embodiments, X₈, X₉, X₁₇, and X₁₈ is, independently, isoleucine. In some embodiments, X₈, X₉, X₁₇, and X₁₈ is, independently, leucine. In some embodiments, X₈, X₉, X₁₇, and X₁₈ is, independently, lysine. In some embodiments, X₈, X₉, X₁₇, and X₁₈ is, independently, phenylalanine. In some embodiments, X₈, X₉, X₁₇, and X₁₈ is, independently serine. In some embodiments, X₈, X₉, X₁₇, and X₁₈ is, independently, threonine. In some embodiments, X₈, X₉, X₁₇, and X₁₈ is, independently, tryptophan. In some embodiments, X₈, X₉, X₁₇, and X₁₈ is, independently, tyrosine. In some embodiments, X₈, X₉, X₁₇, and X₁₈ is, independently valine.

In some embodiments, the sequence is set forth as shown in in the sequence of SEQ ID NO: 288, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 289, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 290, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 291, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, proteins comprising a polypeptide comprising an amino acid sequence that is at least 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to a sequence of SEQ ID NO: 273. In some embodiments, the protein is at least 85%, 86%, 87%, 88%, 89%, 90%, 91%, 92%, 93%, 94%, 95%, 96%, 97%, 98% or 99% identical to a sequence of SEQ ID NO: 273. In some embodiments, the protein is at least 90%, 91%, 92%, 93%, 94%, 95%, 96%, 97%, 98% or 99% identical to a sequence of SEQ ID NO: 273. In some embodiments, the protein is at least 95%, 96%, 97%, 98% or 99% identical to a sequence of SEQ ID NO: 273.

Percent identity can be determined using the default parameters to align two sequences using BlastP available through the NCBI website.

In some embodiments, fibronectin type III (FN3) domains that bind or specifically bind human CD71 protein (SEQ ID Nos: 2 or 5) are provided. As provided herein, the FN3 domains can bind to the CD71 protein. Also provided, even if not explicitly stated is that the domains can also specifically bind to the CD71 protein. Thus, for example, a FN3 domain that binds to CD71 would also encompass a FN3 domain protein that specifically binds to CD71. These molecules can be used, for example, in therapeutic and diagnostic applications and in imaging. In some embodiments, polynucleotides encoding the FN3 domains disclosed herein or complementary nucleic acids thereof, vectors, host cells, and methods of making and using them are provided.

In some embodiments, an isolated FN3 domain that binds or specifically binds CD71 is provided.

In some embodiments, the FN3 domain comprises two FN3 domains connected by a linker. The linker can be a flexible linker. The linker can be a short peptide sequence, such as those described herein. For example, the linker can be a G/S linker and the like.

In some embodiments, the FN3 domain comprising two FN3 domains connected by a linker, such as those provided for herein. Exemplary linker include, but are not limited to, (GS)₂, (SEQ ID NO: 720), (GGGS)₂ (SEQ ID NO: 721), (GGGGS)₁₋₅ (SEQ ID NO: 722), (AP)₁₋₂₀; (AP)₂ (SEQ ID NO: 723), (AP)₅ (SEQ ID NO: 724), (AP)₁₀ (SEQ ID NO: 725), (AP)₂₀ (SEQ ID NO: 726), A(EAAAK)₅AAA (SEQ ID NO: 727), or (EAAAK)₁₋₅ (SEQ ID NO: 728). In some embodiments, the linker comprises or is an amino acid sequence of:

(SEQ ID NO: 729)
EAAAKEAAAKEAAAKEAAAK;
(SEQ ID NO: 730)
GGGGSGGGGSGGGGGGGGS;
(SEQ ID NO: 731)
APAPAPAPAP;
or
(SEQ ID NO: 732)
EAAAK.

In some embodiments, the FN3 domain may bind CD71 with a dissociation constant (K_{D}) of less than about 1×10⁻⁷ M, for example less than about 1×10⁻⁸ M, less than about 1×10⁻⁹ M, less than about 1×10⁻¹⁰ M, less than about 1×10⁻¹¹ M, less than about 1×10⁻¹² M, or less than about 1×10⁻¹³ M as determined by surface plasmon resonance or the Kinexa method, as practiced by those of skill in the art. The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are made with standardized solutions of protein scaffold and antigen, and a standardized buffer, such as the buffers described herein.

In some embodiments, the FN3 domain may bind CD71 at least 5-fold above the signal obtained for a negative control in a standard solution ELISA assay.

In some embodiments, the FN3 domain that binds or specifically binds CD71 comprises an initiator methionine (Met) linked to the N-terminus of the molecule. In some embodiments, the FN3 domain that binds or specifically binds CD71 comprises a cysteine (Cys) linked to a C-terminus of the FN3 domain. The addition of the N-terminal Met and/or the C-terminal Cys may facilitate expression and/or conjugation to extend half-life and to provide other functions of molecules.

The FN3 domain can also contain cysteine substitutions, such as those that are described in U.S. Pat. No. 10,196,446, which is hereby incorporated by reference in its entirety. Briefly, in some embodiments, the polypeptides provided herein can comprise at least one cysteine substitution at a position selected from the group consisting of residues 6, 8, 10, 11, 14, 15, 16, 20, 30, 34, 38, 40, 41, 45, 47, 48, 53, 54, 59, 60, 62, 64, 70, 88, 89, 90, 91, and 93 of the FN3 domain based on SEQ ID NO: 1 of U.S. Pat. No. 10,196,446, or SEQ ID NO: 4, as set forth herein, and the equivalent positions in related FN3 domains.

The amino acid sequence of SEQ ID NO: 1 of U.S. Pat. No. 10,196,446 is:

LPAPKNLVVSEVTEDSLRLSWTAPDAAFDSFLIQYQESEKVGEAINLTV
PGSERSYDLTGLKPGTEYTVSIYGVKGGHRSNPLSAEFTT (herein
assigned, SEQ ID NO: 713)

In some embodiments, the substitution is at residue 6. In some embodiments, the substitution is at residue 8. In some embodiments, the substitution is at residue 10. In some embodiments, the substitution is at residue 11. In some embodiments, the substitution is at residue 14. In some embodiments, the substitution is at residue 15. In some embodiments, the substitution is at residue 16. In some embodiments, the substitution is at residue 20. In some embodiments, the substitution is at residue 30. In some embodiments, the substitution is at residue 34. In some embodiments, the substitution is at residue 38. In some embodiments, the substitution is at residue 40. In some embodiments, the substitution is at residue 41. In some embodiments, the substitution is at residue 45. In some embodiments, the substitution is at residue 47. In some embodiments, the substitution is at residue 48. In some embodiments, the substitution is at residue 53. In some embodiments, the substitution is at residue 54. In some embodiments, the substitution is at residue 59. In some embodiments, the substitution is at residue 60. In some embodiments, the substitution is at residue 62. In some embodiments, the substitution is at residue 64. In some embodiments, the substitution is at residue 70. In some embodiments, the substitution is at residue 88. In some embodiments, the substitution is at residue 89. In some embodiments, the substitution is at residue 90. In some embodiments, the substitution is at residue 91. In some embodiments, the substitution is at residue 93.

A cysteine substitution at a position in the domain or protein comprises a replacement of the existing amino acid residue with a cysteine residue. In some embodiments, instead of a substitution a cysteine is inserted into the sequence adjacent to the positions listed above. Other examples of cysteine modifications can be found in, for example, U.S. Patent Application Publication No. 20170362301, which is hereby incorporated by reference in its entirety. The alignment of the sequences can be performed using BlastP using the default parameters at, for example, the NCBI website.

In some embodiments, a cysteine residue is inserted at any position in the domain or protein.

In some embodiments, the FN3 domain that binds CD71 is internalized into a cell. In some embodiments, internalization of the FN3 domain may facilitate delivery of a detectable label or therapeutic into a cell. In some embodiments, internalization of the FN3 domain may facilitate delivery of a cytotoxic agent into a cell. The cytotoxic agent can act as a therapeutic agent. In some embodiments, internalization of the FN3 domain may facilitate the delivery of any detectable label, therapeutic, and/or cytotoxic agent disclosed herein into a cell. In some embodiments, internalization of the FN3 domain may facilitate delivery of a oligonucleotide into a cell. In some embodiments, the cell is a tumor cell. In some embodiments, the cell is a liver cell. In some embodiments, the cell is a muscle cell. In some embodiments, the cell is an immune cell. In some embodiments, the cell is a dendritic cell. In some embodiments, the cell is a cell of the central nervous system. In some embodiments, the cell is a heart cell.

In some embodiments, an isolated FN3 domain that binds CD71 comprises the amino acid sequence of SEQ ID NOs: 273, 288-291, 301-310, 312-572, 592-599, or 708-710.

In some embodiments, an isolated FN3 domain that binds CD71 comprises the amino acid sequence of SEQ ID NO: 301. In some embodiments, an isolated FN3 domain that binds CD71 comprises the amino acid sequence of SEQ ID NO: 302. In some embodiments, an isolated FN3 domain that binds CD71 comprises the amino acid sequence of SEQ ID NO: 303. In some embodiments, an isolated FN3 domain that binds CD71 comprises the amino acid sequence of SEQ ID NO: 304. In some embodiments, an isolated FN3 domain that binds CD71 comprises the amino acid sequence of SEQ ID NO: 305. In some embodiments, an isolated FN3 domain that binds CD71 comprises the amino acid sequence of SEQ ID NO: 306. In some embodiments, an isolated FN3 domain that binds CD71 comprises the amino acid sequence of SEQ ID NO: 307. In some embodiments, an isolated FN3 domain that binds CD71 comprises the amino acid sequence of SEQ ID NO: 310. In some embodiments, an isolated FN3 domain that binds CD71 comprises the amino acid sequence of SEQ ID NO: 312. In some embodiments, an isolated FN3 domain that binds CD71 comprises the amino acid sequence of SEQ ID NO: 313. In some embodiments, an isolated FN3 domain that binds CD71 comprises the amino acid sequence of SEQ ID NO: 314. In some embodiments, an isolated FN3 domain that binds CD71 comprises the amino acid sequence of SEQ ID NO: 315. In some embodiments, an isolated FN3 domain that binds CD71 comprises the amino acid sequence of SEQ ID NO: 316. In some embodiments, an isolated FN3 domain that binds CD71 comprises the amino acid sequence of SEQ ID NO: 317. In some embodiments, an isolated FN3 domain that binds CD71 comprises the amino acid sequence of SEQ ID NO: 318. In some embodiments, an isolated FN3 domain that binds CD71 comprises the amino acid sequence of SEQ ID NO: 319. In some embodiments, an isolated FN3 domain that binds CD71 comprises the amino acid sequence of SEQ ID NO: 320. In some embodiments, an isolated FN3 domain that binds CD71 comprises the amino acid sequence of SEQ ID NO: 321. In some embodiments, an isolated FN3 domain that binds CD71 comprises the amino acid sequence of SEQ ID NO: 322. In some embodiments, an isolated FN3 domain that binds CD71 comprises the amino acid sequence of SEQ ID NO: 323. In some embodiments, an isolated FN3 domain that binds CD71 comprises the amino acid sequence of SEQ ID NO: 324. In some embodiments, an isolated FN3 domain that binds CD71 comprises the amino acid sequence of SEQ ID NO: 325. In some embodiments, an isolated FN3 domain that binds CD71 comprises the amino acid sequence of SEQ ID NO: 326. In some embodiments, an isolated FN3 domain that binds CD71 comprises the amino acid sequence of SEQ ID NO: 327. In some embodiments, an isolated FN3 domain that binds CD71 comprises the amino acid sequence of SEQ ID NO: 328. In some embodiments, an isolated FN3 domain that binds CD71 comprises the amino acid sequence of SEQ ID NO: 329. In some embodiments, an isolated FN3 domain that binds CD71 comprises the amino acid sequence of SEQ ID NO: 330. In some embodiments, an isolated FN3 domain that binds CD71 comprises the amino acid sequence of SEQ ID NO: 331. In some embodiments, an isolated FN3 domain that binds CD71 comprises the amino acid sequence of SEQ ID NO: 332. In some embodiments, an isolated FN3 domain that binds CD71 comprises the amino acid sequence of SEQ ID NO: 333. In some embodiments, an isolated FN3 domain that binds CD71 comprises the amino acid sequence of SEQ ID NO: 334. In some embodiments, an isolated FN3 domain that binds CD71 comprises the amino acid sequence of SEQ ID NO: 335. In some embodiments, an isolated FN3 domain that binds CD71 comprises the amino acid sequence of SEQ ID NO: 336. In some embodiments, an isolated FN3 domain that binds CD71 comprises the amino acid sequence of SEQ ID NO: 337. In some embodiments, an isolated FN3 domain that binds CD71 comprises the amino acid sequence of SEQ ID NO: 338. In some embodiments, an isolated FN3 domain that binds CD71 comprises the amino acid sequence of SEQ ID NO: 339. In some embodiments, an isolated FN3 domain that binds CD71 comprises the amino acid sequence of SEQ ID NO: 340. In some embodiments, an isolated FN3 domain that binds CD71 comprises the amino acid sequence of SEQ ID NO: 341. In some embodiments, an isolated FN3 domain that binds CD71 comprises the amino acid sequence of SEQ ID NO: 342. In some embodiments, an isolated FN3 domain that binds CD71 comprises the amino acid sequence of SEQ ID NO: 343. In some embodiments, an isolated FN3 domain that binds CD71 comprises the amino acid sequence of SEQ ID NO: 344. In some embodiments, an isolated FN3 domain that binds CD71 comprises the amino acid sequence of SEQ ID NO: 345. In some embodiments, an isolated FN3 domain that binds CD71 comprises the amino acid sequence of SEQ ID NO: 346. In some embodiments, an isolated FN3 domain that binds CD71 comprises the amino acid sequence of SEQ ID NO: 347. In some embodiments, an isolated FN3 domain that binds CD71 comprises the amino acid sequence of SEQ ID NO: 348. In some embodiments, an isolated FN3 domain that binds CD71 comprises the amino acid sequence of SEQ ID NO: 349. In some embodiments, an isolated FN3 domain that binds CD71 comprises the amino acid sequence of SEQ ID NO: 350. In some embodiments, an isolated FN3 domain that binds CD71 comprises the amino acid sequence of SEQ ID NO: 351. In some embodiments, an isolated FN3 domain that binds CD71 comprises the amino acid sequence of SEQ ID NO: 352. In some embodiments, an isolated FN3 domain that binds CD71 comprises the amino acid sequence of SEQ ID NO: 353. In some embodiments, an isolated FN3 domain that binds CD71 comprises the amino acid sequence of SEQ ID NO: 354. In some embodiments, an isolated FN3 domain that binds CD71 comprises the amino acid sequence of SEQ ID NO: 355. In some embodiments, an isolated FN3 domain that binds CD71 comprises the amino acid sequence of SEQ ID NO: 356. In some embodiments, an isolated FN3 domain that binds CD71 comprises the amino acid sequence of SEQ ID NO: 357. In some embodiments, an isolated FN3 domain that binds CD71 comprises the amino acid sequence of SEQ ID NO: 358. In some embodiments, an isolated FN3 domain that binds CD71 comprises the amino acid sequence of SEQ ID NO: 359. In some embodiments, an isolated FN3 domain that binds CD71 comprises the amino acid sequence of SEQ ID NO: 360. In some embodiments, an isolated FN3 domain that binds CD71 comprises the amino acid sequence of SEQ ID NO: 361. In some embodiments, an isolated FN3 domain that binds CD71 comprises the amino acid sequence of SEQ ID NO: 362. In some embodiments, an isolated FN3 domain that binds CD71 comprises the amino acid sequence of SEQ ID NO: 363. In some embodiments, an isolated FN3 domain that binds CD71 comprises the amino acid sequence of SEQ ID NO: 364. In some embodiments, an isolated FN3 domain that binds CD71 comprises the amino acid sequence of SEQ ID NO: 365. In some embodiments, an isolated FN3 domain that binds CD71 comprises the amino acid sequence of SEQ ID NO: 366. In some embodiments, an isolated FN3 domain that binds CD71 comprises the amino acid sequence of SEQ ID NO: 367. In some embodiments, an isolated FN3 domain that binds CD71 comprises the amino acid sequence of SEQ ID NO: 368. In some embodiments, an isolated FN3 domain that binds CD71 comprises the amino acid sequence of SEQ ID NO: 369. In some embodiments, an isolated FN3 domain that binds CD71 comprises the amino acid sequence of SEQ ID NO: 370. In some embodiments, an isolated FN3 domain that binds CD71 comprises the amino acid sequence of SEQ ID NO: 371. In some embodiments, an isolated FN3 domain that binds CD71 comprises the amino acid sequence of SEQ ID NO: 372. In some embodiments, an isolated FN3 domain that binds CD71 comprises the amino acid sequence of SEQ ID NO: 373. In some embodiments, an isolated FN3 domain that binds CD71 comprises the amino acid sequence of SEQ ID NO: 374. In some embodiments, an isolated FN3 domain that binds CD71 comprises the amino acid sequence of SEQ ID NO: 375. In some embodiments, an isolated FN3 domain that binds CD71 comprises the amino acid sequence of SEQ ID NO: 376. In some embodiments, an isolated FN3 domain that binds CD71 comprises the amino acid sequence of SEQ ID NO: 377. In some embodiments, an isolated FN3 domain that binds CD71 comprises the amino acid sequence of SEQ ID NO: 378. In some embodiments, an isolated FN3 domain that binds CD71 comprises the amino acid sequence of SEQ ID NO: 379. In some embodiments, an isolated FN3 domain that binds CD71 comprises the amino acid sequence of SEQ ID NO: 380. In some embodiments, an isolated FN3 domain that binds CD71 comprises the amino acid sequence of SEQ ID NO: 381. In some embodiments, an isolated FN3 domain that binds CD71 comprises the amino acid sequence of SEQ ID NO: 382. In some embodiments, an isolated FN3 domain that binds CD71 comprises the amino acid sequence of SEQ ID NO: 383. In some embodiments, an isolated FN3 domain that binds CD71 comprises the amino acid sequence of SEQ ID NO: 384. In some embodiments, an isolated FN3 domain that binds CD71 comprises the amino acid sequence of SEQ ID NO: 385. In some embodiments, an isolated FN3 domain that binds CD71 comprises the amino acid sequence of SEQ ID NO: 386. In some embodiments, an isolated FN3 domain that binds CD71 comprises the amino acid sequence of SEQ ID NO: 387. In some embodiments, an isolated FN3 domain that binds CD71 comprises the amino acid sequence of SEQ ID NO: 388. In some embodiments, an isolated FN3 domain that binds CD71 comprises the amino acid sequence of SEQ ID NO: 389. In some embodiments, an isolated FN3 domain that binds CD71 comprises the amino acid sequence of SEQ ID NO: 390. In some embodiments, an isolated FN3 domain that binds CD71 comprises the amino acid sequence of SEQ ID NO: 391. In some embodiments, an isolated FN3 domain that binds CD71 comprises the amino acid sequence of SEQ ID NO: 392. In some embodiments, an isolated FN3 domain that binds CD71 comprises the amino acid sequence of SEQ ID NO: 393. In some embodiments, an isolated FN3 domain that binds CD71 comprises the amino acid sequence of SEQ ID NO: 394. In some embodiments, an isolated FN3 domain that binds CD71 comprises the amino acid sequence of SEQ ID NO: 395. In some embodiments, an isolated FN3 domain that binds CD71 comprises the amino acid sequence of SEQ ID NO: 396. In some embodiments, an isolated FN3 domain that binds CD71 comprises the amino acid sequence of SEQ ID NO: 397. In some embodiments, an isolated FN3 domain that binds CD71 comprises the amino acid sequence of SEQ ID NO: 398. In some embodiments, an isolated FN3 domain that binds CD71 comprises the amino acid sequence of SEQ ID NO: 399. In some embodiments, an isolated FN3 domain that binds CD71 comprises the amino acid sequence of SEQ ID NO: 400. In some embodiments, an isolated FN3 domain that binds CD71 comprises the amino acid sequence of SEQ ID NO: 401. In some embodiments, an isolated FN3 domain that binds CD71 comprises the amino acid sequence of SEQ ID NO: 402. In some embodiments, an isolated FN3 domain that binds CD71 comprises the amino acid sequence of SEQ ID NO: 403. In some embodiments, an isolated FN3 domain that binds CD71 comprises the amino acid sequence of SEQ ID NO: 404. In some embodiments, an isolated FN3 domain that binds CD71 comprises the amino acid sequence of SEQ ID NO: 405. In some embodiments, an isolated FN3 domain that binds CD71 comprises the amino acid sequence of SEQ ID NO: 406. In some embodiments, an isolated FN3 domain that binds CD71 comprises the amino acid sequence of SEQ ID NO: 407. In some embodiments, an isolated FN3 domain that binds CD71 comprises the amino acid sequence of SEQ ID NO: 408. In some embodiments, an isolated FN3 domain that binds CD71 comprises the amino acid sequence of SEQ ID NO: 409. In some embodiments, an isolated FN3 domain that binds CD71 comprises the amino acid sequence of SEQ ID NO: 410. In some embodiments, an isolated FN3 domain that binds CD71 comprises the amino acid sequence of SEQ ID NO: 411. In some embodiments, an isolated FN3 domain that binds CD71 comprises the amino acid sequence of SEQ ID NO: 412. In some embodiments, an isolated FN3 domain that binds CD71 comprises the amino acid sequence of SEQ ID NO: 413. In some embodiments, an isolated FN3 domain that binds CD71 comprises the amino acid sequence of SEQ ID NO: 414. In some embodiments, an isolated FN3 domain that binds CD71 comprises the amino acid sequence of SEQ ID NO: 415. In some embodiments, an isolated FN3 domain that binds CD71 comprises the amino acid sequence of SEQ ID NO: 416. In some embodiments, an isolated FN3 domain that binds CD71 comprises the amino acid sequence of SEQ ID NO: 417. In some embodiments, an isolated FN3 domain that binds CD71 comprises the amino acid sequence of SEQ ID NO: 418. In some embodiments, an isolated FN3 domain that binds CD71 comprises the amino acid sequence of SEQ ID NO: 419. In some embodiments, an isolated FN3 domain that binds CD71 comprises the amino acid sequence of SEQ ID NO: 420. In some embodiments, an isolated FN3 domain that binds CD71 comprises the amino acid sequence of SEQ ID NO: 421. In some embodiments, an isolated FN3 domain that binds CD71 comprises the amino acid sequence of SEQ ID NO: 422. In some embodiments, an isolated FN3 domain that binds CD71 comprises the amino acid sequence of SEQ ID NO: 423. In some embodiments, an isolated FN3 domain that binds CD71 comprises the amino acid sequence of SEQ ID NO: 424. In some embodiments, an isolated FN3 domain that binds CD71 comprises the amino acid sequence of SEQ ID NO: 425. In some embodiments, an isolated FN3 domain that binds CD71 comprises the amino acid sequence of SEQ ID NO: 426. In some embodiments, an isolated FN3 domain that binds CD71 comprises the amino acid sequence of SEQ ID NO: 427. In some embodiments, an isolated FN3 domain that binds CD71 comprises the amino acid sequence of SEQ ID NO: 428. In some embodiments, an isolated FN3 domain that binds CD71 comprises the amino acid sequence of SEQ ID NO: 429. In some embodiments, an isolated FN3 domain that binds CD71 comprises the amino acid sequence of SEQ ID NO: 430. In some embodiments, an isolated FN3 domain that binds CD71 comprises the amino acid sequence of SEQ ID NO: 431. In some embodiments, an isolated FN3 domain that binds CD71 comprises the amino acid sequence of SEQ ID NO: 432. In some embodiments, an isolated FN3 domain that binds CD71 comprises the amino acid sequence of SEQ ID NO: 433. In some embodiments, an isolated FN3 domain that binds CD71 comprises the amino acid sequence of SEQ ID NO: 434. In some embodiments, an isolated FN3 domain that binds CD71 comprises the amino acid sequence of SEQ ID NO: 435. In some embodiments, an isolated FN3 domain that binds CD71 comprises the amino acid sequence of SEQ ID NO: 436. In some embodiments, an isolated FN3 domain that binds CD71 comprises the amino acid sequence of SEQ ID NO: 437. In some embodiments, an isolated FN3 domain that binds CD71 comprises the amino acid sequence of SEQ ID NO: 438. In some embodiments, an isolated FN3 domain that binds CD71 comprises the amino acid sequence of SEQ ID NO: 439. In some embodiments, an isolated FN3 domain that binds CD71 comprises the amino acid sequence of SEQ ID NO: 440. In some embodiments, an isolated FN3 domain that binds CD71 comprises the amino acid sequence of SEQ ID NO: 441. In some embodiments, an isolated FN3 domain that binds CD71 comprises the amino acid sequence of SEQ ID NO: 442. In some embodiments, an isolated FN3 domain that binds CD71 comprises the amino acid sequence of SEQ ID NO: 443. In some embodiments, an isolated FN3 domain that binds CD71 comprises the amino acid sequence of SEQ ID NO: 444. In some embodiments, an isolated FN3 domain that binds CD71 comprises the amino acid sequence of SEQ ID NO: 445. In some embodiments, an isolated FN3 domain that binds CD71 comprises the amino acid sequence of SEQ ID NO: 446. In some embodiments, an isolated FN3 domain that binds CD71 comprises the amino acid sequence of SEQ ID NO: 447. In some embodiments, an isolated FN3 domain that binds CD71 comprises the amino acid sequence of SEQ ID NO: 448. In some embodiments, an isolated FN3 domain that binds CD71 comprises the amino acid sequence of SEQ ID NO: 449. In some embodiments, an isolated FN3 domain that binds CD71 comprises the amino acid sequence of SEQ ID NO: 450. In some embodiments, an isolated FN3 domain that binds CD71 comprises the amino acid sequence of SEQ ID NO: 451. In some embodiments, an isolated FN3 domain that binds CD71 comprises the amino acid sequence of SEQ ID NO: 452. In some embodiments, an isolated FN3 domain that binds CD71 comprises the amino acid sequence of SEQ ID NO: 453. In some embodiments, an isolated FN3 domain that binds CD71 comprises the amino acid sequence of SEQ ID NO: 454. In some embodiments, an isolated FN3 domain that binds CD71 comprises the amino acid sequence of SEQ ID NO: 455. In some embodiments, an isolated FN3 domain that binds CD71 comprises the amino acid sequence of SEQ ID NO: 456. In some embodiments, an isolated FN3 domain that binds CD71 comprises the amino acid sequence of SEQ ID NO: 457. In some embodiments, an isolated FN3 domain that binds CD71 comprises the amino acid sequence of SEQ ID NO: 458. In some embodiments, an isolated FN3 domain that binds CD71 comprises the amino acid sequence of SEQ ID NO: 459. In some embodiments, an isolated FN3 domain that binds CD71 comprises the amino acid sequence of SEQ ID NO: 460. In some embodiments, an isolated FN3 domain that binds CD71 comprises the amino acid sequence of SEQ ID NO: 461. In some embodiments, an isolated FN3 domain that binds CD71 comprises the amino acid sequence of SEQ ID NO: 462. In some embodiments, an isolated FN3 domain that binds CD71 comprises the amino acid sequence of SEQ ID NO: 463. In some embodiments, an isolated FN3 domain that binds CD71 comprises the amino acid sequence of SEQ ID NO: 464. In some embodiments, an isolated FN3 domain that binds CD71 comprises the amino acid sequence of SEQ ID NO: 465. In some embodiments, an isolated FN3 domain that binds CD71 comprises the amino acid sequence of SEQ ID NO: 466. In some embodiments, an isolated FN3 domain that binds CD71 comprises the amino acid sequence of SEQ ID NO: 467. In some embodiments, an isolated FN3 domain that binds CD71 comprises the amino acid sequence of SEQ ID NO: 468. In some embodiments, an isolated FN3 domain that binds CD71 comprises the amino acid sequence of SEQ ID NO: 469. In some embodiments, an isolated FN3 domain that binds CD71 comprises the amino acid sequence of SEQ ID NO: 470. In some embodiments, an isolated FN3 domain that binds CD71 comprises the amino acid sequence of SEQ ID NO: 471. In some embodiments, an isolated FN3 domain that binds CD71 comprises the amino acid sequence of SEQ ID NO: 472. In some embodiments, an isolated FN3 domain that binds CD71 comprises the amino acid sequence of SEQ ID NO: 473. In some embodiments, an isolated FN3 domain that binds CD71 comprises the amino acid sequence of SEQ ID NO: 474. In some embodiments, an isolated FN3 domain that binds CD71 comprises the amino acid sequence of SEQ ID NO: 475. In some embodiments, an isolated FN3 domain that binds CD71 comprises the amino acid sequence of SEQ ID NO: 476. In some embodiments, an isolated FN3 domain that binds CD71 comprises the amino acid sequence of SEQ ID NO: 477. In some embodiments, an isolated FN3 domain that binds CD71 comprises the amino acid sequence of SEQ ID NO: 478. In some embodiments, an isolated FN3 domain that binds CD71 comprises the amino acid sequence of SEQ ID NO: 479. In some embodiments, an isolated FN3 domain that binds CD71 comprises the amino acid sequence of SEQ ID NO: 480. In some embodiments, an isolated FN3 domain that binds CD71 comprises the amino acid sequence of SEQ ID NO: 481. In some embodiments, an isolated FN3 domain that binds CD71 comprises the amino acid sequence of SEQ ID NO: 482. In some embodiments, an isolated FN3 domain that binds CD71 comprises the amino acid sequence of SEQ ID NO: 483. In some embodiments, an isolated FN3 domain that binds CD71 comprises the amino acid sequence of SEQ ID NO: 484. In some embodiments, an isolated FN3 domain that binds CD71 comprises the amino acid sequence of SEQ ID NO: 485. In some embodiments, an isolated FN3 domain that binds CD71 comprises the amino acid sequence of SEQ ID NO: 486. In some embodiments, an isolated FN3 domain that binds CD71 comprises the amino acid sequence of SEQ ID NO: 487. In some embodiments, an isolated FN3 domain that binds CD71 comprises the amino acid sequence of SEQ ID NO: 488. In some embodiments, an isolated FN3 domain that binds CD71 comprises the amino acid sequence of SEQ ID NO: 489. In some embodiments, an isolated FN3 domain that binds CD71 comprises the amino acid sequence of SEQ ID NO: 490. In some embodiments, an isolated FN3 domain that binds CD71 comprises the amino acid sequence of SEQ ID NO: 491. In some embodiments, an isolated FN3 domain that binds CD71 comprises the amino acid sequence of SEQ ID NO: 492. In some embodiments, an isolated FN3 domain that binds CD71 comprises the amino acid sequence of SEQ ID NO: 493. In some embodiments, an isolated FN3 domain that binds CD71 comprises the amino acid sequence of SEQ ID NO: 494. In some embodiments, an isolated FN3 domain that binds CD71 comprises the amino acid sequence of SEQ ID NO: 495. In some embodiments, an isolated FN3 domain that binds CD71 comprises the amino acid sequence of SEQ ID NO: 496. In some embodiments, an isolated FN3 domain that binds CD71 comprises the amino acid sequence of SEQ ID NO: 497. In some embodiments, an isolated FN3 domain that binds CD71 comprises the amino acid sequence of SEQ ID NO: 498. In some embodiments, an isolated FN3 domain that binds CD71 comprises the amino acid sequence of SEQ ID NO: 499. In some embodiments, an isolated FN3 domain that binds CD71 comprises the amino acid sequence of SEQ ID NO: 500. In some embodiments, an isolated FN3 domain that binds CD71 comprises the amino acid sequence of SEQ ID NO: 501. In some embodiments, an isolated FN3 domain that binds CD71 comprises the amino acid sequence of SEQ ID NO: 502. In some embodiments, an isolated FN3 domain that binds CD71 comprises the amino acid sequence of SEQ ID NO: 503. In some embodiments, an isolated FN3 domain that binds CD71 comprises the amino acid sequence of SEQ ID NO: 504. In some embodiments, an isolated FN3 domain that binds CD71 comprises the amino acid sequence of SEQ ID NO: 505. In some embodiments, an isolated FN3 domain that binds CD71 comprises the amino acid sequence of SEQ ID NO: 506. In some embodiments, an isolated FN3 domain that binds CD71 comprises the amino acid sequence of SEQ ID NO: 507. In some embodiments, an isolated FN3 domain that binds CD71 comprises the amino acid sequence of SEQ ID NO: 508. In some embodiments, an isolated FN3 domain that binds CD71 comprises the amino acid sequence of SEQ ID NO: 509. In some embodiments, an isolated FN3 domain that binds CD71 comprises the amino acid sequence of SEQ ID NO: 510. In some embodiments, an isolated FN3 domain that binds CD71 comprises the amino acid sequence of SEQ ID NO: 511. In some embodiments, an isolated FN3 domain that binds CD71 comprises the amino acid sequence of SEQ ID NO: 512. In some embodiments, an isolated FN3 domain that binds CD71 comprises the amino acid sequence of SEQ ID NO: 513. In some embodiments, an isolated FN3 domain that binds CD71 comprises the amino acid sequence of SEQ ID NO: 514. In some embodiments, an isolated FN3 domain that binds CD71 comprises the amino acid sequence of SEQ ID NO: 515. In some embodiments, an isolated FN3 domain that binds CD71 comprises the amino acid sequence of SEQ ID NO: 516. In some embodiments, an isolated FN3 domain that binds CD71 comprises the amino acid sequence of SEQ ID NO: 517. In some embodiments, an isolated FN3 domain that binds CD71 comprises the amino acid sequence of SEQ ID NO: 518. In some embodiments, an isolated FN3 domain that binds CD71 comprises the amino acid sequence of SEQ ID NO: 519. In some embodiments, an isolated FN3 domain that binds CD71 comprises the amino acid sequence of SEQ ID NO: 521. In some embodiments, an isolated FN3 domain that binds CD71 comprises the amino acid sequence of SEQ ID NO: 522. In some embodiments, an isolated FN3 domain that binds CD71 comprises the amino acid sequence of SEQ ID NO: 523. In some embodiments, an isolated FN3 domain that binds CD71 comprises the amino acid sequence of SEQ ID NO: 524. In some embodiments, an isolated FN3 domain that binds CD71 comprises the amino acid sequence of SEQ ID NO: 525. In some embodiments, an isolated FN3 domain that binds CD71 comprises the amino acid sequence of SEQ ID NO: 526. In some embodiments, an isolated FN3 domain that binds CD71 comprises the amino acid sequence of SEQ ID NO: 527. In some embodiments, an isolated FN3 domain that binds CD71 comprises the amino acid sequence of SEQ ID NO: 528. In some embodiments, an isolated FN3 domain that binds CD71 comprises the amino acid sequence of SEQ ID NO: 529. In some embodiments, an isolated FN3 domain that binds CD71 comprises the amino acid sequence of SEQ ID NO: 530. In some embodiments, an isolated FN3 domain that binds CD71 comprises the amino acid sequence of SEQ ID NO: 531. In some embodiments, an isolated FN3 domain that binds CD71 comprises the amino acid sequence of SEQ ID NO: 532. In some embodiments, an isolated FN3 domain that binds CD71 comprises the amino acid sequence of SEQ ID NO: 533. In some embodiments, an isolated FN3 domain that binds CD71 comprises the amino acid sequence of SEQ ID NO: 534. In some embodiments, an isolated FN3 domain that binds CD71 comprises the amino acid sequence of SEQ ID NO: 535. In some embodiments, an isolated FN3 domain that binds CD71 comprises the amino acid sequence of SEQ ID NO: 536. In some embodiments, an isolated FN3 domain that binds CD71 comprises the amino acid sequence of SEQ ID NO: 537. In some embodiments, an isolated FN3 domain that binds CD71 comprises the amino acid sequence of SEQ ID NO: 538. In some embodiments, an isolated FN3 domain that binds CD71 comprises the amino acid sequence of SEQ ID NO: 539. In some embodiments, an isolated FN3 domain that binds CD71 comprises the amino acid sequence of SEQ ID NO: 540. In some embodiments, an isolated FN3 domain that binds CD71 comprises the amino acid sequence of SEQ ID NO: 541. In some embodiments, an isolated FN3 domain that binds CD71 comprises the amino acid sequence of SEQ ID NO: 542. In some embodiments, an isolated FN3 domain that binds CD71 comprises the amino acid sequence of SEQ ID NO: 543. In some embodiments, an isolated FN3 domain that binds CD71 comprises the amino acid sequence of SEQ ID NO: 544. In some embodiments, an isolated FN3 domain that binds CD71 comprises the amino acid sequence of SEQ ID NO: 545. In some embodiments, an isolated FN3 domain that binds CD71 comprises the amino acid sequence of SEQ ID NO: 546. In some embodiments, an isolated FN3 domain that binds CD71 comprises the amino acid sequence of SEQ ID NO: 547. In some embodiments, an isolated FN3 domain that binds CD71 comprises the amino acid sequence of SEQ ID NO: 548. In some embodiments, an isolated FN3 domain that binds CD71 comprises the amino acid sequence of SEQ ID NO: 549. In some embodiments, an isolated FN3 domain that binds CD71 comprises the amino acid sequence of SEQ ID NO: 550. In some embodiments, an isolated FN3 domain that binds CD71 comprises the amino acid sequence of SEQ ID NO: 551. In some embodiments, an isolated FN3 domain that binds CD71 comprises the amino acid sequence of SEQ ID NO: 552. In some embodiments, an isolated FN3 domain that binds CD71 comprises the amino acid sequence of SEQ ID NO: 553. In some embodiments, an isolated FN3 domain that binds CD71 comprises the amino acid sequence of SEQ ID NO: 554. In some embodiments, an isolated FN3 domain that binds CD71 comprises the amino acid sequence of SEQ ID NO: 555. In some embodiments, an isolated FN3 domain that binds CD71 comprises the amino acid sequence of SEQ ID NO: 556. In some embodiments, an isolated FN3 domain that binds CD71 comprises the amino acid sequence of SEQ ID NO: 557. In some embodiments, an isolated FN3 domain that binds CD71 comprises the amino acid sequence of SEQ ID NO: 558. In some embodiments, an isolated FN3 domain that binds CD71 comprises the amino acid sequence of SEQ ID NO: 559. In some embodiments, an isolated FN3 domain that binds CD71 comprises the amino acid sequence of SEQ ID NO: 560. In some embodiments, an isolated FN3 domain that binds CD71 comprises the amino acid sequence of SEQ ID NO: 561. In some embodiments, an isolated FN3 domain that binds CD71 comprises the amino acid sequence of SEQ ID NO: 562. In some embodiments, an isolated FN3 domain that binds CD71 comprises the amino acid sequence of SEQ ID NO: 563. In some embodiments, an isolated FN3 domain that binds CD71 comprises the amino acid sequence of SEQ ID NO: 564. In some embodiments, an isolated FN3 domain that binds CD71 comprises the amino acid sequence of SEQ ID NO: 565. In some embodiments, an isolated FN3 domain that binds CD71 comprises the amino acid sequence of SEQ ID NO: 566. In some embodiments, an isolated FN3 domain that binds CD71 comprises the amino acid sequence of SEQ ID NO: 567. In some embodiments, an isolated FN3 domain that binds CD71 comprises the amino acid sequence of SEQ ID NO: 568. In some embodiments, an isolated FN3 domain that binds CD71 comprises the amino acid sequence of SEQ ID NO: 569. In some embodiments, an isolated FN3 domain that binds CD71 comprises the amino acid sequence of SEQ ID NO: 570. In some embodiments, an isolated FN3 domain that binds CD71 comprises the amino acid sequence of SEQ ID NO: 571. In some embodiments, an isolated FN3 domain that binds CD71 comprises the amino acid sequence of SEQ ID NO: 572. In some embodiments, an isolated FN3 domain that binds CD71 comprises the amino acid sequence of SEQ ID NO: 708. In some embodiments, an isolated FN3 domain that binds CD71 comprises the amino acid sequence of SEQ ID NO: 709. In some embodiments, an isolated FN3 domain that binds CD71 comprises the amino acid sequence of SEQ ID NO: 710.

In some embodiments, the isolated FN3 domain that binds CD71 comprises an initiator methionine (Met) linked to the N-terminus of the molecule.

In some embodiments, the isolated FN3 domain that binds CD71 comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to one of the amino acid sequences of SEQ ID NOs: 273, 288-291, 301-310, 312-572, 592-599, or 708-710. Percent identity can be determined using the default parameters to align two sequences using BlastP available through the NCBI website. The sequences of the FN3 domains that bind to CD71 can be found, for example, in Table 6, below.

TABLE 6
CD71-binding FN3 Domain Sequences

| SEQ ID | |
|---|---|
| NO: | Amino Acid sequence of FN3 domains that bind to CD71 |
| 301 | MLPAPKNLVVSRVTEDSARLSWTAPDAAFDSFPITYIEAVVLGEAIVLTVPGSERSYDLTGLKP |
| | GTEYPVGISGVKGGHNSMPLSAIFTT |
| 302 | MLPAPKNLVVSEVTEDSARLSWQGVARAFDSFMINYSELFWMGEAIVLTVPGSERSYDLTGL |
| | KPGTEYVVRIKGVKGGKGSWPLHAHFTT |
| 303 | MLPAPKNLVVSRVTEDSARLSWTAPDAAFDSFNIEYAETRWYGEAIVLTVPGSERSYDLTGLK |
| | PGTEYVVPIDGVKGGIASKPLSAIFTT |
| 304 | MLPAPKNLVVSEVTEDSARLSWQGVARAFDSFLITYRDQIFAGEVIVLTVPGSERSYDLTGLKP |
| | GTEYPVWIQGVKGGSPSAPLSAESTT |
| 305 | MLPAPKNLVVSEVTEDSARLSWQGVARAFDSFLITYREQIFAGEVIVLTVPGSERSYDLTGLKP |
| | GTEYWVYIWGVKGGKPSFPLRAGFTT |
| 306 | MLPAPKNLVVSRVTEDSARLSWTAPDAAFDSFPIIYMETFSRGEAIVLTVPGSERSYDLTGLKP |
| | GTEYRVPIGGVKGGSSSCPLSAIFTT |
| 307 | MLPAPKNLVVSDVTEDSARLSWQGVARAFDSFLITYREQIFAGEVIVLTVPGSERSYDLTGLKP |
| | GTEYPVWIQGVKGGSPSAPLSAEFTT |
| 310 | MLPAPKNLVVSRVTEDSARLSWTAPDAAFDSFKIAYIETATRGEAIVLTVPGSERSYDLTGLKP |
| | GTEYVVPIPGVKGGNTSSPLSAIFTT |
| 312 | MLPAPKNLVVSRVTEDSARLSWTAPDAAFDSFFIPYAEPSPTGEAIVLTVPGSERSYDLTGLKP |
| | GTEYSVLIHGVKGGHLSDPLSAISTT |
| 313 | MLPAPKNLVVSRVTEDSARLSWTAPDAAFDSFLIAYPEDGFRGEAIVLTVPGSERSYDLTGLK |
| | PGTEYPVPILGVKGGGGSGPLSAIFTT |
| 314 | MLPAPKNLVVSRVTEDSARLSWTAPDAAFDSFPIYYVENVVWGEAIVLTVPGSERSYDLTGLK |
| | PGTEYWEVIIGVKGGQCSRPLSAIFTT |
| 315 | MLPAPKNLVVSRVTEDSARLSWQGVARAFDSFLITYREQIFAGEVIVLTVPGSERSYDLTGLKP |
| | GTECPVWIQGVKGGSPSAPLSAEFTT |
| 316 | MLPAPKNLVVSRVTEDSARLSWTAPDAAFDSFGIAYREFRPSGEAIVLTVPGSERSYDLTVET |
| | GYRNEVVICGVKGGPWSGPLSAIFTT |
| 317 | MLPAPKNLVVSRVTEDSARLSWTAPDAAFDSFPILYTECVYRGEAIVLTVPGSERSYDLTGLKP |
| | GTEYHVPITGVKGGGGSWPLSAIFTT |
| 318 | MLPAPKNLVVSRVTEDSARLSWTAPDAAFDSFNIMYHEIIYVGEAIVLTVPGSERSYDLTGLKP |
| | GTEYPVPIEGVKGGGTSGPLSAIFTT |
| 319 | MLPAPKNLVVSRVTEDSARLSWTAPDAAFDSFAITYTEAALCGEAIVLTVPGSERSYDLTGLKP |
| | GTEYPVPINGVKGGGTSGPLSAIFTT |
| 320 | MLPAPKNLVVARVTEDSARLSWTAPDAAIDSFPIDYSEYWWGGEAIVLTVPGSERSYDLTGLK |
| | PGTEYPVLITGVKGGYRSGPLSAIFTT |
| 321 | MLPAPKNLVVSRVTEDSARLSWTAPDAAFDSFSIRYNEFIVAGEAIVLTVPGSERSYDLTGLKP |
| | GTEYDVPIAGVKGGGASWPLSAIVTT |
| 322 | MLPAPKNLVVSRVTEDSARLSWTAPDAAFDSFWYLELQFAGEAIVLTVPGSERSYDLTGLKPG |
| | TEYNVPITGVKGGIISFPLSAIFTT |
| 323 | MLPAPKNLVVSRVTEDSARLSWTAPDAAFDSFAIWYHEWYGDGEAIVLTVPGSERSYDLTGP |
| | KPGTEYRVRISGVKGGFESGPLSAIFTT |
| 324 | MLPAPKNLVVSRVTEDSARLSWTAPDAAFDSFMIRYQEGTRWGEAIVLTVPGSERSYDLTGL |
| | KPGTEYIVMIAGVKGGQISLPLSAIFTT |
| 325 | MLPAPKNLVVSRVTEDSARLSWTAPDAAFDSFPIWYLEKSYQGEAIVLTVPGSERSYDLTGLK |
| | PGTEYVVPIIGVKGGRDSCPLSAIFTT |
| 326 | MLPAPKNLVVSEVTEDSARLSWQGVARAFDSFLITYREQIFAGEVIVLTVPGSERSYDLTGLKP |
| | GTEYPVWIQGVKGGSPSAPLSAEFTT |
| 327 | MLPAPKNLVVSRVTEDSARLSWTAPDAAFDSFRISYAETVRQGEAIVLTVPGSERSYDLTVET |
| | GYRNWVMILGVKGGPGSLPLSAIFTT |
| 328 | MLPAPKNLVVSEVTEDSARLSWQGVVRAFDSFLITYREQIFAGEVIVLTVPGSERSYDLTGLKP |
| | GTEYPVWIQGVKGGSPSAPLSAEFTT |
| 329 | MLPAPKNLVVSRVTEDSARLSWTAPDAAFDSFWIEYWEAVGFGEAIVLTVPGSERSYDLTGLK |
| | PGTEYFVGIYGVKGGYLSAPLSAIFTT |
| 330 | MLPAPKNLVVSRVTEDSARLSWTAPDAAFDSFIIHYVEQQLIGEAIVLTVPGSERSYDLTGLKP |
| | GTEYPVPITGVKGGACSWPLSAIFTT |
| 331 | MLPAPKNLVVSRVTEDSARLSWTAPDAAFDSFTIEYSEHPIDGEAIPLFVPGSERSYDLTGLKP |
| | GTEYYVRIHGVKGGWFSHPLWAFFTT |
| 332 | MLPAPKNLVVSRVTEDSARLSWQGVARAFDSFLITYREQIFAGEVIVLTVPGSERSYDLTGLKP |
| | GTEYGVTIAGVKGGWRSKPLNAESTT |
| 333 | MLPAPKNLVVSRVTEDSARLSWTAPDAAFDSFGIAYVESYWYGEAIVLTVPGSERSYDLTGLK |
| | PGTEYNVPIYGVKGGDGSGPLSAIFTT |
| 334 | MLPAPKNLVVSRVTEDSARLSWTAPDAAFDSFYITYVELNLAGEAIVLTVPGSERSYDLTGLKP |
| | GTEYPVPILGVKGGSLSQPLSAIFTT |
| 335 | MLPAPKNLVVSRVTEDSARLSWTAPDAAFDSFPISYIESIADGEAIVLTVPGSERSYDLTGLKP |
| | GTEYWVAIVGVKGGPFSWSLSAIVTT |
| 336 | MLPAPKNLVVSEVTEDSARLSWQGVARAFDSFLITYREQIFAGEVIVPTVPGSERSYDLTGLKP |
| | GTEYPVPIAGVKGGGPSAPLSAIFTT |
| 337 | MLPAPKNLVVSRVTEDSARLSWTTPDAAFDSFPIYYWEVTITGEAIYLSVPGSERSYDLTGLKP |
| | GTEYPVDIPGVKGGAASPPLSAIFTT |
| 338 | MLPAPKNLVVSRVTEDSARLSWTAPDAAFDSFPILYLEHTVRSEAIVLTVPGSERSYDLTDLKP |
| | GTEYCVPIDGVKGGLRSRPLSAIFTT |
| 339 | MLPAPKNLVVSRVTEDSARLSWTAPDAAFDSFPIPYTEPPDPGEAIVLTVPGSERSYDLTGLK |
| | PGTEYLVTILGVKGGSMSVPLSAIFTT |
| 340 | MLPAPKNLVVSRVTEDSARLSWTAPDAAFDSFTIDYWENRCPGEAIVLTVPGSERSYDLTGLK |
| | PGTEYCVWISGVKGGYSSWPLSAIFTT |
| 341 | MLPAPKNLVVSRVTEDSARLSWQGVARAFDSFLITYREQIFAGEVIVLTVPGSERSYDLTGLKP |
| | GTEYPVWIQGVKGGHLSDPLSAIVTT |
| 342 | MLPAPKNLVVSRVTEDSARLSWTAPDAAFDSFLIPYAETSPSGEAIVLTVPGSERSYDLTGLKP |
| | GTEYSVLIHGVKGGDYSEPLSAIFTT |
| 343 | MLPAPKNLVVSRVTEDSARLSWTAPDAAFDSFMIVYYEYTRFGEAIVLTVPGSERSYDLTGLK |
| | PGTEYTVPIDGVKGGGRSSPLSAIFTT |
| 344 | MLPAPKNLVVSRVTEDSARLSWTAPDAAFDSFFIPYAEPSPTGEAIVLTVPGSERSYDLTGLKP |
| | GTEYSVLIHGVKGGHLSDPLSAIVTT |
| 345 | MLPAPKNLVVSEVTEDSARLSWQGVARAFDSFLITYREQIFAGEVIVLTVPGSERSYDLTGLKP |
| | GTEYPVWIQGVKGGSPSAPLSAEFTT |
| 346 | MLPAPKNLVVSRVTEDSARLSWTAPDAAFDSFIIPYAEVRPDGEAIVLTVPGSERSYDLTGLKP |
| | GTEYSVLIHGVKGGKLSLPLSAIFTT |
| 347 | MLPAPKNLVVSRVTEDSARLSWTAPDAAFDSFGIVYLEMMVTGEAIVLTVPGSERSYDLTGLK |
| | PGTEYDVPILGVKGGTRSVPLSAIFTT |
| 348 | MLPAPKNLVVSRVTEDSARLSWTAPDAAFDSFLIYYEEGYLEYYYSGEAIVLTVPGSERSYDLT |
| | GLKPGTEYYVGIVGVKGGGLSGPLSAISTT |
| 349 | MLPAPKNLVVSRVTEDSARLSWTAPDAAFDSFPIAYAEPRPDGEAIVLTVPGSERSYDLTGLK |
| | PGTEYSVLIHGVKGGDWSLPLSAIFTT |
| 350 | MSLPAPKNLVVSRVTEDSARLSWTAPDAAFDSFTIHYREFQLSGEAIVLTVPGSERSYDLTGL |
| | KPGTEYDVPIEGVKGGPGSRPLSAIFTT |
| 351 | MLPAPKNLVVSEVTEDSARLSWQGVARAFDSFLITYREQIFAGEVIVLTVPGSECSYDLTGLKP |
| | GTEYPVWIQGVKGGSPSAPLSAEFTT |
| 352 | MLPAPKNLVVSRVTEDSARLSWTAPDAAFDSFEIDYDELAIYGEAIVLTVPGSERSYDLTGLKP |
| | GTEYGVRIPGVKGGMPSLPLSAIVTT |
| 353 | MLPAPENLVVSEVTEDSARLSWQGVARAFDSFLITYREQIFAGEVIVLTVPGSERSYDLTGLKP |
| | GTEYPVWIQGVKGGSPSAPLSAESTT |
| 354 | MLPAPKNLVVSRVTEDSARLSWTAPDAAFDSFAIAYGEHIVIGEAIVLTVPGSERSYDLTGLKP |
| | GTEYMVPIAGVKGGPISLPLSAIFTT |
| 355 | MLPAPKNLVVSRVTEDSARLSWTAPDAAFDSFFIPYAEPSPTGEAIVLTVPGSERSYDLTGLKP |
| | GTEYSVLIHGVKGGHLSDPLSAIFTT |
| 356 | MLPAPKNLVVSRVTEDSARLSWTAPDAAFDSFSIGYVELVLLGEAIVLTVPGSERSYDLTGLKP |
| | GTEYDVLIPGVKGGSLSRPLSAIFTT |
| 357 | MLPAPKNLVVSRVTEDSARLSWTAPDAAFDSFAIPYAELSRNGEAIVLTVPGSERSYDLTGLK |
| | PGTEYTVLIHGVKGGCLSDPLSAIFTT |
| 358 | MLPAPKNLVVSRVTEDSARLSWTAPDAAFDSFHIEYLELSRHGEAIVLTVPGSERSYDLTGLKP |
| | GTEYWVMIFGVKGGGPSKPLSAIFTT |
| 359 | MLPAPKNLVVSRVTEDSARLSWTAPDAAFDSFVYNEVHWIGEAIVLTVPGSERSYDLTGLKPG |
| | TEYFVGIYGVKGGHWSKPLSAIFTT |
| 360 | MLPAPKNLVVSRVTEDSARLSWTAPDAAFDSFEIDYDELAIYGEAIVLTVPGSERSYDLTGLKP |
| | GTEYGVRIPGVKGGMPSLPLSAIVTT |
| 361 | MLPAPKNLVVSRVTEDSARLSWTAPDAAFDSFQIVYSELWIKGEAIVLTVPGSERSYDLTGLKP |
| | GTEYQVPIPGVKGGRNSFPLSAIFTT |
| 362 | MLPAPKNLVVSRVTEDSARLSWTAPDAAFDSFKIRYTETRSIGEAIVLTVPGSERSYDLTGLKP |
| | GTEYCVPIGGVKGGDSSWPLSAISTT |
| 363 | MLPAPKNLVVSRVTEDSARLSWTAPDAAFDSFCISYYERMGRGEAIVLTVPGSERSYDLTGLK |
| | PGTEYMVYIFGVKGGLNSLPLSAIFTT |
| 364 | MLPAPKNLVVSRVTEDSARLSWTAPDAAFDSFYIVYAEPIPNGEAIVLTVPGSERSYDLTGLKP |
| | GTEYSVLIHGVKGGRNSDPLSAIFTT |
| 365 | MLPAPKNLVVSRVTKDSARLSWTAPDAAFDSFPIAYAEPRPDGEAIVLTVPGSERSYDLTGLK |
| | PGTEYSVLIHGVKGGLLSSPLSAIFTT |
| 366 | MLPAPKNLVVSRVTEDSARLSWTAPDAAFDSFTIDYDEPRSPGEAIVLTVPGSERSYDLTGLK |
| | PGTEYRVFIWGIKGGDTSFPLSAIFTT |
| 367 | MLPAPKNLVVSRVTEDSARLSWTAPDAAFDSFTILYAEQAQFGEAIVLTVPGSERSYDLTGLK |
| | PGTEYPITGVKGGTRSGPLSAISTT |
| 368 | MLPAPKNLVVSRVTEDSARLSWTAPDAAFDSFIIPYAEVRPDGEAIVLTVPGSERSYDLTGLKP |
| | GTEYSVLIHGVKGGHLSDPLSAISTT |
| 369 | MLPAPKNLVVSRVTEDSARLSWTAPDAAFDSFHIAYEETATSGEAIYLRVPGSERSYDLTGLK |
| | PGTEYGVEIEGVKGGARSRPLYADFTT |
| 370 | MLPAPKNLVVSRVTEDSARLSWQGVARAFDSFLITYREQIFAGEVIVLTVPGSERSYDLTGLKP |
| | GTEYPVWIQGVKGGDLSNPLSAIFTT |
| 371 | MLPAPKNLVVSRVTEDSARLSWTAPDAAFDSFPISYLELSLYGEAIVLTVPGSERSYDLTGLKP |
| | GTEYPVGIAGVKGGVVSRPLSAIFTT |
| 372 | MLPAPKNLVVSRVTEDSARLSWTAPDAAFDSFTIGYREWYWYGEAIVLTVPGSERSYDLTGL |
| | KPGTEYNVPISGVKGGLDSFPLSAIFTT |
| 373 | MLPAPKNLVVSEVTEDSARLSWQGVARAFDSFLITYREQIFAGEVIVLTVPGSERSYDLTGLKP |
| | GTEYPVWIQGVKGGSPSAPLSAESTT |
| 374 | MLPAPKNLVVSRVTEDSARLSWTAPDAAFDSFSITYLEWWNLGEAIVLTVPGSERSYDLTGLK |
| | PGTEYMVTIPGVKGGMSSYPLSAIFTT |
| 375 | MLPAPKNLVVSRVTEDSARLSWTAPDAAFDSFTISYGEEALIGEAIYLRVPGSERSYDLTGLKP |
| | GTEYYVHIEGVKGGSWSQPLAAAFTT |
| 376 | MLPAPKNLVVSRVTEDSARLSWTAPDAAFDSFTIEYYENIGIGEAIVLTVPGSERSYDLTGLKP |
| | GTEYSVPIVGVKGGPYSHPLSAIFTT |
| 377 | MLPAPKNLVVSRVTEDSARLSWTAPDAAFDSFPIAYAEPRPDGEAIVLTVPGSERSYDLTGLK |
| | PGTEYSVLIHGVKGGLLSSPLSAIFTT |
| 378 | MLPAPKNLVVSRVTEDSARLSWTAPDAAFDSFGIGYYEHKRFGEAIQLSVPGSERSYDLTGLK |
| | PGTEYEVDIEGVKGGVLSWPLFAEFTT |
| 379 | MLPAPKNLVVSRVTEDSARLSWTAPDAAFDSFVIEYTERFWSGEAIVLTVPGSERSYDLTGLK |
| | PGTEYSVPIDGVKGGQCSTPLSAIFTT |
| 380 | MLPAPKNLVVSRVTEDSARLSWTAPDAAFDSFWIDYEEEGVIGEAIYLHVPGSERSYDLTGLK |
| | PGTEYVVKIHGVKGGHPSHPLVAVFTT |
| 381 | MLPAPKNLVVSRVTEDSARLSWQGVARAFDSFLITYVELRHLGEAIVLTVPGSERSYDLTGLK |
| | PGTEYPVWIQGVKGGSPSAPLSAEFTT |
| 382 | MLPAPKNLVVSRVTEDSARLSWTAPDAAFDSFLIPYAETSPSGEAIVLTVPGSERSYDLTGLKP |
| | GTEYSVLIHGVKGGDYSSPLSAIFTT |
| 383 | MLPAPKNLVVSRVTEDSARLSWTAPDAAFDSFPIAYAEPRPDGEAIVLTVPGSERSYDLTGLK |
| | PGTEYSVLIHGVKGGLLSSPLSAISTT |
| 384 | MLPAPKNLVVSRVTEDSARLSWQGVARAFDSFSILYLELTPKGEAIVLTVPGSERSYDLTGLKP |
| | GTEYPVWIQGVKGGSPSAPLSAEFTT |
| 385 | MLPAPKNLVVSRVTEDSARLSWTAPDAAFDSFIIEYFEPIPIGEAIVLTVPGSERSYDLTGLKPG |
| | TEYAVNIYGVKGGYLSHPLSAIFTT |
| 386 | MLPAPKNLVVSEVTEDSARLSWQGVARAFDSFLITYREQIFAGEVIVLTVPGSECSYDLTGLKP |
| | GTEYPVWIQGVKGGSPSAPLSAEFTT |
| 387 | MLPAPKNLVVSRVTEDSARLSWTAPDAAFDSFAIEYTEFLYSGEAIVLTVPGSERSYDLTGLKP |
| | GTEYGVPINGVKGGFVSPPLSAIVTT |
| 388 | MLPAPKNLVVSRVTEDSARLSWTAPDAAFDSFPIKYREVLRCGEAIVLTVPGSERSYDLTGLK |
| | PGTEYTVPITGVKGGFGSSPLSAIFTT |
| 389 | MLPAPENLVVSRVTEDSARLSWTAPDAAFDSFWIEYYEGVIQGEAIVLTVPGSERSYDLTGLK |
| | PGTEYFVAIWGVKGGKWSVPLSAIFTT |
| 390 | MLPAPKNLVVSRVTEDSARLSWQGVARAFDSFLITYREQIFAGEVIVLTVPGSERSYDLTGLKP |
| | GTEYPVWIQGVKGGSPSAEFTT |
| 391 | MLPAPKNLVVSRVTEDSARLSWTAPDAAFDSFQIHYWETQGFGEAIVLTVPGSERSYDLTGLK |
| | PGTEYPVLIPGVKGGPSSLPLSAIFTT |
| 392 | MLPAPKNLVVSRVTEDSARLSWTAPDAAFDSFFIPYAEPSPTGEAIVLTVPGSERSYDLTGLKP |
| | GTEYSVLIHGVKGGHLSDPLSAIFTT |
| 393 | MLPAPKNLVVSRVTEDSARLSWTAPDAAFDSFAIEYYEPVPAGEAIYLDVPGSERSYDLTGLK |
| | PGTEYDVTIYGVKGGYYSHPLFASFTT |
| 394 | MLPAPKNLVVSEVTEDSARLSWQGVARAFDSFLITYREQIFAGEVIVLTVPGSERSYDLTGLKP |
| | GTEYPVWIQGVKGGSPSAPLSAIFTT |
| 395 | MLPAPKNLVVSEVTEDSARLSWQGVARAFDSFLITYREQIFAGEVIVLTVPGSERSYDLTGLKP |
| | GTEYPVWIQGVKGGSPSAPLSAEFTT |
| 396 | MSLPAPKNLVVSRVTEDSARLSWTAPDAAFDSFPIAYLEVFYEGEAIVLTVPGSERSYDLTGLK |
| | PGTEYQVPIEGVKGGAMSLPLSAIFTT |
| 397 | MLPAPKNLVVSRVTEDSARLSWTAPDAAFDSFHIWYEEETTIGEAIYLHVPGSERSYDLTGLKP |
| | GTEYEVHITGVKGGPYSRPLFANFTT |
| 398 | MLPAPKNLVVSRVTEDSARLSWTAPDAAFDSFGIAYDEWPEFGEAIVLTVPGSERSYDLTGLK |
| | PDTEYIVEIYGVKGGWFSWPLSAIFTT |
| 399 | MLPAPKNLVVSRVTEDSARLSWTAPDAAFDSFFIPYAEPSPTGEAIVLTVPGSERSYDLTGLKP |
| | GTEYSVLIHGVKGGHLSDPLSVIFTT |
| 400 | MLPAPKNLVVSRVTEDSARLSWTAPDAAFDSFYIWYEEVMYLGEAIVLTVPGSERSYDLTGLK |
| | PGTEYNVPIPGVKGGHSSPPLSAIFTT |
| 401 | MLPAPKNLVVSRVTEDSARLSWTAPDAAFDSFHILYEELFLVGEAIVLTVPGSERSYDLTGLKP |
| | GTEYKVPISGVKGGPVSRPLSAIFTT |
| 402 | MLPAPKNLVVSRVTEDSARLSWQGVARAFDSFLITYREQIFAGEVIVLTVPGSERSYDLTGLKP |
| | GTEYPVWIQGVKGGSPSAPLSAEFTT |
| 403 | MLPAPKNLVVSRVTEDSARLSWTAPDAAFDSFHIVYHEPRPSGEAIWLHVPGSERSYDLTGLK |
| | PGTEYEVGIVSVKGGDLSVPLVAFFTT |
| 404 | MLPAPKNLVVSRVTEDSARLSWTAPDAAFDSFYIAYAEPRPDGEAISLLVPGSERSYDLTGLK |
| | PGTEYSVLIHGVKGGLLSSPLYAVFTT |
| 405 | MLPAPKNLVVSRVTEDSARLSWTAPDAAFDSFYIAYAEPRPDGEAIFLVVPGSERSYDLTGLK |
| | PGTEYSVLIHGVKGGLLSSPLHANFTT |
| 406 | MLPAPKNLVVSRVTEDSARLSWTAPDAAFDSFYIAYAEPRPDGEAILLDVPGSERSYDLTGLK |
| | PGTEYSVLIHGVKGGLLSSPLYASFTT |
| 407 | MSLPAPKNLVVSRVTEDSARLSWTAPDAAFDSFYIAYAEPRPDGEAISLYVPGSERSYDLTGL |
| | KPGTEYSVLIHGVKGGLLSSPLSAISTT |
| 408 | MLPAPKNLVVSRVTEDSARLSWTAPDAAFDSFFIGYLEPQPPGEAIQLRVPGSERSYDLTGLK |
| | PGTEYNVTIQGVKGGFPSMPLSAIFTT |
| 409 | MLPAPKNLVVSRVTEDSARLSWTAPDAAFDSFHISYEEDYTFGEAIYLRVPGSERSYDLTGLK |
| | PGTEYRVVIGGVKGGWFSEPLLAAFTT |
| 410 | MLPAPKNLVVSRVTEDSARLSWTAPDAAFDSFFIGYLEPQPPGEAIYLTVPGSERSYDLTGLK |
| | PGTEYNVTIQGVKGGFPSYPLDASFTT |
| 411 | MLPAPKNLVVSRVTEDSARLSWTAPDAAFDSFFIGYLEPQPPGEAIDLGVPGSERSYDLTGLK |
| | PGTEYNVTIQGVKGGFPSMPLDPLEAYFTT |
| 412 | MLPAPKNLVVSRVTEDSARLSWTAPDAAFDSFYIAYAEPRPDGEAILLLVPGSERSYDLTGLKP |
| | GTEYSVLIHGVKGGLLSSPLSAIFTT |
| 413 | MLPAPKNLVVSRVTEDSARLSWTAPDAAFDSFYIAYAEPRPDGEAINLQVPGSERSYDLTGLK |
| | PGTEYSVLIHGVKGGLLSSPLVAFFTT |
| 414 | MLPAPKNLVVSRVTEDSARLSWTTPDAAFDSFFIGYLEPQPPGEAISLQVPGSERSYDLTGLK |
| | PGTEYNVTIQGVKGGFPSSPLFAVFTT |
| 415 | MLPAPKNLVVSRVTEDSARLSWTAPDAAFDSFYIAYAEPRPDGEAIELHVPGSERSYDLTGLK |
| | PGTEYSVLIHGVKGGLLSSPLFTT |
| 416 | MLPAPKNLVVSRVTEDSARLSWTAPDAAFDSFYIAYAEPRPDGEAIQLVVPGSERSYDLTGLK |
| | PGTEYSVLIHGVKGGLLSSPLSAIFTT |
| 417 | MLPAPKNLVVSRVTEDSARLSWTAPDAAFDSFYIAYAEPRPDGEAITLDVPGSERSYDLTGLK |
| | PGTEYSVLIHGVKGGLLSSPLSAIFTT |
| 418 | MLPAPKNLVVSRVTEDSARLSWTAPDAAFDSFYIAYAEPRPDGEAIWLVVPGSERSYDLTGLK |
| | PGTEYSVLIHGVKGGLLSSPLVASFTT |
| 419 | MLPAPKNLVVSRVTEDSARLSWTAPDAAFDSFYIAYAEPRPDGEAINLDVPGSERSYDLTGLK |
| | PGTEYSVLIHGVKGGLLSSPLVAEFTT |
| 420 | MLPAPKNLVVSRVTEDSARLSWTAPDAAFDSFYIAYAEPRPDGEAIHLSVPGSERSYDLTGLK |
| | PGTEYSVLIHGVKGGLLSSPLVAIFTT |
| 421 | MLPAPKNLVVSRVTEDSARLSWTAPDAAFDSFFIGYLEPQPPGEAIALWVPGSERSYDLTGLK |
| | PGTEYNVTIQGVKGGFPSMPLSAIFTT |
| 422 | MLPAPKNLVVSRVTEDSARLSWTAPDAAFDSFYIAYAEPRPDGEAIILVVPGSERSYDLTGLKP |
| | GTEYSVLIHGVKGGLLSSPLSAHFTT |
| 423 | MLPAPKNLVVSRVTEDSARLSWTAPDAAFDSFFIGYLEPQPPGEAIQLWVPGSERSYDLTGLK |
| | PGTEYNVTIQGVKGGFPSHPLGAVFTT |
| 424 | MLPAPKNLVVSRVTEDSARLSWTAPDAAFDSFYIAYAEPRPDGEAIQLHVPGSERSYDLTGLK |
| | PGTEYSVLIHGVKGGLLSSPLLASFTT |
| 425 | MLPAPKNLVVSRVTEDSARLSWTAPDAAFDSFYIAYAEPRPDGEAIALHVPGSERSYDLTGLK |
| | PGTEYSVLIHGVKGGLLSSPLVAFFTT |
| 426 | MLPAPKNLVVSRVTEDSARLSWTAPDAAFDSFFIGYLEPQPPGEAIHLHVPGSERSYDLTGLK |
| | PGTEYNVTIQGVKGGFPSIPLHANFTT |
| 427 | MLPAPKNLVVSRVTEDSARLSWTAPDAAFDSFYIAYAEPRPDGEAIFLGVPGSERSYDLTGLK |
| | PGTEYSVLIHGVKGGLLSSPLSAIFTT |
| 428 | MLPAPKNLVVSRVTEDSARLSWTAPDAAFDSFYIAYAEPRPDGEAIVLRVPGSERSYDLTGLK |
| | PGTEYSVLIHGVKGGLLSSPLIASFTT |
| 429 | MLPAPKNLVVSRVTEDSARLSWTAPDAAFDSFYIAYAEPRPDGEAINLWVPGSERSYDLTGLK |
| | PGTEYSVLIHGVKGGLLSSPLDASFTT |
| 430 | MLPAPKNLVVSRVTEDSARLSWTAPDAAFDSFVIEYFEWTLNGEAIVLTVPGSERSYDLTGLK |
| | PGTEYSVQIYGVKGGCLSRPLSAIFTT |
| 431 | MLPAPKNLVVSRVTEDSARLSWTAPDAAFDSFYIAYAEPRPDGEAIHLWVPGSERSYDLTGLK |
| | PGTEYSVLIHGVKGGLLSSPLIAHFTT |
| 432 | MLPAPKNLVVSRVTEDSARLSWTAPDAAFDSFFIPYAEPSPTGEAIVLTVPGSERSYDLTGLKP |
| | GTEYSVLIHGVKGGLLSSPLSAHFTT |
| 433 | MLPAPKNLVVSRVTEDSARLSWTAPDAAFDSFYIAYAEPRPDGEAIYLYVPGSERSYDLTGLK |
| | PGTEYSVLIHGVKGGLLSSPLDAFFTT |
| 434 | MLPAPKNLVVSRVTEDSARLSWTAPDAAFDSFYIAYAEPRPDGEAIGLQVPGSERSYDLTGLK |
| | PGTEYSVLIHGVKGGLLSSPLSAIFTT |
| 435 | MLPAPKNLVVSRVTEDSARLSWTAPDAAFDSFYIAYAEPRPDGEAIQLAVPGSERSYDLTGLK |
| | PGTEYSVLIHGVKGGLLSSPLHAFFTT |
| 436 | MLPAPKNLVVSRVTEDSARLSWTAPDAAFDSFFIGYLEPQPPGEAIWLHVPGSERSYDLTGLK |
| | PGTEYNVTIQGVKGGFPSIPLIAIFTT |
| 437 | MLPAPKNLVVSRVTEDSARLSWTAPDAAFDSFYIAYAEPRPDGEAIVLDVPGSERSYDLTGLK |
| | PGTEYSVLIHGVKGGLLSSPLVAEFTT |
| 438 | MLPTPKNLVVSRVTEDSARLSWTAPDAAFDSFYIAYAEPRPDGEAIQLRVPGSERSYDLTGLK |
| | PGTEYSVLIHGVKGGLLSSPLHASFTT |
| 439 | MLPAPKNLVVSRVTEDSARLSWTAPDAAFDSFFIGYLEPQPPGEAIQLGVPGSERSYDLTGLK |
| | PGTEYNVTIQGVKGGFPSHPLNANFTT |
| 440 | MLPAPKNLVVSRVTEDSARLSWTAPDAAFDSFFIGYLEPQPPGEAIQLEVPGSERSYDLTGLK |
| | PGTEYNVTIQGVKGGFPSMPLSAIFTT |
| 441 | MLPAPKNLVVSRVTEDSARLSWTAPDAAFDSFYIAYAEPRPDGEAIFLGVPGSERSYDLTGLK |
| | PGTEYSVLIHGVKGGLLSSPLIAFFTT |
| 442 | MLPAPKNLVVSRVTEDSARLSWTAPDAAFDSFFIGYLEPQPPGEAIGLQVPGSERSYDLTGLK |
| | PGTEYNVTIQGVKGGFPSHPLKAQFTT |
| 443 | MLPAPKNLVVSRVTEDSARLSWTAPDAAFDSFYIAYAEPRPDGEAILLFVPGSERSYDLTGLK |
| | PGTEYSVLIHGVKGGLLSSPLVAHFTT |
| 444 | MLPAPKNLVVSRVTEDSARLSWTAPDAAFDSFYIAYAEPRPDGEAIGLYVPGSERSYDLTGLK |
| | PGTEYSVLIHGVKGGLLSSPLGAFFTT |
| 445 | MLPAPKNLVVSRVTEDSARLSWTAPDAAFDSFYIAYAEPRPDGEAILLQVPGSERSYDLTGLK |
| | PGTEYSVLIHGVKGGLLSSPLTAIFTT |
| 446 | MLPAPKNLVVSRVTEDSARLSWTAPDAAFDSFYIAYAEPRPDGEAITLHVPGSERSYDLTGLK |
| | PGTEYSVLIHGVKGGLLSSPLSAIFTT |
| 447 | MLPAPKNLVVSRVTEDSARLSWTAPDAAFDSFYIAYAEPRPDGEAIVLEVPGSERSYDLTGLK |
| | PGTEYSVLIHGVKGGLLSSPLDAHFTT |
| 448 | MLPAPKNLVVSRVTEDSARLSWTAPDAAFDSFYIAYAEPRPDGEAIALHVPGSERSYDLTGLK |
| | PGTEYSVLIHGVKGGLLSSPLRAVFTT |
| 449 | MLPAPKNLVVSRVTEDSARLSWTAPDAAFDSFYIAYAEPRPDGEAIQLWVPGSERSYDLTGLK |
| | PGTEYSVLIHGVKGGLLSSPLSAIFTT |
| 450 | MLPAPKNLVVSRVTEDSARLSRTAPDAAFDSFYIAYAEPRPDGEAIVLIVPGSERSYDLTGLKP |
| | GTEYSVLIHGVKGGLLSSPLSAIFTT |
| 451 | MLPAPKNLVVSRVTEDSARLSWTAPDAAFDSFFIGYLEPQPPGEAIQLWVPGSERSYDLTGLK |
| | PGTEYNVTIQGVKGGFPSRPLQAHFTT |
| 452 | MLPAPKNLVVSRVTEDSARLSWTAPDAAFDSFYIAYAEPRPDGEAITLDVPGSERSYDLTGLK |
| | PGTEYSVLIHGVKGGLLSSPLYAFFTT |
| 453 | MLPAPKNLVVSRVTEDSARLSWTAPDAAFDSFYIAYAEPRPDGEAIALHVPGSERSYDLTGLK |
| | PGTEYSVLIHGVKGGLLSSPLSAIFTT |
| 454 | MLPAPKNLVVSRVTEDSARLSWTAPDAAFDSFYIAYAEPRPDGEAIGLWVPGSERSYDLTGLK |
| | PGTEYSVLIHGVKGGLLSSPLIAHFTT |
| 455 | MLPAPKNLVVSRVTEDSARLSWTAPDAAFDSFYIAYAEPRPDGEAIWLVVPGSERSYDLTGLK |
| | PGTEYSVLIHGVKGGLLSSPLHARFTT |
| 456 | MLPAPKNLVVSRVTEDSARLSWTAPDAAFDSFYIAYAEPRPDGEAIFLQVPGSERSYDLTGLK |
| | PGTEYSVLIHGVKGGLLSSPLAAVFTT |
| 457 | MLPAPKNLVVSRVTEDSARLSWTAPDAAFDSFYIAYAEPRPDGEAILLHVPGSERSYDLTGLK |
| | PGTEYSVLIHGVKGGLLSSPLSAISTT |
| 458 | MLPAPKNLVVSRVTEDSARLSWTAPDAAFDSFYIAYAEPRPDGEAIILQVPGSERSYDLTGLKP |
| | GTEYSVLIHGVKGGLLSSPLSAVFTT |
| 459 | MLPAPKNLVVSRVTEDSARLSWTAPDAAFDSFYIAYAEPRPDGEAIYLKVPGSERSYDLTGLK |
| | PGTEYSVLIHGVKGGLLSSPLVAHFTT |
| 460 | MLPAPKNLVVSRVTEDSARLSWTAPDAAFDSFYIAYAEPRPDGEAIVLTVPGSERSYDLTGLK |
| | PGTEYSVLIHGVKGGLLSSPLLAYFTT |
| 461 | MLPAPKNLVVSRVTEDSARLSWTAPDAAFDSFYIAYAEPRPDGEAIILHVPGSERSYDLTGLKP |
| | GTEYSVLIHGVKGGLLSSPLEAKFTT |
| 462 | MLPAPKNLVVSRVTEDSARLSWTAPDAAFDSFYIAYAEPRPDGEAIKLEVPGSERSYDLTGLK |
| | PGTEYSVLIHGVKGGLLSSPLAIFTT |
| 463 | MLPAPKNLVVSRVTEDSARLSWTAPDAAFDSFFIGYLEPQPPGEAIYLEVPGSERSYDLTGLK |
| | PGTEYNVTIQGVKGGFPSFPLKAAFTT |
| 464 | MLPAPKNLVVSRVTEDSARLSWTAPDAAFDSFYIAYAEPRPDGEAIILRVPGSERSYDLTGLKP |
| | GTEYSVLIHGVKGGLLSSPLVAIFTT |
| 465 | MLPAPKNLVVSRVTEDSARLSWTAPDAAFDSFYIAYAEPRPDGEAIVLQVPGSERSYDLTGLK |
| | PGTEYSVLIHGVKGGLLSSPLAAWFTT |
| 466 | MLPAPKNLVVSRVTEDSARLSWTAPDAAFDSFYIAYAEPRPDGEAIFLQVPGSERSYDLTGLK |
| | PGTEYSVLIHGVKGGLLSSPLNAFFTT |
| 467 | MLPAPKNLVVSRVTEDSARLSWTAPDAAFDSFFIGYLEPQPPGEAIILGVPGSERSYDLTGLKP |
| | GTEYSVLIHGVKGGLLSSPLHAYSTT |
| 468 | MLPAPKNLVVSRVTEDSARLSWTAPDAAFDSFYIAYAEPRPDGEAILLDVPGSERSYDLTGLK |
| | PGTEYSVLIHGVKGGLLSSPLSAIFTT |
| 469 | MLPAPKNLVVSRVTEDSARLSWTAPDAAFDSFYIAYAEPRPDGEAILLLVPGSERSYDLTGLKP |
| | GTEYSVLIHGVKGGLLSSPLSAVFTT |
| 470 | MLPAPKNLVVSRVTEDSARLSWTAPDAAFDSFYIAYAEPRPDGEAIHLLVPGSERSYDLTGLK |
| | PGTEYSVLIHGVKGGLLSSPLLAHFTT |
| 471 | MLPAPKNLVVSRVTEDSARLSWTAPDAAFDSFYIAYAEPRPDGEAIQLWVPGSERSYDLTGLK |
| | PGTEYSVLIHGVKGGLLSSPLDAYFTT |
| 472 | MLPAPKNLVVSRVTEDSARLSWTAPDAAFDSFFIGYLEPQPPGEAIVLTVPGSERSYDLTGLK |
| | PGTEYNVTIQGVKGGFPSMPLASFTT |
| 473 | MLPAPKNLVVSRVTEDSARLSWTAPDAAFDSFHIVYHEPRPSGEAIHLQVPGSERSYDLTGLK |
| | PGTEYNVTIQGVKGGFPSYPLSAFFTT |
| 474 | MLPAPKNLVVSRVTEDSARLSWTAPDAAFDSFFIGYLEPQPPGEAIQLWVPGSERSYDLTGLK |
| | PGTEYNVTIQGVKGGFPSMPLSAIFTT |
| 475 | MLPAPKNLVVSRVTEDSARLSWTAPDAAFDSFRISYCETFYHGEAIVLTVPGSERSYDLTGLK |
| | PGTEYSVLIHGVKGGLLSSPLIAKFTT |
| 476 | MLPAPKNLVVSRVTEDSARLSWTAPDAAFDSFYIAYAEPRPDGEAIWLKVPGSERSYDLTGLK |
| | PGTEYSVLIHGVKGGLLSSPLQANFTT |
| 477 | MLPAPKNLVVSRVTEDSARLSWTAPDAAFDSFYIAYAEPRPDGEAIWLKVPGSERSYDLTGLK |
| | PGTEYSVLIHGVKGGLLSSPLQANFTT |
| 478 | MLPAPKNLVVSRVTEDSARLSWTAPDAAFDSFYIAYAEPRPDGEAIQLQVPGSERSYDLTGLK |
| | PGTEYSVLIHGVKGGLLSSPLSAIVTT |
| 479 | MLPAPKNLVVSRVTEDSARLSWTAPDAAFDSFPIAYAEPRPDGEAIVLTVPGSERSYDLTGLK |
| | PGTEYSVLIHGVKGGLLSSPLSAFFTT |
| 480 | MLPAPKNLVVSRVTEDSARLSWTAPDAAFDSFYIAYAEPRPDGEAIALLVPGSERSYDLTGLK |
| | PGTEYSVLIHGVKGGLLSSPLVAQFTT |
| 481 | MLPAPKNLVVSRVTEDSARLSWTAPDAAFDSFYIAYAEPRPDGEAIILHVPGSERSYDLTGLKP |
| | GTEYSVLIHGVKGGLLSSPLEAKFTT |
| 482 | MLPAPKNLVVSRVTEDSARLSWTAPDAAFDSFYIAYAEPRPDGEAIDLHVPGSERSYDLTGLK |
| | PGTEYSVLIHGVKGGLLSSPLHALFTT |
| 483 | MLPAPKNLVVSRVTEDSARLSWTAPDAAFDSFYIAYAEPRPDGEAILLDVPGSERSYDLTGLK |
| | PGTEYSVLIHGVKGGFPSMPLSAIFTT |
| 484 | MLPAPKNLVVSRVTEDSARLSWTAPDAAFDSFFIGYLEPQPPGEAIDLAVPGSERSYDLTGLK |
| | PGTEYSVLIHGVKGGLLSSPLSFTT |
| 485 | MLPAPKNLVVSRVTEDSARLSWTAPDAAFDSFYIAYAEPRPDGEAIYLGVPGSERSYDLTGLK |
| | PGTEYSVLIHGVKGGLLSSPLRAKFTT |
| 486 | MLPAPKNLVVSRVTEDSARLSWTAPDAAFDSFYIAYAEPRPDGEAIQLGVPGSERSYDLTGLK |
| | PGTEYSVLIHGVKGGLLSSPLSAIFTT |
| 487 | MLPAPKNLVVSRVTEDSARLSWTAPDAAFDSFFIGYLEPQPPGEAISLLVPDSERSYDLTGLKP |
| | GTEYNVTIQGVKGGFPSMPLKFTT |
| 488 | MLPAPKNLVVSRVTEDSARLSWTAPDAAFDSFYIAYAEPRPDGEAIGLGVPGSERSYDLTGLK |
| | PGTEYSVLIHGVKGGLLSSPLDASFTT |
| 489 | MLPAPKNLVVSRVTEDSARLSWTAPDAAFDSFYIAYAEPRPDGEAIQLTVPGSERSYDLTGPK |
| | PGTEYWVLIQGVKGGGSSVPLVAYFTT |
| 490 | MLPAPKNLVVSRVTEDSARLSWTAPDAAFDSFYIAYAEPRPDGEAISLDVPGSERSYDLTGLK |
| | PGTEYSVLIHGVKGGLLSSPLEASFTT |
| 491 | MLPAPKNLVVSRVTEDSARLSWTAPDAAFDSFFIGYLEPQPPGEAIILAVPGSERSYDLTGLKP |
| | GTEYNVTIQGVKGGFPSLPLVASFTT |
| 492 | MLPAPKNLVVSRVTEDSARLSWTAPDAAFDSFDIGYTEYGGYGEAIYLSVPGSERSYDLTGLK |
| | PGTEYWVLIQGVKGGGSSVPLSAIFTT |
| 493 | MLPAPKNLVVSRVTEDSARLSWTAPDAAFDSFYIAYAEPRPDGEAISLSVPGSERSYDLTGLK |
| | PGTEYSVLIHGVKGGLLSSPLIANFTT |
| 494 | MLPAPKNLVVSRVTEDSARLSWTAPDAAFDSFYIAYAEPRPDGEAIALLVPGSERSYDLTGLK |
| | PGTEYSVLIHGVKGGLLSSPLSAIVTT |
| 495 | MLPAPKNLVVSRVTEDSARLSWTAPDAAFDSFYIAYAEPRPDGEAIILDVPGSERSYDLTGLKP |
| | GTEYSVLIHGVKGGLLSSPLSSIFTT |
| 496 | MLPAPKNLVVSRVTEDSARLSWTAPDAAFDSFYIAYAEPRPDGEAIVLWVPGSERSYDLTGLK |
| | PGTEYSVLIHGVKGGLLSSPLRASFTT |
| 497 | MLPAPKNLVVSRVTEDSARLSWTAPDAAFDSFYIAYAEPRPDGEAIKLDVPGSERSYDLTGLK |
| | PGTEYSVLIHGVKGGLLSSPLVAFFTT |
| 498 | MLPAPKNLVVSRVTEDSARLSWTAPDAAFDSFYIAYAEPRPDGEAIILEVPGSERSYDLTGLKP |
| | GTEYSVLIHGVKGGLLSSPLVAYFTT |
| 499 | MLPAPKNLVVSRVTEDSARLSWTAPDAAFDSFYIAYAEPRPDGEAIHLWVPGSERSYDLTGLK |
| | PGTEYSVLIHGVKGGLLSSPLHADFTT |
| 500 | MLPAPKNLVVSRVTEDSARLSWTAPDAAFDSFYIAYAEPRPDGEAIWLEVPGSERSYDLTGLK |
| | PGTEYSVLIHGVKGGLLSSPLVADFTT |
| 501 | MLPAPKNLVVSRVTEDSARLSWTAPDAAFDSFYIAYAEPRPDGEAISLWVPGSERSYDLTGLK |
| | PGTEYSVLIHGVKGGLLSSPLLAHFTT |
| 502 | MLPAPKNLVVSRVTEDSARLSWTAPDAAFDSFDIGYTEYGGYGEAILHVPGSERSYDLTGLKP |
| | GTEYWVLIQGVKGGGSSVPLSAIFTT |
| 503 | MLPAPKNLVVSRVTEDSARLSWTAPDAAFDSFFIGYLEPQPPGEAIVLLVPGSERSYDLTGLK |
| | PGTEYNVTIQGVKGGFPSVPLAAFFTT |
| 504 | MLPAPKNLVVSRVTEDSARLSWTAPDAAFDSFFIGYLEPQPPGEAILLWVPGSERSYDLTGLK |
| | PGTEYNVTIQGVKGGFPSQFTT |
| 505 | MLPAPKNLVVSRVTEDSARLSWTAPDAAFDSFFIGYLEPQPPGEAILLGVPGSERSYDLTGLK |
| | PGTEYNVTIQGVKGGFPSMPLHPLVALFTT |
| 506 | MLPAPKNLVVSRVTEDSARLSWTAPDAAFDSFYIAYAEPRPDGEAIGLDVPGSERSYDLTGLK |
| | PGTEYSVLIHGVKGGLLSSPLSAIFTT |
| 507 | MLPAPKNLVVSRVTEDSARLSWTAPDAAFDSFYIAYAEPRPDGEAIHLSVPGSERSYDLTGLK |
| | PGTEYSVLIHGVKGGLLSSPLAAYFTT |
| 508 | MLPAPKNLVVSRVTEDSARLSWTAPDAAFDSFFIGYLEPQPPGEAIVLAVPGSERSYDLTGLK |
| | PGTEYNVTIQGVKGGFPSYPLVAAFTT |
| 509 | MLPAPKNLVVSRVTEDSARLSWTAPDAAFDSFYIAYAEPRPDGEAILLQVPGSCRSYDLTGLK |
| | PGTEYSVLIHGVKGGLLSSPLTAIFTT |
| 510 | MLPAPKNLVVSRVTEDSARLSWTAPDAAFDSFFIGYLEPQPPGEAINLQVPGSERSYDLTGLK |
| | PGTEYNVTIQGVKGGFPSFPLSAVFTT |
| 511 | MLPAPKNLVVSRVTEDSARLSWTAPDAAFDSFYIAYAEPRPDGEAIQLHVPGSERSYDLTGLK |
| | PGTEYSVLIHGVKGGLLSSPLVAIFTT |
| 512 | MLPAPKNLVVSRVTEDSARLSWTAPDAAFDSFYIAYAEPRPDGEAIWLAVPGSERSYDLTGLK |
| | PGTEYSVLIHGVKGGLLSSPLHAQFTT |
| 513 | MLPAPKNLVVSRVTEDSARLSWTAPDAAFDSFYIAYAEPRPDGEAILLGVPGSERSYDLTGLK |
| | PGTEYSVLIHGVKGGLLSSPLFTT |
| 514 | MLPAPKNLVVSRVTEDSARLSWTAPDAAFDSFYIAYAEPRPDGEAIGLQVPGSERSYDLTGLK |
| | PGTEYSVLIHGVKGGLLSSPLCAEFTT |
| 515 | MLPAPKNLVVSRVTEDSARLSWTAPDAAFDSFYIAYAEPRPDGEAIVLWVPGSERSYDLTGLK |
| | PGTEYSVLIHGVKGGLLSSPLIAEFTT |
| 516 | MLPAPKNLVVSRVTEDSARLSWTAPDAAFDSFYIAYAEPRPDGEAISLSVPGSERSYDLTGLK |
| | PGTEYSVLIHGVKGGLLSSPPKFTT |
| 517 | MLPAPKNLVVSRVTEDSARLSWTAPDAAFDSFYIAYAEPRPDGEAIILEVPGSERSYDLTGLKP |
| | GTEYSVLIHGVKGGLLSSPLRAVFTT |
| 518 | MLPAPKNLVVSRVTEDSARLSWTAPDAAFDSFYIAYAEPRPDGEAIHLVVPGSERSYDLTGLK |
| | PGTEYSVLIHGVKGGLLSSPLSAIFTT |
| 519 | MLPAPKNLVVSRVTEDSARLSWTAPDAAFDSFYIAYAEPRPDGEAISLKVPGSERSYDLTGLK |
| | PGTEYSVLIHGVKGGLLSSPLEAIFTT |
| 520 | MLPAPKNPVVSRVTEDSARLSWTAPDAAFDSFYIAYAEPRPDGEAIHLLVPGSERSYDLTGLK |
| | PGTEYSVLIHGVKGGLLSSPLKRLSPPVVTITITMAVCRKPVAENLSQTLS |
| 521 | MLPAPKNLVVSRVTEDSARLSWTAPDAAFDSFFIGYLEPQPPGEAIFLDVPGSERSYDLTGLK |
| | PGTEYNVTIQGVKGGFPSPLTAFFTT |
| 522 | MLPAPKNLVVSRVTEDSARLSWTAPDAAFDSFFIGYLEPQPPGEAIVLDVPGSERSYDLTGLK |
| | PGTEYNVTIQGVKGGFPSHPLAAAFTT |
| 523 | MLPAPKNLVVSRVTEDSARLSWTAPDAAFDSFFIGYLEPQPPGEAIGLAVPGSERSYDLTGLK |
| | PGTEYNVTIQGVKGGFPSVPLQANFTT |
| 524 | MLPAPKNLVVSRVTEDSARLSWTAPDAAFDSFYIAYAEPRPDGEAILLRVPGSERSYDLTGLK |
| | PGTEYSVLIHGVKGGLLSSPLVAEFTT |
| 525 | MLPAPKNLVVSRVTEDSARLSWTAPDAAFDSFYIAYAEPRPDGEAILLQVPGSERSYDLTGLK |
| | PGTEYSVLIHGVKGGLLSSPLSASFTT |
| 526 | MLPAPKNLVVSRVTEDSARLSWTAPDAAFDSFYIAYAEPRPDGEAIGLHVPGSERSYDLTGLK |
| | PGTEYSVLIHGVKGGLLSSPLTASFTT |
| 527 | MLPAPKNLVVSRVTEDSARLSWTAPDAAFDSFYIAYAEPRPDGEAIGLRVPGSERSYDLTGLK |
| | PGTEYSVLIHGVKGGLLSSPLSAIFTT |
| 528 | MLPAPKNLVVSRVTEDSARLSWTAPDAAFDSFYIAYAEPRPDGEAIVLRVPGSERSYDLTGLK |
| | PGTEYSVLIHGVKGGLLSSPLAASFTT |
| 529 | MLPAPKNLVVSRVTEDSARLSWTAPDAAFDSFYIAYAEPRPDGEAIQLLVPGSERSYDLTGLK |
| | PGTEYSVLIHGVKGGLLSSPLVAHFTT |
| 530 | MLPAPKNLVVSRVTEDSARLSWTAPDAAFDSFYIAYAEPRPDGEAIWLLVPGSERSYDLTGLK |
| | PGTEYSVLIHGVKGGLLSSPLVAFFTT |
| 531 | MLPAPKNLVVSRVTEDSARLSWTAPDAAFDSFFIGYLEPQPPGEAIHLYVPGSERSYDLTGLK |
| | PGTEYNVTIQGVKGGFPSDPLDAVFTT |
| 532 | MLPAPKNLVVSRVTEDSARLSWTAPDAAFDSFFIGYLEPQPPGEAIYLDVPGSERSYDLTGLK |
| | PGTEYNVTIQGVKGGFPSTFTT |
| 533 | MLPAPKNLVVSRVTEDSARLSWTAPDAAFDSFYIAYAEPRPDGEAIQLFVPGSERSYDLTGLK |
| | PGTEYSVLIHGVKGGLLSSPLKAYFTT |
| 534 | MLPAPKNLVVSRVTEDSARLSWTAPDAAFDSFYIAYAEPRPDGEAIVLVVPGSERSYDLTGLK |
| | PGTEYSVLIHGVKGGLLSSPLSAIFTT |
| 535 | MLPAPKNLVVSRVTEDSARLSWTAPDAAFDSFFIGYLEPQPPGEAIQLTVPGSERSYDLTGLK |
| | PGTEYNVTIQGVKGGFPSLPLSADFTT |
| 536 | MLPAPKNLVVSRVTEDSARLSWTAPDAAFDSFYIAYAEPRPDGEAILLQVPGSERSYDLTGLK |
| | PGTEYSVLIHGVKGGLLSSPLDAEFTT |
| 537 | MLPAPKNLVVSRVTEDSARLSWTAPDAAFDSFYIAYAEPRPDGEAIVLAVPGSERSYDLTGLK |
| | PGTEYSVLIHGVKGGLLSSPLYASFTT |
| 538 | MLPAPKNLVVSRVTEDSARLSWTTPDAAFDSFYIAYAEPRPDGEAIRLQVPGSERSYDLTGLK |
| | PGTEYSVLIHGVKGGLLSSPLGFTT |
| 539 | MLPAPKNLVVSRVTEDSARLSWTAPDAAFDSFYIAYAEPRPDGEAIQLVVPGSERSYDLTGLK |
| | PGTEYSVLIHGVKGGLLSSPLYAIFTT |
| 540 | MLPAPKNLVVSRVTEDSARLSWTAPDAAFDSFYIAYAEPRPDGEAISLSVPGSERSYDLTGLK |
| | PGTEYSVLIHGVKGGLLSSPLHAKFTT |
| 541 | MLPAPKNLVVSRVTEDSARLSWTAPDAAFDSFFIGYLEPQPPGEAIHLGVPGSERSYDLTGLK |
| | PGTEYNVTIQGVKGGFPSIPLFASFTT |
| 542 | MLPAPKNLVVSRVTEDSARLSWTAPDAAFDSFYIAYAEPRPDGEAIQLLVPGSERSYDLTGLK |
| | PGTEYSVLIHGVKGGLLSSPLYAAFTT |
| 543 | MLPAPKNLVVSRVTEDSARLSWTAPDAAFDSFFIGYLEPQPPGEAIHLAVPGSERSYDLTGLK |
| | PGTEYNVTIQGVKGGFPSVPLAAVFTT |
| 544 | MLPAPKNLVVSRVTEDSARLSWTAPDAAFDSFYIAYAEPRPDGEAISLQVPGSERSYDLTGLK |
| | PGTEYSVLIHGVKGGLLSSPLGAHFTT |
| 545 | MLPAPKNLVVSRVTEDSARLSWTAPDAAFDSFYIAYAEPRPDGEAIALWVPGSERSYDLTGLK |
| | PGTEYSVLIHGVKGGLLSSPLVASFTT |
| 546 | MLPAPKNLVVSRVTEDSARLSWTAPDAAFDSFYIAYAEPRPDGEAIQLHVPGSERSYDLTGLK |
| | PGTEYSVLIHGVKGGLLSSPLYAFFTT |
| 547 | MLPAPKNLVVSRVTEDSARLSWTAPDAAFDSFYIAYAEPRPDGEAILLHVPGSERSYDLTGLK |
| | PGTEYSVLIHGVKGGLLSSPLRASFTT |
| 548 | MLPAPKNLVVSRVTEDSARLSWTAPDAAFDSFYIAYAEPRPDGEAIWLGVPGSERSYDLTGLK |
| | PGTEYSVLIHGVKGGLLSSPLHATFTT |
| 549 | MLPAPKNLVVSRVTEDSARLSWTAPDAAFDSFYIAYAEPRPDGEAIVLEVPGSERSYDLTGLK |
| | PGTEYSVLIHGVKGGLLSSPLHANFTT |
| 550 | MLPAPKNLVVSRVTEDSARLSWTAPDAAFDSFYIAYAEPRPDGEAILLRVPGSERSYDLTGLK |
| | PGTEYSVLIHGVKGGLLSSPLYAKFTT |
| 551 | MLPAPKNLVVSRVTEDSARLSWTAPDAAFDSFFIGYLEPQPPGEAIGLWVPGSERSYDLTGLK |
| | PGTEYNVTIQGVKGGFPSDPLQAVFTT |
| 552 | MLPAPKNLVVSRVTEDSARLSWTAPDAAFDSFYIAYAEPRPDGEAIVLHVPGSERSYDLTGLK |
| | PGTEYSVLIHGVKGGLLSSPLDAFFTT |
| 553 | MLPAPKNLVVSRVTEDSARLSWTAPDAAFDSFYIAYAEPRPDGEAIILHVPGSERSYDLTGLKP |
| | GTEYSVLIHGVKGGLLSSPLDAYFTT |
| 554 | MLPAPKNLVVSRVTEDSARLSWTAPDAAFDSFYIAYAEPRPDGEAILLAVPGSERSYDLTGLK |
| | PGTEYSVLIHGVKGGLLSSPLSAKFTT |
| 555 | MLPAPKNLVVSRVTEDSARLSWTAPDAAFDSFFIGYLEPQPPGEAILLFVPGSERSYDLTGLKP |
| | GTEYNVTIQGVKGGFPSTPLSASFTT |
| 556 | MLPAPKNLVVSRVTEDSARLSWTAPDAAFDSFYIAYAEPRPDGEAIQLTVPGSERSYDLTGLK |
| | PGTEYSVLIHGVKGGLLSSPLHAYFTT |
| 557 | MLPAPKNLVVSRVTEDSARLSWTAPDAAFDSFYIAYAEPRPDGEAIQLGVPGSERSYDLTGLK |
| | PGTEYSVLIHGVKGGLLSSPLRAYFTT |
| 558 | MLPAPKNLVVSRVTEDSARLSWTAPDAAFDSFYIAYAEPRPDGEAILLEVPGSERSYDLTGLK |
| | PGTEYSVLIHGVKGGLLSSPLVAFFTT |
| 559 | MLPAPKNLVVSRVTEDSARLSWTAPDAAFDSFYIAYAEPRPDGEAIQLGVPGSERSYDLTGLK |
| | PGTEYSVLIHGVKGGLLSSPLLAVFTT |
| 560 | MLPAPKNLVVSRVTEDSARLSWTAPDAAFDSFYIAYAEPRPDGEAIHLRVPGSERSYDLTGLK |
| | PGTEYNVTIQGVKGGFPSMPLSAIFTT |
| 561 | MLPAPKNLVVSRVTEDSARLSWTAPDAAFDSFYIAYAEPRPDGEAILLQVPGSERSYDLTGLK |
| | PGTEYSVLIHGVKGGLLSSPLIAKFTT |
| 562 | MLPAPKNLVVSRVTEDSARLSWTAPDAAFDSFYIAYAEPRPDGEAIVLHVPGSERSYDLTGLK |
| | PGTEYSVLIHGVKGGLLSSPLQAIFTT |
| 563 | MLPAPKNLVVSRVTEDSARLSWTAPDAAFDSFYIAYAEPRPDGEAIALVVPGSERSYDLTGLK |
| | PGTEYSVLIHGVKGGLLSSPLAANFTT |
| 564 | MLPAPKNLVVSRVTEDSARLSWTAPDAAFDSFYIAYAEPRPDGEAINLSVPGSERSYDLTGLK |
| | PGTEYSVLIHGVKGGLLSSPLDAYFTT |
| 565 | MLPAPKNLVVSRVTEDSARLSWTAPDAAFDSFYIAYAEPRPDGEAIVLEVPGSERSYDLTGLK |
| | PGTEYSVLIHGVKGGLLSSPLTASFTT |
| 566 | MLPAPKNLVVSRVTEDSARLSWTAPDAAFDSFYIAYAEPRPDGEAIRLQVPGSERSYDLTGLK |
| | PGTEYSVLIHGVKGGLLSSPLGASFTT |
| 567 | MLPAPKNLVVSRVTEDSARLSWTAPDAAFDSFYIAYAEPRPDGEAIGLWVPGSERSYDLTGLK |
| | PGTEYSVLIHGVKGGLLSSPLVAYFTT |
| 568 | MLPAPKNLVVSRVTEDSARLSWTAPDAAFDSFYIAYAEPRPDGEAIYLEVPGSERSYDLTGLK |
| | PGTEYSVLIHGVKGGLLSSPLFTT |
| 569 | MLPAPKNLVVSRVTEDSARLSWTAPDAAFDSFYIAYAEPRPDGEAIWLDVPGSERSYDLTGLK |
| | PGTEYSVLIHGVKGGLLSSPLDAYFTT |
| 570 | MLPAPKNLVVSRVTEDSARLSWTAPDAAFDSFAIEYCETKMCGEAIVLTVPGSERSYDLTGLK |
| | PGTEYRVPIPGVKGGTASLPLSAIFTT |
| 571 | MLPAPKNLVVSRVTEDSARLSWTAPDAAFDSFFIYYIESYPAGEAIVLTVPGSERSYDLTGLKP |
| | GTEYWVGIDGVKGGRWSTPLSAIFTT |
| 572 | MLPAPKNLVVSRVTEDSARLSWTAPDAAFDSFFIYYIESYPAGEAIVLTVPGSCRSYDLTGLKP |
| | GTEYWVGIDGVKGGRWSTPLSAIFTT |
| 592 | MLPAPKNLVVSRVTEDSARLSWTAPDAAFDSFAIVYHEPRPDGEAIVLTVPGSCRSYDLTGLK |
| | PGTEYEVVILGVKGGVHSYPLSAIFTTAPAPAPAPAPLPAPKNLVVSRVTEDSARLSWTAPDAA |
| | FDSFAIVYHEPRPDGEAIVLTVPGSERSYDLTGLKPGTEYEVVILGVKGGVHSYPLSAIFTT |
| 593 | MLPAPKNLVVSRVTEDSARLSWTAPDAAFDSFAIVYHEPRPDGEAIVLTVPGSCRSYDLTGLK |
| | PGTEYEVVILGVKGGVHSYPLSAIFTTGGGGSGGGGSGGGGSGGGGSLPAPKNLVVSRVTE |
| | DSARLSWTAPDAAFDSFAIVYHEPRPDGEAIVLTVPGSERSYDLTGLKPGTEYEVVILGVKGG |
| | VHSYPLSAIFTT |
| 594 | MLPAPKNLVVSRVTEDSARLSWTAPDAAFDSFAIVYHEPRPDGEAIVLTVPGSCRSYDLTGLK |
| | PGTEYEVVILGVKGGVHSYPLSAIFTTEAAAKEAAAKEAAAKEAAAKLPAPKNLVVSRVTEDSA |
| | RLSWTAPDAAFDSFAIVYHEPRPDGEAIVLTVPGSERSYDLTGLKPGTEYEVVILGVKGGVHS |
| | YPLSAIFTT |
| 595 | MLPAPKNLVVSRVTEDSARLSWTAPDAAFDSFAIVYHEPRPDGEAIVLTVPGSCRSYDLTGLK |
| | PGTEYEVVILGVKGGVHSYPLSAIFTTEAAAKLPAPKNLVVSRVTEDSARLSWTAPDAAFDSFA |
| | IVYHEPRPDGEAIVLTVPGSERSYDLTGLKPGTEYEVVILGVKGGVHSYPLSAIFTT |
| 596 | MLPAPKNLVVSRVTEDSARLSWTAPDAAFDSFKIEYFEYVGYGEAIVLTVPGSCRSYDLTGLK |
| | PGTEYYVAIYGVKGGWYSRPLSAIFTTAPAPAPAPAPLPAPKNLVVSRVTEDSARLSWTAPDA |
| | AFDSFKIEYFEYVGYGEAIVLTVPGSERSYDLTGLKPGTEYYVAIYGVKGGWYSRPLSAIFTT |
| 597 | MLPAPKNLVVSRVTEDSARLSWTAPDAAFDSFKIEYFEYVGYGEAIVLTVPGSCRSYDLTGLK |
| | PGTEYYVAIYGVKGGWYSRPLSAIFTTGGGGSGGGGSGGGGSGGGGSLPAPKNLVVSRVTE |
| | DSARLSWTAPDAAFDSFKIEYFEYVGYGEAIVLTVPGSERSYDLTGLKPGTEYYVAIYGVKGG |
| | WYSRPLSAIFTT |
| 598 | MLPAPKNLVVSRVTEDSARLSWTAPDAAFDSFKIEYFEYVGYGEAIVLTVPGSCRSYDLTGLK |
| | PGTEYYVAIYGVKGGWYSRPLSAIFTTEAAAKEAAAKEAAAKEAAAKLPAPKNLVVSRVTEDS |
| | ARLSWTAPDAAFDSFKIEYFEYVGYGEAIVLTVPGSERSYDLTGLKPGTEYYVAIYGVKGGWY |
| | SRPLSAIFTT |
| 599 | MLPAPKNLVVSRVTEDSARLSWTAPDAAFDSFKIEYFEYVGYGEAIVLTVPGSCRSYDLTGLK |
| | PGTEYYVAIYGVKGGWYSRPLSAIFTTEAAAKLPAPKNLVVSRVTEDSARLSWTAPDAAFDSF |
| | KIEYFEYVGYGEAIVLTVPGSERSYDLTGLKPGTEYYVAIYGVKGGWYSRPLSAIFTT |
| 708 | MLPAPKNLVVSRVTEDSARLSWTAPDAAFDSFAIVYHEPRPDGEAIVLTVPGSCRSYDLTGLK |
| | PGTEYEVVILGVKGGVHSYPLSAIFTT |
| 709 | MLPAPKNLVVSRVTEDSARLSWTAPDAAFDSFAIVYHEPRPDGEAIVLTVPGSERSYDLTGLK |
| | PGTEYEVVILGVKGGVHSYPLSAIFTT |
| 710 | MLPAPKNLVVSRVTEDSARLSWTAPDAAFDSFFIGYLEPQPPGEAIHLGVPGSCRSYDLTGLK |
| | PGTEYNVTIQGVKGGFPSIPLFASFTT |

As provided herein, in some embodiments, the FN3 domain that binds to CD71 binds to SEQ ID NO: 2 (human mature CD71) or SEQ ID NO: 5 (human mature CD71 extracellular domain), sequence of each provided in Table 7, below.

TABLE 7

| SEQ ID | |
|---|---|
| NO: | SEQUENCE |
| 2 | MTKEYQDLQHLDNEESDHHQLRKGPPPPQPLLQRLCSGPRLLLLSLGLSLLLLVVVCVIGSQN |
| | SQLQEELRGLRETFSNFTASTEAQVKGLSTQGGNVGRKMKSLESQLEKQQKDLSEDHSSLLL |
| | HVKQFVSDLRSLSCQMAALQGNGSERTCCPVNWVEHERSCYWFSRSGKAWADADNYCRLE |
| | DAHLVVVTSWEEQKFVQHHIGPVNTWMGLHDQNGPWKWVDGTDYETGFKNWRPEQPDDW |
| | YGHGLGGGEDCAHFTDDGRWNDDVCQRPYRWVCETELDKASQEPPLL |
| 5 | QNSQLQEELRGLRETFSNFTASTEAQVKGLSTQGGNVGRKMKSLESQLEKQQKDLSEDHSS |
| | LLLHVKQFVSDLRSLSCQMAALQGNGSERTCCPVNWVEHERSCYWFSRSGKAWADADNYC |
| | RLEDAHLVVVTSWEEQKFVQHHIGPVNTWMGLHDQNGPWKWVDGTDYETGFKNWRPEQP |
| | DDWYGHGLGGGEDCAHFTDDGRWNDDVCQRPYRWVCETELDKASQEPPLL |

In some embodiments, the FN3 domain comprises two FN3 domains connected by a linker. The linker can be a flexible linker. The linker can be a short peptide sequence, such as those described herein. For example, the linker can be a G/S or G/A linker and the like. As provided herein, the linker can be, for example, (GS)₂, (SEQ ID NO:720), (GGGS)₂ (SEQ ID NO:721), (GGGGS)₅ (SEQ ID NO:722), (AP)₂₋₂₀, (AP)₂ (SEQ ID NO:723), (AP)₅ (SEQ ID NO:724), (AP)₁₀ (SEQ ID NO:725), (AP)₂₀ (SEQ ID NO:726) and A(EAAAK)₅AAA (SEQ ID NO:727) or (EAAAK)₁₋₅ (SEQ ID NO: 728). These are non-limiting examples and other linkers can also be used. The number of GGGGS or GGGGA repeats can also be 1, 2, 3, 4, or 5. In some embodiments, the linker comprises one or more GGGGS repeats and one or more GGGGA repeats. In some embodiments, the linker comprises EAAAKEAAAKEAAAKEAAAK (SEQ ID NO: 729); GGGGSGGGGSGGGGSGGGGS (SEQ ID NO: 730); APAPAPAPAP (SEQ ID NO: 731); or EAAAK (SEQ ID NO: 732).

In some embodiments, the FN3 domain comprising two FN3 domains connected by a linker have the amino acid sequence of SEQ ID NO: 592. In some embodiments, the FN3 domain comprising two FN3 domains connected by a linker have the amino acid sequence of SEQ ID NO: 593. In some embodiments, the FN3 domain comprising two FN3 domains connected by a linker have the amino acid sequence of SEQ ID NO: 594, In some embodiments, the FN3 domain comprising two FN3 domains connected by a linker have the amino acid sequence of SEQ ID NO: 595, In some embodiments, the FN3 domain comprising two FN3 domains connected by a linker have the amino acid sequence of SEQ ID NO: 596. In some embodiments, the FN3 domain comprising two FN3 domains connected by a linker have the amino acid sequence of SEQ ID NO: 597. In some embodiments, the FN3 domain comprising two FN3 domains connected by a linker have the amino acid sequence of SEQ ID NO: 598. In some embodiments, the FN3 domain comprising two FN3 domains connected by a linker have the amino acid sequence of SEQ ID NO: 599. In some embodiments, the FN3 domain comprising two FN3 domains connected by a linker have the amino acid sequence of one of SEQ ID NOs: 592-599.

In some embodiments, the FN3 domains may bind CD71, as applicable, with a dissociation constant (K_{D}) of less than about 1×10⁻⁷ M, for example less than about 1×10⁻⁸ M, less than about 1×10⁻⁹ M, less than about 1×10⁻¹⁰ M, less than about 1×10⁻¹¹ M, less than about 1×10⁻¹² M, or less than about 1×10⁻¹³ M as determined by surface plasmon resonance or the Kinexa method, as practiced by those of skill in the art. The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are made with standardized solutions of protein scaffold and antigen, and a standardized buffer, such as the buffers described herein.

In some embodiments, the FN3 domain may bind to its target protein at least 5-fold above the signal obtained for a negative control in a standard solution ELISA assay.

In some embodiments, the FN3 domain that binds or specifically binds its target protein comprises an initiator methionine (Met) linked to the N-terminus of the molecule. In some embodiments, the FN3 domain that binds or specifically binds to its target protein comprises a cysteine (Cys) linked to a C-terminus of the FN3 domain. The addition of the N-terminal Met and/or the C-terminal Cys may facilitate expression and/or conjugation of half-life extending molecules.

The FN3 domain can also contain cysteine substitutions, such as those that are described in U.S. Pat. No. 10,196,446, which is hereby incorporated by reference in its entirety. Briefly, in some embodiments, the polypeptide comprising an FN3 domain can have an FN3 domain that has a residue substituted with a cysteine, which can be referred to as a cysteine engineered fibronectin type III (FN3) domain. In some embodiments, the FN3 domain comprises at least one cysteine substitution at a position selected from the group consisting of residues 6, 8, 10, 11, 14, 15, 16, 20, 30, 34, 38, 40, 41, 45, 47, 48, 53, 54, 59, 60, 62, 64, 70, 88, 89, 90, 91, and 93 of the FN3 domain based on SEQ ID NO: 1 (LPAPKNLVVSEVTEDSLRLSWTAPDAAFDSFLIQYQESEKVGEAINLTVPGSERSYDLTG LKPGTEYTVSIYGVKGGHRSNPLSAEFTT; SEQ ID NO: 713) of U.S. Pat. No. 10,196,446, which is hereby incorporated by reference in its entirety, and the equivalent positions in related FN3 domains. A cysteine substitution at a position in the domain or protein comprises a replacement of the existing amino acid residue with a cysteine residue. Other examples of cysteine modifications can be found in, for example, U.S. Patent Application Publication No. 20170362301, which is hereby incorporated by reference in its entirety. The alignment of the sequences can be performed using BlastP using the default parameters at, for example, the NCBI website.

In some embodiments, the FN3 domain that binds to the target protein is internalized into a cell. In some embodiments, internalization of the FN3 domain may facilitate delivery of a detectable label or therapeutic into a cell. In some embodiments, internalization of the FN3 domain may facilitate delivery of a cytotoxic agent into a cell. The cytotoxic agent can act as a therapeutic agent. In some embodiments, internalization of the FN3 domain may facilitate the delivery of any detectable label, therapeutic, and/or cytotoxic agent disclosed herein into a cell. In some embodiments, the cell is a tumor cell. In some embodiments, the cell is a liver cell, a lung cell, muscle cell, an immune cell, a dendritic cell, a cell of the CNS, or a heart cell. In some embodiments, the therapeutic is a siRNA molecule as provided for herein. The FN3 domains that bind CD71 conjugated to a detectable label can be used to evaluate expression of CD71 on samples such as tumor tissue in vivo or in vitro. The FN3 domains that bind CD71 conjugated to a detectable label can be used to evaluate expression of CD71 on samples blood, immune cells, muscle cells, or dendritic cells in vivo or in vitro.

As provided herein, the different FN3 domains that are linked to the siRNA molecule can also be conjugated or linked to another FN3 domain that binds to a different target. This would enable the molecule to be multi-specific (e.g. bi-specific, tri-specific, etc.), such that it binds to a first target and another, for example, target. In some embodiments, the first FN3 binding domain is linked to another FN3 domain that binds to an antigen expressed by a tumor cell (tumor antigen).

In some embodiments, FN3 domains can be linked together by a linker to form a bivalent FN3 domain. The linker can be a flexible linker. In some embodiments, the linker is a G/S linker. In some embodiments the linker has 1, 2, 3, or 4 G/S repeats. A G/S repeat unit is four glycines followed by a serine, e.g. GGGGS. Other examples of linkers are provided herein and can also be used.

In some embodiments, the linker is a polypeptide of (GS)₂, (SEQ ID NO:720), (GGGS)₂ (SEQ ID NO:721), (GGGGS)₅ (SEQ ID NO:722), (AP)₂₋₂₀, (AP)₂ (SEQ ID NO:723), (AP)₅ (SEQ ID NO:724), (AP)₁₀ (SEQ ID NO:725), (AP)₂₀ (SEQ ID NO:726) and A(EAAAK)₅AAA (SEQ ID NO:727) or (EAAAK)₁₋₅ (SEQ ID NO: 728). These are non-limiting examples and other linkers can also be used. The number of GGGGS or GGGGA repeats can also be 1, 2, 3, 4, or 5. In some embodiments, the linker comprises one or more GGGGS repeats and one or more GGGGA repeats. In some embodiments, the linker comprises one or more GGGGS repeats and one or more EAAAK repeats. In some embodiments, the linker comprises one or more GGGGS repeats and one or more “AP” repeats. In some embodiments, the linker comprises EAAAKEAAAKEAAAKEAAAK (SEQ ID NO: 729); GGGGSGGGGSGGGGSGGGGS (SEQ ID NO: 730); APAPAPAPAP (SEQ ID NO: 731); or EAAAK (SEQ ID NO: 732).

Without being bound to any particular theory, in some embodiments, the FN3 domains that are linked to the nucleic acid molecule may be used in the targeted delivery of the therapeutic agent to cells that express the binding partner of the one or more FN3 domains (e.g. tumor cells), and lead intracellular accumulation of the nucleic acid molecule therein. This can allow the siRNA molecule to properly interact with the cell machinery to inhibit the expression of the target gene, improve efficacy, and also avoid, in some embodiments, toxicity that may arise with untargeted administration of the same siRNA molecule.

The FN3 domain described herein that bind to their specific target protein may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline. Exemplary linker include (GS)₂, (SEQ ID NO:720), (GGGS)₂ (SEQ ID NO:721), (GGGGS)₅ (SEQ ID NO:722), (AP)₂₋₂₀, (AP)₂ (SEQ ID NO:723), (AP)₅ (SEQ ID NO:724), (AP)₁₀ (SEQ ID NO:725), (AP)₂₀ (SEQ ID NO:726) and A(EAAAK)₅AAA (SEQ ID NO:727) or (EAAAK)₁₋₅ (SEQ ID NO: 728). In some embodiments, the linker comprises or is an amino acid sequence of: EAAAKEAAAKEAAAKEAAAK (SEQ ID NO: 729); GGGGSGGGGSGGGGSGGGGS (SEQ ID NO: 730); APAPAPAPAP (SEQ ID NO: 731); or EAAAK (SEQ ID NO: 732).

The dimers and multimers may be linked to each other in a N-to C-direction. The use of naturally occurring as well as synthetic peptide linkers to connect polypeptides into novel linked fusion polypeptides is well known in the literature. See, e.g., Hallewell et al. (J Biol Chem., 1989, 264:5260-5268); Alfthan et al. (Protein Eng., 1995, 8:725-731); Robinson & Sauer (Biochemistry, 1996, 35:109-116); and U.S. Pat. No. 5,856,456. The linkers described in this paragraph may be also be used to link the domains provided in the formula provided herein and above.

Half-Life Extending Moieties

The FN3 domains may also, in some embodiments, incorporate other subunits for example via covalent interaction. In some embodiments, the FN3 domains that further comprise a half-life extending moiety. Exemplary half-life extending moieties are albumin, albumin variants, albumin-binding proteins and/or domains, transferrin and fragments and analogues thereof, and Fc regions. Amino acid sequences of the human Fc regions are well known, and include IgG1, IgG2, IgG3, IgG4, IgM, IgA and IgE Fc regions. In some embodiments, the FN3 domain binds to albumin, albumin variants, albumin-binding proteins and/or domains, and fragments and analogues thereof. extending the half-life of the entire molecule.

In some embodiments, the albumin binding domain comprises the amino acid sequence of SEQ ID NOs: 101, 102, 103, 104, 105, 106, 107, 108, 109, 110, 111, 112, 113, 114, 115, 116, 117, 118, or 119. In some embodiments, the albumin binding domain (protein) is isolated. In some embodiments, the albumin binding domain comprises an amino acid sequence that is at least, or is, 85%, 86%, 87%, 88%, 89%, 90%, 91%, 92%, 93%, 94%, 95%, 96%, 97%, 98%, or 99% identical to the amino acid sequence of SEQ ID NO: 101, 102, 103, 104, 105, 106, 107, 108, 109, 110, 111, 112, 113, 114, 115, 116, 117, 118, or 119. In some embodiments, the albumin binding domain comprises an amino acid sequence that is at least, or is, 85%, 86%, 87%, 88%, 89%, 90%, 901%, 92%, 93%, 94%, 95%, 96%, 97%, 98%, or 99% identical to the amino acid sequence of SEQ ID NO: 101, 102, 103, 104, 105, 106, 107, 108, 109, 110, 111, 112, 113, 114, 115, 116, 117, 118, or 119 provided that the protein has a substitution that corresponds to position 10 of SEQ ID NO: 101, 102, 103, 104, 105, 106, 107, 108, 109, 110, 111, 112, 113, 114, 115, 116, 117, 118, or 119. In some embodiments, the substitution is A10V. In some embodiments, the substitution is A10G, A10L, A10I, A10T, or A10S. In some embodiments, the substitution at position 10 is any naturally occurring amino acid. In some embodiments, the isolated albumin binding domain comprises an amino acid sequence that has 1, 2, 3, 4, 5, 6, 7, 8, 9, 10, 11, 12, 13, or 14 substitutions when compared to the amino acid sequence of SEQ ID NOs: 101, 102, 103, 104, 105, 106, 107, 108, 109, 110, 111, 112, 113, 114, 115, 116, 117, 118, or 119. In some embodiments, the substitution is at a position that corresponds to position 10 of SEQ ID NOs: 101, 102, 103, 104, 105, 106, 107, 108, 109, 110, 111, 112, 113, 114, 115, 116, 117, 118, or 119. In some embodiments, FN3 domains provided comprises a cysteine residue in at least one residue position corresponding to residue positions 6, 11, 22, 25, 26, 52, 53, 61, 88 or positions 6, 8, 10, 11, 14, 15, 16, 20, 30, 34, 38, 40, 41, 45, 47, 48, 53, 54, 59, 60, 62, 64, 70, 88, 89, 90, 91, or 93 of SEQ ID NO: 101, 102, 103, 104, 105, 106, 107, 108, 109, 110, 111, 112, 113, 114, 115, 116, 117, 118, or 119, or at a C-terminus. Although the positions are listed in a series, each position can also be chosen individually. In some embodiments, the cysteine is at a position that corresponds to position 6, 53, or 88. In some embodiments, additional examples of albumin binding domains can be found in U.S. Pat. No. 10,925,932, which hereby incorporated by reference.

All or a portion of an antibody constant region may be attached to the FN3 domain to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, down regulation of cell surface receptors (e.g., B cell receptor; BCR), and may be further modified by modifying residues in the Fc responsible for these activities (for review; see Strohl, Curr Opin Biotechnol. 20, 685-691, 2009).

Additional moieties may be incorporated into the FN3 domains such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules disclosed herein.

A PEG moiety may for example be added to the FN3 domain t by incorporating a cysteine residue to the C-terminus of the molecule, or engineering cysteines into residue positions that face away from the binding face of the molecule, and attaching a PEG group to the cysteine using well known methods.

FN3 domains incorporating additional moieties may be compared for functionality by several well-known assays. For example, altered properties due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating pharmacokinetic properties of the molecules disclosed herein in in vivo models.

The compositions provided herein can be prepared by preparing the FN3 proteins and the nucleic acid molecules and linking them together. The techniques for linking the proteins to a nucleic acid molecule are known and any method can be used. For example, in some embodiments, the nucleic acid molecule is modified with a linker, such as the linker provided herein, and then the protein is mixed with the nucleic acid molecule comprising the linker to form the composition. For example, in some embodiments, a FN3 domains is conjugated to a siRNA a cysteine using thiol-maleimide chemistry. In some embodimetns, a cysteine-containing FN3 domain can be reduced in, for example, phosphate buffered saline (or any other appropriate buffer) with a reducing agent (e.g. tris(2-carboxyethyl) phosphine (TCEP)) to yield a free thiol. Then, in some embodiments, the free thiol containing FN3 domain was mixed with a maleimide linked-modified siRNA duplex and incubated under conditions to form the linked complex. In some embodiments, the mixture is incubated for 0-5 hr, or about 1, 2, 3, 4 or 5 hr at RT. The reaction can be, for example, quenched with N-ethyl maleimide. In some embodiments, the conjugates can be purified using affinity chromatography and ion exchange. Other methods can also be used and this is simply one non-limiting embodiment.

Methods of making FN3 proteins are known and any method can be used to produce the protein. Examples are provided in the references incorporated by reference herein.

In some embodiments, the FN3 domain specifically binding CD71 comprises the amino acid sequence of SEQ ID NOs: 301-301, 310, 312-519, 521-572, 592-599, or 708-710, wherein a histidine tag has been appended to the N-terminal or C-terminal end of the polypeptide for ease of purification. In some embodiments, the histidine tag (His-tag) comprises six histidine residues. In further embodiments, the His-tag to connected to the FN3 domain by at least one glycine residue or about 2 to about 4 glycine residues. Accordingly, after purification of the FN3 domain and cleavage of the His-tag from the polypeptide one or more glycine may be left on the N-terminus or C-terminus. In some embodiments, if the His-tag is removed from the N-terminus all of the glycines are removed. In some embodiments, if the His-tag is removed from the C-terminus one or more of the glycines are retained.

In some embodiments, the FN3 domain specifically binding CD71 comprises the amino acid sequence of SEQ ID NOs: 301-301, 310, 312-519, 521-572, 592-599, or 708-710, wherein the N-terminal methionine is retained after purification of the FN3 domain.

Kits

In some embodiments, a kit comprising the compositions described herein are provided.

The kit may be used for therapeutic uses and as a diagnostic kit.

In some embodiments, the kit comprises the FN3 domain conjugated to the nucleic acid molecule.

In some embodiments, kits are provided for treating a glycogen storage diseases. In some embodiments, the kits comprise a first container comprising a pharmaceutical composition comprising one or more FN3 domains linked to an siRNA comprising a sense strand and antisense strand; and a second container comprising a pharmaceutical composition comprising an enzyme replacement therapy (ERT) to treat the glycogen storage disease. In some embodiments, the one or more FN3 domains comprise a FN3 domain that binds to CD71. In some embodiments, the siRNA targets Gys1. In some embodiments, the one or more FN3 domains comprise a FN3 domain that binds to CD71. In some embodiments, the glycogen storage disease is selected from the group consisting of Pompe Disease (GSD2, glucosidase alpha acid (GAA) deficiency), Cori's disease or Forbes' disease (GSD3, Glycogen debranching enzyme (AGL) deficiency), Andersen's disease (GSD4, glycogen branching enzyme (GBE1) deficiency), McArdle disease (GSD5, Muscle glycogen phosphorylase (PYGM) deficiency), Tarui's Disease (GSD7, Muscle phosphofructokinase (PFKM) deficiency), Aldolase A Deficiency (GSD12, aldolase A (ALDOA) deficiency), type II Diabetes/diabetic nephropathy, Lafora Disease, hypoxia, and adult polyglucosan body disease. In some embodiments, the ERT comprises glucosidase alpha acid (GAA), glycogen debranching enzyme (AGL), glycogen branching enzyme (BGE1), muscle glycogen phosphorylase (PYGM), muscle phosphofructokinase (PFKM), aldolase A (ALDOA), malin, laforin, glycogen synthase (GYS2), glucose-6-phosphatase (G6PC/SLC37A4), phosphorylase kinase (PHKA2/PHKB/PHKG2/PHKA1), phosphoglycerate mutase (PGAM2), muscle lactate dehydrogenase (LDHA), glucose transporter (GLUT2), β-enolase (ENO3), and glycogenin-1 (GYG1), or a combination thereof. In some embodiments, the ERT comprises GAA, malin, laforin, or a combination thereof. In some embodiments, the first container comprises a pharmaceutical composition comprising a FN3 domain that binds CD71 linked to a siRNA that targets Gys1 and the second container comprises a pharmaceutical composition comprising the enzyme of GAA.

Uses of the Conjugates FN3 Domains

The compositions provided for herein may be used to diagnose, monitor, modulate, treat, alleviate, help prevent the incidence of, or reduce the symptoms of human disease or specific pathologies in cells, tissues, organs, fluid, or, generally, a host.

In some embodiments, methods of selectively reducing GYS1 mRNA and protein in skeletal muscle. In certain embodiments, GYS1 mRNA and protein is not reduced in the liver and/or the kidney.

In some embodiments, the reduction in the GYS1 mRNA and protein is sustained for about 1 week, about 2 weeks, about 3 weeks, about 4 weeks, about 5 weeks, or greater than 5 weeks after administration of the conjugate described herein.

In some embodiments, the FN3 domain can facilitate delivery into CD71 positive tissues (e.g., skeletal muscle, smooth muscle) for treatment of muscle diseases.

In some embodiments, the FN3 domain can facilitate delivery to activated lymphocytes, dendritic cells, or other immune cells for treatment of immunological diseases.

In some embodiments, the polypeptide that binds to CD71 is directed to immune cells. In some embodiments, the polypeptide that binds to CD71 is directed to dendritic cells. In some embodiments, methods of treating an autoimmune disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID NOs: 301-301, 310, 312-519, 521-572, 592-599, or 708-710, or a polypeptide as provided herein that is linked to or conjugated to a therapeutic agent. In some embodiments, the autoimmune disease is selected from the group consisting of rheumatoid arthritis, Hashimoto's autoimmune thyroiditis, celiac disease, diabetes mellitus type 1, vitiligo, rheumatic fever, pernicious anemia/atrophic gastritis, alopecia areata, and immune thrombocytopenic purpura.

In some embodiments, a method of treating a subject having Pompe Disease (GSD2, acid alpha-glucosidase (GAA) deficiency) is provided, the method comprising administering to the subject a composition provided for herein. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 301-301, 310, 312-519, 521-572, 592-599, or 708-710, or a polypeptide as provided herein that is linked to or conjugated to a therapeutic agent.

In some embodiments, methods of treating glycogen storage disease in a subject in need thereof, the method comprising administering a composition provided herein are provided. In some embodiments, the glycogen storage disease is selected from the group consisting of Cori's disease or Forbes' disease (GSD3, Glycogen debranching enzyme (AGL) deficiency), McArdle disease (GSD5, Muscle glycogen phosphorylase (PYGM) deficiency), type II Diabetes/diabetic nephropathy, Aldolase A Deficiency GSD12, Lafora Disease, hypoxia, Andersen disease (GSD4, Glycogen debranching enzyme (GBE1) deficiency), Tarui's Disease (GSD7, Muscle phosphofructokinase (PFKM) deficiency), and adult polyglucosan body disease. In some embodiments, the glycogen storage disease is selected from the group consisting of Glycogen synthase (GYS2) deficiency (GSD0), Glucose-6-phosphatase (G6PC/SLC37A4) deficiency (GSD1, von Gierke's disease), Hers' disease (GSD6, Liver glycogen phosphorylase (PYGL) or Muscle phosphoglycerate mutase (PGAM2) deficiency), Phosphorylase kinase (PHKA2/PHKB/PHKG2/PHKA1) deficiency (GSD9), Phosphoglycerate mutase (PGAM2) deficiency (GSD10), Muscle lactate dehydrogenase (LDHA) deficiency (GSD11), Fanconi-Bickel syndrome (GSD 11, Glucose transporter (GLUT2) deficiency, Aldolase A deficiency (GSD 12), β-enolase (ENO3) deficiency (GSD13), and Glycogenin-1 (GYG1) deficiency (GSD15).

In some embodiments, a use of a composition as provided herein or of any of in the preparation of a pharmaceutical composition or medicament for treating cancer are provided. In some embodiments, the cancer is selected from the group consisting of acute myeloid leukemia, myelodysplastic syndromes, gastric cancer, clear cell renal cell carcinoma, clear cell carcinomas of the breast, clear cell carcinomas of the endometrium, clear cell carcinomas of the ovary, clear cell carcinomas of the uterus, hepatocellular carcinoma, pancreatic cancer, prostate cancer, soft tissue cancer, Ewings sarcoma, and non-small cell lung cancer

In some embodiments, the CD71 cell is a cell involved in a CNS diseases, inflammatory/immune diseases, such as MS & infectious diseases of the brain. In some embodiments, the polypeptide that binds to CD71 is directed to the central nervous system. In some embodiments, methods of treating a neurological condition and/or a brain tumor in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID NOs: 301-301, 310, 312-519, 521-572, 592-599, or 708-710, or a polypeptide as provided herein that is linked to or conjugated to a therapeutic agent. In some embodiments, the brain tumor is selected from the group consisting of nonmalignant, benign, and malignant brain tumors. In some embodiments, the neurological condition is selected from the group consisting of Alzheimer's Disease, Amyotrophic Lateral Sclerosis, Parkinson's Disease, Lafora Disease, Pompe Disease, adult polyglucosan body disease, stroke, spinal cord injury, ataxia, Bell's Palsy, cerebral aneurysm, epilepsy, seizures, Guillain-Barre Syndrome, multiple sclerosis, muscular dystrophy, neurocutaneous syndromes, migraine, encephalitis, septicemia, and myasthenia gravis.

In some embodiments, a method of treating a subject having cancer is provided, the method comprising administering to the subject a composition provided for herein. In some embodiments, the method comprises administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 301-301, 310, 312-519, 521-572, 592-599, or 708-710, or a polypeptide as provided herein that is linked to or conjugated to a therapeutic agent.

In some embodiments, the subject has a solid tumor.

In some embodiments, the solid tumor is a melanoma.

In some embodiments, the solid tumor is a lung cancer. In some embodiments, the solid tumor is a non-small cell lung cancer (NSCLC). In some embodiments, the solid tumor is a squamous non-small cell lung cancer (NSCLC). In some embodiments, the solid tumor is a non-squamous NSCLC. In some embodiments, the solid tumor is a lung adenocarcinoma.

In some embodiments, the solid tumor is a renal cell carcinoma (RCC).

In some embodiments, the solid tumor is a mesothelioma.

In some embodiments, the solid tumor is a nasopharyngeal carcinoma (NPC).

In some embodiments, the solid tumor is a colorectal cancer.

In some embodiments, the solid tumor is a prostate cancer. In some embodiments, the solid tumor is castration-resistant prostate cancer.

In some embodiments, the solid tumor is a stomach cancer.

In some embodiments, the solid tumor is an ovarian cancer.

In some embodiments, the solid tumor is a gastric cancer.

In some embodiments, the solid tumor is a liver cancer.

In some embodiments, the solid tumor is pancreatic cancer.

In some embodiments, the solid tumor is a thyroid cancer.

In some embodiments, the solid tumor is a squamous cell carcinoma of the head and neck.

In some embodiments, the solid tumor is a carcinomas of the esophagus or gastrointestinal tract.

In some embodiments, the solid tumor is a breast cancer.

In some embodiments, the solid tumor is a fallopian tube cancer.

In some embodiments, the solid tumor is a brain cancer.

In some embodiments, the solid tumor is an urethral cancer.

In some embodiments, the solid tumor is a genitourinary cancer.

In some embodiments, the solid tumor is an endometriosis.

In some embodiments, the solid tumor is a cervical cancer.

In some embodiments, the solid tumor is a metastatic lesion of the cancer.

In some embodiments, the subject has a hematological malignancy.

In some embodiments, the hematological malignancy is a lymphoma, a myeloma or a leukemia. In some embodiments, the hematological malignancy is a B cell lymphoma. In some embodiments, the hematological malignancy is Burkitt's lymphoma. In some embodiments, the hematological malignancy is Hodgkin's lymphoma. In some embodiments, the hematological malignancy is a non-Hodgkin's lymphoma.

In some embodiments, the hematological malignancy is a myelodysplastic syndrome.

In some embodiments, the hematological malignancy is an acute myeloid leukemia (AML). In some embodiments, the hematological malignancy is a chronic myeloid leukemia (CML). In some embodiments, the hematological malignancy is a chronic myelomoncytic leukemia (CMML).

In some embodiments, the hematological malignancy is a multiple myeloma (MM).

In some embodiments, the hematological malignancy is a plasmacytoma.

In some embodiments, the cancer is a soft tissue cancer. In some embodiments, the soft tissue cancer is Ewings sarcoma.

In some embodiments, methods of treating cancer in a subject in need thereof are provided. In some embodiments, the methods comprise administering to the subject any composition provided herein. In some embodiments, a use of a composition as provided herein are provided in the preparation of a pharmaceutical composition or medicament for treating cancer. In some embodiments, the composition can be used for treating cancer.

In some embodiments, methods of treating Pompe Disease (GSD2, acid alpha-glucosidase (GAA) deficiency) in a subject in need thereof are provided. In some embodiments, the methods comprise administering to the subject any composition provided herein. In some embodiments, a use of a composition as provided herein are provided in the preparation of a pharmaceutical composition or medicament for treating Pompe Disease (GSD2, acid alpha-glucosidase (GAA) deficiency). In some embodiments, the composition can be used for treating Pompe Disease (GSD2, acid alpha-glucosidase (GAA) deficiency).

In some embodiments, methods of treating glycogen storage disease in a subject in need thereof are provided. In some embodiments, the methods comprise administering to the subject any composition provided herein. In some embodiments, a use of a composition as provided herein are provided in the preparation of a pharmaceutical composition or medicament for treating glycogen storage disease. In some embodiments, the composition can be used for treating glycogen storage disease.

In some embodiments, methods of treating glycogen storage disease in a subject in need thereof, the method comprising administering a composition provided herein are provided. In some embodiments, the glycogen storage disease is selected from the group consisting of Cori's disease or Forbes' disease (GSD3, Glycogen debranching enzyme (AGL) deficiency), McArdle disease (GSD5, Muscle glycogen phosphorylase (PYGM) deficiency), type II Diabetes/diabetic nephropathy, Aldolase A Deficiency GSD12, Lafora Disease, hypoxia, Andersen disease (GSD4, Glycogen debranching enzyme (GBE1) deficiency), Tarui's Disease (GSD7, Muscle phosphofructokinase (PFKM) deficiency), and adult polyglucosan body disease. In some embodiments, the glycogen storage disease is selected from the group consisting of Glycogen synthase (GYS2) deficiency (GSD0), Glucose-6-phosphatase (G6PC/SLC37A4) deficiency (GSD1, von Gierke's disease), Hers' disease (GSD6, Liver glycogen phosphorylase (PYGL) or Muscle phosphoglycerate mutase (PGAM2) deficiency), Phosphorylase kinase (PHKA2/PHKB/PHKG2/PHKA1) deficiency (GSD9), Phosphoglycerate mutase (PGAM2) deficiency (GSD10), Muscle lactate dehydrogenase (LDHA) deficiency (GSD11), Fanconi-Bickel syndrome (GSD 11, Glucose transporter (GLUT2) deficiency, Aldolase A deficiency (GSD 12), β-enolase (ENO3) deficiency (GSD13), and Glycogenin-1 (GYG1) deficiency (GSD15).

In some embodiments, the polypeptide that binds to CD71 is directed to the central nervous system. In some embodiments, methods of treating a neurological condition and/or a brain tumor in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 301-301, 310, 312-519, 521-572, 592-599, or 708-710, or a polypeptide as provided herein that is linked to or conjugated to a therapeutic agent. In some embodiments, the brain tumor is selected from the group consisting of nonmalignant, benign, and malignant brain tumors. In some embodiments, the neurological condition is selected from the group consisting of Alzheimer's Disease, Amyotrophic Lateral Sclerosis, Parkinson's Disease, Lafora Disease, Pompe Disease, adult polyglucosan body disease, stroke, spinal cord injury, ataxia, Bell's Palsy, cerebral aneurysm, epilepsy, seizures, Guillain-Barre Syndrome, multiple sclerosis, muscular dystrophy, neurocutaneous syndromes, migraine, encephalitis, septicemia, and myasthenia gravis. In some embodiments, a method of treating a neurological condition and/or a brain tumor in a subject, the method comprising administering to the subject a FN3 domain that binds CD71 and the FN3 domain is conjugated to a therapeutic agent (e.g. cytotoxic agent, an oligonucleotide, such as a siRNA, ASO, and the like, a FN3 domain that binds to another target, and the like).

In some embodiments, methods of treating Pompe disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 301-301, 310, 312-519, 521-572, 592-599, or 708-710, or a polypeptide as provided herein that is linked to or conjugated to a therapeutic agent. In some embodiments, a method of treating a Pompe disease in a subject, the method comprising administering to the subject a FN3 domain that binds CD71 and the FN3 domain is conjugated to a therapeutic agent (e.g. cytotoxic agent, an oligonucleotide, such as a siRNA, ASO, and the like, a FN3 domain that binds to another target, and the like).

In some embodiments, the polypeptide that binds to CD71 is directed to immune cells. In some embodiments, the polypeptide that binds to CD71 is directed to dendritic cells. In some embodiments, methods of treating an autoimmune disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 301-301, 310, 312-519, 521-572, 592-599, or 708-710, or a polypeptide as provided herein that is linked to or conjugated to a therapeutic agent. In some embodiments, the autoimmune disease is selected from the group consisting of rheumatoid arthritis, Hashimoto's autoimmune thyroiditis, celiac disease, diabetes mellitus type 1, vitiligo, rheumatic fever, pernicious anemia/atrophic gastritis, alopecia areata, and immune thrombocytopenic purpura. In some embodiments, a method of treating an autoimmune disease in a subject, the method comprising administering to the subject a FN3 domain that binds CD71 and the FN3 domain is conjugated to a therapeutic agent (e.g. cytotoxic agent, an oligonucleotide, such as a siRNA, ASO, and the like, a FN3 domain that binds to another target, and the like).

In some embodiments, methods of reducing the expression of a target gene in a cell are provided. In some embodiments, the methods comprise delivering to the cell with a composition or a pharmaceutical composition as provided herein. In some embodiments, the cell is ex-vivo. In some embodiments, the cell is in-vivo. In some embodiments, the target gene is GYS1. The target gene, however, can be any target gene as the evidence provided herein demonstrates that siRNA molecules can be delivered efficiently when conjugated to a FN3 domain. In some embodiments, the siRNA targeting GYS1 is linked to a FN3 domain. In some embodiments, the FN3 polypeptide (domain) is one that binds to CD71. In some embodiments, the FN3 polypeptide is as provided for herein or as provided for in PCT Application No. PCT/US20/55509, U.S. application Ser. No. 17/070,337, PCT Application No. PCT/US20/55470, or U.S. application Ser. No. 17/070,020, each of which is hereby incorporated by reference in its entirety. In some embodiments, the siRNA is not conjugated to a FN3 domain.

In some embodiments, methods of reducing the expression of a target gene in a cell are provided. In some embodiments, the methods comprise delivering to the cell with a composition or a pharmaceutical composition as provided herein. In some embodiments, the cell is ex-vivo. In some embodiments, the cell is in-vivo. In some embodiments, a method of reducing the expression of a target gene results in a reduction of about 99%, 90-99%, 50-90%, or 10-50% in the expression of the target gene.

In some embodiments, a method of reducing the expression of GYS1 is provided. In some embodiments, the reduced expression is the expression (amount) of GYS1 mRNA. In some embodiments, a method of reducing the expression of GYS1 results in a reduction of about 99%, 90-99%, 50-90%, or 10-50% in the expression of GYS1. In some embodiments, the reduced expression is the expression (amount) of GYS1 protein. In some embodiments, the reduced protein is glycogen. In some embodiments, reduction of glycogen occurs in muscle cells. In some embodiments, reduction of glycogen occurs in heart cells. In some embodiments, the method comprises delivering to a cell with a siRNA molecule as provided herein that targets GYS1. In some embodiments, the siRNA is conjugated to a FN3 domain. In some embodiments, the FN3 domain is a FN3 domain that binds to CD71. In some embodiments, the FN3 domain is as provided for herein. In some embodiments, the FN3 domain is a dimer of two FN3 domains that bind to CD71. In some embodiments, the FN3 domains are the same. In some embodiments, the two FN3 domains are different, i.e., bind to different regions or amino acid residues of CD71, i.e. a different epitope. In some embodiments, the method comprises administering to a subject (patient) a GYS1 siRNA molecule, such as those provided herein. In some embodiments, the GYS1 siRNA administered to the subject is conjugated or linked to a FN3 domain. In some embodiments, the FN3 domain is a FN3 domain that binds to CD71. In some embodiments, the FN3 domain is as provided for herein. In some embodiments, the FN3 domain is a dimer of two FN3 domains that bind to CD71. In some embodiments, the FN3 domains are the same. In some embodiments, the two FN3 domains are different, i.e., bind to different regions or amino acid residues of CD71, i.e. a different epitope. In some embodiments, the CD71 binding domain is a polypeptide as provided for herein.

In some embodiments, the siRNA is conjugated to a cyclic peptide of Chemical Formula III:

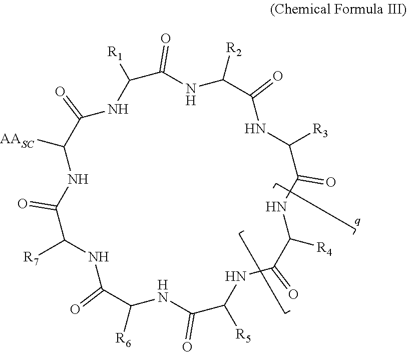

or a protonated form thereof, wherein: R₁, R₂, and R₃ are each independently H or an aromatic or heteroaromatic side chain of an amino acid; at least one of R₁, R₂, and R₃ is an aromatic or heteroaromatic side chain of an amino acid; R₄, R₅, R₆, R₇ are independently H or an amino acid side chain; at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine N,N,N-trimethyllysine, 4-guanidinophenylalanine, citrulline, N,N-dimethyllysine, b-homoarginine, 3-(1-piperidinyl)alanine; AAsc is an amino acid side chain; and q is 1, 2, 3 or 4; wherein the cyclic peptide of the formula @@ is not FfΦ RrRrE, as disclosed in International Patent Publication No. WO2022/213118, hereby incorporated by reference in its entirety. “FfΦ RrRrE” refers to a structure wherein “F” is L-Phe; “f” is D-Phe; “Φ” is L-2-naphthylalanine; “R” is L-Arginine; and “r” is D-Arginine. “FfΦ RrRrE” is also described in Soudah et al. (“AntimiR-155 Cyclic Peptide-PNA Conjugate: Synthesis, Cellular Uptake, and Biological Activity”, ACS Omega, 2019, 4(9):13954-13961), hereby incorporated by reference in its entirety.

In some embodiments, methods of delivering a siRNA molecule to a cell in a subject are provided. In some embodiments, the methods comprise administering to the subject a pharmaceutical composition comprising a composition as provided for herein. In some embodiments, the cell is a CD71 positive cell. The term “positive cell” in reference to a protein refers to a cell that expresses the protein. In some embodiments, the protein is expressed on the cell surface. In some embodiments, the cell is a tumor cell, a liver cell, an immune cell, a dendritic cell, a heart cell, a muscle cell, a cell of the CNS, or a cell inside the blood brain barrier. In some embodiments, the siRNA downregulates the expression of a target gene in the cell. In some embodiments, the target gene is GYS1.

In some embodiments, provided herein is a composition comprising the compound of Chemical Formula IV:

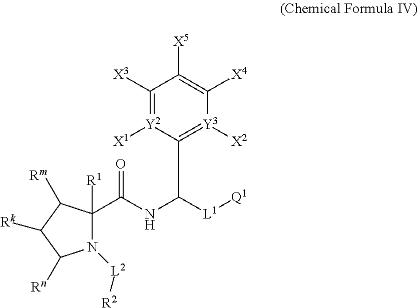

or a stereoisomer or tautomer thereof, or a pharmaceutically acceptable salt of any of the foregoing, wherein: Y² and Y³ are each C, or one of Y² and Y³ is N and the other of Y² and Y³ is C: X¹ and X² are each independently H, C₁₋₆alkyl, or C₁₋₆alkoxy; X³ and X⁴ are each independently H, halo, C1-6alkyl, C1-6alkoxy, or 5-20 membered heteroaryl, wherein the C₁₋₆alkyl of X³ and X⁴ is optionally substituted with one of more halo; X⁵ is H, C₁₋₆alkyl, C₁₋₆alkoxy, or C₃₋₁₀cycloalkyl; either (1) L¹ is absent; and Q¹ is selected from (i) to (iv): (i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋10cycloalkyl, or 5-20 membered heteroaryl, wherein the C1-6alkyl is optionally substituted with one or more halo. —NH—C(O)—NH(C1-6alkyl), —NH—C(O)—C₁₋₆alkyl, or —NH—C(O)—C₁₋₆alkoxy, the C3-10cycloalkyl is optionally substituted with one or more halo or C1-6alkyl, and the 5-20 membered heteroaryl is optionally substituted with one or more C1-6alkyl. (ii) 3-15 membered heterocyclyl, wherein the 3-15 membered heterocyclyl of Q¹ is optionally substituted with one or more oxo, or C₁₋₆alkyl, (iii) 5-20 membered heteroaryl, wherein the 5-20 membered heteroaryl of Q¹ is optionally substituted with one or more halo, C₁₋₆alkyl, C₁₋₆alkenyl. C₁₋₆alkoxy, —NH₂, or C3-10cycloalkyl, wherein, the C1-6alkyl is optionally substituted with one or more halo, and the C3-10cycloalkyl is optionally substituted with one or more halo or C1-6alkyl, and (iv) C₃₋₁₀cycloalkyl; or (2) L¹ is —CH2-; and Q¹ is C₃₋₁₀cycloalkyl; L² is —C(O)— or —S(O)₂—R¹ is H or C1-6alkyl; R^{k} is H, halo, —OH, —NH₂, or —NH—C(O)C₁₋₆alkyl; R^{m} is H, —OH, or C₁₋₆alkyl; Rⁿ is H, C₁₋₆alkyl, or C₃₋₁₀cycloalkyl or Rⁿ taken together with the carbon atom to which it is attached forms C3-5 cycloalkyl; or R^{k} is taken together with either R^{m} or Rⁿ and the atoms to which they are attached, to form cyclopropyl; and R² is selected from (i) to (vii): (i) C1-6alkyl, wherein the C1-6alkyl of R² is optionally substituted with one or more R^{a}, wherein R^{a} is: (a) —OH, (b) cyano, (c) C₂₋₆alkynyl, (d) C₆₋₂₀aryl, wherein the C₆₋₂₀aryl of R^{a} is optionally substituted with one or more halo, cyano. C1-6alkoxy, or —NH—C(O)—C1-6alkyl, (e) 3-15 membered heterocyclyl, wherein the 3-15 membered heterocyclyl of R^{a} is optionally substituted with one or more R^{c}, wherein R^{c} is halo, oxo, C1-6alkyl, CL-6alkoxy, —C(O)—C1-6alkyl, or —C(O)—C1-6alkoxy, wherein the C₁₋₆alkyl of R^{c} is optionally substituted with one or more halo or C2-6alkynyl, and the —C(O)—C1-6alkoxy of R^{c} is optionally substituted with one or more halo, (f) —N(R^{c})R^{d}), wherein R^{c} and R^{d} of —N(R^{c})(R^{d}) are, independently of each other, H, C1-6alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein the C₁₋₆ alkyl of R^{c} or R^{d} is optionally substituted with one or more —C(O)—NH₂, the —C(O)—C₁₋₆alkyl of R^{c} or R^{d} is optionally substituted with one or more halo, the 3-15 membered heterocyclyl and the 5-20 membered heteroaryl of R^{c} or R^{d} are independently optionally substituted with one or more C1-6alkyl, the —C(O)-(3-15 membered heterocyclyl) of R^{c} or R^{d} is optionally substituted with one or more halo, —C(O)—C1-6alkoxy, or C1-6alkyl, wherein the C1-6alkyl is optionally substituted with one or more halo, C1-6alkoxy, or C3-1₀cycloalkyl, and the C₁₋₆alkyl of the —C(O)—N(C₁₋₆alkyl)₂ of R^{c} or R^{d} are, independently of each other, optionally substituted with one or more halo or C6-20aryl, (g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein the C1-6alkyl of R^{e} is optionally substituted with one or more C1-6alkoxy, wherein the C1-6alkoxy is optionally substituted with one or more C2-6alkynyl, the C₆₋₂₀aryl of R^{e} is optionally substituted with one or more C₁₋₆alkyl, and the —C(O)-(3-15 membered heterocyclyl) of R^{e} is optionally substituted with one or more C1-6alkyl, C1-6alkoxy, or —C(O)—C1-6alkoxy, wherein the C1-6alkyl is optionally substituted with one or more halo, C₁₋₆alkoxy, or C₃₋₁₀cycloalkyl, (h) —C(O)—R^{e}, wherein R^{e} is —NH₂, —OH, or 3-15 membered heterocyclyl, or (i) —S(O)₂—R^{f}, wherein R^{f} is C₁₋₆alkyl or 3-15 membered heterocyclyl, provided that, when R² is unsubstituted methyl, then either (1) Q¹ is 5-20 membered heteroaryl, wherein the 5-20 membered heteroaryl of Q¹ is optionally substituted with one or more halo, C1-6alkyl, C2-6alkenyl, C1-6alkoxy, —NH2, C3-₁₀cycloalkyl, or —OH, and wherein Q¹ is not unsubstituted pyridyl, or (2) Q¹ is phenyl, wherein the phenyl of Q¹ is substituted with (i) at least one C₃₋₆alkyl, wherein the at least one C₃₋₆ alkyl is optionally substituted with one or more halo, or (ii) at least one C₃₋₁₀cycloalkyl, wherein the at least one C₃₋₁₀cycloalkyl is optionally substituted with one or more halo or C₁₋₆alkyl, or (iii) at least one 5-20 membered heteroaryl, wherein the at least one 5-20 membered heteroaryl is optionally substituted with one or more C1-6alkyl, (ii) C₃₋₁₀cycloalkyl, wherein the C₃₋₁₀cycloalkyl of R² is optionally substituted with one or more R⁴, wherein R⁴ is 5-20 membered heteroaryl or C6-20aryl, wherein the C6-20aryl of R4 is optionally substituted with one or more C1-6alkoxy, (iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl, (iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}, wherein R^{s} is halo, C₁₋₆alkyl, C₁₋₆alkoxy. —NH₂, —NH(C₁₋₆alkyl), —N(C₁₋₆alkyl)₂, —NH—C(O)—C₁₋6alkyl, C6-20aryl, C3-10cycloalkyl, 3-15 membered heterocyclyl, 5-20 membered heteroaryl, or —C(O)—C1-6alkoxy, wherein the C₁₋₆alkyl of R^{s} is optionally substituted with one or more halo, C₁₋₆alkoxy, —NH₂, —NH(C₁₋₄alkyl), —N(C₁₋₆alkyl)₂, —NH—C(O)C₁₋₆alkyl, or —NH—C(O)—C₁₋₆alkoxy, and the 3-15-membered heterocyclyl of R^{s} is optionally substituted with one or more halo or —C(O)—C₁₋₆alkoxy, (v) —N(R^{g})(R^{h}), wherein R^{g} and R^{h} are independently H or C₁₋₆alkyl, (vi) —C(O)—R^{j}, wherein R^{j} is C₃₋₁₀cycloalkyl, —NH(C₁₋₆alkyl), —N(C₁₋₆alkyl)₂, or —NH(5-20 membered heteroaryl), and (vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C1-6alkyl, as described in International Patent Publication No. WO2022/198196, which is hereby incorporated by reference in its entirety.

In some embodiments, the compositions provided herein may be used to diagnose, monitor, modulate, treat, alleviate, help prevent the incidence of, or reduce the symptoms of human disease or specific pathologies in cells, tissues, organs, fluid, or, generally, a host, also exhibit the property of being able to cross the blood brain barrier. The blood-brain barrier (BBB) prevents most macromolecules (e.g., DNA, RNA, and polypeptides) and many small molecules from entering the brain. The BBB is principally composed of specialized endothelial cells with highly restrictive tight junctions, consequently, passage of substances, small and large, from the blood into the central nervous system is controlled by the BBB. This structure makes treatment and management of patients with neurological diseases and disorders (e.g., brain cancer) difficult as many therapeutic agents cannot be delivered across the BBB with desirable efficiency. Additional conditions that involve disruptions of the BBB include: stroke, diabetes, seizures, hypertensive encephalopathy, acquired immunodeficiency syndrome, traumatic brain injuries, multiple sclerosis, Lafora Disease, Pompe Disease, adult polyglucosan body disease, Parkinson's disease (PD) and Alzheimer disease. This ability is especially useful for treating brain cancers including for example: astrocytoma, medulloblastoma, glioma, ependymoma, germinoma (pinealoma), glioblastoma multiform, oligodendroglioma, schwannoma, retinoblastoma, and congenital tumors; or a cancer of the spinal cord, e.g., neurofibroma, meningioma, glioma, and sarcoma. This is further useful in treating Pompe Disease and/or glycogen storage disease. In certain embodiments, the compositions provided for herein can be used to deliver a therapeutic or cytotoxic agent, for example, across the blood brain barrier. In certain embodiments, the compositions provided for herein can be used to deliver a therapeutic or cytotoxic agent, for example, into the muscle.

In some embodiments, methods of treating glycogen storage disease in a subject in need thereof, the method comprising administering a composition provided herein are provided. In some embodiments, the glycogen storage disease is selected from the group consisting of Cori's disease or Forbes' disease (GSD3, Glycogen debranching enzyme (AGL) deficiency), McArdle disease (GSD5, Muscle glycogen phosphorylase (PYGM) deficiency), type II Diabetes/diabetic nephropathy, Aldolase A Deficiency GSD12, Lafora Disease, hypoxia, Andersen disease (GSD4, Glycogen debranching enzyme (GBE1) deficiency), Tarui's Disease (GSD7, Muscle phosphofructokinase (PFKM) deficiency), and adult polyglucosan body disease. In some embodiments, the glycogen storage disease is selected from the group consisting of Glycogen synthase (GYS2) deficiency (GSD0), Glucose-6-phosphatase (G6PC/SLC37A4) deficiency (GSD1, von Gierke's disease), Hers' disease (GSD6, Liver glycogen phosphorylase (PYGL) or Muscle phosphoglycerate mutase (PGAM2) deficiency), Phosphorylase kinase (PHKA2/PHKB/PHKG2/PHKA1) deficiency (GSD9), Phosphoglycerate mutase (PGAM2) deficiency (GSD10), Muscle lactate dehydrogenase (LDHA) deficiency (GSD11), Fanconi-Bickel syndrome (GSD 11, Glucose transporter (GLUT2) deficiency, Aldolase A deficiency (GSD 12), β-enolase (ENO3) deficiency (GSD13), and Glycogenin-1 (GYG1) deficiency (GSD15).

In some embodiments, the compositions or pharmaceutical compositions provided herein can be used to treat muscle diseases, such as muscular dystrophy, DMD, and the like.

In some embodiments, the compositions or pharmaceutical compositions provided herein may be administered alone or in combination with other therapeutics, that is, simultaneously or sequentially. In some embodiments, the other or additional therapeutics are other anti-tumor agent or therapeutics. Different tumor types and stages of tumors can require the use of various auxiliary compounds useful for treatment of cancer. For example, the compositions provided herein can be used in combination with various chemotherapeutics such as taxol, tyrosine kinase inhibitors, leucovorin, fluorouracil, irinotecan, phosphatase inhibitors, MEK inihibitors, among others. The composition may also be used in combination with drugs which modulate the immune response to the tumor such as anti-PD-1 or anti-CTLA-4, among others. Additional treatments can be agents that modulate the immune system, such antibodies that target PD-1 or PD-L1.

In some embodiments, the compositions or pharmaceutical compositions provided herein may be administered in combination with an enzyme replacement therapy (ERT). In some embodiments, the ERT is an ERT designed to treat a glycogen storage disease. In some embodiments, the glycogen storage disease is selected from the group consisting of Pompe Disease (GSD2, glucosidase alpha acid (GAA) deficiency), Cori's disease or Forbes' disease (GSD3, Glycogen debranching enzyme (AGL) deficiency), Andersen's disease (GSD4, glycogen branching enzyme (GBE1) deficiency), McArdle disease (GSD5, Muscle glycogen phosphorylase (PYGM) deficiency), Tarui's Disease (GSD7, Muscle phosphofructokinase (PFKM) deficiency), Aldolase A Deficiency (GSD12, aldolase A (ALDOA) deficiency), type II Diabetes/diabetic nephropathy, Lafora Disease, hypoxia, and adult polyglucosan body disease. In some embodiments, the glycogen storage disease is selected from the group consisting of Glycogen synthase (GYS2) deficiency (GSD0), Glucose-6-phosphatase (G6PC/SLC37A4) deficiency (GSD1, von Gierke's disease), Hers' disease (GSD6, Liver glycogen phosphorylase (PYGL) or Muscle phosphoglycerate mutase (PGAM2) deficiency), Phosphorylase kinase (PHKA2/PHKB/PHKG2/PHKA1) deficiency (GSD9), Phosphoglycerate mutase (PGAM2) deficiency (GSD10), Muscle lactate dehydrogenase (LDHA) deficiency (GSD11), Fanconi-Bickel syndrome (GSD 11, Glucose transporter (GLUT2) deficiency, Aldolase A deficiency (GSD 12), β-enolase (ENO3) deficiency (GSD13), and Glycogenin-1 (GYG1) deficiency (GSD15).

Thus, in some embodiments, the ERT comprises the administration of one or more enzymes or gene replacement products selected from the group consisting of glucosidase alpha acid (GAA), glycogen debranching enzyme (AGL), glycogen branching enzyme (BGE1), muscle glycogen phosphorylase (PYGM), muscle phosphofructokinase (PFKM), aldolase A (ALDOA), malin, laforin, glycogen synthase (GYS2), glucose-6-phosphatase (G6PC/SLC37A4), phosphorylase kinase (PHKA2/PHKB/PHKG2/PHKA1), phosphoglycerate mutase (PGAM2), muscle lactate dehydrogenase (LDHA), glucose transporter (GLUT2), 0-enolase (ENO3), glycogenin-1 (GYG1), or any combination thereof. In some embodiments, the ERT comprises administering glucosidase alpha acid (GAA). In some embodiments, the ERT comprises administering glycogen debranching enzyme (AGL). In some embodiments, the ERT comprises administering glycogen branching enzyme (BGE1). In some embodiments, the ERT comprises administering muscle glycogen phosphorylase (PYGM). In some embodiments, the ERT comprises administering muscle phosphofructokinase (PFKM). In some embodiments, the ERT comprises administering aldolase A (ALDOA). In some embodiments, the ERT comprises administering malin. In some embodiments, the ERT comprises administering laforin. In some embodiments, the ERT comprises administering glycogen synthase (GYS2). In some embodiments, the ERT comprises administering glucose-6-phosphatase (G6PC/SLC37A4). In some embodiments, the ERT comprises administering phosphorylase kinase (PHKA2/PHKB/PHKG2/PHKA1). In some embodiments, the ERT comprises administering phosphoglycerate mutase (PGAM2). In some embodiments, the ERT comprises administering, muscle lactate dehydrogenase (LDHA). In some embodiments, the ERT comprises administering glucose transporter (GLUT2). In some embodiments, the ERT comprises administering β-enolase (ENO3). In some embodiments, the ERT comprises administering glycogenin-1 (GYG1).

In some embodiments, methods of treating a glycogen storage disease in a subject in need thereof are provided. In some embodiments, the methods comprise administering to a subject any composition provided herein and an ERT. In some embodiments, a use of a composition as provided herein and an ERT are provided in the preparation of a pharmaceutical composition or medicament for treating a glycogen storage disease. In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating Pompe Disease in a subject in need thereof are provided. In some embodiments, the methods comprise administering to a subject any composition provided herein and an ERT comprising glucosidase alpha acid (GAA). In some embodiments, a use of a composition as provided herein and an ERT comprising glucosidase alpha acid (GAA) are provided in the preparation of a pharmaceutical composition or medicament for treating Pompe Disease. In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating Cori's disease or Forbes' disease (GSD3) in a subject in need thereof are provided. In some embodiments, the methods comprise administering to a subject any composition provided herein and an ERT comprising glycogen debranching enzyme (AGL). In some embodiments, a use of a composition as provided herein and an ERT comprising glycogen debranching enzyme (AGL) are provided in the preparation of a pharmaceutical composition or medicament for treating Cori's disease or Forbes' disease (GSD3). In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating Andersen's disease (GSD4) in a subject in need thereof are provided. In some embodiments, the methods comprise administering to a subject any composition provided herein and an ERT comprising glycogen branching enzyme (GBE1). In some embodiments, a use of a composition as provided herein and an ERT comprising glycogen branching enzyme (GBE1) are provided in the preparation of a pharmaceutical composition or medicament for treating Andersen's disease (GSD4). In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating McArdle disease (GSD5) in a subject in need thereof are provided. In some embodiments, the methods comprise administering to a subject any composition provided herein and an ERT comprising muscle glycogen phosphorylase (PYGM). In some embodiments, a use of a composition as provided herein and an ERT comprising muscle glycogen phosphorylase (PYGM) are provided in the preparation of a pharmaceutical composition or medicament for treating McArdle disease (GSD5). In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating Tarui's Disease (GSD7) in a subject in need thereof are provided. In some embodiments, the methods comprise administering to a subject any composition provided herein and an ERT comprising muscle phosphofructokinase (PFKM). In some embodiments, a use of a composition as provided herein and an ERT comprising muscle phosphofructokinase (PFKM) are provided in the preparation of a pharmaceutical composition or medicament for treating Tarui's Disease (GSD7). In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating Aldolase A Deficiency (GSD12) in a subject in need thereof are provided. In some embodiments, the methods comprise administering to a subject any composition provided herein and an ERT comprising aldolase A (ALDOA). In some embodiments, a use of a composition as provided herein and an ERT comprising aldolase A (ALDOA) are provided in the preparation of a pharmaceutical composition or medicament for treating Aldolase A Deficiency (GSD12). In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating Lafora Disease in a subject in need thereof are provided. In some embodiments, the methods comprise administering to a subject any composition provided herein and an ERT comprising malin, laforin or both. In some embodiments, a use of a composition as provided herein and an ERT comprising malin, laforin or both are provided in the preparation of a pharmaceutical composition or medicament for treating Lafora Disease. In some embodiments, the composition is a composition comprising and FN3 domain, which can be linked to an additional therapeutic, such as, but not limited to an oligonucleotide (e.g. siRNA, antisense, mRNA, miRNA, cDNA, and the like). In some embodiments, the composition is administered to the subject before the administration of the ERT. In some embodiments, the composition is administered to the subject during the administration of the ERT. In some embodiments, the composition is administered to the subject after the administration of the ERT.

In some embodiments, methods of treating adult polyglucosan body disease in a subject in need thereof are provided. In some embodiments, the methods comprise administering to a subject any composition provided herein and an ERT comprising glycogen branching enzyme (BGE1). In some embodiments, a use of a composition as provided herein and an ERT comprising glycogen branching enzyme (BGE1) are provided in the preparation of a pharmaceutical composition or medicament for treating adult polyglucosan body disease. In some embodiments, the composition is a composition comprising and FN3 domain. In some embodiments, the composition is administered to the subject before the administration of the ERT. In some embodiments, the composition is administered to the subject during the administration of the ERT. In some embodiments, the composition is administered to the subject after the administration of the ERT.

“Treat” or “treatment” refers to the therapeutic treatment and prophylactic measures, wherein the object is to prevent or slow down (lessen) an undesired physiological change or disorder, such as the development or spread of cancer. In some embodiments, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the compositions provided herein may vary according to factors such as the disease state, age, sex, and weight of the individual. Exemplary indicators of an effective amount is improved well-being of the patient, decrease or shrinkage of the size of a tumor, arrested or slowed growth of a tumor, and/or absence of metastasis of cancer cells to other locations in the body.

Administration/Pharmaceutical Compositions

In some embodiments, pharmaceutical compositions of the compositions provided herein and a pharmaceutically acceptable carrier, are provided. For therapeutic use, the compositions may be prepared as pharmaceutical compositions containing an effective amount of the domain or molecule as an active ingredient in a pharmaceutically acceptable carrier. “Carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules disclosed herein in such pharmaceutical formulation can vary widely, i.e., from less than about 0.5%, usually at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, See especially pp. 958-989.

The mode of administration for therapeutic use of the compositions disclosed herein may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

Additionally, the following embodiments are also provided:

1. A method of treating a glycogen storage disease in a subject in need thereof, the method comprising the administration of:
a composition comprising one or more FN3 domains linked to an siRNA molecule (or other oligonucleotide, such as an antisense oligonucleotide or as otherwise provided for herein) comprising a sense strand and antisense strand, such as provided herein; and
an enzyme replacement therapy (ERT).

2. The method of embodiment 1, wherein the glycogen storage disease is selected from the group consisting of Pompe Disease (GSD2, glucosidase alpha acid (GAA) deficiency), Cori's disease or Forbes' disease (GSD3, Glycogen debranching enzyme (AGL) deficiency), Andersen's disease (GSD4, glycogen branching enzyme (GBE1) deficiency), McArdle disease (GSD5, Muscle glycogen phosphorylase (PYGM) deficiency), Tarui's Disease (GSD7, Muscle phosphofructokinase (PFKM) deficiency), Aldolase A Deficiency (GSD12, aldolase A (ALDOA) deficiency), type II Diabetes/diabetic nephropathy, Lafora Disease, hypoxia, and adult polyglucosan body disease.

3. The method of embodiments 1 or 2, wherein the ERT comprises one or more enzymes selected from the group consisting of glucosidase alpha acid (GAA), glycogen debranching enzyme (AGL), glycogen branching enzyme (BGE1), muscle glycogen phosphorylase (PYGM), muscle phosphofructokinase (PFKM), aldolase A (ALDOA), malin, laforin, glycogen synthase (GYS2), glucose-6-phosphatase (G6PC/SLC37A4), phosphorylase kinase (PHKA2/PHKB/PHKG2/PHKA1), phosphoglycerate mutase (PGAM2), muscle lactate dehydrogenase (LDHA), glucose transporter (GLUT2), β-enolase (ENO3), and glycogenin-1 (GYG1).

4. The method of any one of embodiments 1-3, wherein the composition comprising an FN3 domain linked to an siRNA is administered to the subject prior to the administration of the ERT.

5. The method of any one of embodiments 1-3, wherein the composition comprising an FN3 domain linked to an siRNA is administered to the subject during the administration of the ERT.

6. The method of any one of embodiments 1-3, wherein the composition comprising an FN3 domain linked to an siRNA is administered to the subject after the administration of the ERT.

7. A method of treating Pompe Disease in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising glucosidase alpha acid (GAA).

8. A method of treating Cori's disease (also known as Forbes' disease) in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising glycogen debranching enzyme (AGL).

9. A method of treating Andersen's disease (GSD4) in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising glycogen branching enzyme (GBE1).

10. A method of treating McArdle disease (GSD5) in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising glycogen phosphorylase (PYGM).

11. A method of treating Tarui's Disease (GSD7) in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising muscle phosphofructokinase (PFKM).

12. A method of treating Aldolase A Deficiency (GSD12) in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising aldolase A (ALDOA).

13. A method of treating Lafora Disease in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising malin, laforin or both.

14. A method of treating adult polyglucosan body disease in a subject in need thereof, the method comprising the administration of:
a composition comprising an FN3 domain linked to an siRNA (or other oligonucleotide, such as an antisense oligonucleotide or as otherwise provided for herein) comprising a sense strand and antisense strand, as provided herein; and
an ERT comprising glycogen branching enzyme (BGE1).

15. The method of any one of embodiments 7-14, further comprising at least one additional ERT.

16. The method of any one of embodiments 7-15, wherein the composition comprising an FN3 domain linked to an siRNA is administered to the subject prior to the administration of the ERT.

17. The method of any one of embodiments 7-15, wherein the composition comprising an FN3 domain linked to an siRNA is administered to the subject during the administration of the ERT.

18. The method of any one of embodiments 7-15, wherein the composition comprising an FN3 domain linked to an siRNA is administered to the subject after the administration of the ERT.

19. The method of any one of embodiments 1-18, wherein the one or more FN3 domains comprises a FN3 domain that binds to CD71.

20. The method of any one of embodiments 1-19, wherein the siRNA (or other oligonucleotide, such as an antisense oligonucleotide or as otherwise provided for herein) molecule is an siRNA that reduces the expression of GYS1.

21. The method of any one of embodiments 1-20, wherein the siRNA does not contain any modified nucleobases.

22. The method of any one of embodiments 1-21, wherein the siRNA further comprises a linker covalently attached to the sense strand or the antisense strand of the siRNA.

23. The method of embodiment 22, wherein the linker is attached to the 5′ end or the 3′ end of the sense strand or the antisense strand.

24. The method of any one of embodiments 1-23, wherein the siRNA further comprises a vinyl phosphonate modification on the sense strand or the antisense strand.

25. The method of embodiment 24, wherein the vinyl phosphonate modification is attached to the 5′ end or the 3′ end of the sense strand or the anti-sense strand.

26. The method of any one of embodiments 1-25, wherein the sense strand comprises a sequence of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 801-860, 921-980, or as set forth in Table 3A, Table 3B, or Table 4.

27. The method of any one of embodiments 1-26, wherein the antisense strand comprises a sequence of SEQ ID NO: 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 690, 691, 693, 695, 697, 699, 701, 703, 705, 707, 861-920, 981-1042, or as set forth in Table 3A, Table 3B, or Table 4.

28. The method of any one of the preceding embodiments, wherein the siRNA molecule comprises the siRNA pair of A, B, C, D, E, F, G, H, I, J, K, L, M, N, O, P, Q, R, S, T, U, V, W, X, Y, Z, AA, BB, CC, DD, EE, FF, GG, HH, II, JJ, KK, LL, MM, NN, OO, PP, QQ, RR, SS, TT, UU, VV, WW, XX, YY, ZZ, AAA, BBB, CCC, DDD, EEE, FFF, GGG, HHH, III, JJJ, KKK, LLL, MMM, NNN, OOO, PPP, QQQ, RRR, SSS, TTT, UUU, VVV, WWW, XXX, YYY, ZZZ, AAAA, BBBB, CCCC, DDDD, EEEE, FFFF, GGGG, HHHH, IIII, JJJJ, KKKK, LLLL, MMMM, NNNN, OOOO, PPPP, or as set forth in Table 3A, Table 3B, or Table 4.

29. The method of any one of the preceding embodiments, wherein the sense strand comprises 19 nucleotides.

30. The method of any one of the preceding embodiments, wherein the antisense strand comprises 21 nucleotides.

31. The method of any one of embodiments 1-30, wherein the composition comprises the siRNA pair as provided for in Table 4, with a linker and/or vinyl phosphonate modification as set forth in Table 5.

32. The method of any one of the preceding embodiments, wherein the siRNA molecule has the formula as illustrated in Formula I:

Sense-strand (SS)
N₁N₂N₃N₄N₅N₆N₇N₈N₉N₁₀N₁₁N₁₂N₁₃N₁₄N₁₅N₁₆N₁₇N₁₈N₁₉
Antisense-strand (AS)
N₂₁N₁₉N₁₈N₁₇N₁₆N₁₅N₁₄N₁₃N₁₂N₁₁N₁₀N₉N₈N₇N₆N₅N₄N₃N₂N₁,

wherein each nucleotide represented by N, is independently, A, U, C, or G or a modified nucleotide base, such as those provided for herein.

33. The method of embodiment 32, wherein the sense strand comprises a 2′O-methyl modified nucleotide with a phosphorothioate (PS) modified backbone at N₁ and N₂, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, N₁₈, and N₁₉.

34. The method of embodiment 32, wherein the antisense strand comprises a vinylphosphonate moiety attached to N₁, a 2′fluoro-modified nucleotide with a phosphorothioate (PS) modified backbone at N₂, a 2′O-methyl modified nucleotide at N₃, N₄, N₅, N₆, N₇, N₈, N₉, N₁₀, N₁₁, N₁₂, N₁₃, N₁₅, N₁₆, N₁₇, N₁₈, and N₁₉, a 2′fluoro-modified nucleotide at N₁₄, and a 2′O-methyl modified nucleotide with a phosphorothioate (PS) modified backbone at N₂₀ and N₂₁.

35. The method of embodiment 32, wherein a vinylphosphonate moiety attached to N₁ of the antisense strand.

36. The method of any one of the preceding embodiments, wherein the siRNA molecule has the formula as illustrated in Formula I:

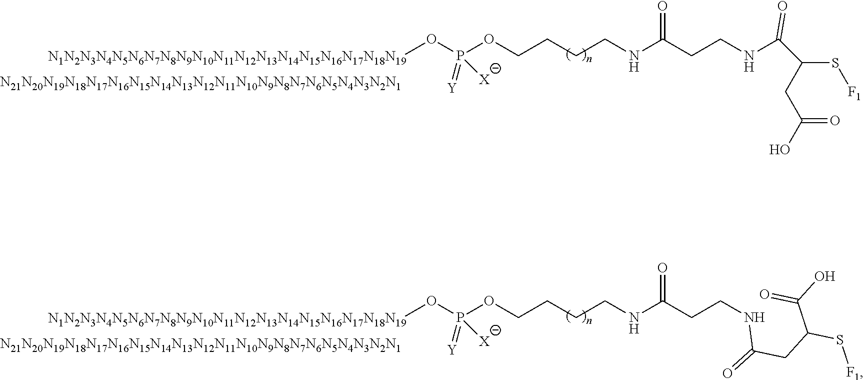

wherein F₁ is a polypeptide comprising at least one FN3 domain and is conjugated to a linker, L₁, L₁ is linked to X_{S}, wherein X_{S} is a 5′ to 3′ oligonucleotide sense strand of a double stranded siRNA molecule and X_{AS} is a 3′ to 5′ oligonucleotide antisense strand of a double stranded siRNA molecule; and wherein
X_{S} and X_{AS} form a double stranded siRNA molecule.

37. The method of embodiment 36, wherein F₁ comprises polypeptide having a formula of (X₁)ₙ—(X₂)_{q}—(X₃)_{y}, wherein X1 is a first FN3 domain; X₂ is a second FN3 domain; X₃ is a third FN3 domain or half-life extender molecule; wherein n, q, and y are each independently 0 or 1, provided that at least one of n, q, and y is 1.

38. The method of any one of the preceding embodiments, wherein the FN3 domain is conjugated to the siRNA molecule through a cysteine on the FN3 domain.

39. The method of embodiment 38, wherein the cysteine is at a position as described herein.

40. The method of embodiments 38 or 39, wherein the cysteine in the FN3 domain is at a position that corresponds to residue 6, 8, 10, 11, 14, 15, 16, 20, 30, 34, 38, 40, 41, 45, 47, 48, 53, 54, 59, 60, 62, 64, 70, 88, 89, 90, 91, or 93 of the FN3 domain comprising the amino acid sequence based on SEQ ID NO: 713.

41. The method of embodiment 40, wherein the cysteine is located at a position that corresponds to residue 6, 53, or 88.

42. The method of any one of the preceding embodiments, wherein the FN3 domain has a sequence selected from the group consisting of SEQ ID NOs: 509, 708, and 710.

43. The method of any one of the preceding embodiments, wherein one or more FN3 domains comprises at least two FN3 domains linked by a peptide linker.

44. The method of any one of the preceding embodiments, wherein the composition comprises a first FN3 domain and a second FN3 domain.

45. The method of embodiment 44, wherein the first FN3 domain and the second FN3 domain bind to different proteins.

46. The composition of embodiment 44, wherein the first FN3 domain and the second FN3 domain bind to the same protein.

47. The method of any one of embodiments 44-46, wherein the first FN3 domain binds to CD71.

48. The method of any one of embodiments 44-46, wherein the second FN3 domain binds to a different target that does not bind CD71.

49. The method of any one of the preceding embodiments, wherein the FN3 domain comprises a sequence that is at least 87% identical to or is identical to a sequence of SEQ ID NO: 273, 288-291, 301-310, 312-572, 592-599, or 708-710.

50. The method of any one of embodiments 43-49, further comprising a third FN3 domain.

51. The method of embodiment 50, wherein the third FN3 domain is a FN3 domain that binds to CD71 or albumin.

52. The method of embodiment 51, wherein the FN3 domain that binds CD71 has an amino acid sequence as provided herein, including but not limited to SEQ ID NO: 273, 288-291, 301-310, 312-572, 592-599, or 708-710, or a binding fragment thereof.

53. The method of embodiment 51, wherein the FN3 that binds albumin has an amino acid sequence as provided herein, including but not limited to SEQ ID NO: 101-119, or a binding fragment thereof.

54. The method of embodiment 1, wherein the composition comprising an FN3 domain linked to an siRNA has a formula selected from (X₁)ₙ—(X₂)_{q}—(X₃)_{y}-L-X₄, C—(X₁)ₙ—(X₂)_{q}-L-X₄—(X₃)_{y}, (X₁)ₙ—(X₂)_{q}-L-X₄—(X₃)_{y}—C, C—(X₁)ₙ—(X₂)_{q}-L-X₄-L-(X₃)_{y}, or (X₁)ₙ—(X₂)_{q}-L-X₄-L-(X₃)_{y}—C,
wherein:
X₁ is a first FN3 domain;
X₂ is a second FN3 domain;
X₃ is a third FN3 domain or half-life extender molecule;
L is a linker;
X₄ is a nucleic acid molecule (e.g. a strand or strands of the siRNA); and
C is a polymer, such as PEG, albumin binding protein, and
wherein n, q, and y are each independently 0 or 1.

55. The method of embodiment 54, wherein X1, X₂, and X₃ bind to the same or different target proteins.

56. The method of embodiments 54 or 55, wherein y is 0.

57. The method of embodiments 54 or 55, wherein n is 1, q is 0, and y is 0.

58. The method of embodiments 54 or 55, wherein n is 1, q is 1, and y is 0.

59. The method of embodiments 54 or 55, wherein n is 1, q is 1, and y is 1.

60. The method of any one of embodiments 54-59, wherein the third FN3 domain increases the half-life of the molecule as a whole as compared to a molecule without X₃.

61. The method of embodiment 54, wherein the third FN3 domain is a FN3 domain that binds to albumin.

62. The method of any one of embodiments 54-61, wherein the linker is a linker as provided herein.

63. The method of any one of embodiments 54-62, wherein the FN3 domains are connected by a peptide linker.

64. The method of embodiment 63, wherein the peptide linker is (GS)₂, (SEQ ID NO: 720), (GGGS)₂ (SEQ ID NO:721), (GGGGS)₅ (SEQ ID NO: 722), (AP)₂₋₂₀, (AP)₂ (SEQ ID NO: 723), (AP)₅ (SEQ ID NO: 724), (AP)₁₀ (SEQ ID NO: 725), (AP)₂₀ (SEQ ID NO: 726) and A(EAAAK)₅AAA (SEQ ID NO: 727) or (EAAAK)₁₋₅ (SEQ ID NO: 728), or any combination thereof.

65. The method of any one of embodiments 54-64, wherein the first, second, or third FN3 domain has an amino acid sequence as provided herein.

66. The method of any one of embodiments 54-65, wherein X₄ is the siRNA molecule.

67. The method of embodiment 66, wherein the siRNA molecule is a siRNA molecule provided herein.

68. The method of embodiment 66, wherein the siRNA molecule is a siRNA that reduces the expression of GYS1.

69. The method of embodiment 66, wherein the siRNA molecule is a siRNA that specifically reduces the expression of GYS1.

70. The method of embodiment 66, wherein the siRNA molecule is a siRNA that reduces the expression of GYS1 and does not significantly reduce the expression of other RNAs.

71. The method of embodiment 66, wherein the siRNA molecule is a siRNA that reduces the expression of GYS1 and does not reduce the expression of other RNAs by more than 50% in an assay described herein at a concentration of no more than 200 nm as described herein.

72. The method of embodiment 66, wherein the siRNA molecule is a siRNA that reduces the expression of GYS1 and reduces the concentration of GYS1 protein.

73. The method of embodiment 66, wherein the siRNA molecule is a siRNA that reduces the expression of GYS1 and reduces the concentration of glycogen in a muscle cell.

74. The method of any one of embodiments 54-73, wherein the siRNA is a siRNA pair as provided in the following formula:

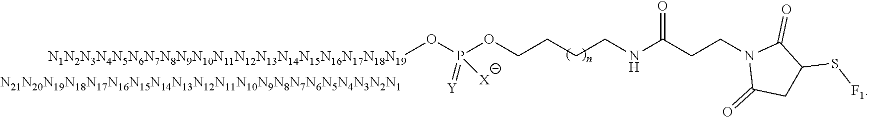

75. The method of embodiment 74, wherein N₁ of the anti-sense strand comprises a vinyl phosphonate modification.

76. The method of embodiment 74, wherein maleimide is hydrolyzed to form the following mixture of compounds, or one or both of each compound, or exclusively one of the compounds

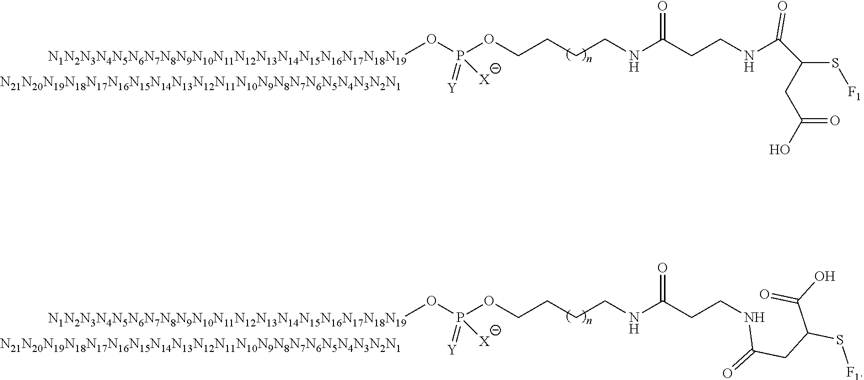

77. The method of any one of embodiments 54-76, wherein the siRNA is a siRNA Pair as provided herein or a siRNA pair selected from the group consisting as provided for in Table 3A, Table 3B, or Table 4.

78. The method of embodiment 1, wherein the composition comprising the FN3 domain linked to an siRNA has a formula of A₁-B₁, wherein A₁ has a formula of (C)ₙ-(L₁)ₜ-Xₛ and B₁ has a formula of X_{As}-(L₂)_{q}-(F₁)_{y}, wherein:
C is a polymer, such as PEG, albumin binding protein;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
wherein n, t, q, and y are each independently 0 or 1;
wherein X_{S} and X_{AS} form a double stranded oligonucleotide molecule to form the composition/complex.

79. The method of embodiment 1, wherein the composition comprising the FN3 domain linked to an siRNA has a formula of A₁-B₁, wherein A₁ has a formula of (F₁)ₙ-(L₁)ₜ-Xₛ and B₁ has a formula of X_{AS}-(L₂)_{q}-(C)_{y}, wherein:
C is a polymer, such as PEG, albumin binding protein;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
wherein n, t, q, and y are each independently 0 or 1;
wherein X_{S} and X_{AS} form a double stranded oligonucleotide molecule to form the composition/complex.

80. The method of embodiments 78 or 79, wherein L₁ has the formula:

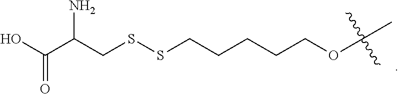 [SMILES: *OCCCCCSSCC(N)C(=O)O]

81. The method of embodiments 78 or 79, wherein L₂ has the formula:

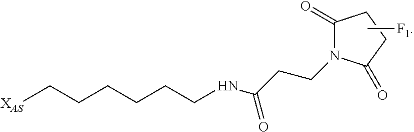

82. The method of embodiments 78 or 79, wherein A₁-B₁ has a formula of:

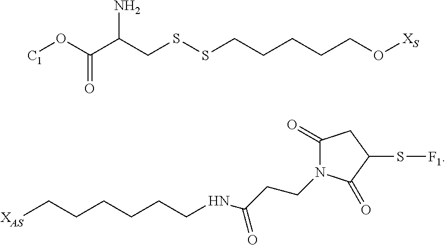

83. The method of embodiments 78 or 79, where in A1-B1 has a formula of:

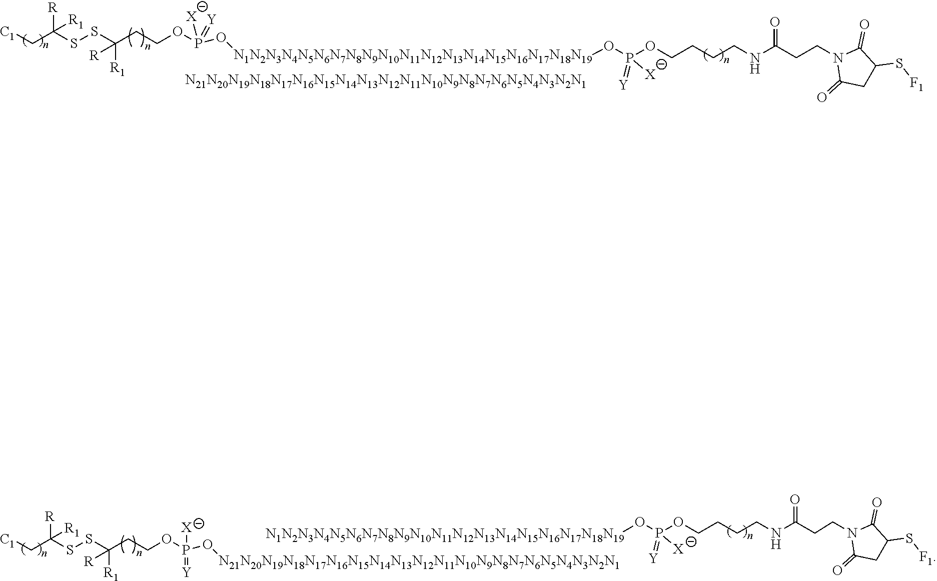

84. The method of embodiments 78 or 79, wherein F₁ comprises polypeptide having a formula of (X₁)ₙ—(X₂)_{q}—(X₃)_{y}, wherein X1 is a first FN3 domain; X₂ is a second FN3 domain; X₃ is a third FN3 domain or half-life extender molecule; wherein n, q, and y are each independently 0 or 1, provided that at least one of n, q, and y is 1.

85. The method of embodiments 78 or 79, wherein X1 is a CD71 FN3 binding domain.

86. The method of embodiments 78 or 79, wherein X₂ is a CD71 FN3 binding domain.

87. The method of embodiments 78 or 79, wherein X₃ is a FN3 domain that binds to human serum albumin.

88. The method of embodiments 78 or 79, wherein X₃ is a Fc domain without effector function that extends the half-life of a protein.

89. The method of any one of embodiments 78-88, wherein X_{S} comprises a sequence of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 801-860, 921-980, or as set forth in Table 3A, Table 3B, or Table 4.

90. The method of any one of embodiments 78-88, wherein X_{AS} comprises a sequence of SEQ ID NO: 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 690, 691, 693, 695, 697, 699, 701, 703, 705, 707, 861-920, 981-1042, or as set forth in Table 3A, Table 3B, or Table 4.

91. The method of any one of embodiments 78-88, wherein X_{S} and X_{AS} form a siRNA pair selected from the group consisting of A, B, C, D, E, F, G, H, I, J, K, L, M, N, O, P, Q, R, S, T, U, V, W, X, Y, Z, AA, BB, CC, DD, EE, FF, GG, HH, II, JJ, KK, LL, MM, NN, OO, PP, QQ, RR, SS, TT, UU, VV, WW, XX, YY, ZZ, AAA, BBB, CCC, DDD, EEE, FFF, GGG, HHH, III, JJJ, KKK, LLL, MMM, NNN, OOO, PPP, QQQ, RRR, SSS, TTT, UUU, VVV, WWW, XXX, YYY, ZZZ, AAAA, BBBB, CCCC, DDDD, EEEE, FFFF, GGGG, HHHH, IIII, JJJJ, KKKK, LLLL, MMMM, NNNN, OOOO, PPPP, or as set forth in Table 3A, Table 3B, or Table 4.

92. The method of any one of embodiments 78-88, wherein F₁ comprises a sequence that is at least 87% identical to or is identical to a sequence of SEQ ID NO: 273, 288-291, 301-310, 312-572, 592-599, or 708-710.

93. The method of any one of embodiments 78-88, wherein F₁ comprises a polypeptide that binds to albumin.

### EXAMPLES

The following examples are illustrative of the embodiments disclosed herein. These examples are provided for the purpose of illustration only and the embodiments should in no way be construed as being limited to these examples, but rather should be construed to encompass any and all variations which become evidence as a result of the teaching provided herein. Those of skill in the art will readily recognize a variety of non-critical parameters that could be changed or modified to yield essentially similar results.

### Example 1: GYS1 siRNA Sequence ID and Characterization

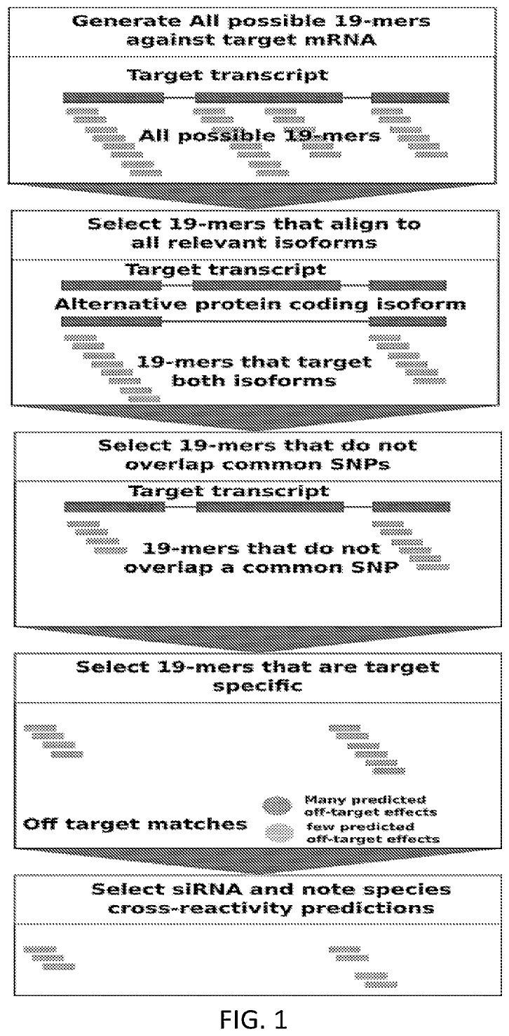

siRNA In Silico Screening: In silico siRNA screening was performed to identify a human siRNA complementary to the human GYS1 mRNA, see FIG. 1. All possible 19-mer antisense sequences were generated from the human GYS1 siRNA sequence (NM_001161587) and each 19-mer was assessed for activity against other human GYS1 isoforms as well as potential cross-reactivity mouse, rat, and Cynomolgus macaque. Human siRNA target sites were assessed for common human SNPs (MAF>1%) using dbSNP (b155 v2). Sequences that targeted a common allele were discarded. Next, siRNA off-target genes were assessed for sense and antisense strands in all relevant model organisms. This selection yielded 200 potential candidates, that were further defined by and in vitro knockdown screen. Lead siRNA candidates are in Tables 3A and 3B. GYS1 siRNA linkers for conjugation to cysteine engineered Centyrins were prepared as described in Table 5.

HEK293T cells were lipofected with 10 nM ABXO-HHH (siRNA Pair HHH) for 24 hours alongside untreated control cells (6 replicates per treatment group). mRNA was polyA+ selected from cells and subjected to unstranded 2×150 bp paired end sequencing on the Illumina HiSeq platform to an average depth of >30 million reads.

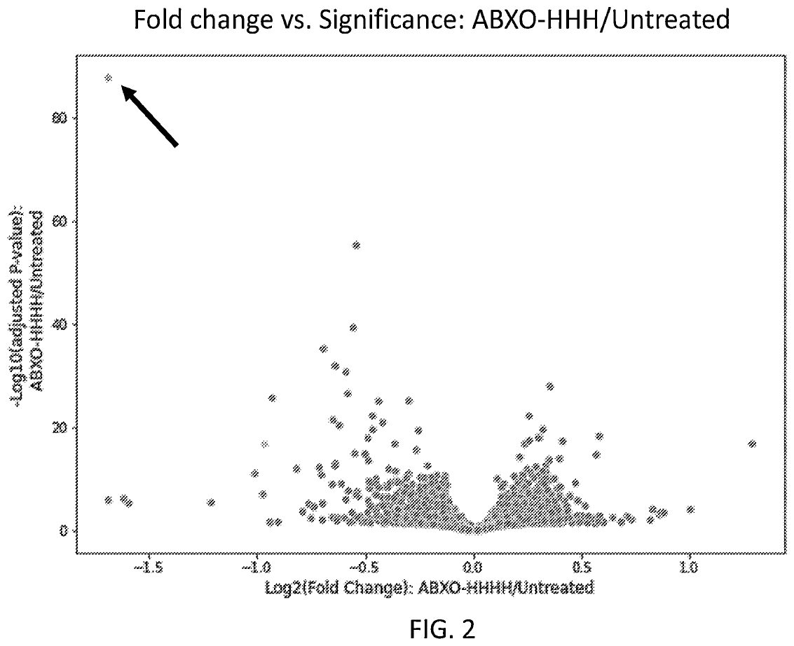

FIG. 2 Volcano plot showing the DESeq2 results of ABXO-HHH versus untreated cells. The X axis represents the log 2 fold change in gene expression (ABXO-HHH divided by untreated) for all genes. The Y axis shows the negative log₁₀ transformed adjusted P value. Colored dots represent genes with significant complementarity to ABXO-HHH detected via BLAST.

RNA-seq library quality was inspected using FastQC. Libraries were pseudo aligned to the GrCH38 human transcriptome using Kallisto. We achieved exceptional read alignment with an average of >90% of reads aligning to the transcriptome. Differential expression was then assessed using DESeq2 comparing ABXO-HHH treated cells to lipofectamine only treated cells.

DESeq results showed substantial and significant knockdown of the GYS1 target. GYS1 expression in ABXO-HHH treated samples was downregulated to 31% of untreated samples (FIG. 2). GYS1 was the most downregulated protein coding gene for all genes that were differentially expressed (DE) under ABXO-HHH treatment. GYS1 was also by far the DE gene with the lowest p-value, with an adjusted p-value of 1e-87.7, over 30 orders of magnitude smaller than the next lowest adjusted p-value.

In silico prediction of potential off-target effects was performed using BLAST. The ABXO-HHH sense and antisense sequences were BLAST aligned against an internal BLAST database built from the GRCh38 transcriptome. Of all the potential off-target effects identified by BLAST, only RAP2C was significantly downregulated in the presence of ABXO-HHH (FIG. 2) and failed to surpass the DE threshold of log₂ f fold change <−1.

Together these data demonstrate that ABXO-HHH is a highly specific siRNA even at relatively high concentrations in the human transcriptome.

Oligonucleotide synthesis, deprotection and annealing protocol:

Synthesis of oligonucleotides was performed on Mermade 12 synthesizer using standard phosphoramidite chemistry on 500 Å controlled pore glass (CPG) with phosphoramidites at a 0.1 M concentration in acetonitrile. 12 in THF/pyridine/water (0.02M) was the oxidizing agent with 0.6 M ETT (5-ethylthiotetrazole) as the activation agent. N,N-dimethyl-N′-(3-thioxo-3H-1,2,4-dithiazol-5-yl)methanimidamide (DDTT), 0.09 M in pyridine, was used as the sulfurizing reagent for the introduction of phosphorothioate (PS) bonds. 3% (v/v) dichloroacetic acid in dichloromethane was used the deblocking solution. All single strands without maleimide were purified by ion exchange chromatography (IEX) with 20 mM phosphate at pH 8.5 as Buffer A and 20 mM phosphate pH 8.5 and 1 M sodium bromide as Buffer B. After purification, the oligonucleotide fractions were pooled, concentrated, and desalted. Desalted samples were then lyophilized to dryness and stored at −20° C.

Deprotection of Antisense Strands

After synthesis, the support was washed with acetonitrile (ACN) and dried in the column under vacuum and transferred into a 1 mL screw cap that could be tightly sealed and shaken with a solution of 5% diethylamine in aqueous ammonia at 65° C. for 5 h. Cleavage and deprotection of crude oligo was checked by liquid chromatography-mass spectrometry (LC-MS) and was subsequently purified by IEX-HPLC.

Synthesis and Deprotection of Maleimide-Containing Oligonucleotides

Maleimide containing oligos were made using either a 3′ amino-modified CPG solid support or a 5′ amino modifier phosphoramidite. The support was transferred into a 1 mL vial that could be tightly sealed and incubated with 50/50 v/v 40% aqueous methyl amine and aqueous ammonia (AMA) at room temperature for 2 h or 65° C. for 10 minutes to cleave and deprotect. The single strand was purified by IEX chromatography and desalted the same conditions as the antisense before maleimide addition.

Approximately 20 mg/mL of the amine-modified sense strand in 0.05 M phosphate buffer at pH 7.1 was made to which 10 equivalents of the maleimide N-hydroxysuccinimide (NHS) ester, dissolved in ACN was added. The NHS ester solution was added to the aqueous oligonucleotide solution and shaken for 3 h at room temperature. The now maleimide conjugated oligonucleotides were purified by reverse-phase chromatography (20 mM triethyl ammonium acetate with 80% acetonitrile in Buffer B) to prevent the maleimide hydrolysis under ion exchange buffer conditions.

After purification, the oligonucleotide fractions were pooled, concentrated, and desalted.

To avoid hydrolysis of maleimide, duplexing of the sense and antisense strands was performed via freeze-drying using equimolar amounts of each desalted single strand.

Centyrin Conjugation to siRNA, Conjugate Purification and Analysis:

Centyrins were conjugated to siRNA through cysteine-specific chemistry via

maleimide. Cysteine-containing Centyrin in PBS at 50-200 μM were reduced with 10 mM tris(2-carboxyethyl) phosphine (TCEP) at room temperature (30 mins) to yield a free thiol. The free thiol containing Centyrin is then mixed with maleimide containing siRNA duplex in water immediately prior at a molar ratio of ˜1.5:1 Centyrin:siRNA. After 2 hr incubation at RT or 37° C., reaction was quenched with N-ethyl maleimide (1 mM final NEM concentration in the reaction mixture) The conjugate was purified in two steps. Step I either immobilized metal affinity chromatography (for tagged proteins) or Hydrophobic interaction chromatography (for tagless proteins); to remove un-reacted SiRNA linker. Step II-Capto-DEAE; to remove un-reacted centyrin. Fractions containing conjugate were pooled, exchanged into HBS by desalting using dialysis, and concentrated if necessary.

Analytical Characterization of Centyrin-siRNA Conjugates:

Centyrin—siRNA conjugates were characterized by a combination of analytical techniques. SDS-PAGE was used to compare amounts of conjugate to free protein. For SDS-PAGE, 4-20% Mini-PROTEAN® TGX Stain-Free™ Protein Gels (BioRad) were run in SDS buffer for one hour at 100 V. Gels were visualized under UV light. Analytical SEC (Superdex-75 5/150 GL column-GE) was used to analyze purity and aggregation state of Centyrin-siRNA conjugates. Liquid chromatography/mass spectrometry (LCMS) was used to confirm identity and purity of the conjugates. Samples were analyzed using a Waters Acuity UPLC/Xevo G2-XS TOF mass spectrometer system. The instrument was operated in negative electro-spray ionization mode and scanned from m/z 200 to 3000. Conjugate was seen as two fragments; Antisense and Sense-Centyrin.

Centyrin conjugation to siRNA, conjugate purification and analysis: Centyrins were conjugated to siRNA through cysteine-specific chemistry via maleimide. Cysteine-containing Centyrin in PBS at 50-200 μM were reduced with 10 mM tris(2-carboxyethyl) phosphine (TCEP) at room temperature (30 mins) to yield a free thiol. The free thiol containing Centyrin is then mixed with maleimide containing siRNA duplex in water immediately prior at a molar ratio of ˜1.5:1 Centyrin:siRNA. After 2 hr incubation at RT or 37° C., reaction was quenched with N-ethyl maleimide (1 mM final NEM concentration in the reaction mixture) The conjugate was purified in two steps. Step I either immobilized metal affinity chromatography (for tagged proteins) or Hydrophobic interaction chromatography (for tagless proteins); to remove un-reacted SiRNA linker. Step II-Capto-DEAE; to remove un-reacted centyrin. Fractions containing conjugate were pooled, exchanged into HBS by desalting using dialysis, and concentrated if necessary.

FIG. 6: IMAC chromatography for Tagged protein. For IMAC, Histrap HP Columns (1 ml, 5 ml) from Cytiva were used. Histrap Buffer A (Binding Buffer) was 50 mM Tris pH7.4, 500 mM NaCl and 10 mM imidazole in Type 1 H2O, Histrap Buffer B (Elution Buffer): 50 mM Tris pH7.4, 500 mM NaCl and 250 mM imidazole in Type 1 H2O. Reaction sample was directly injected onto the column through sample loop or sample pump. Followed by sample application, column is washed with 5-10 CV of Buffer A. Elution is typically started with step gradient (0-100%). Fractions were collected when UV reading is 50 mAU and above using fraction collector and peak fragmentation.

FIG. 7: HIC—for tagless proteins (removal of excess of siRNA). For HIC, HiTrap Butyl HP Columns (1 ml, 5 ml) from Cytiva were used. HIC Buffer A (Binding Buffer) was 2M Ammonium Sulfate, 25 mM Sodium Phosphate pH 7.0 in Type 1 H2O, while, HIC Buffer B (Elution Buffer): 25 mM Sodium Phosphate pH 7.0 in Type 1 H2O. Conjugation reaction sample is diluted with Buffer A in ratio of 1:1. Sample prepared above was injected onto the column through sample loop or sample pump. Followed by sample application, column is washed with 5 CV of Buffer A. Elution is typically started with 0.0% B and then a gradient as Table 8, below. Fractions collected when UV reading is 50 mAU and above using fraction collector and peak fragmentation.

TABLE 8

| | Type | % B | Length (CV) |
|---|---|---|---|
| 1 | Linear | 30 to 90 | 3.00 |
| 2 | Linear | 35.0 | 10.00 |
| 3 | Linear | 100.0 | 10.00 |
| 4 | Linear | 100.0 | 5.00 |

Ring-opening—To avoid loss of cargo via retro-Michael rection, maleimide ring hydrolysis is performed. Pooled fractions from either histrap (tagged protein) or from HIC (tagless protein) is dialyzed into 25 mM TRIS pH 8.9 buffer. In this buffer the reaction is placed in an incubator shaker at 37 degrees C. for 72 hrs. Reaction monitored for completion by LC-MS.

FIG. 8: Ion-exchange chromatography (IEX)—for tagged and tagless proteins (removal of excess unreacted centyrin). For IEX chromatography, HiTrap Capto DEAE Columns (1 ml, 5 ml) from Cytiva were used. Capto DEAE Buffer A (Binding Buffer) was 25 mM Tris pH 8.8 in Type 1 H2O, while, Capto DEAE Buffer B (Elution Buffer) used was 25 mM Tris pH 8.8, 1M NaCl in Type 1 H2O.

Sample after ring opening was directly injected onto the column through sample loop or sample pump. Followed by sample application, column is washed with 5-10 CV of Buffer A. Elution is typically started with 0.0% B and then a gradient as Table 9, below.

Unreacted protein is eluted in the flowthrough which is typically the first peak and second peak is typically pure conjugate. All the fractions are collected and pooled. Concentration of the pool is determined by measuring A260 using Nanodrop for yield calculation.

TABLE 9

| | | | |
|---|---|---|---|
| 1 | Linear | 20 | 5 |
| 2 | Linear | 100 | 15 |
| 3 | Linear | 100 | 5 |

FIG. 9: Example of an analytical SEC of the centyrin-oligonucleotide conjugate. Centyrin-siRNA conjugates were characterized by a combination of analytical techniques. Analytical SEC was used to analyze purity and aggregation state of Centyrin-siRNA conjugates. Waters H-class UPLC along with Waters ACQUITY UPLC Protein BEH SEC Column, 125 Å, 1.7 μm, 4.6×150 mm was used for routine SEC analysis. Typically, 2-5 ul sample was injected using the autosampler, flow rate was 0.25 ml/min and mobile phase used was either 1×PBS or 100 mM, pH 7.2 phosphate buffer.

FIG. 10: Example of SDS PAGE Gel of the conjugate. SDS-PAGE was used to compare amounts of conjugate to free protein. For performing the SDS-PAGE Gel; Invitrogen Mini Gel tank. PowerEase™ Touch 600 W Power Supply and 115 VAC was used. Invitrogen—NuPAGE™ 4-12% Bis-Tris Protein Gels, 1.0 mm were used along with 20× NuPAGE® MES SDS Running Buffer and SeeBlue™ Pre-stained Protein Standard. Samples normalized to around 0.5 mg/mL by ultrapure water. For non-reducing gel, 10 μL of normalized samples are mixed with 10 μL of 2× Laemmli Sample Buffer in 1 to 1 ratio. For reducing gel, 2× Laemmli Sample Buffer is mixed with β-Mercaptoethanol in 95 to 1 ratio and then mixed with 10 μL of samples in 1 to 1 ratio. The resulting sample mixtures were boiled at 95° C. for 5 minutes and then cool down in the Thermocycler. 15 μL of samples and Protein Ladder into appropriate wells were loaded, the voltage was set at 200V and run time was set for around 30 minutes. Once the gel run was finished, it was visualized by Coomassie blue or SYBR Green or methylene blue. Left most lane is Protein Ladder, next is Centyrin by itself and last is the centyrin-siRNA conjugate.

Candidate siRNA sequences were transfected into human cells (H358) at a range of concentrations. RNA was harvested 24 hours post-transfection and analyzed for GYS1 levels via quantitative reverse transcription polymerase chain reaction (RT-PCR). 18S ribosomal RNA was used as the RT-PCR endogenous control gene. Levels of knockdown were compared to untreated cells. EC50 values were calculated using Graphpad Prism software and Emax values represent the maximum percent knockdown of GYS1 mRNA observed (Table 10).

TABLE 10

| | | | Emax |
|---|---|---|---|
| SIRNA | Ref. | EC50 (pM) | (%) |
| A | ABXO8 | 3.841 | 70 |
| B | ABXO9 | 10.02 | 70.2 |
| C | ABXO12 | 12.62 | 71.5 |
| D | ABXO110 | 21.73 | 87.9 |
| E | ABXO113 | 23.74 | 82.1 |
| F | ABXO114 | 6.716 | 75.5 |
| G | ABXO115 | 4.5 | 75.2 |
| H | ABXO116 | 0.7542 | 69.4 |
| I | ABXO117 | 20.87 | 93.8 |
| J | ABXO122 | 8.93 | 65.6 |
| K | ABXO123 | 4.049 | 75.5 |
| L | ABXO124 | 2.642 | 70.5 |
| M | ABXO125 | 0.8761 | 53.3 |
| N | ABXO128 | 2.549 | 76.3 |
| O | ABXO129 | 0.4961 | 64.3 |
| P | ABXO139 | 0.6143 | 70.6 |
| Q | ABXO142 | 23.19 | 80.3 |
| R | ABXO150 | 58.88 | 96.1 |
| S | ABXO151 | 1.502 | 75.6 |
| T | ABXO152 | 24.76 | 91.1 |
| U | ABXO155 | 5.893 | 60.2 |
| V | ABXO156 | 0.6893 | 57.6 |
| W | ABXO160 | 0.787 | 80.1 |
| X | ABXO168 | 8.662 | 75.7 |
| Y | ABXO171 | 38.82 | 69.2 |
| Z | ABXO191 | 54.2 | 75.4 |
| AA | ABXO197 | 19.8 | 77.1 |
| BB | ABXO201 | 7.1 | 75.6 |
| CC | ABXO203 | 5.7 | 74.9 |
| DD | ABXO205 | 9.8 | 80.4 |

Other siRNAs have also been tested as described above and their EC50 are provided in Table 11.

TABLE 11

| SIRNA Pair | EC50 (pM) |
|---|---|
| EE | 1.8 |
| FF | 6 |
| GG | 5.9 |
| HH | 15.1 |
| II | 8.2 |
| JJ | 25.2 |
| KK | |
| LL | 1.35 |
| MM | 3.74 |
| NN | 5.66 |
| OO | 5.56 |
| PP | 6.34 |
| QQ | 11.76 |
| RR | 2.85 |
| SS | 3.35 |
| TT | 5.67 |
| UU | 0.4 |
| VV | 3.72 |
| WW | 3.15 |
| XX | 2.57 |
| YY | 1.64 |
| ZZ | 1.56 |
| AAA | ND |
| BBB | 4.87 |
| CCC | 2.65 |
| DDD | 2.66 |
| EEE | 1.21 |
| FFF | 1.31 |
| GGG | NA |

Selectivity of candidate siRNA sequences was evaluated by transfection of siRNAs into human cells (HEK-293) at a range of concentrations. Cell viability was assessed 72 hours post-transfection using CellTiterGlo. Emax values are reported as the maximum percent reduction in cell viability at the highest siRNA concentration tested (10 nM) (Table 12).

TABLE 12

| SIRNA Pair | Ref. | Emax, % (at 10 nM) |
|---|---|---|
| A | ABXO8 | 5.52 |
| B | ABXO9 | −0.3 |
| C | ABXO12 | 31.67 |
| D | ABXO110 | 25.68 |
| E | ABXO113 | 22.83 |
| F | ABXO114 | −1.1 |
| G | ABXO115 | 30.85 |
| H | ABXO116 | 29.27 |
| I | ABXO117 | 35.19 |
| J | ABXO122 | 28.25 |
| K | ABXO123 | 17.21 |
| L | ABXO124 | 24.09 |
| M | ABXO125 | 71.45 |
| N | ABXO128 | 42.41 |
| O | ABXO129 | 58.02 |
| P | ABXO139 | 34.44 |
| Q | ABXO142 | 8.94 |
| R | ABXO150 | 16.06 |
| S | ABXO151 | 60.95 |
| T | ABXO152 | 42.84 |
| U | ABXO155 | 54.4 |
| V | ABXO156 | 34.1 |
| W | ABXO160 | −4.59 |
| X | ABXO168 | 51.66 |
| Y | ABXO171 | 4.79 |
| Z | ABXO191 | 46.41 |
| AA | ABXO197 | 25.01 |
| BB | ABXO201 | 46.92 |
| CC | ABXO203 | 15.18 |
| DD | ABXO205 | 60.13 |

### Example 2: Selection of Fibronectin Type III (FN3) Domains that Bind CD71

Panning and Biochemical Screening Methods for Identifying FN3 domains that bind to CD71 that do not inhibit transferrin binding to CD71. To screen for FN3 domains that specifically bind CD71 and do not inhibit transferring binding to CD71, streptavidin-coated Maxisorp plates (Nunc catalog 436110) are blocked for 1 hour in Starting Block T20 (Pierce) and then are coated with biotinylated CD71 (using same antigen as in panning) or negative controls (an unrelated Fc-fused recombinant protein and human serum albumin) for 1 hour in the presence of transferring or with FN3 protein that binds to the CD71 transferrin binding site. The concentration of transferrin is up to 35 μM. Without being bound to any particular theory, the inclusion of the transferrin or the FN3 protein that binds to the CD71 transferrin binding site pushes the selection of the FN3 domains to those that do not compete or inhibit with transferrin binding to CD71. Plates are rinsed with TBST and diluted lysate is applied to plates for 1 hour. Following additional rinses, wells are treated with HRP-conjugated anti-V5 tag antibody (Abcam, ab1325), for 1 hour and then are assayed with POD (Roche, 11582950001). The DNA from FN3 domain lysates with signals at least 10-fold ELISA signal above that of streptavidin controls are sequenced resulting in FN3 domain sequences isolated from the screening.

### Example 3: Selection of Fibronectin Type III (FN3) Domains that Bind CD71 and are not Competitive with Transferrin

To identify CD71 binding FN3 domains that were either not competitive or minimally competitive with transferrin a biased CIS-display strategy was designed. In short, using the output recovered after 5 rounds of panning on the ECD of human CD71 (Example 3). Additional rounds of off-rate selection were performed as described in Example 3 with the addition of either 1) a wash step with human holo transferrin to elute Centyrins that bound at the same site as transferrin before the final elution step or 2) elution of FN3 domain binders with monoclonal antibody OKT9. FN3 domains recovered from the transferrin wash strategy and the OKT9 elution strategy were PCR amplified and cloned into pET vector as previously described. 228 FN3 domains that specifically bound huCD71 were confirmed by ELISA for binding to huCD71 ECD. A subset of the unique binders was analyzed by SEC, conjugated to MMAF and assessed for internalization via cell viability assay in SKBR-3 cells+/−holo human transferrin. The polypeptides were found to be internalized by the receptor.

A membrane proteome array (MPA) assay was performed to profile the specificity of the polypeptide comprising the sequence of SEQ ID NO: 509 and the polypeptide comprising SEQ ID NO: 509 linked to the siRNA pair with linker number comprising the sequence and linker as set forth in siRNA Pair 0000 against a library of human membrane proteins. The MPA library contains over 6000 human membrane proteins, including 94% of all single-pass, multi-pass and GPI anchored proteins including GPCRs, ion channels and transporters with each membrane protein uniquely expressed in an avian QT6 cell background. Flow cytometry is used to directly detect ligand (FN3 domain) binding to membrane proteins individually expressed in unfixed cells.

The polypeptide comprising of SEQ ID NO: 509 and the polypeptide comprising the amino acids sequence of SEQ ID NO: 509 linked to the siRNA pair with linker number comprising the sequence and linker as set forth in siRNA Pair 0000 were screened at the concentration with optimal signal/background noise ratio, 1.25 ug/ml, 1.25 ug/ml and 0.31 ug/ml respectively, against the MPA. Membrane protein targets identified in screening were followed up in validation procedure using ligand serial dilution and cells individually transfected with identified targets. The screen determined that the polypeptides were specific to binding to CD71 and the addition of the siRNA conjugated to the polypeptide did not change the specificity.

### Example 4: Knockdown of mRNA in Muscle Cells Using CD71 FN3 Domain-Oligonucleotide Conjugates

muCD71 binding FN3 domains are conjugated to siRNA oligonucleotides or antisense oligonucleotides (ASOs) using maleimide chemistry via a cysteine that is uniquely engineered into the FN3 domain. The cysteine substitutions can be one such as those provided for herein and also as provided for in U.S. Patent Application Publication No. 20150104808, which is hereby incorporated by reference in its entirety. siRNAs or ASOs are modified with standard chemical modifications and confirmed to enable knockdown of the targeted mRNA in vitro. FN3 domain-oligonucleotide conjugates are dosed intravenously in mice at doses up to 10 mg/kg oligonucleotide payload. At various time points following dosing, mice are sacrificed; skeletal muscle, heart muscle and various other tissues will be recovered and stored in RNAlater™ (Sigma Aldrich) until needed. Target gene knockdown is assessed using standard qPCR ΔΔC_{T} methods and primers specific for the target gene and a control gene. The target gene is found to be knock downed in the muscles and such knockdown is enhanced by conjugating the siRNA or ASO to the CD71 FN3 binding domain.

FN3-siRNA conjugates tested are as described in Table 13, below.

TABLE 13

| | CD71 FN3 domain | |
|---|---|---|
| Conjugate | (Centyrin) | GYS1 siRNA Pair |
| ABXC-1 | SEQ ID NO: 572 | RRRR |
| ABXC-2 | SEQ ID NO: 572 | SSSS |
| ABXC-3 | SEQ ID NO: 572 | TTTT |
| ABX1005 | SEQ ID NO: 572 | Non-GYS1 |
| | | target/control |
| ABXC-27 | SEQ ID NO: 572 | A |
| ABXC-28 | SEQ ID NO: 572 | QQQQ |
| ABXC-29 | SEQ ID NO: 572 | PPPP |

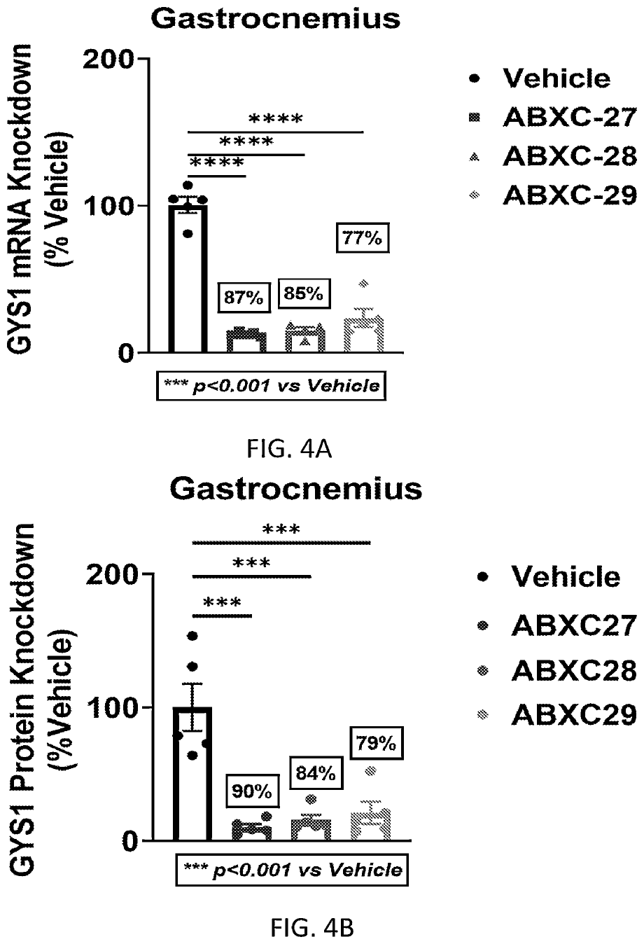

FIG. 4A demonstrates the knockdown of GYS1 mRNA in mouse gastrocnemius muscle using 3 different FN3 domain-siRNA conjugates compared with vehicle alone. For efficacy studies, male GAA−/− mice (at the ages of 4-5 weeks) were obtained from Jackson Laboratories. All animals were treated in accordance with IACUC protocols. Five animals received a single tail vein intravenous bolus injection of either 5.4 mg/kg of three different FN3 domain-siRNA conjugates (3 mpk Gys1siRNA) or vehicle. Four weeks after the single dose, the mice were euthanized, gastrocnemius muscle were collected in RNAlater, stored at 4 C overnight and were frozen at −80 C. Total RNA was isolated from the gastrocnemius using Qiagen's RNeasy Fibrous Tissue kit. Expression levels of the target Gys1 and the endogenous controls (Pgk1, Ubc, Hprt1 and Aha1) were analyzed using real-time, quantitative PCR. Data were analyzed using the ΔΔCt method normalizing to control animals dosed with vehicle alone. The percentage knockdown of Gys1 mRNA in the FN3 domain-siRNA conjugate treatment groups were measured by subtracting the percentage remaining Gys1 mRNA levels by 100. Statistical significance was calculated using One-way ANOVA with Dunnett's multiple comparison tests in the GraphPad Prism software. Statistical significance is displayed on the figure with asterisk ***p<0.001.

FIG. 4B demonstrates the knockdown of GYS1 protein in mouse gastroenemius muscle using 3 different FN3 domain-siRNA conjugates compared with vehicle alone. Gys1 protein quantification in gastrocnemius was performed by homogenizing gastrocnemius in RIPA buffer. Protein concentrations in the gastrocnemius were measured using the Bradford assay. Gys1 levels were quantified using the manufacturer's standard method for 12-230 kDa Jess separation modules (SM-W004). The proteins were separated by immobilizing on capillaries using protein Simple's proprietary photoactivated capture chemistry. Anti-Gys1 primary antibody were used at 1:100 dilution. The chemiluminescent revelations were established using peroxide/luminol-S. A digital image of the capillaries' chemiluminescence was captured using Compass' Simple Western software, which automatically measures height (chemiluminescence intensity), area, and signal/noise ratio. An internal system was included in each run. The peak area values of FN3 domain-siRNA conjugate treatment groups were normalized to the vehicle treated tissues and the percentage knockdown of Gys1 protein in the treatment groups were measured by subtracting the percentage remaining Gys1 protein levels by 100. Statistical significance was calculated using One-way ANOVA with Dunnett's multiple comparison tests in the GraphPad Prism software. Statistical significance is displayed on the figure with asterisk ***p<0.001.

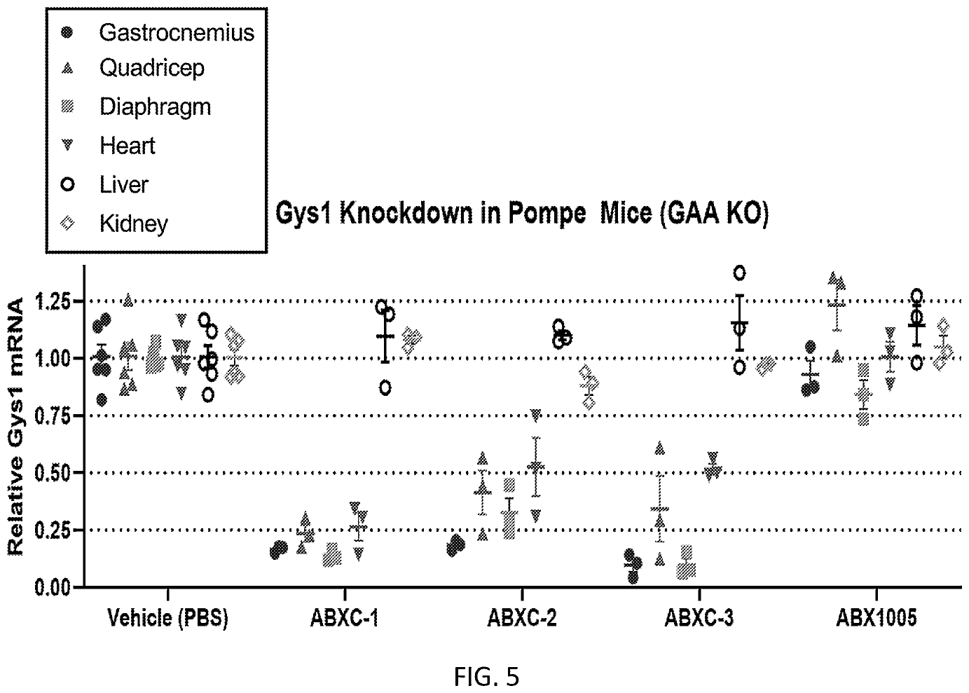

FIG. 5 demonstrates the GYS1 knockdown is highly specific for skeletal muscle using 3 different FN3 domain-siRNA conjugates compared with a siRNA to a different target (AHA-1). Male GAA−/− mice (at the ages of 8-9 weeks) were obtained from Jackson Laboratories. All animals were treated in accordance with IACUC protocols. Three animals received a single tail vein intravenous bolus injection of either 17.9 mg/kg of three different FN3 domain-siRNA conjugates (10 mpk Gys1siRNA), 17.9 mg/kg of one FN3 domain-siRNA conjugate (10 mpk Aha1 siRNA) or vehicle. Two weeks after the single dose, the mice were euthanized. Gastrocnemius, quadriceps, diaphragm, heart, liver, and kidney tissues were collected in RNAlater, stored at 4 C overnight and were frozen at −80 C. Total RNA was isolated from the tissues using Qiagen's RNeasy Fibrous Tissue kit. Expression levels of the target Gys1/Aha1 and the endogenous control (Pgk1) were analyzed using real-time, quantitative PCR. Data were analyzed using the ΔΔCt method normalizing to control animals dosed with vehicle alone.

### Example 5: RNA-Seq Experiments Comparing ABX-HHH Treated and Untreated Cells

mRNA sequencing was performed on polyA+ selected mRNA after HEK293T cells were lipofected with 10 nM ABXO-HHH for 24 h. 6 replicates per treatment were sequenced to an average depth >30 million reads. Libraries were pseudoaligned to the GRCh38 transcriptome using Kallisto with a mapping rate >90%. The values in this volcano plot were generated using DESeq2 to compare ABXO-HHH treated RNA-seq libraries versus untreated cells. Each dot in the plot in FIG. 2 represents the measured expression change in a gene. The X axis represents the log₂ fold change in gene expression (ABXO-HHH divided by untreated) for all genes. The Y axis shows the negative log₁₀ transformed adjusted P value. The black arrow points to GYS1 on the plot.

### Example 6: Binding Specificity of CD71 FN3 Domain siRNA Conjugate

A membrane proteome array (MPA) assay was performed to profile the specificity of CD71 FN3 domain and CD71 FN3 domain siRNA conjugate against the library of human membrane proteins (FIG. 3). The MPA contains over 6000 human membrane proteins, covering 94% of all single-pass, multi-pass and GPI anchored proteins including GPCRs, ion channels and transporters with each membrane protein uniquely expressed in an avian QT6 cell background. Flow cytometry was used to directly detect FN3 domain binding to membrane proteins individually expressed in unfixed cells.

FN3 domain and FN3 domain-siRNA conjugate were screened at the concentration with optimal signal/background noise ratio, 1.25 ug/ml or 0.31 ug/ml respectively, against the MPA. Membrane protein targets identified in screening were validated using ligand serial dilution on cells uniquely expressing the identified targets.

### Example 7: Treatment of Glycogen Storage Disease with a Composition Comprising Fibronectin Type III (FN3) Domains that Bind CD71 Co-Administrated with an Enzyme Replacement Therapy (ERT)

A subject with Pompe Disease is co-administered a composition comprising a CD71-binding FN3 domain linked to a siRNA targeting GYS1, along with the enzyme glucosidase alpha acid (GAA). Compared to subjects receiving only the enzyme replacement therapy, the subject receiving both the CD71-binding FN3 domain linked to a siRNA targeting GYS1 and the glucosidase alpha acid ERT has an enhanced response as compared to the administration of only GAA as ERT and greater reduction of symptoms.

### Example 8: Treatment of Glycogen Storage Disease with a Composition Comprising Fibronectin Type III (FN3) Domains that Bind CD71 Co-Administrated with an Enzyme Replacement Therapy (ERT)

A subject with Cori's Disease is co-administered a composition comprising a CD71-binding FN3 domain linked to a siRNA targeting GYS1, along with glycogen debranching enzyme (AGL). Compared to subjects receiving only the enzyme replacement therapy, the subject receiving both the CD71-binding FN3 domain linked to a siRNA targeting GYS1 and the glycogen debranching enzyme ERT has an enhanced response as compared to the adminisratoin of the AGL ERT and greater reduction of symptoms.

### Example 9: Repeat-Dose Study to Assess Pharmacodynamic Effect of Co-Administering a Composition Comprising Fibronectin Type III (FN3) Domains that Bind CD71 with an Enzyme Replacement Therapy (ERT)

A repeat-dose study was performed to assess the pharmacodynamic effect of CD71-binding FN3 domains as a monotherapy or in combination with enzyme replacement therapy.

Experimental Conditions: ABXC-29 is composed of a rodent CD71-binding Centyrin (SEQ ID NO: 572), that is chemically conjugated to a rodent-specific GYS1 siRNA ABXO-371 (sense strand of SEQ ID NO: 711 and antisense strand of SEQ ID NO: 712). ABXC-29 was used in mice as a surrogate to macromolecule comprising a polypeptide that binds to human CD71, which is composed of a human CD71-binding Centyrin (SEQ ID NO: 509), that is chemically conjugated to a human-specific GYS1 siRNA (sense strand of SEQ ID NO: 706 and antisense strand of SEQ ID NO: 707). Recombinant human acid alpha-glucosidase (rhGAA) is used in enzyme replacement therapy to treat GAA-mediated or GAA-associated diseases. The study was performed in twenty (20) male GAA −/− mice (B6;129-Gaa^{tm1Rabn}; J strain, stock number 004154; 6 ^{ne}; Jackson Laboratories, Bar Harbor, ME) and five (5) wild-type mice (C57BL/6J, stock number 000664; Jackson Laboratories, Bar Harbor, ME). Five wild-type mice and five GAA −/− mice were treated with vehicle (HBS) only. Five GAA −/− mice were treated with repeat intravenous doses of ABXC-29 as monotherapy. Five GAA −/− mice were treated with repeat doses of rhGAA as monotherapy. Five GAA −/− mice were treated with ABXC-29 in combination with rhGAA as combination therapy.

Glycogen Synthase 1 mRNA expression in muscle cells and heart cells after therapy: Results are shown in Table 14, below, and are represented as percent of vehicle in the GAA −/− mice. Two weeks after administration of repeat doses of 3 mg/kg of ABXC-29, expression of Gys1 mRNA was reduced by 83% in the diaphragm, 81% in the quadriceps, and 84% in both the gastrocnemius and the heart. When rhGAA was administered as a monotherapy, no effect on Gys1 mRNA expression was observed. When ABXC-29 and rhGAA were administered as a combination therapy, expression of Gys1 mRNA was reduced by 79% in the diaphragm, 84% in the gastrocnemius, and 83% in the quadriceps, indicating that administration of rhGAA did not impact the activity of ABXC-29 in muscle cells. By contrast, in heart cells, co-administration of ABXC-29 and rhGAA resulted in a reduction of 22% in Gys1 mRNA expression, suggesting a lesser impact on the activity of ABCX-29 in heart cells which could be clinically relevant.

TABLE 14

| | Gastrocnemius | | Heart % | |
|---|---|---|---|---|
| | % decrease | Quadriceps % | decrease in | Diaphragm % |
| | in Gys1 | decrease in | Gys1 | decrease in |
| | mRNA | Gys1 mRNA | mRNA | Gys1 mRNA |
| ABXC-29 | 84% | 81% | 76% | NA |
| (10 mg/kg) | | | | |
| ABXC-29 | 84% | 81% | 84% | 83% |
| (3 mg/kg) | | | | |
| rhGAA | NS | NS | NS | NS |
| ABXC-29 | 84% | 83% | 22% | 79% |
| (3 mg/kg) + | | | | |
| rhGAA | | | | |

NS = not significant; NA = not available

Glycogen levels in muscle cells after therapy: Results are shown in Table 15, below, and are represented as μg glycogen/mg tissue. Administration of repeat doses of 3 mg/kg of ABXC-29 as a monotherapy reduced glycogen levels by 23% in the gastrocnemius and by 67% in the quadriceps. Administration of rhGAA as a monotherapy reduced glycogen by 33% in the gastrocnemius and by 35% in the quadriceps. Administration of ABXC-29 (at 3 mg/kg) and rhGAA as a combination therapy reduced glycogen by 48% in the gastrocnemius and by 42% in the quadriceps. These results demonstrate a synergistic effect of the two therapeutics. As illustrated below, the combination of the two therapeutics generated a response that was similar to the monotherapy dose of 10 mg/kg dose of ABXC-29 and was above the response of monotherapy dose the 3 mg/kg dose of ABXC-29 and above the rhGAA monotherapy dose. These results could not have been predicted.

TABLE 15

| | Gastrocnemius % | Quadriceps % |
|---|---|---|
| | decrease in | decrease in |
| | glycogen levels | glycogen levels |
| ABXC-29 (10 mg/kg) | 53% | 67% |
| ABXC-29 (3 mg/kg) | 23% | 27% |
| rhGAA | 33% | 35% |
| ABXC-29 (3 mg/kg) + rhGAA | 48% | 42% |

General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification, including immunoprecipitation, chromatography, electrophoresis, centrifugation, and crystallization, are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications of the embodiments in addition to those described herein will become apparent to those skilled in the art from the foregoing description. Such modifications are intended to fall within the scope of the appended claims.

The foregoing written specification is considered to be sufficient to enable one skilled in the art to practice the embodiments. Various modifications of the embodiments in addition to those shown and described herein will become apparent to those skilled in the art from the foregoing description and fall within the scope of the appended claims.

What is claimed is:

1. A method of treating a glycogen storage disease in a subject in need thereof, the method comprising the administration of:
a composition comprising one or more FN3 domains linked to an siRNA comprising a sense strand and antisense strand, wherein the siRNA targets Gys1; and
an enzyme replacement therapy (ERT) used to treat the glycogen storage disease.

2. The method of claim 1, wherein the glycogen storage disease is selected from the group consisting of Pompe Disease (GSD2, glucosidase alpha acid (GAA) deficiency), Cori's disease or Forbes' disease (GSD3, Glycogen debranching enzyme (AGL) deficiency), Andersen's disease (GSD4, glycogen branching enzyme (GBE1) deficiency), McArdle disease (GSD5, Muscle glycogen phosphorylase (PYGM) deficiency), Tarui's Disease (GSD7, Muscle phosphofructokinase (PFKM) deficiency), Aldolase A Deficiency (GSD12, aldolase A (ALDOA) deficiency), type II Diabetes/diabetic nephropathy, Lafora Disease, hypoxia, and adult polyglucosan body disease.

3. The method of claim 1, wherein the ERT comprises one or more enzymes selected from the group consisting of glucosidase alpha acid (GAA), glycogen debranching enzyme (AGL), glycogen branching enzyme (BGE1), muscle glycogen phosphorylase (PYGM), muscle phosphofructokinase (PFKM), aldolase A (ALDOA), malin, laforin, glycogen synthase (GYS2), glucose-6-phosphatase (G6PC/SLC37A4), phosphorylase kinase (PHKA2/PHKB/PHKG2/PHKA1), phosphoglycerate mutase (PGAM2), muscle lactate dehydrogenase (LDHA), glucose transporter (GLUT2), β-enolase (ENO3), and glycogenin-1 (GYG1).

4. The method of claim 1, wherein the one or more FN3 domains comprises a FN3 domain that binds to CD71.

5. The method of claim 1, wherein the siRNA that targets Gys1 is an siRNA that reduces the expression of GYS1.

6. The method of claim 4, wherein the siRNA that targets Gys1 is an siRNA that reduces the expression of GYS1.

7. The method of claim 1, wherein:
the glycogen storage disease is Pompe Disease;
the FN3 domain is a polypeptide that binds to CD71; and
the ERT comprises administering glucosidase alpha acid (GAA).

8. The method of claim 1, wherein:
the glycogen storage disease is Lafora Disease;
the FN3 domain is a polypeptide that binds to CD71; and
the ERT comprises administering malin, laforin, or both.

9. The method of claim 1, wherein the siRNA is covalently linked to the FN3 domain through a chemical linker covalently attached to the sense strand or the antisense strand of the siRNA.

10. The method claim 1, wherein the siRNA molecule comprises the siRNA pair comprising a sense strand and antisense strand as set forth of OOOO, A, B, C, D, E, F, G, H, I, J, K, L, M, N, O, P, Q, R, S, T, U, V, W, X, Y, Z, AA, BB, CC, DD, EE, FF, GG, HH, II, JJ, KK, LL, MM, NN, OO, PP, QQ, RR, SS, TT, UU, VV, WW, XX, YY, ZZ, AAA, BBB, CCC, DDD, EEE, FFF, GGG, HHH, III, JJJ, KKK, LLL, MMM, NNN, OOO, PPP, QQQ, RRR, SSS, TTT, UUU, VVV, WWW, XXX, YYY, ZZZ, AAAA, BBBB, CCCC, DDDD, EEEE, FFFF, GGGG, HHHH, IIII, JJJJ, KKKK, LLLL, MMMM, NNNN, PPPP, or as set forth in Table 3A, Table 3B, or Table 4.

11. The method of claim 1, wherein the siRNA linked to the FN3 domain is conjugated to a cysteine residue of the FN3 domain.

12. The method of claim 11, wherein the cysteine in the FN3 domain is at a position that corresponds to residue 6, 8, 10, 11, 14, 15, 16, 20, 30, 34, 38, 40, 41, 45, 47, 48, 53, 54, 59, 60, 62, 64, 70, 88, 89, 90, 91, or 93 of the FN3 domain comprising the amino acid sequence based on SEQ ID NO: 713.

13. The method of claim 12, wherein the cysteine is located at a position that corresponds to residue 6, 53, or 88 of the amino acid sequence of SEQ ID NO: 713.

14. The method of claim 1, wherein the FN3 domain has an amino acid sequence selected from the group consisting of SEQ ID NOs: 509, 708, and 710.

15. The method of claim 1, wherein the FN3 domain comprises an amino acid sequence that is at least 95% identical to an amino acid sequence of SEQ ID NOs: 273, 288-291, 301-310, 312-572, 592-599, or 708-710.

16. The method of claim 1, wherein the FN3 domain that binds CD71 has an amino acid of SEQ ID NOs: 273, 288-291, 301-310, 312-572, 592-599, or 708-710.

17. A pharmaceutical composition for treating a glycogen storage disease comprising a composition comprising one or more FN3 domains linked to an siRNA comprising a sense strand and antisense strand; and an enzyme replacement therapy (ERT) to treat the glycogen storage disease.

18. A kit for treating a glycogen storage disease, the kit comprising a first container comprising a pharmaceutical composition comprising one or more FN3 domains linked to an siRNA comprising a sense strand and antisense strand; and a second container comprising a pharmaceutical composition comprising an enzyme replacement therapy (ERT) to treat the glycogen storage disease.

19. The kit of claim 18, wherein the one or more FN3 domains comprise a FN3 domain that binds to CD71.

20. The kit of claim 18, wherein siRNA targets Gys1.

21. The kit of claim 18, wherein the one or more FN3 domains comprise a FN3 domain that binds to CD71.

22. The kit of claim 18, wherein the glycogen storage disease is selected from the group consisting of Pompe Disease (GSD2, glucosidase alpha acid (GAA) deficiency), Cori's disease or Forbes' disease (GSD3, Glycogen debranching enzyme (AGL) deficiency), Andersen's disease (GSD4, glycogen branching enzyme (GBE1) deficiency), McArdle disease (GSD5, Muscle glycogen phosphorylase (PYGM) deficiency), Tarui's Disease (GSD7, Muscle phosphofructokinase (PFKM) deficiency), Aldolase A Deficiency (GSD12, aldolase A (ALDOA) deficiency), type II Diabetes/diabetic nephropathy, Lafora Disease, hypoxia, and adult polyglucosan body disease.

23. The kit of claim 18, wherein the ERT comprises glucosidase alpha acid (GAA), glycogen debranching enzyme (AGL), glycogen branching enzyme (BGE1), muscle glycogen phosphorylase (PYGM), muscle phosphofructokinase (PFKM), aldolase A (ALDOA), malin, laforin, glycogen synthase (GYS2), glucose-6-phosphatase (G6PC/SLC37A4), phosphorylase kinase (PHKA2/PHKB/PHKG2/PHKA1), phosphoglycerate mutase (PGAM2), muscle lactate dehydrogenase (LDHA), glucose transporter (GLUT2), β-enolase (ENO3), and glycogenin-1 (GYG1), or a combination thereof.

24. The kit of claim 18, wherein the ERT comprises GAA, malin, laforin, or a combination thereof.

25. The kit of claim 18, wherein the first container comprises a pharmaceutical composition comprising a FN3 domain that binds CD71 linked to a siRNA that targets Gys1 and the second container comprises a pharmaceutical composition comprising the enzyme of GAA.


SEQUENCE LISTING (1075 sequences)

SEQ ID NO: 1 - protein, 87 aa, synthetic construct
LPAPKNLVVSEVTEDSLRLSWTAPDAADSLIQYQESEKVGEAINLTVPGSERSYDLTGLKPGTEYTVSIYGVKGGHRSNPLSAEFTT

SEQ ID NO: 2 - protein, 291 aa, Homo sapiens
MTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 3 - skipped

SEQ ID NO: 4 - protein, 87 aa, Homo sapiens
LPAPKNLVVSRVTEDSARLSWTAPDAADSLIQYQESEKVGEAIVLTVPGSERSYDLTGLKPGTEYTVSIYGVKGGHRSNPLSAIFTT

SEQ ID NO: 5 - protein, 227 aa, synthetic construct
QNSQLQEELRGLRETSNTASTEAQVKGLSTQGGNVGRKKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 6-9 - skipped (4 entries)

SEQ ID NO: 10 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
ctgggaggatgaattcgaat

SEQ ID NO: 11 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttcgaattcatcctcccagtt

SEQ ID NO: 12 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
caaggtgggtggcatctaat

SEQ ID NO: 13 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 14 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
tccgcagcctggatgattat

SEQ ID NO: 15 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
taatcatccaggctgcggatt

SEQ ID NO: 16 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gcgactgcctgtagcaacat

SEQ ID NO: 17 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgttgctacaggcagtcgctt

SEQ ID NO: 18 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
tttatgggcatctggactat

SEQ ID NO: 19 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagtccagatgcccataaatt

SEQ ID NO: 20 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gcgcggaccaacaatttcat

SEQ ID NO: 21 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgaaattgttggtccgcgctt

SEQ ID NO: 22 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ggaccaacaatttcaacgat

SEQ ID NO: 23 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tcgttgaaattgttggtcctt

SEQ ID NO: 24 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
accaacaatttcaacgtgat

SEQ ID NO: 25 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tcacgttgaaattgttggttt

SEQ ID NO: 26 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gcgcaaacagctttgggaat

SEQ ID NO: 27 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttcccaaagctgtttgcgctt

SEQ ID NO: 28 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
agagccatctttgcaacgat

SEQ ID NO: 29 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tcgttgcaaagatggctcttt

SEQ ID NO: 30 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaacgcat

SEQ ID NO: 31 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 32 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
agccatctttgcaacgcaat

SEQ ID NO: 33 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttgcgttgcaaagatggcttt

SEQ ID NO: 34 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ccatctttgcaacgcagcat

SEQ ID NO: 35 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgctgcgttgcaaagatggtt

SEQ ID NO: 36 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
acgcagcggcagtctttcat

SEQ ID NO: 37 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgaaagactgccgctgcgttt

SEQ ID NO: 38 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxynucleotide
  misc_feature 1..19: note = RNA
  misc_feature 20: note = DNA
tgaccaccatccgccgaaat

SEQ ID NO: 39 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tttcggcggatggtggtcatt

SEQ ID NO: 40 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxynucleotide
  misc_feature 1..19: note = RNA
  misc_feature 20: note = DNA
cgaatcggcctcttcaatat

SEQ ID NO: 41 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tattgaagaggccgattcgtt

SEQ ID NO: 42 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxynucleotide
  misc_feature 1..19: note = RNA
  misc_feature 20: note = DNA
cttcaatagcagtgccgaat

SEQ ID NO: 43 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttcggcactgctattgaagtt

SEQ ID NO: 44 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
cagtatctccaccaatctat

SEQ ID NO: 45 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagattggtggagatactgtt

SEQ ID NO: 46 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ccaccaatctctccggctat

SEQ ID NO: 47 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagccggagagattggtggtt

SEQ ID NO: 48 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caccaatctctccggcttat

SEQ ID NO: 49 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
taagccggagagattggtgtt

SEQ ID NO: 50 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
cccctcagcttacggtatat

SEQ ID NO: 51 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tataccgtaagctgaggggtt

SEQ ID NO: 52 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ccctcagcttacggtatcat

SEQ ID NO: 53 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgataccgtaagctgagggtt

SEQ ID NO: 54 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattctat

SEQ ID NO: 55 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 56 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ggaaccgcacggagcgccat

SEQ ID NO: 57 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tggcgctccgtgcggttcctt

SEQ ID NO: 58 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
acggagcgcctctccgacat

SEQ ID NO: 59 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgtcggagaggcgctccgttt

SEQ ID NO: 60 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
agcaagcgcaactctgtgat

SEQ ID NO: 61 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tcacagagttgcgcttgcttt

SEQ ID NO: 62 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
cagatggtcctccatttcat

SEQ ID NO: 63 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgaaatggaggaccatctgtt

SEQ ID NO: 64 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
tctagatctggaaccttaat

SEQ ID NO: 65 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttaaggttccagatctagatt

SEQ ID NO: 66 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
cccacatttacctcgaggat

SEQ ID NO: 67 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tcctcgaggtaaatgtgggtt

SEQ ID NO: 68 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
accgcttggtttgcagttat

SEQ ID NO: 69 - RNA, 21 nt, synthetic construct
  source 1..21: mol_type = other RNA
  modified_base 1..2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
taactgcaaaccaagcggttt

SEQ ID NO: 70 - RNA, 18 nt, synthetic construct
  source 1..18: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
tctcgtggccttaatgaa

SEQ ID NO: 71 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6: mod_base = OTHER; note = 2'Fluoro ribonucleotide
  modified_base 7: mod_base = OTHER; note = 2'O-Methyl ribonucleotide
  modified_base 8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11: mod_base = OTHER; note = 2'O-Methyl ribonucleotide
  modified_base 12: mod_base = OTHER; note = 2'Fluoro ribonucleotide
  modified_base 13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tttcattaaggccacgagatt

SEQ ID NO: 72 - RNA, 19 nt, synthetic construct
  source 1..19: mol_type = other RNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
ctgggaggatgaattcgaa

SEQ ID NO: 73 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttcgaattcatcctcccagtt

SEQ ID NO: 74 - RNA, 19 nt, synthetic construct
  source 1..19: mol_type = other RNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
caaggtgggtggcatctaa

SEQ ID NO: 75 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 76 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
gcgcggaccaacaatttca

SEQ ID NO: 77 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgaaattgttggtccgcgctt

SEQ ID NO: 78 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
gagccatctttgcaacgca

SEQ ID NO: 79 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 80 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
agccatctttgcaacgcaa

SEQ ID NO: 81 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttgcgttgcaaagatggcttt

SEQ ID NO: 82 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
ttacggtatctacattcta

SEQ ID NO: 83 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 84 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
cccacatttacctcgagga

SEQ ID NO: 85 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tcctcgaggtaaatgtgggtt

SEQ ID NO: 86 - RNA, 18 nt, synthetic construct
  source 1..18: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16..18: mod_base = OTHER; note = 2'O-Methyl ribonucleotide
tctcgtggccttaatgga

SEQ ID NO: 87 - RNA, 21 nt, synthetic construct
  source 1..21: mol_type = other RNA
  modified_base 1: mod_base = OTHER; note = 2'O methyl vinyl phosphonate uridine phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6: mod_base = OTHER; note = 2'Fluoro ribonucleotide
  modified_base 7: mod_base = OTHER; note = 2'O-Methyl ribonucleotide
  modified_base 8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11: mod_base = OTHER; note = 2'O-Methyl ribonucleotide
  modified_base 12: mod_base = OTHER; note = 2'Fluoro ribonucleotide
  modified_base 13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide phosphorothioate
tttcattaaggccacgagatt

SEQ ID NO: 88-100 - skipped (13 entries)

SEQ ID NO: 101 - protein, 58 aa, synthetic construct
MLPAPKNLVASRVTEDSARLSWTAPDAAFDSFNIAYWEPGIGGEAIWLRVPGSERSYD

SEQ ID NO: 102 - protein, 32 aa, synthetic construct
LTGLKPGTEYKVWIHGVKGGASSPPLIARFTT

SEQ ID NO: 103 - protein, 58 aa, synthetic construct
MLPAPKNLVVSRVTEDSARLSWTAPDAAFDSFNIAYWEPGIGGEAIWLRVPGSERSYD

SEQ ID NO: 104 - protein, 32 aa, synthetic construct; identical to SEQ ID NO: 102

SEQ ID NO: 105 - protein, 58 aa, synthetic construct
MLPAPKNLVVSRVTEDSARLSWTAPDAAFDSFAIAYWEPGIGGEAIWLRVPGSERSYD

SEQ ID NO: 106 - protein, 32 aa, synthetic construct; identical to SEQ ID NO: 102

SEQ ID NO: 107 - protein, 58 aa, synthetic construct
MLPAPKNLVVSRVTEDSARLSWTAPDAAFDSFNISYWEPGIGGEAIWLRVPGSERSYD

SEQ ID NO: 108 - protein, 32 aa, synthetic construct; identical to SEQ ID NO: 102

SEQ ID NO: 109 - protein, 58 aa, synthetic construct
MLPAPKNLVVSRVTEDSARLSWTAPDAAFDSFNIAYAEPGIGGEAIWLRVPGSRSYDL

SEQ ID NO: 110 - protein, 31 aa, synthetic construct
TGLKPGTEYKVWIHGVKGGASSPPLIARFTT

SEQ ID NO: 111 - protein, 58 aa, synthetic construct
MLPAPKNLVVSRVTEDSARLSWTAPDAAFDSFNIAYWEAGIGGEAIWLRVPGSERSYD

SEQ ID NO: 112 - protein, 32 aa, synthetic construct; identical to SEQ ID NO: 102

SEQ ID NO: 113 - protein, 58 aa, synthetic construct
MLPAPKNLVVSRVTEDSARLSWTAPDAAFDSFNIAYWEPAIGGEAIWLRVPGSERSYD

SEQ ID NO: 114 - protein, 32 aa, synthetic construct; identical to SEQ ID NO: 102

SEQ ID NO: 115 - protein, 58 aa, synthetic construct
MLPAPKNLVVSRVTEDSARLSWTAPDAAFDSFNIAYWEPGAGGEAIWLRVPGSERSYD

SEQ ID NO: 116 - protein, 32 aa, synthetic construct; identical to SEQ ID NO: 102

SEQ ID NO: 117 - protein, 59 aa, synthetic construct
MLPAPKNLVVSRVTEDSARLSWTAPDAAFDSFNIAYWEPGIAGEAIWLRVPGSERSYDL

SEQ ID NO: 118 - protein, 31 aa, synthetic construct; identical to SEQ ID NO: 110

SEQ ID NO: 119 - protein, 59 aa, synthetic construct
MLPAPKNLVVSRVTEDSARLSWTAPDAAFDSFNIAYWEPGIGGEAIALRVPGSERSYDL

SEQ ID NO: 120-272 - skipped (153 entries)

SEQ ID NO: 273 - protein, 90 aa, synthetic construct
  VARIANT 33: note = D, F, Y or H
  VARIANT 35: note = Y, G, A or W
  VARIANT 37: note = I, T, L, A or H
  VARIANT 39: note = S, Y or P
  VARIANT 40: note = Y, G, Q or R
  VARIANT 41: note = G or P
  VARIANT 42: note = A, Y, P, D or S
  VARIANT 47: note = Any amino acid other than M or P
  VARIANT 49: note = Any amino acid other than M or P
  VARIANT 69: note = W, N, S or E
  VARIANT 71: note = L, Y or G
  VARIANT 73: note = D, Q, H or V
  VARIANT 74: note = G or S
  VARIANT 79: note = R, G, F, L or D
  VARIANT 80: note = W, S, P or L
  VARIANT 82: note = T, V, M or S
  VARIANT 85: note = Any amino acid other than M or P
  VARIANT 87: note = Any amino acid other than M or P
MLPAPKNLVVSRVTEDSARLSWTAPDAAFDSFXIXYXEXXXXGEAIXLXVPGSERSYDLTGLKPGTEYXVXIXXVKGGXXSXPLXAXFTT

SEQ ID NO: 274-300 - skipped (27 entries)

SEQ ID NO: 301 - protein, 90 aa, synthetic construct
MLPAPKNLVVSRVTEDSARLSWTAPDAAFDSFPITYIEAVVLGEAIVLTVPGSERSYDLTGLKPGTEYPVGISGVKGGHNSMPLSAIFTT

SEQ ID NO: 302 - protein, 90 aa, synthetic construct
MLPAPKNLVVSEVTEDSARLSWQGVARAFDSFMINYSELFWMGEAIVLTVPGSERSYDLTGLKPGTEYVVRIKGVKGGKGSWPLHAHFTT

SEQ ID NO: 303 - protein, 90 aa, synthetic construct
MLPAPKNLVVSRVTEDSARLSWTAPDAAFDSFNIEYAETRWYGEAIVLTVPGSERSYDLTGLKPGTEYVVPIDGVKGGIASKPLSAIFTT

SEQ ID NO: 304 - protein, 90 aa, synthetic construct
MLPAPKNLVVSEVTEDSARLSWQGVARAFDSFLITYRDQIFAGEVIVLTVPGSERSYDLTGLKPGTEYPVWIQGVKGGSPSAPLSAESTT

SEQ ID NO: 305 - protein, 90 aa, synthetic construct
MLPAPKNLVVSEVTEDSARLSWQGVARAFDSFLITYREQIFAGEVIVLTVPGSERSYDLTGLKPGTEYWVYIWGVKGGKPSFPLRAGFTT

SEQ ID NO: 306 - protein, 90 aa, synthetic construct
MLPAPKNLVVSRVTEDSARLSWTAPDAAFDSFPIIYMETFSRGEAIVLTVPGSERSYDLTGLKPGTEYRVPIGGVKGGSSSCPLSAIFTT

SEQ ID NO: 307 - protein, 90 aa, synthetic construct
MLPAPKNLVVSDVTEDSARLSWQGVARAFDSFLITYREQIFAGEVIVLTVPGSERSYDLTGLKPGTEYPVWIQGVKGGSPSAPLSAEFTT

SEQ ID NO: 308-309 - skipped (2 entries)

SEQ ID NO: 310 - protein, 90 aa, synthetic construct
MLPAPKNLVVSRVTEDSARLSWTAPDAAFDSFLIAYPEDGFRGEAIVLTVPGSERSYDLTGLKPGTEYPVPILGVKGGGGSGPLSAIFTT

SEQ ID NO: 311 - skipped

SEQ ID NO: 312 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSAISTT

SEQ ID NO: 313 - protein, 90 aa, synthetic construct; identical to SEQ ID NO: 310

SEQ ID NO: 314 - protein, 90 aa, synthetic construct
MLPAPKNLVVSRVTEDSARLSWTAPDAAFDSFPIYYVENVVWGEAIVLTVPGSERSYDLTGLKPGTEYWEVIIGVKGGQCSRPLSAIFTT

SEQ ID NO: 315 - protein, 90 aa, synthetic construct
MLPAPKNLVVSRVTEDSARLSWQGVARAFDSFLITYREQIFAGEVIVLTVPGSERSYDLTGLKPGTECPVWIQGVKGGSPSAPLSAEFTT

SEQ ID NO: 316 - protein, 89 aa, synthetic construct
MLPAPKNLVVSRVTEDSARLSWTAPDAAFDSFGIAYREFRPSGEAIVLTVPGSERSYDLTVETGYRNEVVICGVKGGPWSGPLSAIFTT

SEQ ID NO: 317 - protein, 90 aa, synthetic construct
MLPAPKNLVVSRVTEDSARLSWTAPDAAFDSFPILYTECVYRGEAIVLTVPGSERSYDLTGLKPGTEYHVPITGVKGGGGSWPLSAIFTT

SEQ ID NO: 318 - protein, 90 aa, synthetic construct
MLPAPKNLVVSRVTEDSARLSWTAPDAAFDSFNIMYHEIIYVGEAIVLTVPGSERSYDLTGLKPGTEYPVPIEGVKGGGTSGPLSAIFTT

SEQ ID NO: 319 - protein, 90 aa, synthetic construct
MLPAPKNLVVSRVTEDSARLSWTAPDAAFDSFAITYTEAALCGEAIVLTVPGSERSYDLTGLKPGTEYPVPINGVKGGGTSGPLSAIFTT

SEQ ID NO: 320 - protein, 90 aa, synthetic construct
MLPAPKNLVVARVTEDSARLSWTAPDAAIDSFPIDYSEYWWGGEAIVLTVPGSERSYDLTGLKPGTEYPVLITGVKGGYRSGPLSAIFTT

SEQ ID NO: 321 - protein, 90 aa, synthetic construct
MLPAPKNLVVSRVTEDSARLSWTAPDAAFDSFSIRYNEFIVAGEAIVLTVPGSERSYDLTGLKPGTEYDVPIAGVKGGGASWPLSAIVTT

SEQ ID NO: 322 - protein, 88 aa, synthetic construct
MLPAPKNLVVSRVTEDSARLSWTAPDAAFDSFWYLELQFAGEAIVLTVPGSERSYDLTGLKPGTEYNVPITGVKGGIISFPLSAIFTT

SEQ ID NO: 323 - protein, 90 aa, synthetic construct
MLPAPKNLVVSRVTEDSARLSWTAPDAAFDSFAIWYHEWYGDGEAIVLTVPGSERSYDLTGPKPGTEYRVRISGVKGGFESGPLSAIFTT

SEQ ID NO: 324 - protein, 90 aa, synthetic construct
MLPAPKNLVVSRVTEDSARLSWTAPDAAFDSFMIRYQEGTRWGEAIVLTVPGSERSYDLTGLKPGTEYIVMIAGVKGGQISLPLSAIFTT

SEQ ID NO: 325 - protein, 90 aa, synthetic construct
MLPAPKNLVVSRVTEDSARLSWTAPDAAFDSFPIWYLEKSYQGEAIVLTVPGSERSYDLTGLKPGTEYVVPIIGVKGGRDSCPLSAIFTT

SEQ ID NO: 326 - protein, 90 aa, synthetic construct
MLPAPKNLVVSEVTEDSARLSWQGVARAFDSFLITYREQIFAGEVIVLTVPGSERSYDLTGLKPGTEYPVWIQGVKGGSPSAPLSAEFTT

SEQ ID NO: 327 - protein, 89 aa, synthetic construct
MLPAPKNLVVSRVTEDSARLSWTAPDAAFDSFRISYAETVRQGEAIVLTVPGSERSYDLTVETGYRNWVMILGVKGGPGSLPLSAIFTT

SEQ ID NO: 328 - protein, 90 aa, synthetic construct
MLPAPKNLVVSEVTEDSARLSWQGVVRAFDSFLITYREQIFAGEVIVLTVPGSERSYDLTGLKPGTEYPVWIQGVKGGSPSAPLSAEFTT

SEQ ID NO: 329 - protein, 90 aa, synthetic construct
MLPAPKNLVVSRVTEDSARLSWTAPDAAFDSFWIEYWEAVGFGEAIVLTVPGSERSYDLTGLKPGTEYFVGIYGVKGGYLSAPLSAIFTT

SEQ ID NO: 330 - protein, 90 aa, synthetic construct
MLPAPKNLVVSRVTEDSARLSWTAPDAAFDSFIIHYVEQQLIGEAIVLTVPGSERSYDLTGLKPGTEYPVPITGVKGGACSWPLSAIFTT

SEQ ID NO: 331 - protein, 90 aa, synthetic construct
MLPAPKNLVVSRVTEDSARLSWTAPDAAFDSFTIEYSEHPIDGEAIPLFVPGSERSYDLTGLKPGTEYYVRIHGVKGGWFSHPLWAFFTT

SEQ ID NO: 332 - protein, 90 aa, synthetic construct
MLPAPKNLVVSRVTEDSARLSWQGVARAFDSFLITYREQIFAGEVIVLTVPGSERSYDLTGLKPGTEYGVTIAGVKGGWRSKPLNAESTT

SEQ ID NO: 333 - protein, 90 aa, synthetic construct
MLPAPKNLVVSRVTEDSARLSWTAPDAAFDSFGIAYVESYWYGEAIVLTVPGSERSYDLTGLKPGTEYNVPIYGVKGGDGSGPLSAIFTT

SEQ ID NO: 334 - protein, 90 aa, synthetic construct
MLPAPKNLVVSRVTEDSARLSWTAPDAAFDSFYITYVELNLAGEAIVLTVPGSERSYDLTGLKPGTEYPVPILGVKGGSLSQPLSAIFTT

SEQ ID NO: 335 - protein, 90 aa, synthetic construct
MLPAPKNLVVSRVTEDSARLSWTAPDAAFDSFPISYIESIADGEAIVLTVPGSERSYDLTGLKPGTEYWVAIVGVKGGPFSWSLSAIVTT

SEQ ID NO: 336 - protein, 90 aa, synthetic construct
MLPAPKNLVVSEVTEDSARLSWQGVARAFDSFLITYREQIFAGEVIVPTVPGSERSYDLTGLKPGTEYPVPIAGVKGGGPSAPLSAIFTT

SEQ ID NO: 337 - protein, 90 aa, synthetic construct
MLPAPKNLVVSRVTEDSARLSWTTPDAAFDSFPIYYWEVTITGEAIYLSVPGSERSYDLTGLKPGTEYPVDIPGVKGGAASPPLSAIFTT

SEQ ID NO: 338 - protein, 90 aa, synthetic construct
MLPAPKNLVVSRVTEDSARLSWTAPDAAFDSFPILYLEHTVRSEAIVLTVPGSERSYDLTDLKPGTEYCVPIDGVKGGLRSRPLSAIFTT

SEQ ID NO: 339 - protein, 90 aa, synthetic construct
MLPAPKNLVVSRVTEDSARLSWTAPDAAFDSFPIPYTEPPDPGEAIVLTVPGSERSYDLTGLKPGTEYLVTILGVKGGSMSVPLSAIFTT

SEQ ID NO: 340 - protein, 90 aa, synthetic construct
MLPAPKNLVVSRVTEDSARLSWTAPDAAFDSFTIDYWENRCPGEAIVLTVPGSERSYDLTGLKPGTEYCVWISGVKGGYSSWPLSAIFTT

SEQ ID NO: 341 - protein, 90 aa, synthetic construct
MLPAPKNLVVSRVTEDSARLSWQGVARAFDSFLITYREQIFAGEVIVLTVPGSERSYDLTGLKPGTEYPVWIQGVKGGHLSDPLSAIVTT

SEQ ID NO: 342 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGDYSEPLSAIFTT

SEQ ID NO: 343 - protein, 90 aa, synthetic construct
MLPAPKNLVVSRVTEDSARLSWTAPDAAFDSFMIVYYEYTRFGEAIVLTVPGSERSYDLTGLKPGTEYTVPIDGVKGGGRSSPLSAIFTT

SEQ ID NO: 344 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSAIVTT

SEQ ID NO: 345 - protein, 90 aa, synthetic construct; identical to SEQ ID NO: 326

SEQ ID NO: 346 - protein, 90 aa, synthetic construct
MLPAPKNLVVSRVTEDSARLSWTAPDAAFDSFIIPYAEVRPDGEAIVLTVPGSERSYDLTGLKPGTEYSVLIHGVKGGKLSLPLSAIFTT

SEQ ID NO: 347 - protein, 90 aa, synthetic construct
MLPAPKNLVVSRVTEDSARLSWTAPDAAFDSFGIVYLEMMVTGEAIVLTVPGSERSYDLTGLKPGTEYDVPILGVKGGTRSVPLSAIFTT

SEQ ID NO: 348 - protein, 94 aa, synthetic construct
MLPAPKNLVVSRVTEDSARLSWTAPDAAFDSFLIYYEEGYLEYYYSGEAIVLTVPGSERSYDLTGLKPGTEYYVGIVGVKGGGLSGPLSAISTT

SEQ ID NO: 349 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGDWSLPLSAIFTT

SEQ ID NO: 350 - protein, 91 aa, synthetic construct
MSLPAPKNLVVSRVTEDSARLSWTAPDAAFDSFTIHYREFQLSGEAIVLTVPGSERSYDLTGLKPGTEYDVPIEGVKGGPGSRPLSAIFTT

SEQ ID NO: 351 - protein, 90 aa, synthetic construct
MLPAPKNLVVSEVTEDSARLSWQGVARAFDSFLITYREQIFAGEVIVLTVPGSECSYDLTGLKPGTEYPVWIQGVKGGSPSAPLSAEFTT

SEQ ID NO: 352 - protein, 90 aa, synthetic construct
MLPAPKNLVVSRVTEDSARLSWTAPDAAFDSFEIDYDELAIYGEAIVLTVPGSERSYDLTGLKPGTEYGVRIPGVKGGMPSLPLSAIVTT

SEQ ID NO: 353 - protein, 90 aa, synthetic construct
MLPAPENLVVSEVTEDSARLSWQGVARAFDSFLITYREQIFAGEVIVLTVPGSERSYDLTGLKPGTEYPVWIQGVKGGSPSAPLSAESTT

SEQ ID NO: 354 - protein, 90 aa, synthetic construct
MLPAPKNLVVSRVTEDSARLSWTAPDAAFDSFAIAYGEHIVIGEAIVLTVPGSERSYDLTGLKPGTEYMVPIAGVKGGPISLPLSAIFTT

SEQ ID NO: 355 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSAIFTT

SEQ ID NO: 356 - protein, 90 aa, synthetic construct
MLPAPKNLVVSRVTEDSARLSWTAPDAAFDSFSIGYVELVLLGEAIVLTVPGSERSYDLTGLKPGTEYDVLIPGVKGGSLSRPLSAIFTT

SEQ ID NO: 357 - protein, 90 aa, synthetic construct
MLPAPKNLVVSRVTEDSARLSWTAPDAAFDSFAIPYAELSRNGEAIVLTVPGSERSYDLTGLKPGTEYTVLIHGVKGGCLSDPLSAIFTT

SEQ ID NO: 358 - protein, 90 aa, synthetic construct
MLPAPKNLVVSRVTEDSARLSWTAPDAAFDSFHIEYLELSRHGEAIVLTVPGSERSYDLTGLKPGTEYWVMIFGVKGGGPSKPLSAIFTT

SEQ ID NO: 359 - protein, 88 aa, synthetic construct
MLPAPKNLVVSRVTEDSARLSWTAPDAAFDSFVYNEVHWIGEAIVLTVPGSERSYDLTGLKPGTEYFVGIYGVKGGHWSKPLSAIFTT

SEQ ID NO: 360 - protein, 90 aa, synthetic construct; identical to SEQ ID NO: 352

SEQ ID NO: 361 - protein, 90 aa, synthetic construct
MLPAPKNLVVSRVTEDSARLSWTAPDAAFDSFQIVYSELWIKGEAIVLTVPGSERSYDLTGLKPGTEYQVPIPGVKGGRNSFPLSAIFTT

SEQ ID NO: 362 - protein, 90 aa, synthetic construct
MLPAPKNLVVSRVTEDSARLSWTAPDAAFDSFKIRYTETRSIGEAIVLTVPGSERSYDLTGLKPGTEYCVPIGGVKGGDSSWPLSAISTT

SEQ ID NO: 363 - protein, 90 aa, synthetic construct
MLPAPKNLVVSRVTEDSARLSWTAPDAAFDSFCISYYERMGRGEAIVLTVPGSERSYDLTGLKPGTEYMVYIFGVKGGLNSLPLSAIFTT

SEQ ID NO: 364 - protein, 90 aa, synthetic construct
MLPAPKNLVVSRVTEDSARLSWTAPDAAFDSFYIVYAEPIPNGEAIVLTVPGSERSYDLTGLKPGTEYSVLIHGVKGGRNSDPLSAIFTT

SEQ ID NO: 365 - protein, 90 aa, synthetic construct
MLPAPKNLVVSRVTKDSARLSWTAPDAAFDSFPIAYAEPRPDGEAIVLTVPGSERSYDLTGLKPGTEYSVLIHGVKGGLLSSPLSAIFTT

SEQ ID NO: 366 - protein, 90 aa, synthetic construct
MLPAPKNLVVSRVTEDSARLSWTAPDAAFDSFTIDYDEPRSPGEAIVLTVPGSERSYDLTGLKPGTEYRVFIWGIKGGDTSFPLSAIFTT

SEQ ID NO: 367 - protein, 88 aa, synthetic construct
MLPAPKNLVVSRVTEDSARLSWTAPDAAFDSFTILYAEQAQFGEAIVLTVPGSERSYDLTGLKPGTEYPITGVKGGTRSGPLSAISTT

SEQ ID NO: 368 - protein, 90 aa, synthetic construct
MLPAPKNLVVSRVTEDSARLSWTAPDAAFDSFIIPYAEVRPDGEAIVLTVPGSERSYDLTGLKPGTEYSVLIHGVKGGHLSDPLSAISTT

SEQ ID NO: 369 - protein, 90 aa, synthetic construct
MLPAPKNLVVSRVTEDSARLSWTAPDAAFDSFHIAYEETATSGEAIYLRVPGSERSYDLTGLKPGTEYGVEIEGVKGGARSRPLYADFTT

SEQ ID NO: 370 - protein, 90 aa, synthetic construct
MLPAPKNLVVSRVTEDSARLSWQGVARAFDSFLITYREQIFAGEVIVLTVPGSERSYDLTGLKPGTEYPVWIQGVKGGDLSNPLSAIFTT

SEQ ID NO: 371 - protein, 90 aa, synthetic construct
MLPAPKNLVVSRVTEDSARLSWTAPDAAFDSFPISYLELSLYGEAIVLTVPGSERSYDLTGLKPGTEYPVGIAGVKGGVVSRPLSAIFTT

SEQ ID NO: 372 - protein, 90 aa, synthetic construct
MLPAPKNLVVSRVTEDSARLSWTAPDAAFDSFTIGYREWYWYGEAIVLTVPGSERSYDLTGLKPGTEYNVPISGVKGGLDSFPLSAIFTT

SEQ ID NO: 373 - protein, 90 aa, synthetic construct
MLPAPKNLVVSEVTEDSARLSWQGVARAFDSFLITYREQIFAGEVIVLTVPGSERSYDLTGLKPGTEYPVWIQGVKGGSPSAPLSAESTT

SEQ ID NO: 374 - protein, 90 aa, synthetic construct
MLPAPKNLVVSRVTEDSARLSWTAPDAAFDSFSITYLEWWNLGEAIVLTVPGSERSYDLTGLKPGTEYMVTIPGVKGGMSSYPLSAIFTT

SEQ ID NO: 375 - protein, 90 aa, synthetic construct
MLPAPKNLVVSRVTEDSARLSWTAPDAAFDSFTISYGEEALIGEAIYLRVPGSERSYDLTGLKPGTEYYVHIEGVKGGSWSQPLAAAFTT

SEQ ID NO: 376 - protein, 90 aa, synthetic construct
MLPAPKNLVVSRVTEDSARLSWTAPDAAFDSFTIEYYENIGIGEAIVLTVPGSERSYDLTGLKPGTEYSVPIVGVKGGPYSHPLSAIFTT

SEQ ID NO: 377 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAIFTT

SEQ ID NO: 378 - protein, 90 aa, synthetic construct
MLPAPKNLVVSRVTEDSARLSWTAPDAAFDSFGIGYYEHKRFGEAIQLSVPGSERSYDLTGLKPGTEYEVDIEGVKGGVLSWPLFAEFTT

SEQ ID NO: 379 - protein, 90 aa, synthetic construct
MLPAPKNLVVSRVTEDSARLSWTAPDAAFDSFVIEYTERFWSGEAIVLTVPGSERSYDLTGLKPGTEYSVPIDGVKGGQCSTPLSAIFTT

SEQ ID NO: 380 - protein, 90 aa, synthetic construct
MLPAPKNLVVSRVTEDSARLSWTAPDAAFDSFWIDYEEEGVIGEAIYLHVPGSERSYDLTGLKPGTEYVVKIHGVKGGHPSHPLVAVFTT

SEQ ID NO: 381 - protein, 90 aa, synthetic construct
MLPAPKNLVVSRVTEDSARLSWQGVARAFDSFLITYVELRHLGEAIVLTVPGSERSYDLTGLKPGTEYPVWIQGVKGGSPSAPLSAEFTT

SEQ ID NO: 382 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGDYSSPLSAIFTT

SEQ ID NO: 383 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAISTT

SEQ ID NO: 384 - protein, 90 aa, synthetic construct
MLPAPKNLVVSRVTEDSARLSWQGVARAFDSFSILYLELTPKGEAIVLTVPGSERSYDLTGLKPGTEYPVWIQGVKGGSPSAPLSAEFTT

SEQ ID NO: 385 - protein, 90 aa, synthetic construct
MLPAPKNLVVSRVTEDSARLSWTAPDAAFDSFIIEYFEPIPIGEAIVLTVPGSERSYDLTGLKPGTEYAVNIYGVKGGYLSHPLSAIFTT

SEQ ID NO: 386 - protein, 90 aa, synthetic construct; identical to SEQ ID NO: 351

SEQ ID NO: 387 - protein, 90 aa, synthetic construct
MLPAPKNLVVSRVTEDSARLSWTAPDAAFDSFAIEYTEFLYSGEAIVLTVPGSERSYDLTGLKPGTEYGVPINGVKGGFVSPPLSAIVTT

SEQ ID NO: 388 - protein, 90 aa, synthetic construct
MLPAPKNLVVSRVTEDSARLSWTAPDAAFDSFPIKYREVLRCGEAIVLTVPGSERSYDLTGLKPGTEYTVPITGVKGGFGSSPLSAIFTT

SEQ ID NO: 389 - protein, 90 aa, synthetic construct
MLPAPENLVVSRVTEDSARLSWTAPDAAFDSFWIEYYEGVIQGEAIVLTVPGSERSYDLTGLKPGTEYFVAIWGVKGGKWSVPLSAIFTT

SEQ ID NO: 390 - protein, 86 aa, synthetic construct
MLPAPKNLVVSRVTEDSARLSWQGVARAFDSFLITYREQIFAGEVIVLTVPGSERSYDLTGLKPGTEYPVWIQGVKGGSPSAEFTT

SEQ ID NO: 391 - protein, 90 aa, synthetic construct
MLPAPKNLVVSRVTEDSARLSWTAPDAAFDSFQIHYWETQGFGEAIVLTVPGSERSYDLTGLKPGTEYPVLIPGVKGGPSSLPLSAIFTT

SEQ ID NO: 392 - protein, 90 aa, synthetic construct; identical to SEQ ID NO: 355

SEQ ID NO: 393 - protein, 90 aa, synthetic construct
MLPAPKNLVVSRVTEDSARLSWTAPDAAFDSFAIEYYEPVPAGEAIYLDVPGSERSYDLTGLKPGTEYDVTIYGVKGGYYSHPLFASFTT

SEQ ID NO: 394 - protein, 90 aa, synthetic construct
MLPAPKNLVVSEVTEDSARLSWQGVARAFDSFLITYREQIFAGEVIVLTVPGSERSYDLTGLKPGTEYPVWIQGVKGGSPSAPLSAIFTT

SEQ ID NO: 395 - protein, 90 aa, synthetic construct; identical to SEQ ID NO: 326

SEQ ID NO: 396 - protein, 91 aa, synthetic construct
MSLPAPKNLVVSRVTEDSARLSWTAPDAAFDSFPIAYLEVFYEGEAIVLTVPGSERSYDLTGLKPGTEYQVPIEGVKGGAMSLPLSAIFTT

SEQ ID NO: 397 - protein, 90 aa, synthetic construct
MLPAPKNLVVSRVTEDSARLSWTAPDAAFDSFHIWYEEETTIGEAIYLHVPGSERSYDLTGLKPGTEYEVHITGVKGGPYSRPLFANFTT

SEQ ID NO: 398 - protein, 90 aa, synthetic construct
MLPAPKNLVVSRVTEDSARLSWTAPDAAFDSFGIAYDEWPEFGEAIVLTVPGSERSYDLTGLKPDTEYIVEIYGVKGGWFSWPLSAIFTT

SEQ ID NO: 399 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSVIFTT

SEQ ID NO: 400 - protein, 90 aa, synthetic construct
MLPAPKNLVVSRVTEDSARLSWTAPDAAFDSFYIWYEEVMYLGEAIVLTVPGSERSYDLTGLKPGTEYNVPIPGVKGGHSSPPLSAIFTT

SEQ ID NO: 401 - protein, 90 aa, synthetic construct
MLPAPKNLVVSRVTEDSARLSWTAPDAAFDSFHILYEELFLVGEAIVLTVPGSERSYDLTGLKPGTEYKVPISGVKGGPVSRPLSAIFTT

SEQ ID NO: 402 - protein, 90 aa, synthetic construct
MLPAPKNLVVSRVTEDSARLSWQGVARAFDSFLITYREQIFAGEVIVLTVPGSERSYDLTGLKPGTEYPVWIQGVKGGSPSAPLSAEFTT

SEQ ID NO: 403 - protein, 90 aa, synthetic construct
MLPAPKNLVVSRVTEDSARLSWTAPDAAFDSFHIVYHEPRPSGEAIWLHVPGSERSYDLTGLKPGTEYEVGIVSVKGGDLSVPLVAFFTT

SEQ ID NO: 404 - protein, 90 aa, synthetic construct
MLPAPKNLVVSRVTEDSARLSWTAPDAAFDSFYIAYAEPRPDGEAISLLVPGSERSYDLTGLKPGTEYSVLIHGVKGGLLSSPLYAVFTT

SEQ ID NO: 405 - protein, 90 aa, synthetic construct
MLPAPKNLVVSRVTEDSARLSWTAPDAAFDSFYIAYAEPRPDGEAIFLVVPGSERSYDLTGLKPGTEYSVLIHGVKGGLLSSPLHANFTT

SEQ ID NO: 406 - protein, 90 aa, synthetic construct
MLPAPKNLVVSRVTEDSARLSWTAPDAAFDSFYIAYAEPRPDGEAILLDVPGSERSYDLTGLKPGTEYSVLIHGVKGGLLSSPLYASFTT

SEQ ID NO: 407 - protein, 91 aa, synthetic construct
MSLPAPKNLVVSRVTEDSARLSWTAPDAAFDSFYIAYAEPRPDGEAISLYVPGSERSYDLTGLKPGTEYSVLIHGVKGGLLSSPLSAISTT

SEQ ID NO: 408 - protein, 90 aa, synthetic construct
MLPAPKNLVVSRVTEDSARLSWTAPDAAFDSFFIGYLEPQPPGEAIQLRVPGSERSYDLTGLKPGTEYNVTIQGVKGGFPSMPLSAIFTT

SEQ ID NO: 409 - protein, 90 aa, synthetic construct
MLPAPKNLVVSRVTEDSARLSWTAPDAAFDSFHISYEEDYTFGEAIYLRVPGSERSYDLTGLKPGTEYRVVIGGVKGGWFSEPLLAAFTT

SEQ ID NO: 410 - protein, 90 aa, synthetic construct
MLPAPKNLVVSRVTEDSARLSWTAPDAAFDSFFIGYLEPQPPGEAIYLTVPGSERSYDLTGLKPGTEYNVTIQGVKGGFPSYPLDASFTT

SEQ ID NO: 411 - protein, 93 aa, synthetic construct
MLPAPKNLVVSRVTEDSARLSWTAPDAAFDSFFIGYLEPQPPGEAIDLGVPGSERSYDLTGLKPGTEYNVTIQGVKGGFPSMPLDPLEAYFTT

SEQ ID NO: 412 - protein, 90 aa, synthetic construct
MLPAPKNLVVSRVTEDSARLSWTAPDAAFDSFYIAYAEPRPDGEAILLLVPGSERSYDLTGLKPGTEYSVLIHGVKGGLLSSPLSAIFTT

SEQ ID NO: 413 - protein, 90 aa, synthetic construct
MLPAPKNLVVSRVTEDSARLSWTAPDAAFDSFYIAYAEPRPDGEAINLQVPGSERSYDLTGLKPGTEYSVLIHGVKGGLLSSPLVAFFTT

SEQ ID NO: 414 - protein, 90 aa, synthetic construct
MLPAPKNLVVSRVTEDSARLSWTTPDAAFDSFFIGYLEPQPPGEAISLQVPGSERSYDLTGLKPGTEYNVTIQGVKGGFPSSPLFAVFTT

SEQ ID NO: 415 - protein, 87 aa, synthetic construct
MLPAPKNLVVSRVTEDSARLSWTAPDAAFDSFYIAYAEPRPDGEAIELHVPGSERSYDLTGLKPGTEYSVLIHGVKGGLLSSPLFTT

SEQ ID NO: 416 - protein, 90 aa, synthetic construct
MLPAPKNLVVSRVTEDSARLSWTAPDAAFDSFYIAYAEPRPDGEAIQLVVPGSERSYDLTGLKPGTEYSVLIHGVKGGLLSSPLSAIFTT

SEQ ID NO: 417 - protein, 90 aa, synthetic construct
MLPAPKNLVVSRVTEDSARLSWTAPDAAFDSFYIAYAEPRPDGEAITLDVPGSERSYDLTGLKPGTEYSVLIHGVKGGLLSSPLSAIFTT

SEQ ID NO: 418 - protein, 90 aa, synthetic construct
MLPAPKNLVVSRVTEDSARLSWTAPDAAFDSFYIAYAEPRPDGEAIWLVVPGSERSYDLTGLKPGTEYSVLIHGVKGGLLSSPLVASFTT

SEQ ID NO: 419 - protein, 90 aa, synthetic construct
MLPAPKNLVVSRVTEDSARLSWTAPDAAFDSFYIAYAEPRPDGEAINLDVPGSERSYDLTGLKPGTEYSVLIHGVKGGLLSSPLVAEFTT

SEQ ID NO: 420 - protein, 90 aa, synthetic construct
MLPAPKNLVVSRVTEDSARLSWTAPDAAFDSFYIAYAEPRPDGEAIHLSVPGSERSYDLTGLKPGTEYSVLIHGVKGGLLSSPLVAIFTT

SEQ ID NO: 421 - protein, 90 aa, synthetic construct
MLPAPKNLVVSRVTEDSARLSWTAPDAAFDSFFIGYLEPQPPGEAIALWVPGSERSYDLTGLKPGTEYNVTIQGVKGGFPSMPLSAIFTT

SEQ ID NO: 422 - protein, 90 aa, synthetic construct
MLPAPKNLVVSRVTEDSARLSWTAPDAAFDSFYIAYAEPRPDGEAIILVVPGSERSYDLTGLKPGTEYSVLIHGVKGGLLSSPLSAHFTT

SEQ ID NO: 423 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSHPLGAVFTT

SEQ ID NO: 424 - protein, 90 aa, synthetic construct
MLPAPKNLVVSRVTEDSARLSWTAPDAAFDSFYIAYAEPRPDGEAIQLHVPGSERSYDLTGLKPGTEYSVLIHGVKGGLLSSPLLASFTT

SEQ ID NO: 425 - protein, 90 aa, synthetic construct
MLPAPKNLVVSRVTEDSARLSWTAPDAAFDSFYIAYAEPRPDGEAIALHVPGSERSYDLTGLKPGTEYSVLIHGVKGGLLSSPLVAFFTT

SEQ ID NO: 426 - protein, 90 aa, synthetic construct
MLPAPKNLVVSRVTEDSARLSWTAPDAAFDSFFIGYLEPQPPGEAIHLHVPGSERSYDLTGLKPGTEYNVTIQGVKGGFPSIPLHANFTT

SEQ ID NO: 427 - protein, 90 aa, synthetic construct
MLPAPKNLVVSRVTEDSARLSWTAPDAAFDSFYIAYAEPRPDGEAIFLGVPGSERSYDLTGLKPGTEYSVLIHGVKGGLLSSPLSAIFTT

SEQ ID NO: 428 - protein, 90 aa, synthetic construct
MLPAPKNLVVSRVTEDSARLSWTAPDAAFDSFYIAYAEPRPDGEAIVLRVPGSERSYDLTGLKPGTEYSVLIHGVKGGLLSSPLIASFTT

SEQ ID NO: 429 - protein, 90 aa, synthetic construct
MLPAPKNLVVSRVTEDSARLSWTAPDAAFDSFYIAYAEPRPDGEAINLWVPGSERSYDLTGLKPGTEYSVLIHGVKGGLLSSPLDASFTT

SEQ ID NO: 430 - protein, 90 aa, synthetic construct
MLPAPKNLVVSRVTEDSARLSWTAPDAAFDSFVIEYFEWTLNGEAIVLTVPGSERSYDLTGLKPGTEYSVQIYGVKGGCLSRPLSAIFTT

SEQ ID NO: 431 - protein, 90 aa, synthetic construct
MLPAPKNLVVSRVTEDSARLSWTAPDAAFDSFYIAYAEPRPDGEAIHLWVPGSERSYDLTGLKPGTEYSVLIHGVKGGLLSSPLIAHFTT

SEQ ID NO: 432 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGLLSSPLSAHFTT

SEQ ID NO: 433 - protein, 90 aa, synthetic construct
MLPAPKNLVVSRVTEDSARLSWTAPDAAFDSFYIAYAEPRPDGEAIYLYVPGSERSYDLTGLKPGTEYSVLIHGVKGGLLSSPLDAFFTT

SEQ ID NO: 434 - protein, 90 aa, synthetic construct
MLPAPKNLVVSRVTEDSARLSWTAPDAAFDSFYIAYAEPRPDGEAIGLQVPGSERSYDLTGLKPGTEYSVLIHGVKGGLLSSPLSAIFTT

SEQ ID NO: 435 - protein, 90 aa, synthetic construct
MLPAPKNLVVSRVTEDSARLSWTAPDAAFDSFYIAYAEPRPDGEAIQLAVPGSERSYDLTGLKPGTEYSVLIHGVKGGLLSSPLHAFFTT

SEQ ID NO: 436 - protein, 90 aa, synthetic construct
MLPAPKNLVVSRVTEDSARLSWTAPDAAFDSFFIGYLEPQPPGEAIWLHVPGSERSYDLTGLKPGTEYNVTIQGVKGGFPSIPLIAIFTT

SEQ ID NO: 437 - protein, 90 aa, synthetic construct
MLPAPKNLVVSRVTEDSARLSWTAPDAAFDSFYIAYAEPRPDGEAIVLDVPGSERSYDLTGLKPGTEYSVLIHGVKGGLLSSPLVAEFTT

SEQ ID NO: 438 - protein, 90 aa, synthetic construct
MLPTPKNLVVSRVTEDSARLSWTAPDAAFDSFYIAYAEPRPDGEAIQLRVPGSERSYDLTGLKPGTEYSVLIHGVKGGLLSSPLHASFTT

SEQ ID NO: 439 - protein, 90 aa, synthetic construct
MLPAPKNLVVSRVTEDSARLSWTAPDAAFDSFFIGYLEPQPPGEAIQLGVPGSERSYDLTGLKPGTEYNVTIQGVKGGFPSHPLNANFTT

SEQ ID NO: 440 - protein, 90 aa, synthetic construct
MLPAPKNLVVSRVTEDSARLSWTAPDAAFDSFFIGYLEPQPPGEAIQLEVPGSERSYDLTGLKPGTEYNVTIQGVKGGFPSMPLSAIFTT

SEQ ID NO: 441 - protein, 90 aa, synthetic construct
MLPAPKNLVVSRVTEDSARLSWTAPDAAFDSFYIAYAEPRPDGEAIFLGVPGSERSYDLTGLKPGTEYSVLIHGVKGGLLSSPLIAFFTT

SEQ ID NO: 442 - protein, 90 aa, synthetic construct
MLPAPKNLVVSRVTEDSARLSWTAPDAAFDSFFIGYLEPQPPGEAIGLQVPGSERSYDLTGLKPGTEYNVTIQGVKGGFPSHPLKAQFTT

SEQ ID NO: 443 - protein, 90 aa, synthetic construct
MLPAPKNLVVSRVTEDSARLSWTAPDAAFDSFYIAYAEPRPDGEAILLFVPGSERSYDLTGLKPGTEYSVLIHGVKGGLLSSPLVAHFTT

SEQ ID NO: 444 - protein, 90 aa, synthetic construct
MLPAPKNLVVSRVTEDSARLSWTAPDAAFDSFYIAYAEPRPDGEAIGLYVPGSERSYDLTGLKPGTEYSVLIHGVKGGLLSSPLGAFFTT

SEQ ID NO: 445 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLTAIFTT

SEQ ID NO: 446 - protein, 90 aa, synthetic construct
MLPAPKNLVVSRVTEDSARLSWTAPDAAFDSFYIAYAEPRPDGEAITLHVPGSERSYDLTGLKPGTEYSVLIHGVKGGLLSSPLSAIFTT

SEQ ID NO: 447 - protein, 90 aa, synthetic construct
MLPAPKNLVVSRVTEDSARLSWTAPDAAFDSFYIAYAEPRPDGEAIVLEVPGSERSYDLTGLKPGTEYSVLIHGVKGGLLSSPLDAHFTT

SEQ ID NO: 448 - protein, 90 aa, synthetic construct
MLPAPKNLVVSRVTEDSARLSWTAPDAAFDSFYIAYAEPRPDGEAIALHVPGSERSYDLTGLKPGTEYSVLIHGVKGGLLSSPLRAVFTT

SEQ ID NO: 449 - protein, 90 aa, synthetic construct
MLPAPKNLVVSRVTEDSARLSWTAPDAAFDSFYIAYAEPRPDGEAIQLWVPGSERSYDLTGLKPGTEYSVLIHGVKGGLLSSPLSAIFTT

SEQ ID NO: 450 - protein, 90 aa, synthetic construct
MLPAPKNLVVSRVTEDSARLSRTAPDAAFDSFYIAYAEPRPDGEAIVLIVPGSERSYDLTGLKPGTEYSVLIHGVKGGLLSSPLSAIFTT

SEQ ID NO: 451 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSRPLQAHFTT

SEQ ID NO: 452 - protein, 90 aa, synthetic construct
MLPAPKNLVVSRVTEDSARLSWTAPDAAFDSFYIAYAEPRPDGEAITLDVPGSERSYDLTGLKPGTEYSVLIHGVKGGLLSSPLYAFFTT

SEQ ID NO: 453 - protein, 90 aa, synthetic construct
MLPAPKNLVVSRVTEDSARLSWTAPDAAFDSFYIAYAEPRPDGEAIALHVPGSERSYDLTGLKPGTEYSVLIHGVKGGLLSSPLSAIFTT

SEQ ID NO: 454 - protein, 90 aa, synthetic construct
MLPAPKNLVVSRVTEDSARLSWTAPDAAFDSFYIAYAEPRPDGEAIGLWVPGSERSYDLTGLKPGTEYSVLIHGVKGGLLSSPLIAHFTT

SEQ ID NO: 455 - protein, 90 aa, synthetic construct
MLPAPKNLVVSRVTEDSARLSWTAPDAAFDSFYIAYAEPRPDGEAIWLVVPGSERSYDLTGLKPGTEYSVLIHGVKGGLLSSPLHARFTT

SEQ ID NO: 456 - protein, 90 aa, synthetic construct
MLPAPKNLVVSRVTEDSARLSWTAPDAAFDSFYIAYAEPRPDGEAIFLQVPGSERSYDLTGLKPGTEYSVLIHGVKGGLLSSPLAAVFTT

SEQ ID NO: 457 - protein, 90 aa, synthetic construct
MLPAPKNLVVSRVTEDSARLSWTAPDAAFDSFYIAYAEPRPDGEAILLHVPGSERSYDLTGLKPGTEYSVLIHGVKGGLLSSPLSAISTT

SEQ ID NO: 458 - protein, 90 aa, synthetic construct
MLPAPKNLVVSRVTEDSARLSWTAPDAAFDSFYIAYAEPRPDGEAIILQVPGSERSYDLTGLKPGTEYSVLIHGVKGGLLSSPLSAVFTT

SEQ ID NO: 459 - protein, 90 aa, synthetic construct
MLPAPKNLVVSRVTEDSARLSWTAPDAAFDSFYIAYAEPRPDGEAIYLKVPGSERSYDLTGLKPGTEYSVLIHGVKGGLLSSPLVAHFTT

SEQ ID NO: 460 - protein, 90 aa, synthetic construct
MLPAPKNLVVSRVTEDSARLSWTAPDAAFDSFYIAYAEPRPDGEAIVLTVPGSERSYDLTGLKPGTEYSVLIHGVKGGLLSSPLLAYFTT

SEQ ID NO: 461 - protein, 90 aa, synthetic construct
MLPAPKNLVVSRVTEDSARLSWTAPDAAFDSFYIAYAEPRPDGEAIILHVPGSERSYDLTGLKPGTEYSVLIHGVKGGLLSSPLEAKFTT

SEQ ID NO: 462 - protein, 89 aa, synthetic construct
MLPAPKNLVVSRVTEDSARLSWTAPDAAFDSFYIAYAEPRPDGEAIKLEVPGSERSYDLTGLKPGTEYSVLIHGVKGGLLSSPLAIFTT

SEQ ID NO: 463 - protein, 90 aa, synthetic construct
MLPAPKNLVVSRVTEDSARLSWTAPDAAFDSFFIGYLEPQPPGEAIYLEVPGSERSYDLTGLKPGTEYNVTIQGVKGGFPSFPLKAAFTT

SEQ ID NO: 464 - protein, 90 aa, synthetic construct
MLPAPKNLVVSRVTEDSARLSWTAPDAAFDSFYIAYAEPRPDGEAIILRVPGSERSYDLTGLKPGTEYSVLIHGVKGGLLSSPLVAIFTT

SEQ ID NO: 465 - protein, 90 aa, synthetic construct
MLPAPKNLVVSRVTEDSARLSWTAPDAAFDSFYIAYAEPRPDGEAIVLQVPGSERSYDLTGLKPGTEYSVLIHGVKGGLLSSPLAAWFTT

SEQ ID NO: 466 - protein, 90 aa, synthetic construct
MLPAPKNLVVSRVTEDSARLSWTAPDAAFDSFYIAYAEPRPDGEAIFLQVPGSERSYDLTGLKPGTEYSVLIHGVKGGLLSSPLNAFFTT

SEQ ID NO: 467 - protein, 90 aa, synthetic construct
MLPAPKNLVVSRVTEDSARLSWTAPDAAFDSFFIGYLEPQPPGEAIILGVPGSERSYDLTGLKPGTEYSVLIHGVKGGLLSSPLHAYSTT

SEQ ID NO: 468 - protein, 90 aa, synthetic construct
MLPAPKNLVVSRVTEDSARLSWTAPDAAFDSFYIAYAEPRPDGEAILLDVPGSERSYDLTGLKPGTEYSVLIHGVKGGLLSSPLSAIFTT

SEQ ID NO: 469 - protein, 90 aa, synthetic construct
MLPAPKNLVVSRVTEDSARLSWTAPDAAFDSFYIAYAEPRPDGEAILLLVPGSERSYDLTGLKPGTEYSVLIHGVKGGLLSSPLSAVFTT

SEQ ID NO: 470 - protein, 90 aa, synthetic construct
MLPAPKNLVVSRVTEDSARLSWTAPDAAFDSFYIAYAEPRPDGEAIHLLVPGSERSYDLTGLKPGTEYSVLIHGVKGGLLSSPLLAHFTT

SEQ ID NO: 471 - protein, 90 aa, synthetic construct
MLPAPKNLVVSRVTEDSARLSWTAPDAAFDSFYIAYAEPRPDGEAIQLWVPGSERSYDLTGLKPGTEYSVLIHGVKGGLLSSPLDAYFTT

SEQ ID NO: 472 - protein, 89 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLASFTT

SEQ ID NO: 473 - protein, 90 aa, synthetic construct
MLPAPKNLVVSRVTEDSARLSWTAPDAAFDSFHIVYHEPRPSGEAIHLQVPGSERSYDLTGLKPGTEYNVTIQGVKGGFPSYPLSAFFTT

SEQ ID NO: 474 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSMPLSAIFTT

SEQ ID NO: 475 - protein, 90 aa, synthetic construct
MLPAPKNLVVSRVTEDSARLSWTAPDAAFDSFRISYCETFYHGEAIVLTVPGSERSYDLTGLKPGTEYSVLIHGVKGGLLSSPLIAKFTT

SEQ ID NO: 476 - protein, 90 aa, synthetic construct
MLPAPKNLVVSRVTEDSARLSWTAPDAAFDSFYIAYAEPRPDGEAIWLKVPGSERSYDLTGLKPGTEYSVLIHGVKGGLLSSPLQANFTT

SEQ ID NO: 477 - protein, 90 aa, synthetic construct; identical to SEQ ID NO: 476

SEQ ID NO: 478 - protein, 90 aa, synthetic construct
MLPAPKNLVVSRVTEDSARLSWTAPDAAFDSFYIAYAEPRPDGEAIQLQVPGSERSYDLTGLKPGTEYSVLIHGVKGGLLSSPLSAIVTT

SEQ ID NO: 479 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAFFTT

SEQ ID NO: 480 - protein, 90 aa, synthetic construct
MLPAPKNLVVSRVTEDSARLSWTAPDAAFDSFYIAYAEPRPDGEAIALLVPGSERSYDLTGLKPGTEYSVLIHGVKGGLLSSPLVAQFTT

SEQ ID NO: 481 - protein, 90 aa, synthetic construct; identical to SEQ ID NO: 461

SEQ ID NO: 482 - protein, 90 aa, synthetic construct
MLPAPKNLVVSRVTEDSARLSWTAPDAAFDSFYIAYAEPRPDGEAIDLHVPGSERSYDLTGLKPGTEYSVLIHGVKGGLLSSPLHALFTT

SEQ ID NO: 483 - protein, 90 aa, synthetic construct
MLPAPKNLVVSRVTEDSARLSWTAPDAAFDSFYIAYAEPRPDGEAILLDVPGSERSYDLTGLKPGTEYSVLIHGVKGGFPSMPLSAIFTT

SEQ ID NO: 484 - protein, 88 aa, synthetic construct
MLPAPKNLVVSRVTEDSARLSWTAPDAAFDSFFIGYLEPQPPGEAIDLAVPGSERSYDLTGLKPGTEYSVLIHGVKGGLLSSPLSFTT

SEQ ID NO: 485 - protein, 90 aa, synthetic construct
MLPAPKNLVVSRVTEDSARLSWTAPDAAFDSFYIAYAEPRPDGEAIYLGVPGSERSYDLTGLKPGTEYSVLIHGVKGGLLSSPLRAKFTT

SEQ ID NO: 486 - protein, 90 aa, synthetic construct
MLPAPKNLVVSRVTEDSARLSWTAPDAAFDSFYIAYAEPRPDGEAIQLGVPGSERSYDLTGLKPGTEYSVLIHGVKGGLLSSPLSAIFTT

SEQ ID NO: 487 - protein, 88 aa, synthetic construct
MLPAPKNLVVSRVTEDSARLSWTAPDAAFDSFFIGYLEPQPPGEAISLLVPDSERSYDLTGLKPGTEYNVTIQGVKGGFPSMPLKFTT

SEQ ID NO: 488 - protein, 90 aa, synthetic construct
MLPAPKNLVVSRVTEDSARLSWTAPDAAFDSFYIAYAEPRPDGEAIGLGVPGSERSYDLTGLKPGTEYSVLIHGVKGGLLSSPLDASFTT

SEQ ID NO: 489 - protein, 90 aa, synthetic construct
MLPAPKNLVVSRVTEDSARLSWTAPDAAFDSFYIAYAEPRPDGEAIQLTVPGSERSYDLTGPKPGTEYWVLIQGVKGGGSSVPLVAYFTT

SEQ ID NO: 490 - protein, 90 aa, synthetic construct
MLPAPKNLVVSRVTEDSARLSWTAPDAAFDSFYIAYAEPRPDGEAISLDVPGSERSYDLTGLKPGTEYSVLIHGVKGGLLSSPLEASFTT

SEQ ID NO: 491 - protein, 90 aa, synthetic construct
MLPAPKNLVVSRVTEDSARLSWTAPDAAFDSFFIGYLEPQPPGEAIILAVPGSERSYDLTGLKPGTEYNVTIQGVKGGFPSLPLVASFTT

SEQ ID NO: 492 - protein, 90 aa, synthetic construct
MLPAPKNLVVSRVTEDSARLSWTAPDAAFDSFDIGYTEYGGYGEAIYLSVPGSERSYDLTGLKPGTEYWVLIQGVKGGGSSVPLSAIFTT

SEQ ID NO: 493 - protein, 90 aa, synthetic construct
MLPAPKNLVVSRVTEDSARLSWTAPDAAFDSFYIAYAEPRPDGEAISLSVPGSERSYDLTGLKPGTEYSVLIHGVKGGLLSSPLIANFTT

SEQ ID NO: 494 - protein, 90 aa, synthetic construct
MLPAPKNLVVSRVTEDSARLSWTAPDAAFDSFYIAYAEPRPDGEAIALLVPGSERSYDLTGLKPGTEYSVLIHGVKGGLLSSPLSAIVTT

SEQ ID NO: 495 - protein, 90 aa, synthetic construct
MLPAPKNLVVSRVTEDSARLSWTAPDAAFDSFYIAYAEPRPDGEAIILDVPGSERSYDLTGLKPGTEYSVLIHGVKGGLLSSPLSSIFTT

SEQ ID NO: 496 - protein, 90 aa, synthetic construct
MLPAPKNLVVSRVTEDSARLSWTAPDAAFDSFYIAYAEPRPDGEAIVLWVPGSERSYDLTGLKPGTEYSVLIHGVKGGLLSSPLRASFTT

SEQ ID NO: 497 - protein, 90 aa, synthetic construct
MLPAPKNLVVSRVTEDSARLSWTAPDAAFDSFYIAYAEPRPDGEAIKLDVPGSERSYDLTGLKPGTEYSVLIHGVKGGLLSSPLVAFFTT

SEQ ID NO: 498 - protein, 90 aa, synthetic construct
MLPAPKNLVVSRVTEDSARLSWTAPDAAFDSFYIAYAEPRPDGEAIILEVPGSERSYDLTGLKPGTEYSVLIHGVKGGLLSSPLVAYFTT

SEQ ID NO: 499 - protein, 90 aa, synthetic construct
MLPAPKNLVVSRVTEDSARLSWTAPDAAFDSFYIAYAEPRPDGEAIHLWVPGSERSYDLTGLKPGTEYSVLIHGVKGGLLSSPLHADFTT

SEQ ID NO: 500 - protein, 90 aa, synthetic construct
MLPAPKNLVVSRVTEDSARLSWTAPDAAFDSFYIAYAEPRPDGEAIWLEVPGSERSYDLTGLKPGTEYSVLIHGVKGGLLSSPLVADFTT

SEQ ID NO: 501 - protein, 90 aa, synthetic construct
MLPAPKNLVVSRVTEDSARLSWTAPDAAFDSFYIAYAEPRPDGEAISLWVPGSERSYDLTGLKPGTEYSVLIHGVKGGLLSSPLLAHFTT

SEQ ID NO: 502 - protein, 89 aa, synthetic construct
MLPAPKNLVVSRVTEDSARLSWTAPDAAFDSFDIGYTEYGGYGEAILHVPGSERSYDLTGLKPGTEYWVLIQGVKGGGSSVPLSAIFTT

SEQ ID NO: 503 - protein, 90 aa, synthetic construct
MLPAPKNLVVSRVTEDSARLSWTAPDAAFDSFFIGYLEPQPPGEAIVLLVPGSERSYDLTGLKPGTEYNVTIQGVKGGFPSVPLAAFFTT

SEQ ID NO: 504 - protein, 85 aa, synthetic construct
MLPAPKNLVVSRVTEDSARLSWTAPDAAFDSFFIGYLEPQPPGEAILLWVPGSERSYDLTGLKPGTEYNVTIQGVKGGFPSQFTT

SEQ ID NO: 505 - protein, 93 aa, synthetic construct
MLPAPKNLVVSRVTEDSARLSWTAPDAAFDSFFIGYLEPQPPGEAILLGVPGSERSYDLTGLKPGTEYNVTIQGVKGGFPSMPLHPLVALFTT

SEQ ID NO: 506 - protein, 90 aa, synthetic construct
MLPAPKNLVVSRVTEDSARLSWTAPDAAFDSFYIAYAEPRPDGEAIGLDVPGSERSYDLTGLKPGTEYSVLIHGVKGGLLSSPLSAIFTT

SEQ ID NO: 507 - protein, 90 aa, synthetic construct
MLPAPKNLVVSRVTEDSARLSWTAPDAAFDSFYIAYAEPRPDGEAIHLSVPGSERSYDLTGLKPGTEYSVLIHGVKGGLLSSPLAAYFTT

SEQ ID NO: 508 - protein, 90 aa, synthetic construct
MLPAPKNLVVSRVTEDSARLSWTAPDAAFDSFFIGYLEPQPPGEAIVLAVPGSERSYDLTGLKPGTEYNVTIQGVKGGFPSYPLVAAFTT

SEQ ID NO: 509 - protein, 90 aa, synthetic construct
MLPAPKNLVVSRVTEDSARLSWTAPDAAFDSFYIAYAEPRPDGEAILLQVPGSCRSYDLTGLKPGTEYSVLIHGVKGGLLSSPLTAIFTT

SEQ ID NO: 510 - protein, 90 aa, synthetic construct
MLPAPKNLVVSRVTEDSARLSWTAPDAAFDSFFIGYLEPQPPGEAINLQVPGSERSYDLTGLKPGTEYNVTIQGVKGGFPSFPLSAVFTT

SEQ ID NO: 511 - protein, 90 aa, synthetic construct
MLPAPKNLVVSRVTEDSARLSWTAPDAAFDSFYIAYAEPRPDGEAIQLHVPGSERSYDLTGLKPGTEYSVLIHGVKGGLLSSPLVAIFTT

SEQ ID NO: 512 - protein, 90 aa, synthetic construct
MLPAPKNLVVSRVTEDSARLSWTAPDAAFDSFYIAYAEPRPDGEAIWLAVPGSERSYDLTGLKPGTEYSVLIHGVKGGLLSSPLHAQFTT

SEQ ID NO: 513 - protein, 87 aa, synthetic construct
MLPAPKNLVVSRVTEDSARLSWTAPDAAFDSFYIAYAEPRPDGEAILLGVPGSERSYDLTGLKPGTEYSVLIHGVKGGLLSSPLFTT

SEQ ID NO: 514 - protein, 90 aa, synthetic construct
MLPAPKNLVVSRVTEDSARLSWTAPDAAFDSFYIAYAEPRPDGEAIGLQVPGSERSYDLTGLKPGTEYSVLIHGVKGGLLSSPLCAEFTT

SEQ ID NO: 515 - protein, 90 aa, synthetic construct
MLPAPKNLVVSRVTEDSARLSWTAPDAAFDSFYIAYAEPRPDGEAIVLWVPGSERSYDLTGLKPGTEYSVLIHGVKGGLLSSPLIAEFTT

SEQ ID NO: 516 - protein, 88 aa, synthetic construct
MLPAPKNLVVSRVTEDSARLSWTAPDAAFDSFYIAYAEPRPDGEAISLSVPGSERSYDLTGLKPGTEYSVLIHGVKGGLLSSPPKFTT

SEQ ID NO: 517 - protein, 90 aa, synthetic construct
MLPAPKNLVVSRVTEDSARLSWTAPDAAFDSFYIAYAEPRPDGEAIILEVPGSERSYDLTGLKPGTEYSVLIHGVKGGLLSSPLRAVFTT

SEQ ID NO: 518 - protein, 90 aa, synthetic construct
MLPAPKNLVVSRVTEDSARLSWTAPDAAFDSFYIAYAEPRPDGEAIHLVVPGSERSYDLTGLKPGTEYSVLIHGVKGGLLSSPLSAIFTT

SEQ ID NO: 519 - protein, 90 aa, synthetic construct
MLPAPKNLVVSRVTEDSARLSWTAPDAAFDSFYIAYAEPRPDGEAISLKVPGSERSYDLTGLKPGTEYSVLIHGVKGGLLSSPLEAIFTT

SEQ ID NO: 520 - protein, 114 aa, synthetic construct
MLPAPKNPVVSRVTEDSARLSWTAPDAAFDSFYIAYAEPRPDGEAIHLLVPGSERSYDLTGLKPGTEYSVLIHGVKGGLLSSPLKRLSPPVVTITITMAVCRKPVAENLSQTLS

SEQ ID NO: 521 - protein, 89 aa, synthetic construct
MLPAPKNLVVSRVTEDSARLSWTAPDAAFDSFFIGYLEPQPPGEAIFLDVPGSERSYDLTGLKPGTEYNVTIQGVKGGFPSPLTAFFTT

SEQ ID NO: 522 - protein, 90 aa, synthetic construct
MLPAPKNLVVSRVTEDSARLSWTAPDAAFDSFFIGYLEPQPPGEAIVLDVPGSERSYDLTGLKPGTEYNVTIQGVKGGFPSHPLAAAFTT

SEQ ID NO: 523 - protein, 90 aa, synthetic construct
MLPAPKNLVVSRVTEDSARLSWTAPDAAFDSFFIGYLEPQPPGEAIGLAVPGSERSYDLTGLKPGTEYNVTIQGVKGGFPSVPLQANFTT

SEQ ID NO: 524 - protein, 90 aa, synthetic construct
MLPAPKNLVVSRVTEDSARLSWTAPDAAFDSFYIAYAEPRPDGEAILLRVPGSERSYDLTGLKPGTEYSVLIHGVKGGLLSSPLVAEFTT

SEQ ID NO: 525 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLSASFTT

SEQ ID NO: 526 - protein, 90 aa, synthetic construct
MLPAPKNLVVSRVTEDSARLSWTAPDAAFDSFYIAYAEPRPDGEAIGLHVPGSERSYDLTGLKPGTEYSVLIHGVKGGLLSSPLTASFTT

SEQ ID NO: 527 - protein, 90 aa, synthetic construct
MLPAPKNLVVSRVTEDSARLSWTAPDAAFDSFYIAYAEPRPDGEAIGLRVPGSERSYDLTGLKPGTEYSVLIHGVKGGLLSSPLSAIFTT

SEQ ID NO: 528 - protein, 90 aa, synthetic construct
MLPAPKNLVVSRVTEDSARLSWTAPDAAFDSFYIAYAEPRPDGEAIVLRVPGSERSYDLTGLKPGTEYSVLIHGVKGGLLSSPLAASFTT

SEQ ID NO: 529 - protein, 90 aa, synthetic construct
MLPAPKNLVVSRVTEDSARLSWTAPDAAFDSFYIAYAEPRPDGEAIQLLVPGSERSYDLTGLKPGTEYSVLIHGVKGGLLSSPLVAHFTT

SEQ ID NO: 530 - protein, 90 aa, synthetic construct
MLPAPKNLVVSRVTEDSARLSWTAPDAAFDSFYIAYAEPRPDGEAIWLLVPGSERSYDLTGLKPGTEYSVLIHGVKGGLLSSPLVAFFTT

SEQ ID NO: 531 - protein, 90 aa, synthetic construct
MLPAPKNLVVSRVTEDSARLSWTAPDAAFDSFFIGYLEPQPPGEAIHLYVPGSERSYDLTGLKPGTEYNVTIQGVKGGFPSDPLDAVFTT

SEQ ID NO: 532 - protein, 85 aa, synthetic construct
MLPAPKNLVVSRVTEDSARLSWTAPDAAFDSFFIGYLEPQPPGEAIYLDVPGSERSYDLTGLKPGTEYNVTIQGVKGGFPSTFTT

SEQ ID NO: 533 - protein, 90 aa, synthetic construct
MLPAPKNLVVSRVTEDSARLSWTAPDAAFDSFYIAYAEPRPDGEAIQLFVPGSERSYDLTGLKPGTEYSVLIHGVKGGLLSSPLKAYFTT

SEQ ID NO: 534 - protein, 90 aa, synthetic construct
MLPAPKNLVVSRVTEDSARLSWTAPDAAFDSFYIAYAEPRPDGEAIVLVVPGSERSYDLTGLKPGTEYSVLIHGVKGGLLSSPLSAIFTT

SEQ ID NO: 535 - protein, 90 aa, synthetic construct
MLPAPKNLVVSRVTEDSARLSWTAPDAAFDSFFIGYLEPQPPGEAIQLTVPGSERSYDLTGLKPGTEYNVTIQGVKGGFPSLPLSADFTT

SEQ ID NO: 536 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLDAEFTT

SEQ ID NO: 537 - protein, 90 aa, synthetic construct
MLPAPKNLVVSRVTEDSARLSWTAPDAAFDSFYIAYAEPRPDGEAIVLAVPGSERSYDLTGLKPGTEYSVLIHGVKGGLLSSPLYASFTT

SEQ ID NO: 538 - protein, 88 aa, synthetic construct
MLPAPKNLVVSRVTEDSARLSWTTPDAAFDSFYIAYAEPRPDGEAIRLQVPGSERSYDLTGLKPGTEYSVLIHGVKGGLLSSPLGFTT

SEQ ID NO: 539 - protein, 90 aa, synthetic construct
MLPAPKNLVVSRVTEDSARLSWTAPDAAFDSFYIAYAEPRPDGEAIQLVVPGSERSYDLTGLKPGTEYSVLIHGVKGGLLSSPLYAIFTT

SEQ ID NO: 540 - protein, 90 aa, synthetic construct
MLPAPKNLVVSRVTEDSARLSWTAPDAAFDSFYIAYAEPRPDGEAISLSVPGSERSYDLTGLKPGTEYSVLIHGVKGGLLSSPLHAKFTT

SEQ ID NO: 541 - protein, 90 aa, synthetic construct
MLPAPKNLVVSRVTEDSARLSWTAPDAAFDSFFIGYLEPQPPGEAIHLGVPGSERSYDLTGLKPGTEYNVTIQGVKGGFPSIPLFASFTT

SEQ ID NO: 542 - protein, 90 aa, synthetic construct
MLPAPKNLVVSRVTEDSARLSWTAPDAAFDSFYIAYAEPRPDGEAIQLLVPGSERSYDLTGLKPGTEYSVLIHGVKGGLLSSPLYAAFTT

SEQ ID NO: 543 - protein, 90 aa, synthetic construct
MLPAPKNLVVSRVTEDSARLSWTAPDAAFDSFFIGYLEPQPPGEAIHLAVPGSERSYDLTGLKPGTEYNVTIQGVKGGFPSVPLAAVFTT

SEQ ID NO: 544 - protein, 90 aa, synthetic construct
MLPAPKNLVVSRVTEDSARLSWTAPDAAFDSFYIAYAEPRPDGEAISLQVPGSERSYDLTGLKPGTEYSVLIHGVKGGLLSSPLGAHFTT

SEQ ID NO: 545 - protein, 90 aa, synthetic construct
MLPAPKNLVVSRVTEDSARLSWTAPDAAFDSFYIAYAEPRPDGEAIALWVPGSERSYDLTGLKPGTEYSVLIHGVKGGLLSSPLVASFTT

SEQ ID NO: 546 - protein, 90 aa, synthetic construct
MLPAPKNLVVSRVTEDSARLSWTAPDAAFDSFYIAYAEPRPDGEAIQLHVPGSERSYDLTGLKPGTEYSVLIHGVKGGLLSSPLYAFFTT

SEQ ID NO: 547 - protein, 90 aa, synthetic construct
MLPAPKNLVVSRVTEDSARLSWTAPDAAFDSFYIAYAEPRPDGEAILLHVPGSERSYDLTGLKPGTEYSVLIHGVKGGLLSSPLRASFTT

SEQ ID NO: 548 - protein, 90 aa, synthetic construct
MLPAPKNLVVSRVTEDSARLSWTAPDAAFDSFYIAYAEPRPDGEAIWLGVPGSERSYDLTGLKPGTEYSVLIHGVKGGLLSSPLHATFTT

SEQ ID NO: 549 - protein, 90 aa, synthetic construct
MLPAPKNLVVSRVTEDSARLSWTAPDAAFDSFYIAYAEPRPDGEAIVLEVPGSERSYDLTGLKPGTEYSVLIHGVKGGLLSSPLHANFTT

SEQ ID NO: 550 - protein, 90 aa, synthetic construct
MLPAPKNLVVSRVTEDSARLSWTAPDAAFDSFYIAYAEPRPDGEAILLRVPGSERSYDLTGLKPGTEYSVLIHGVKGGLLSSPLYAKFTT

SEQ ID NO: 551 - protein, 90 aa, synthetic construct
MLPAPKNLVVSRVTEDSARLSWTAPDAAFDSFFIGYLEPQPPGEAIGLWVPGSERSYDLTGLKPGTEYNVTIQGVKGGFPSDPLQAVFTT

SEQ ID NO: 552 - protein, 90 aa, synthetic construct
MLPAPKNLVVSRVTEDSARLSWTAPDAAFDSFYIAYAEPRPDGEAIVLHVPGSERSYDLTGLKPGTEYSVLIHGVKGGLLSSPLDAFFTT

SEQ ID NO: 553 - protein, 90 aa, synthetic construct
MLPAPKNLVVSRVTEDSARLSWTAPDAAFDSFYIAYAEPRPDGEAIILHVPGSERSYDLTGLKPGTEYSVLIHGVKGGLLSSPLDAYFTT

SEQ ID NO: 554 - protein, 90 aa, synthetic construct
MLPAPKNLVVSRVTEDSARLSWTAPDAAFDSFYIAYAEPRPDGEAILLAVPGSERSYDLTGLKPGTEYSVLIHGVKGGLLSSPLSAKFTT

SEQ ID NO: 555 - protein, 90 aa, synthetic construct
MLPAPKNLVVSRVTEDSARLSWTAPDAAFDSFFIGYLEPQPPGEAILLFVPGSERSYDLTGLKPGTEYNVTIQGVKGGFPSTPLSASFTT

SEQ ID NO: 556 - protein, 90 aa, synthetic construct
MLPAPKNLVVSRVTEDSARLSWTAPDAAFDSFYIAYAEPRPDGEAIQLTVPGSERSYDLTGLKPGTEYSVLIHGVKGGLLSSPLHAYFTT

SEQ ID NO: 557 - protein, 90 aa, synthetic construct
MLPAPKNLVVSRVTEDSARLSWTAPDAAFDSFYIAYAEPRPDGEAIQLGVPGSERSYDLTGLKPGTEYSVLIHGVKGGLLSSPLRAYFTT

SEQ ID NO: 558 - protein, 90 aa, synthetic construct
MLPAPKNLVVSRVTEDSARLSWTAPDAAFDSFYIAYAEPRPDGEAILLEVPGSERSYDLTGLKPGTEYSVLIHGVKGGLLSSPLVAFFTT

SEQ ID NO: 559 - protein, 90 aa, synthetic construct
MLPAPKNLVVSRVTEDSARLSWTAPDAAFDSFYIAYAEPRPDGEAIQLGVPGSERSYDLTGLKPGTEYSVLIHGVKGGLLSSPLLAVFTT

SEQ ID NO: 560 - protein, 90 aa, synthetic construct
MLPAPKNLVVSRVTEDSARLSWTAPDAAFDSFYIAYAEPRPDGEAIHLRVPGSERSYDLTGLKPGTEYNVTIQGVKGGFPSMPLSAIFTT

SEQ ID NO: 561 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLIAKFTT

SEQ ID NO: 562 - protein, 90 aa, synthetic construct
MLPAPKNLVVSRVTEDSARLSWTAPDAAFDSFYIAYAEPRPDGEAIVLHVPGSERSYDLTGLKPGTEYSVLIHGVKGGLLSSPLQAIFTT

SEQ ID NO: 563 - protein, 90 aa, synthetic construct
MLPAPKNLVVSRVTEDSARLSWTAPDAAFDSFYIAYAEPRPDGEAIALVVPGSERSYDLTGLKPGTEYSVLIHGVKGGLLSSPLAANFTT

SEQ ID NO: 564 - protein, 90 aa, synthetic construct
MLPAPKNLVVSRVTEDSARLSWTAPDAAFDSFYIAYAEPRPDGEAINLSVPGSERSYDLTGLKPGTEYSVLIHGVKGGLLSSPLDAYFTT

SEQ ID NO: 565 - protein, 90 aa, synthetic construct
MLPAPKNLVVSRVTEDSARLSWTAPDAAFDSFYIAYAEPRPDGEAIVLEVPGSERSYDLTGLKPGTEYSVLIHGVKGGLLSSPLTASFTT

SEQ ID NO: 566 - protein, 90 aa, synthetic construct
MLPAPKNLVVSRVTEDSARLSWTAPDAAFDSFYIAYAEPRPDGEAIRLQVPGSERSYDLTGLKPGTEYSVLIHGVKGGLLSSPLGASFTT

SEQ ID NO: 567 - protein, 90 aa, synthetic construct
MLPAPKNLVVSRVTEDSARLSWTAPDAAFDSFYIAYAEPRPDGEAIGLWVPGSERSYDLTGLKPGTEYSVLIHGVKGGLLSSPLVAYFTT

SEQ ID NO: 568 - protein, 87 aa, synthetic construct
MLPAPKNLVVSRVTEDSARLSWTAPDAAFDSFYIAYAEPRPDGEAIYLEVPGSERSYDLTGLKPGTEYSVLIHGVKGGLLSSPLFTT

SEQ ID NO: 569 - protein, 90 aa, synthetic construct
MLPAPKNLVVSRVTEDSARLSWTAPDAAFDSFYIAYAEPRPDGEAIWLDVPGSERSYDLTGLKPGTEYSVLIHGVKGGLLSSPLDAYFTT

SEQ ID NO: 570 - protein, 90 aa, synthetic construct
MLPAPKNLVVSRVTEDSARLSWTAPDAAFDSFAIEYCETKMCGEAIVLTVPGSERSYDLTGLKPGTEYRVPIPGVKGGTASLPLSAIFTT

SEQ ID NO: 571 - protein, 90 aa, synthetic construct
MLPAPKNLVVSRVTEDSARLSWTAPDAAFDSFFIYYIESYPAGEAIVLTVPGSERSYDLTGLKPGTEYWVGIDGVKGGRWSTPLSAIFTT

SEQ ID NO: 572 - protein, 90 aa, synthetic construct
MLPAPKNLVVSRVTEDSARLSWTAPDAAFDSFFIYYIESYPAGEAIVLTVPGSCRSYDLTGLKPGTEYWVGIDGVKGGRWSTPLSAIFTT

SEQ ID NO: 573-591 - skipped (19 entries)

SEQ ID NO: 592 - protein, 189 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTAPAPAPAPAPLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 593 - protein, 199 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTGGGGSGGGGSGGGGSGGGGSLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 594 - protein, 199 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTEAAAKEAAAKEAAAKEAAAKLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 595 - protein, 184 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTEAAAKLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 596 - protein, 189 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTAPAPAPAPAPLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 597 - protein, 199 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTGGGGSGGGGSGGGGSGGGGSLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 598 - protein, 199 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTEAAAKEAAAKEAAAKEAAAKLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 599 - protein, 184 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTEAAAKLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 600 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
ctgggaggatgaattcggat

SEQ ID NO: 601 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttcgaattcatcctcccagtt

SEQ ID NO: 602 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
ctgggaggatgaattaggat

SEQ ID NO: 603 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttcgaattcatcctcccagtt

SEQ ID NO: 604 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
ctgggaggatgaatgaggat

SEQ ID NO: 605 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttcgaattcatcctcccagtt

SEQ ID NO: 606 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
ctgggaggatgaattcgaat

SEQ ID NO: 607 - RNA, 21 nt, synthetic construct
  source 1..21: mol_type = other RNA
  modified_base 1..2: mod_base = OTHER; note = 2'Fluoro ribonucleotide phosphorothioate
  modified_base 3..18: mod_base = OTHER; note = 2'Fluoro ribonucleotide
  modified_base 19..20: mod_base = OTHER; note = 2'Fluoro ribonucleotide phosphorothioate
  modified_base 21: mod_base = OTHER; note = 2'Fluoro ribonucleotide
ttcgaattcatcctcccagtt

SEQ ID NO: 608 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
ctgggaggatgaattcgaat

SEQ ID NO: 609 - RNA, 21 nt, synthetic construct
  source 1..21: mol_type = other RNA
  modified_base 1: mod_base = OTHER; note = 2'O-Methyl ribonucleotide phosphorothioate
  modified_base 2..8: mod_base = OTHER; note = 2'Fluoro ribonucleotide phosphorothioate
  modified_base 9: mod_base = OTHER; note = 2'O-Methyl ribonucleotide
  modified_base 10: mod_base = OTHER; note = 2'Fluoro ribonucleotide
  modified_base 11: mod_base = OTHER; note = 2'O-Methyl ribonucleotide
  modified_base 12..18: mod_base = OTHER; note = 2'Fluoro ribonucleotide
  modified_base 19..20: mod_base = OTHER; note = 2'Fluoro ribonucleotide phosphorothioate
  modified_base 21: mod_base = OTHER; note = 2'O-Methyl ribonucleotide
ttcgaattcatcctcccagtt

SEQ ID NO: 610 - DNA, 15 bp, synthetic construct
  source 1..15: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..13: mod_base = OTHER; note = 2'O-Methyl ribonucleotide
  modified_base 14: mod_base = OTHER; note = 2'Fluoro ribonucleotide
  modified_base 15: mod_base = OTHER; note = Inverted deoxythymidine
  misc_feature 1..14: note = RNA
  misc_feature 15: note = DNA
ctgggaggatgaatt

SEQ ID NO: 611 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttcgaattcatcctcccagtt

SEQ ID NO: 612 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
caaggtgggtggcatctaat

SEQ ID NO: 613 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 614 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  misc_feature 1..19: note = RNA
  misc_feature 20: note = DNA
caaggtgggtggcatctagt

SEQ ID NO: 615 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 616 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatctgat

SEQ ID NO: 617 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 618 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatcgaat

SEQ ID NO: 619 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 620 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatataat

SEQ ID NO: 621 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 622 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcagctaat

SEQ ID NO: 623 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 624 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcagctagt

SEQ ID NO: 625 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 626 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaacgcat

SEQ ID NO: 627 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 628 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaacgcgt

SEQ ID NO: 629 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 630 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
gagccatctttgcaacgaat

SEQ ID NO: 631 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
tgcgttgcaaagatggctctt

SEQ ID NO: 632 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaacacat

SEQ ID NO: 633 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 634 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaaagcat

SEQ ID NO: 635 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 636 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcagcgcat

SEQ ID NO: 637 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 638 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcagcgcgt

SEQ ID NO: 639 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 640 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattctat

SEQ ID NO: 641 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 642 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattctgt

SEQ ID NO: 643 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 644 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattcgat

SEQ ID NO: 645 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 646 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattgtat

SEQ ID NO: 647 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 648 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacatgctat

SEQ ID NO: 649 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 650 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacatgctat

SEQ ID NO: 651 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 652 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacagtctat

SEQ ID NO: 653 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 654 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacagtctgt

SEQ ID NO: 655 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 656 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatctagt

SEQ ID NO: 657 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3: mod_base = OTHER; note = 2'O-Methyl ribonucleotide
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 658 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatctaat

SEQ ID NO: 659 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 660 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatctagt

SEQ ID NO: 661 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 662 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatctgat

SEQ ID NO: 663 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 664 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatcgaat

SEQ ID NO: 665 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 666 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatataat

SEQ ID NO: 667 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 668 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcagctaat

SEQ ID NO: 669 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 670 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcagctagt

SEQ ID NO: 671 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 672 - RNA, 22 nt, synthetic construct
  source 1..22: mol_type = other RNA
  modified_base 1: mod_base = OTHER; note = 2'O methyl vinyl phosphonate uridine phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 673 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttgcgttgcaaagatggctctt

SEQ ID NO: 674 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaacgcgt

SEQ ID NO: 675 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttgcgttgcaaagatggctctt

SEQ ID NO: 676 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaacgaat

SEQ ID NO: 677 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttgcgttgcaaagatggctctt

SEQ ID NO: 678 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaacacat

SEQ ID NO: 679 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttgcgttgcaaagatggctctt

SEQ ID NO: 680 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcaaagcat

SEQ ID NO: 681 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttgcgttgcaaagatggctctt

SEQ ID NO: 682 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcagcgcat

SEQ ID NO: 683 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttgcgttgcaaagatggctctt

SEQ ID NO: 684 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
gagccatctttgcagcgcgt

SEQ ID NO: 685 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttgcgttgcaaagatggctctt

SEQ ID NO: 686 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattctat

SEQ ID NO: 687 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 688 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattctgt

SEQ ID NO: 689 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 690 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattcgat

SEQ ID NO: 691 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 692 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacattgtat

SEQ ID NO: 693 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 694 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacatgctat

SEQ ID NO: 695 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 696 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacatgctat

SEQ ID NO: 697 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 698 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacagtctat

SEQ ID NO: 699 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 700 - DNA, 20 bp, synthetic construct
  source 1..20: mol_type = other DNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
ttacggtatctacagtctgt

SEQ ID NO: 701 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 702 - DNA, 20 bp, synthetic construct
  source 1..20: mol_type = other DNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
  modified_base 20: mod_base = OTHER; note = Inverted deoxythymidine
  misc_feature 1..19: note = RNA
  misc_feature 20: note = DNA
caaggtgggtggcatctagt

SEQ ID NO: 703 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7..9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12..14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 704 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
ttacggtatctacattcta

SEQ ID NO: 705 - RNA, 21 nt, synthetic construct
  source 1..21: mol_type = other RNA
  modified_base 1: mod_base = OTHER; note = 2'O-Methyl ribonucleotide phosphorothioate
  modified_base 2: mod_base = OTHER; note = 2'Fluoro ribonucleotide phosphorothioate
  modified_base 3..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 706 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
ttacggtatctacattcta

SEQ ID NO: 707 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagaatgtagataccgtaatt

SEQ ID NO: 708 - protein, 87 aa, synthetic construct
LPAPKNLVVSRVTEDSARLSWTAPDAADSAIVYHEPRPDGEAIVLTVPGSCRSYDLTGLKPGTEYEVVILGVKGGVHSYPLSAIFTT

SEQ ID NO: 709 - protein, 87 aa, synthetic construct
LPAPKNLVVSRVTEDSARLSWTAPDAADSAIVYHEPRPDGEAIVLTVPGSERSYDLTGLKPGTEYEVVILGVKGGVHSYPLSAIFTT

SEQ ID NO: 710 - protein, 86 aa, synthetic construct
LPAPKNLVVSRVTEDSARLSWTAPDAADSIGYLEPQPPGEAIHLGVPGSCRSYDLTGLKPGTEYNVTIQGVKGGFPSIPLFASFTT

SEQ ID NO: 711 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
tggacttcaacctagacaa

SEQ ID NO: 712 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
tttgtctaggttgaagtccatt

SEQ ID NO: 713 - protein, 89 aa, synthetic construct
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 714-719 - skipped (6 entries)

SEQ ID NO: 720 - protein, 4 aa, synthetic construct
GSGS

SEQ ID NO: 721 - protein, 8 aa, synthetic construct
GGGSGGGS

SEQ ID NO: 722 - protein, 25 aa, synthetic construct
  VARIANT 6..25: note = GGGGS repeats may be deleted
GGGGSGGGGSGGGGSGGGGSGGGGS

SEQ ID NO: 723 - protein, 4 aa, synthetic construct
APAP

SEQ ID NO: 724 - protein, 10 aa, synthetic construct
APAPAPAPAP

SEQ ID NO: 725 - protein, 20 aa, synthetic construct
APAPAPAPAPAPAPAPAPAP

SEQ ID NO: 726 - protein, 40 aa, synthetic construct
APAPAPAPAPAPAPAPAPAPAPAPAPAPAPAPAPAPAPAP

SEQ ID NO: 727 - protein, 29 aa, synthetic construct
AEAAAKEAAAKEAAAKEAAAKEAAAKAAA

SEQ ID NO: 728 - protein, 25 aa, synthetic construct
  VARIANT 6..25: note = EAAAK repeats may be deleted
EAAAKEAAAKEAAAKEAAAKEAAAK

SEQ ID NO: 729 - protein, 20 aa, synthetic construct
EAAAKEAAAKEAAAKEAAAK

SEQ ID NO: 730 - protein, 20 aa, synthetic construct
GGGGSGGGGSGGGGSGGGGS

SEQ ID NO: 731 - protein, 10 aa, synthetic construct
APAPAPAPAP

SEQ ID NO: 732 - protein, 5 aa, synthetic construct
EAAAK

SEQ ID NO: 733-782 - skipped (50 entries)

SEQ ID NO: 783 - RNA, 19 nt, synthetic construct
  source 1..19: mol_type = other RNA
ctgggaggatgaattcgaa

SEQ ID NO: 784 - RNA, 21 nt, synthetic construct
  source 1..21: mol_type = other RNA
ttcgaattcatcctcccagtt

SEQ ID NO: 785 - RNA, 19 nt, synthetic construct
  source 1..19: mol_type = other RNA
gggtgacaactactatcta

SEQ ID NO: 786 - RNA, 21 nt, synthetic construct
  source 1..21: mol_type = other RNA
tagatagtagttgtcaccctt

SEQ ID NO: 787 - RNA, 19 nt, synthetic construct
  source 1..19: mol_type = other RNA
ctgggaggatgaattcgaa

SEQ ID NO: 788 - RNA, 21 nt, synthetic construct
  source 1..21: mol_type = other RNA
ttcgaattcatcctcccagtt

SEQ ID NO: 789 - RNA, 19 nt, synthetic construct
  source 1..19: mol_type = other RNA
caaggtgggtggcatctaa

SEQ ID NO: 790 - RNA, 21 nt, synthetic construct
  source 1..21: mol_type = other RNA
ttagatgccacccaccttgtt

SEQ ID NO: 791 - RNA, 19 nt, synthetic construct
  source 1..19: mol_type = other RNA
tccgcagcctggatgatta

SEQ ID NO: 792 - RNA, 21 nt, synthetic construct
  source 1..21: mol_type = other RNA
taatcatccaggctgcggatt

SEQ ID NO: 793 - RNA, 19 nt, synthetic construct
  source 1..19: mol_type = other RNA
  modified_base 1..2: mod_base = OTHER; note = 2'O-Methyl ribonucleotide phosphorothioate
  modified_base 3: mod_base = OTHER; note = 2'Fluoro ribonucleotide
  modified_base 4..6: mod_base = OTHER; note = 2'O-Methyl ribonucleotide
  modified_base 7..9: mod_base = OTHER; note = 2'Fluoro ribonucleotide
  modified_base 10..11: mod_base = OTHER; note = 2'O-Methyl ribonucleotide
  modified_base 12: mod_base = OTHER; note = 2'Fluoro ribonucleotide
  modified_base 13..16: mod_base = OTHER; note = 2'O-Methyl ribonucleotide
  modified_base 17: mod_base = OTHER; note = 2'Fluoro ribonucleotide
  modified_base 18..19: mod_base = OTHER; note = 2'O-Methyl ribonucleotide
gggtgacaactactatcta

SEQ ID NO: 794 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4..14: mod_base = OTHER; note = 2'O-Methyl ribonucleotide
  modified_base 15: mod_base = OTHER; note = 2'Fluoro ribonucleotide
  modified_base 16..19: mod_base = OTHER; note = 2'O-Methyl ribonucleotide
  modified_base 20..21: mod_base = OTHER; note = 2'O-Methyl ribonucleotide phosphorothioate
  modified_base 22: mod_base = OTHER; note = 2'O-Methyl ribonucleotide
ttagatagtagttgtcaccctt

SEQ ID NO: 795 - RNA, 19 nt, synthetic construct
  source 1..19: mol_type = other RNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
ctgggaggatgaattcgaa

SEQ ID NO: 796 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttcgaattcatcctcccagtt

SEQ ID NO: 797 - RNA, 19 nt, synthetic construct
  source 1..19: mol_type = other RNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
caaggtgggtggcatctaa

SEQ ID NO: 798 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
tttagatgccacccaccttgtt

SEQ ID NO: 799 - RNA, 19 nt, synthetic construct
  source 1..19: mol_type = other RNA
  modified_base 1: mod_base = OTHER; note = 2'Fluoro ribonucleotide phosphorothioate
  modified_base 2: mod_base = OTHER; note = 2'O-Methyl ribonucleotide phosphorothioate
  modified_base 3: mod_base = OTHER; note = 2'Fluoro ribonucleotide
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
tccgcagcctggatgatta

SEQ ID NO: 800 - RNA, 22 nt, synthetic construct
  source 1..22: mol_type = other RNA
  modified_base 1: mod_base = OTHER; note = 2'O methyl vinyl phosphonate uridine
  modified_base 2: mod_base = OTHER; note = 2'O-Methyl ribonucleotide phosphorothioate
  modified_base 3: mod_base = OTHER; note = 2'Fluoro ribonucleotide phosphorothioate
  modified_base 4: mod_base = OTHER; note = 2'O-Methyl ribonucleotide
  modified_base 5: mod_base = OTHER; note = 2'Fluoro ribonucleotide
  modified_base 6: mod_base = OTHER; note = 2'O-Methyl ribonucleotide
  modified_base 7: mod_base = OTHER; note = 2'Fluoro ribonucleotide
  modified_base 8: mod_base = OTHER; note = 2'O-Methyl ribonucleotide
  modified_base 9: mod_base = OTHER; note = 2'Fluoro ribonucleotide
  modified_base 10: mod_base = OTHER; note = 2'O-Methyl ribonucleotide
  modified_base 11: mod_base = OTHER; note = 2'Fluoro ribonucleotide
  modified_base 12: mod_base = OTHER; note = 2'O-Methyl ribonucleotide
  modified_base 13: mod_base = OTHER; note = 2'Fluoro ribonucleotide
  modified_base 14: mod_base = OTHER; note = 2'O-Methyl ribonucleotide
  modified_base 15: mod_base = OTHER; note = 2'Fluoro ribonucleotide
  modified_base 16: mod_base = OTHER; note = 2'O-Methyl ribonucleotide
  modified_base 17: mod_base = OTHER; note = 2'Fluoro ribonucleotide
  modified_base 18: mod_base = OTHER; note = 2'O-Methyl ribonucleotide
  modified_base 19: mod_base = OTHER; note = 2'Fluoro ribonucleotide
  modified_base 20: mod_base = OTHER; note = 2'O-Methyl ribonucleotide phosphorothioate
  modified_base 21: mod_base = OTHER; note = 2'Fluoro ribonucleotide phosphorothioate
  modified_base 22: mod_base = OTHER; note = 2'O-Methyl ribonucleotide
ttaatcatccaggctgcggatt

SEQ ID NO: 801 - RNA, 19 nt, synthetic construct
  source 1..19: mol_type = other RNA
caggggtgcggtcttgcaa

SEQ ID NO: 802 - RNA, 19 nt, synthetic construct
  source 1..19: mol_type = other RNA
ggtgcggtcttgcaatagg

SEQ ID NO: 803 - RNA, 19 nt, synthetic construct
  source 1..19: mol_type = other RNA
gtgcggtcttgcaatagga

SEQ ID NO: 804 - RNA, 19 nt, synthetic construct
  source 1..19: mol_type = other RNA
agccatgcctttaaaccgc

SEQ ID NO: 805 - RNA, 19 nt, synthetic construct
  source 1..19: mol_type = other RNA
atgcctttaaaccgcactt

SEQ ID NO: 806 - RNA, 19 nt, synthetic construct
  source 1..19: mol_type = other RNA
tgggaggatgaattcgacc

SEQ ID NO: 807 - RNA, 19 nt, synthetic construct
  source 1..19: mol_type = other RNA
aacgcagtgctcttcgaag

SEQ ID NO: 808 - RNA, 19 nt, synthetic construct
  source 1..19: mol_type = other RNA
ggacgaatggggcgacaac

SEQ ID NO: 809 - RNA, 19 nt, synthetic construct
  source 1..19: mol_type = other RNA
ggcgactgcctgtagcaac

SEQ ID NO: 810 - RNA, 19 nt, synthetic construct
  source 1..19: mol_type = other RNA
cacgctgctggggcgctac

SEQ ID NO: 811 - RNA, 19 nt, synthetic construct
  source 1..19: mol_type = other RNA
tgcgctcacgtcttcacta

SEQ ID NO: 812 - RNA, 19 nt, synthetic construct
  source 1..19: mol_type = other RNA
ccttatacttctttatcgc

SEQ ID NO: 813 - RNA, 19 nt, synthetic construct
  source 1..19: mol_type = other RNA
ctttatcgccggccgctat

SEQ ID NO: 814 - RNA, 19 nt, synthetic construct
  source 1..19: mol_type = other RNA
tcgccggccgctatgagtt

SEQ ID NO: 815 - RNA, 19 nt, synthetic construct
  source 1..19: mol_type = other RNA
gccgctatgagttctccaa

SEQ ID NO: 816 - RNA, 19 nt, synthetic construct
  source 1..19: mol_type = other RNA
ccgctatgagttctccaac

SEQ ID NO: 817 - RNA, 19 nt, synthetic construct
  source 1..19: mol_type = other RNA
ggctcggctcaactatctg

SEQ ID NO: 818 - RNA, 19 nt, synthetic construct
  source 1..19: mol_type = other RNA
gctcggctcaactatctgc

SEQ ID NO: 819 - RNA, 19 nt, synthetic construct
  source 1..19: mol_type = other RNA
tcggctcaactatctgctc

SEQ ID NO: 820 - RNA, 19 nt, synthetic construct
  source 1..19: mol_type = other RNA
ccagcgcggaccaacaatt

SEQ ID NO: 821 - RNA, 19 nt, synthetic construct
  source 1..19: mol_type = other RNA
agcgcggaccaacaatttc

SEQ ID NO: 822 - RNA, 19 nt, synthetic construct
  source 1..19: mol_type = other RNA
tcgggaggaagctttatga

SEQ ID NO: 823 - RNA, 19 nt, synthetic construct
  source 1..19: mol_type = other RNA
acgcagcggcagtctttcc

SEQ ID NO: 824 - RNA, 19 nt, synthetic construct
  source 1..19: mol_type = other RNA
gaccaccatccgccgaatc

SEQ ID NO: 825 - RNA, 19 nt, synthetic construct
  source 1..19: mol_type = other RNA
ccgaatcggcctcttcaat

SEQ ID NO: 826 - RNA, 19 nt, synthetic construct
  source 1..19: mol_type = other RNA
aatcggcctcttcaatagc

SEQ ID NO: 827 - RNA, 19 nt, synthetic construct
  source 1..19: mol_type = other RNA
caatagcagtgccgacagg

SEQ ID NO: 828 - RNA, 19 nt, synthetic construct
  source 1..19: mol_type = other RNA
aggagtttgtccgtggctg

SEQ ID NO: 829 - RNA, 19 nt, synthetic construct
  source 1..19: mol_type = other RNA
cggctgagtgcacggttat

SEQ ID NO: 830 - RNA, 19 nt, synthetic construct
  source 1..19: mol_type = other RNA
tgggaatccccagtatctc

SEQ ID NO: 831 - RNA, 19 nt, synthetic construct
  source 1..19: mol_type = other RNA
cagacccctcagcttacgg

SEQ ID NO: 832 - RNA, 19 nt, synthetic construct
  source 1..19: mol_type = other RNA
cccctcagcttacggtatc

SEQ ID NO: 833 - RNA, 19 nt, synthetic construct
  source 1..19: mol_type = other RNA
ctcagcttacggtatctac

SEQ ID NO: 834 - RNA, 19 nt, synthetic construct
  source 1..19: mol_type = other RNA
gcttacggtatctacattc

SEQ ID NO: 835 - RNA, 19 nt, synthetic construct
  source 1..19: mol_type = other RNA
ccggcggcagcgtatcatc

SEQ ID NO: 836 - RNA, 19 nt, synthetic construct
  source 1..19: mol_type = other RNA
agcgtatcatccagcggaa

SEQ ID NO: 837 - RNA, 19 nt, synthetic construct
  source 1..19: mol_type = other RNA
gcgtatcatccagcggaac

SEQ ID NO: 838 - RNA, 19 nt, synthetic construct
  source 1..19: mol_type = other RNA
tacctaggccggtactata

SEQ ID NO: 839 - RNA, 19 nt, synthetic construct
  source 1..19: mol_type = other RNA
ctaggccggtactatatgt

SEQ ID NO: 840 - RNA, 19 nt, synthetic construct
  source 1..19: mol_type = other RNA
aggccggtactatatgtct

SEQ ID NO: 841 - RNA, 19 nt, synthetic construct
  source 1..19: mol_type = other RNA
ggccggtactatatgtctg

SEQ ID NO: 842 - RNA, 19 nt, synthetic construct
  source 1..19: mol_type = other RNA
gccggtactatatgtctgc

SEQ ID NO: 843 - RNA, 19 nt, synthetic construct
  source 1..19: mol_type = other RNA
cggtactatatgtctgcgc

SEQ ID NO: 844 - RNA, 19 nt, synthetic construct
  source 1..19: mol_type = other RNA
ggtactatatgtctgcgcg

SEQ ID NO: 845 - RNA, 19 nt, synthetic construct
  source 1..19: mol_type = other RNA
ctatatgtctgcgcgccac

SEQ ID NO: 846 - RNA, 19 nt, synthetic construct
  source 1..19: mol_type = other RNA
tatgtctgcgcgccacatg

SEQ ID NO: 847 - RNA, 19 nt, synthetic construct
  source 1..19: mol_type = other RNA
atgtctgcgcgccacatgg

SEQ ID NO: 848 - RNA, 19 nt, synthetic construct
  source 1..19: mol_type = other RNA
tcgccctcgctgtcacgac

SEQ ID NO: 849 - RNA, 19 nt, synthetic construct
  source 1..19: mol_type = other RNA
cggcgagcgctacgatgag

SEQ ID NO: 850 - RNA, 19 nt, synthetic construct
  source 1..19: mol_type = other RNA
caaggaccggcgcaacatc

SEQ ID NO: 851 - RNA, 19 nt, synthetic construct
  source 1..19: mol_type = other RNA
acatccgtgcaccagagtg

SEQ ID NO: 852 - RNA, 19 nt, synthetic construct
  source 1..19: mol_type = other RNA
ctgggcgaggagcgtaact

SEQ ID NO: 853 - RNA, 19 nt, synthetic construct
  source 1..19: mol_type = other RNA
gggcgaggagcgtaactaa

SEQ ID NO: 854 - RNA, 19 nt, synthetic construct
  source 1..19: mol_type = other RNA
gcgaggagcgtaactaagt

SEQ ID NO: 855 - RNA, 19 nt, synthetic construct
  source 1..19: mol_type = other RNA
gaggagcgtaactaagtcc

SEQ ID NO: 856 - RNA, 19 nt, synthetic construct
  source 1..19: mol_type = other RNA
agtccgccaaacactccac

SEQ ID NO: 857 - RNA, 19 nt, synthetic construct
  source 1..19: mol_type = other RNA
ggcgatcaagtccagagcc

SEQ ID NO: 858 - RNA, 19 nt, synthetic construct
  source 1..19: mol_type = other RNA
ccctaacctggcttattcc

SEQ ID NO: 859 - RNA, 19 nt, synthetic construct
  source 1..19: mol_type = other RNA
tgtgaaaccactaggttct

SEQ ID NO: 860 - RNA, 19 nt, synthetic construct
  source 1..19: mol_type = other RNA
accactaggttctaggtcc

SEQ ID NO: 861 - RNA, 21 nt, synthetic construct
  source 1..21: mol_type = other RNA
ttgcaagaccgcacccctgtt

SEQ ID NO: 862 - RNA, 21 nt, synthetic construct
  source 1..21: mol_type = other RNA
cctattgcaagaccgcacctt

SEQ ID NO: 863 - RNA, 21 nt, synthetic construct
  source 1..21: mol_type = other RNA
tcctattgcaagaccgcactt

SEQ ID NO: 864 - RNA, 21 nt, synthetic construct
  source 1..21: mol_type = other RNA
gcggtttaaaggcatggcttt

SEQ ID NO: 865 - RNA, 21 nt, synthetic construct
  source 1..21: mol_type = other RNA
aagtgcggtttaaaggcattt

SEQ ID NO: 866 - RNA, 21 nt, synthetic construct
  source 1..21: mol_type = other RNA
ggtcgaattcatcctcccatt

SEQ ID NO: 867 - RNA, 21 nt, synthetic construct
  source 1..21: mol_type = other RNA
cttcgaagagcactgcgtttt

SEQ ID NO: 868 - RNA, 21 nt, synthetic construct
  source 1..21: mol_type = other RNA
gttgtcgccccattcgtcctt

SEQ ID NO: 869 - RNA, 21 nt, synthetic construct
  source 1..21: mol_type = other RNA
gttgctacaggcagtcgcctt

SEQ ID NO: 870 - RNA, 21 nt, synthetic construct
  source 1..21: mol_type = other RNA
gtagcgccccagcagcgtgtt

SEQ ID NO: 871 - RNA, 21 nt, synthetic construct
  source 1..21: mol_type = other RNA
tagtgaagacgtgagcgcatt

SEQ ID NO: 872 - RNA, 21 nt, synthetic construct
  source 1..21: mol_type = other RNA
gcgataaagaagtataaggtt

SEQ ID NO: 873 - RNA, 21 nt, synthetic construct
  source 1..21: mol_type = other RNA
atagcggccggcgataaagtt

SEQ ID NO: 874 - RNA, 21 nt, synthetic construct
  source 1..21: mol_type = other RNA
aactcatagcggccggcgatt

SEQ ID NO: 875 - RNA, 21 nt, synthetic construct
  source 1..21: mol_type = other RNA
ttggagaactcatagcggctt

SEQ ID NO: 876 - RNA, 21 nt, synthetic construct
  source 1..21: mol_type = other RNA
gttggagaactcatagcggtt

SEQ ID NO: 877 - RNA, 21 nt, synthetic construct
  source 1..21: mol_type = other RNA
cagatagttgagccgagcctt

SEQ ID NO: 878 - RNA, 21 nt, synthetic construct
  source 1..21: mol_type = other RNA
gcagatagttgagccgagctt

SEQ ID NO: 879 - RNA, 21 nt, synthetic construct
  source 1..21: mol_type = other RNA
gagcagatagttgagccgatt

SEQ ID NO: 880 - RNA, 21 nt, synthetic construct
  source 1..21: mol_type = other RNA
aattgttggtccgcgctggtt

SEQ ID NO: 881 - RNA, 21 nt, synthetic construct
  source 1..21: mol_type = other RNA
gaaattgttggtccgcgcttt

SEQ ID NO: 882 - RNA, 21 nt, synthetic construct
  source 1..21: mol_type = other RNA
tcataaagcttcctcccgatt

SEQ ID NO: 883 - RNA, 21 nt, synthetic construct
  source 1..21: mol_type = other RNA
ggaaagactgccgctgcgttt

SEQ ID NO: 884 - RNA, 21 nt, synthetic construct
  source 1..21: mol_type = other RNA
gattcggcggatggtggtctt

SEQ ID NO: 885 - RNA, 21 nt, synthetic construct
  source 1..21: mol_type = other RNA
attgaagaggccgattcggtt

SEQ ID NO: 886 - RNA, 21 nt, synthetic construct
  source 1..21: mol_type = other RNA
gctattgaagaggccgatttt

SEQ ID NO: 887 - RNA, 21 nt, synthetic construct
  source 1..21: mol_type = other RNA
cctgtcggcactgctattgtt

SEQ ID NO: 888 - RNA, 21 nt, synthetic construct
  source 1..21: mol_type = other RNA
cagccacggacaaactccttt

SEQ ID NO: 889 - RNA, 21 nt, synthetic construct
  source 1..21: mol_type = other RNA
ataaccgtgcactcagccgtt

SEQ ID NO: 890 - RNA, 21 nt, synthetic construct
  source 1..21: mol_type = other RNA
gagatactggggattcccatt

SEQ ID NO: 891 - RNA, 21 nt, synthetic construct
  source 1..21: mol_type = other RNA
ccgtaagctgaggggtctgtt

SEQ ID NO: 892 - RNA, 21 nt, synthetic construct
  source 1..21: mol_type = other RNA
gataccgtaagctgaggggtt

SEQ ID NO: 893 - RNA, 21 nt, synthetic construct
  source 1..21: mol_type = other RNA
gtagataccgtaagctgagtt

SEQ ID NO: 894 - RNA, 21 nt, synthetic construct
  source 1..21: mol_type = other RNA
gaatgtagataccgtaagctt

SEQ ID NO: 895 - RNA, 21 nt, synthetic construct
  source 1..21: mol_type = other RNA
gatgatacgctgccgccggtt

SEQ ID NO: 896 - RNA, 21 nt, synthetic construct
  source 1..21: mol_type = other RNA
ttccgctggatgatacgcttt

SEQ ID NO: 897 - RNA, 21 nt, synthetic construct
  source 1..21: mol_type = other RNA
gttccgctggatgatacgctt

SEQ ID NO: 898 - RNA, 21 nt, synthetic construct
  source 1..21: mol_type = other RNA
tatagtaccggcctaggtatt

SEQ ID NO: 899 - RNA, 21 nt, synthetic construct
  source 1..21: mol_type = other RNA
acatatagtaccggcctagtt

SEQ ID NO: 900 - RNA, 21 nt, synthetic construct
  source 1..21: mol_type = other RNA
agacatatagtaccggccttt

SEQ ID NO: 901 - RNA, 21 nt, synthetic construct
  source 1..21: mol_type = other RNA
cagacatatagtaccggcctt

SEQ ID NO: 902 - RNA, 21 nt, synthetic construct
  source 1..21: mol_type = other RNA
gcagacatatagtaccggctt

SEQ ID NO: 903 - RNA, 21 nt, synthetic construct
  source 1..21: mol_type = other RNA
gcgcagacatatagtaccgtt

SEQ ID NO: 904 - RNA, 21 nt, synthetic construct
  source 1..21: mol_type = other RNA
cgcgcagacatatagtacctt

SEQ ID NO: 905 - RNA, 21 nt, synthetic construct
  source 1..21: mol_type = other RNA
gtggcgcgcagacatatagtt

SEQ ID NO: 906 - RNA, 21 nt, synthetic construct
  source 1..21: mol_type = other RNA
catgtggcgcgcagacatatt

SEQ ID NO: 907 - RNA, 21 nt, synthetic construct
  source 1..21: mol_type = other RNA
ccatgtggcgcgcagacattt

SEQ ID NO: 908 - RNA, 21 nt, synthetic construct
  source 1..21: mol_type = other RNA
gtcgtgacagcgagggcgatt

SEQ ID NO: 909 - RNA, 21 nt, synthetic construct
  source 1..21: mol_type = other RNA
ctcatcgtagcgctcgccgtt

SEQ ID NO: 910 - RNA, 21 nt, synthetic construct
  source 1..21: mol_type = other RNA
gatgttgcgccggtccttgtt

SEQ ID NO: 911 - RNA, 21 nt, synthetic construct
  source 1..21: mol_type = other RNA
cactctggtgcacggatgttt

SEQ ID NO: 912 - RNA, 21 nt, synthetic construct
  source 1..21: mol_type = other RNA
agttacgctcctcgcccagtt

SEQ ID NO: 913 - RNA, 21 nt, synthetic construct
  source 1..21: mol_type = other RNA
ttagttacgctcctcgccctt

SEQ ID NO: 914 - RNA, 21 nt, synthetic construct
  source 1..21: mol_type = other RNA
acttagttacgctcctcgctt

SEQ ID NO: 915 - RNA, 21 nt, synthetic construct
  source 1..21: mol_type = other RNA
ggacttagttacgctcctctt

SEQ ID NO: 916 - RNA, 21 nt, synthetic construct
  source 1..21: mol_type = other RNA
gtggagtgtttggcggacttt

SEQ ID NO: 917 - RNA, 21 nt, synthetic construct
  source 1..21: mol_type = other RNA
ggctctggacttgatcgcctt

SEQ ID NO: 918 - RNA, 21 nt, synthetic construct
  source 1..21: mol_type = other RNA
ggaataagccaggttagggtt

SEQ ID NO: 919 - RNA, 21 nt, synthetic construct
  source 1..21: mol_type = other RNA
agaacctagtggtttcacatt

SEQ ID NO: 920 - RNA, 21 nt, synthetic construct
  source 1..21: mol_type = other RNA
ggacctagaacctagtggttt

SEQ ID NO: 921 - RNA, 19 nt, synthetic construct
  source 1..19: mol_type = other RNA
caggggtgcggtcttgcaa

SEQ ID NO: 922 - RNA, 19 nt, synthetic construct
  source 1..19: mol_type = other RNA
ggtgcggtcttgcaataga

SEQ ID NO: 923 - RNA, 19 nt, synthetic construct
  source 1..19: mol_type = other RNA
gtgcggtcttgcaatagga

SEQ ID NO: 924 - RNA, 19 nt, synthetic construct
  source 1..19: mol_type = other RNA
agccatgcctttaaaccga

SEQ ID NO: 925 - RNA, 19 nt, synthetic construct
  source 1..19: mol_type = other RNA
atgcctttaaaccgcacta

SEQ ID NO: 926 - RNA, 19 nt, synthetic construct
  source 1..19: mol_type = other RNA
tgggaggatgaattcgaca

SEQ ID NO: 927 - RNA, 19 nt, synthetic construct
  source 1..19: mol_type = other RNA
aacgcagtgctcttcgaaa

SEQ ID NO: 928 - RNA, 19 nt, synthetic construct
  source 1..19: mol_type = other RNA
ggacgaatggggcgacaaa

SEQ ID NO: 929 - RNA, 19 nt, synthetic construct
  source 1..19: mol_type = other RNA
ggcgactgcctgtagcaaa

SEQ ID NO: 930 - RNA, 19 nt, synthetic construct
  source 1..19: mol_type = other RNA
cacgctgctggggcgctaa

SEQ ID NO: 931 - RNA, 19 nt, synthetic construct
  source 1..19: mol_type = other RNA
tgcgctcacgtcttcacta

SEQ ID NO: 932 - RNA, 19 nt, synthetic construct
  source 1..19: mol_type = other RNA
ccttatacttctttatcga

SEQ ID NO: 933 - RNA, 19 nt, synthetic construct
  source 1..19: mol_type = other RNA
ctttatcgccggccgctaa

SEQ ID NO: 934 - RNA, 19 nt, synthetic construct
  source 1..19: mol_type = other RNA
tcgccggccgctatgagta

SEQ ID NO: 935 - RNA, 19 nt, synthetic construct
  source 1..19: mol_type = other RNA
gccgctatgagttctccaa

SEQ ID NO: 936 - RNA, 19 nt, synthetic construct
  source 1..19: mol_type = other RNA
ccgctatgagttctccaaa

SEQ ID NO: 937 - RNA, 19 nt, synthetic construct
  source 1..19: mol_type = other RNA
ggctcggctcaactatcta

SEQ ID NO: 938 - RNA, 19 nt, synthetic construct
  source 1..19: mol_type = other RNA
gctcggctcaactatctga

SEQ ID NO: 939 - RNA, 19 nt, synthetic construct
  source 1..19: mol_type = other RNA
tcggctcaactatctgcta

SEQ ID NO: 940 - RNA, 19 nt, synthetic construct
  source 1..19: mol_type = other RNA
ccagcgcggaccaacaata

SEQ ID NO: 941 - RNA, 19 nt, synthetic construct
  source 1..19: mol_type = other RNA
agcgcggaccaacaattta

SEQ ID NO: 942 - RNA, 19 nt, synthetic construct
  source 1..19: mol_type = other RNA
tcgggaggaagctttatga

SEQ ID NO: 943 - RNA, 19 nt, synthetic construct
  source 1..19: mol_type = other RNA
acgcagcggcagtctttca

SEQ ID NO: 944 - RNA, 19 nt, synthetic construct
  source 1..19: mol_type = other RNA
gaccaccatccgccgaata

SEQ ID NO: 945 - RNA, 19 nt, synthetic construct
  source 1..19: mol_type = other RNA
ccgaatcggcctcttcaaa

SEQ ID NO: 946 - RNA, 19 nt, synthetic construct
  source 1..19: mol_type = other RNA
aatcggcctcttcaataga

SEQ ID NO: 947 - RNA, 19 nt, synthetic construct
  source 1..19: mol_type = other RNA
caatagcagtgccgacaga

SEQ ID NO: 948 - RNA, 19 nt, synthetic construct
  source 1..19: mol_type = other RNA
aggagtttgtccgtggcta

SEQ ID NO: 949 - RNA, 19 nt, synthetic construct
  source 1..19: mol_type = other RNA
cggctgagtgcacggttaa

SEQ ID NO: 950 - RNA, 19 nt, synthetic construct
  source 1..19: mol_type = other RNA
tgggaatccccagtatcta

SEQ ID NO: 951 - RNA, 19 nt, synthetic construct
  source 1..19: mol_type = other RNA
cagacccctcagcttacga

SEQ ID NO: 952 - RNA, 19 nt, synthetic construct
  source 1..19: mol_type = other RNA
cccctcagcttacggtata

SEQ ID NO: 953 - RNA, 19 nt, synthetic construct
  source 1..19: mol_type = other RNA
ctcagcttacggtatctaa

SEQ ID NO: 954 - RNA, 19 nt, synthetic construct
  source 1..19: mol_type = other RNA
gcttacggtatctacatta

SEQ ID NO: 955 - RNA, 19 nt, synthetic construct
  source 1..19: mol_type = other RNA
ccggcggcagcgtatcata

SEQ ID NO: 956 - RNA, 19 nt, synthetic construct
  source 1..19: mol_type = other RNA
agcgtatcatccagcggaa

SEQ ID NO: 957 - RNA, 19 nt, synthetic construct
  source 1..19: mol_type = other RNA
gcgtatcatccagcggaaa

SEQ ID NO: 958 - RNA, 19 nt, synthetic construct
  source 1..19: mol_type = other RNA
tacctaggccggtactata

SEQ ID NO: 959 - RNA, 19 nt, synthetic construct
  source 1..19: mol_type = other RNA
ctaggccggtactatatga

SEQ ID NO: 960 - RNA, 19 nt, synthetic construct
  source 1..19: mol_type = other RNA
aggccggtactatatgtca

SEQ ID NO: 961 - RNA, 19 nt, synthetic construct
  source 1..19: mol_type = other RNA
ggccggtactatatgtcta

SEQ ID NO: 962 - RNA, 19 nt, synthetic construct
  source 1..19: mol_type = other RNA
gccggtactatatgtctga

SEQ ID NO: 963 - RNA, 19 nt, synthetic construct
  source 1..19: mol_type = other RNA
cggtactatatgtctgcga

SEQ ID NO: 964 - RNA, 19 nt, synthetic construct
  source 1..19: mol_type = other RNA
ggtactatatgtctgcgca

SEQ ID NO: 965 - RNA, 19 nt, synthetic construct
  source 1..19: mol_type = other RNA
ctatatgtctgcgcgccaa

SEQ ID NO: 966 - RNA, 19 nt, synthetic construct
  source 1..19: mol_type = other RNA
tatgtctgcgcgccacata

SEQ ID NO: 967 - RNA, 19 nt, synthetic construct
  source 1..19: mol_type = other RNA
atgtctgcgcgccacatga

SEQ ID NO: 968 - RNA, 19 nt, synthetic construct
  source 1..19: mol_type = other RNA
tcgccctcgctgtcacgaa

SEQ ID NO: 969 - RNA, 19 nt, synthetic construct
  source 1..19: mol_type = other RNA
cggcgagcgctacgatgaa

SEQ ID NO: 970 - RNA, 19 nt, synthetic construct
  source 1..19: mol_type = other RNA
caaggaccggcgcaacata

SEQ ID NO: 971 - RNA, 19 nt, synthetic construct
  source 1..19: mol_type = other RNA
acatccgtgcaccagagta

SEQ ID NO: 972 - RNA, 19 nt, synthetic construct
  source 1..19: mol_type = other RNA
ctgggcgaggagcgtaaca

SEQ ID NO: 973 - RNA, 19 nt, synthetic construct
  source 1..19: mol_type = other RNA
gggcgaggagcgtaactaa

SEQ ID NO: 974 - RNA, 19 nt, synthetic construct
  source 1..19: mol_type = other RNA
gcgaggagcgtaactaaga

SEQ ID NO: 975 - RNA, 19 nt, synthetic construct
  source 1..19: mol_type = other RNA
gaggagcgtaactaagtca

SEQ ID NO: 976 - RNA, 19 nt, synthetic construct
  source 1..19: mol_type = other RNA
agtccgccaaacactccaa

SEQ ID NO: 977 - RNA, 19 nt, synthetic construct
  source 1..19: mol_type = other RNA
ggcgatcaagtccagagca

SEQ ID NO: 978 - RNA, 19 nt, synthetic construct
  source 1..19: mol_type = other RNA
ccctaacctggcttattca

SEQ ID NO: 979 - RNA, 19 nt, synthetic construct
  source 1..19: mol_type = other RNA
tgtgaaaccactaggttca

SEQ ID NO: 980 - RNA, 19 nt, synthetic construct
  source 1..19: mol_type = other RNA
accactaggttctaggtca

SEQ ID NO: 981 - RNA, 21 nt, synthetic construct
  source 1..21: mol_type = other RNA
ttgcaagaccgcacccctgtt

SEQ ID NO: 982 - RNA, 21 nt, synthetic construct
  source 1..21: mol_type = other RNA
tctattgcaagaccgcacctt

SEQ ID NO: 983 - RNA, 21 nt, synthetic construct
  source 1..21: mol_type = other RNA
tcctattgcaagaccgcactt

SEQ ID NO: 984 - RNA, 21 nt, synthetic construct
  source 1..21: mol_type = other RNA
tcggtttaaaggcatggcttt

SEQ ID NO: 985 - RNA, 21 nt, synthetic construct
  source 1..21: mol_type = other RNA
tagtgcggtttaaaggcattt

SEQ ID NO: 986 - RNA, 21 nt, synthetic construct
  source 1..21: mol_type = other RNA
tgtcgaattcatcctcccatt

SEQ ID NO: 987 - RNA, 21 nt, synthetic construct
  source 1..21: mol_type = other RNA
tttcgaagagcactgcgtttt

SEQ ID NO: 988 - RNA, 21 nt, synthetic construct
  source 1..21: mol_type = other RNA
tttgtcgccccattcgtcctt

SEQ ID NO: 989 - RNA, 21 nt, synthetic construct
  source 1..21: mol_type = other RNA
tttgctacaggcagtcgcctt

SEQ ID NO: 990 - RNA, 21 nt, synthetic construct
  source 1..21: mol_type = other RNA
ttagcgccccagcagcgtgtt

SEQ ID NO: 991 - RNA, 21 nt, synthetic construct
  source 1..21: mol_type = other RNA
tagtgaagacgtgagcgcatt

SEQ ID NO: 992 - RNA, 21 nt, synthetic construct
  source 1..21: mol_type = other RNA
tcgataaagaagtataaggtt

SEQ ID NO: 993 - RNA, 21 nt, synthetic construct
  source 1..21: mol_type = other RNA
ttagcggccggcgataaagtt

SEQ ID NO: 994 - RNA, 21 nt, synthetic construct
  source 1..21: mol_type = other RNA
tactcatagcggccggcgatt

SEQ ID NO: 995 - RNA, 21 nt, synthetic construct
  source 1..21: mol_type = other RNA
ttggagaactcatagcggctt

SEQ ID NO: 996 - RNA, 21 nt, synthetic construct
  source 1..21: mol_type = other RNA
tttggagaactcatagcggtt

SEQ ID NO: 997 - RNA, 21 nt, synthetic construct
  source 1..21: mol_type = other RNA
tagatagttgagccgagcctt

SEQ ID NO: 998 - RNA, 21 nt, synthetic construct
  source 1..21: mol_type = other RNA
tcagatagttgagccgagctt

SEQ ID NO: 999 - RNA, 21 nt, synthetic construct
  source 1..21: mol_type = other RNA
tagcagatagttgagccgatt

SEQ ID NO: 1000 - RNA, 21 nt, synthetic construct
  source 1..21: mol_type = other RNA
tattgttggtccgcgctggtt

SEQ ID NO: 1001 - RNA, 21 nt, synthetic construct
  source 1..21: mol_type = other RNA
taaattgttggtccgcgcttt

SEQ ID NO: 1002 - RNA, 21 nt, synthetic construct
  source 1..21: mol_type = other RNA
tcataaagcttcctcccgatt

SEQ ID NO: 1003 - RNA, 21 nt, synthetic construct
  source 1..21: mol_type = other RNA
tgaaagactgccgctgcgttt

SEQ ID NO: 1004 - RNA, 21 nt, synthetic construct
  source 1..21: mol_type = other RNA
tattcggcggatggtggtctt

SEQ ID NO: 1005 - RNA, 21 nt, synthetic construct
  source 1..21: mol_type = other RNA
tttgaagaggccgattcggtt

SEQ ID NO: 1006 - RNA, 21 nt, synthetic construct
  source 1..21: mol_type = other RNA
tctattgaagaggccgatttt

SEQ ID NO: 1007 - RNA, 21 nt, synthetic construct
  source 1..21: mol_type = other RNA
tctgtcggcactgctattgtt

SEQ ID NO: 1008 - RNA, 21 nt, synthetic construct
  source 1..21: mol_type = other RNA
tagccacggacaaactccttt

SEQ ID NO: 1009 - RNA, 21 nt, synthetic construct
  source 1..21: mol_type = other RNA
ttaaccgtgcactcagccgtt

SEQ ID NO: 1010 - RNA, 21 nt, synthetic construct
  source 1..21: mol_type = other RNA
tagatactggggattcccatt

SEQ ID NO: 1011 - RNA, 21 nt, synthetic construct
  source 1..21: mol_type = other RNA
tcgtaagctgaggggtctgtt

SEQ ID NO: 1012 - RNA, 21 nt, synthetic construct
  source 1..21: mol_type = other RNA
tataccgtaagctgaggggtt

SEQ ID NO: 1013 - RNA, 21 nt, synthetic construct
  source 1..21: mol_type = other RNA
ttagataccgtaagctgagtt

SEQ ID NO: 1014 - RNA, 21 nt, synthetic construct
  source 1..21: mol_type = other RNA
taatgtagataccgtaagctt

SEQ ID NO: 1015 - RNA, 21 nt, synthetic construct
  source 1..21: mol_type = other RNA
tatgatacgctgccgccggtt

SEQ ID NO: 1016 - RNA, 21 nt, synthetic construct
  source 1..21: mol_type = other RNA
ttccgctggatgatacgcttt

SEQ ID NO: 1017 - RNA, 21 nt, synthetic construct
  source 1..21: mol_type = other RNA
tttccgctggatgatacgctt

SEQ ID NO: 1018 - RNA, 21 nt, synthetic construct
  source 1..21: mol_type = other RNA
tatagtaccggcctaggtatt

SEQ ID NO: 1019 - RNA, 21 nt, synthetic construct
  source 1..21: mol_type = other RNA
tcatatagtaccggcctagtt

SEQ ID NO: 1020 - RNA, 21 nt, synthetic construct
  source 1..21: mol_type = other RNA
tgacatatagtaccggccttt

SEQ ID NO: 1021 - RNA, 21 nt, synthetic construct
  source 1..21: mol_type = other RNA
tagacatatagtaccggcctt

SEQ ID NO: 1022 - RNA, 21 nt, synthetic construct
  source 1..21: mol_type = other RNA
tcagacatatagtaccggctt

SEQ ID NO: 1023 - RNA, 21 nt, synthetic construct
  source 1..21: mol_type = other RNA
tcgcagacatatagtaccgtt

SEQ ID NO: 1024 - RNA, 21 nt, synthetic construct
  source 1..21: mol_type = other RNA
tgcgcagacatatagtacctt

SEQ ID NO: 1025 - RNA, 21 nt, synthetic construct
  source 1..21: mol_type = other RNA
ttggcgcgcagacatatagtt

SEQ ID NO: 1026 - RNA, 21 nt, synthetic construct
  source 1..21: mol_type = other RNA
tatgtggcgcgcagacatatt

SEQ ID NO: 1027 - RNA, 21 nt, synthetic construct
  source 1..21: mol_type = other RNA
tcatgtggcgcgcagacattt

SEQ ID NO: 1028 - RNA, 21 nt, synthetic construct
  source 1..21: mol_type = other RNA
ttcgtgacagcgagggcgatt

SEQ ID NO: 1029 - RNA, 21 nt, synthetic construct
  source 1..21: mol_type = other RNA
ttcatcgtagcgctcgccgtt

SEQ ID NO: 1030 - RNA, 21 nt, synthetic construct
  source 1..21: mol_type = other RNA
tatgttgcgccggtccttgtt

SEQ ID NO: 1031 - RNA, 21 nt, synthetic construct
  source 1..21: mol_type = other RNA
tactctggtgcacggatgttt

SEQ ID NO: 1032 - RNA, 21 nt, synthetic construct
  source 1..21: mol_type = other RNA
tgttacgctcctcgcccagtt

SEQ ID NO: 1033 - RNA, 21 nt, synthetic construct
  source 1..21: mol_type = other RNA
ttagttacgctcctcgccctt

SEQ ID NO: 1034 - RNA, 21 nt, synthetic construct
  source 1..21: mol_type = other RNA
tcttagttacgctcctcgctt

SEQ ID NO: 1035 - RNA, 21 nt, synthetic construct
  source 1..21: mol_type = other RNA
tgacttagttacgctcctctt

SEQ ID NO: 1036 - RNA, 21 nt, synthetic construct
  source 1..21: mol_type = other RNA
ttggagtgtttggcggacttt

SEQ ID NO: 1037 - RNA, 21 nt, synthetic construct
  source 1..21: mol_type = other RNA
tgctctggacttgatcgcctt

SEQ ID NO: 1038 - RNA, 21 nt, synthetic construct
  source 1..21: mol_type = other RNA
tgaataagccaggttagggtt

SEQ ID NO: 1039 - RNA, 21 nt, synthetic construct
  source 1..21: mol_type = other RNA
tgaacctagtggtttcacatt

SEQ ID NO: 1040 - RNA, 21 nt, synthetic construct
  source 1..21: mol_type = other RNA
tgacctagaacctagtggttt

SEQ ID NO: 1041 - RNA, 19 nt, synthetic construct
  source 1..19: mol_type = other RNA
tggacttcaacctagacaa

SEQ ID NO: 1042 - RNA, 21 nt, synthetic construct
  source 1..21: mol_type = other RNA
ttgtctaggttgaagtccatt

SEQ ID NO: 1043 - protein, 9 aa, synthetic construct
RKKRRQRRR

SEQ ID NO: 1044 - protein, 14 aa, synthetic construct
RQIKIWQNRRKWKK

SEQ ID NO: 1045 - protein, 26 aa, synthetic construct
GWTLNSAGYLLGKINKALAALAKKIL

SEQ ID NO: 1046 - protein, 18 aa, synthetic construct
KLALKLALKALKAALKLA

SEQ ID NO: 1047 - protein, 21 aa, synthetic construct
KETWWETWWTEWSQPKKKRKV

SEQ ID NO: 1048 - protein, 4 aa, synthetic construct
KDEL

SEQ ID NO: 1049 - protein, 29 aa, synthetic construct
WEAALAEALAELAEHLAEALAEALEALAA

SEQ ID NO: 1050 - protein, 21 aa, synthetic construct
GDIMGEWGNEIFGAIAGFLGC

SEQ ID NO: 1051 - protein, 13 aa, synthetic construct
GLFDIIKKIAESF

SEQ ID NO: 1052 - protein, 24 aa, synthetic construct
GALFLGWLGAAGSTMGAPKSKRKV

SEQ ID NO: 1053 - protein, 21 aa, synthetic construct
AGYLLGKINLKALAALAKKIL

SEQ ID NO: 1054 - protein, 23 aa, synthetic construct
LIRLWSHLIHIWFQNRRLKWKKK

SEQ ID NO: 1055 - protein, 36 aa, synthetic construct
GLFGAIAGFIENGWEGMIDGRQIKIWFQNRRMKWKK

SEQ ID NO: 1056 - protein, 26 aa, synthetic construct
FFKKLAHALHLLALLALHLAHALKKA

SEQ ID NO: 1057 - protein, 21 aa, synthetic construct
LFEAIEGFIENGWGMIDGWYG

SEQ ID NO: 1058 - protein, 31 aa, synthetic construct
LFEAIEGFIENGWEGMIDGWYGRKKRRQRRR

SEQ ID NO: 1059 - protein, 25 aa, synthetic construct
IGAVLKVLTTGLPALISWIKRKRQQ

SEQ ID NO: 1060 - protein, 21 aa, synthetic construct
MKLAVTLTLVTLALSSSSASA

SEQ ID NO: 1061 - protein, 22 aa, synthetic construct
RLIEDICLPRWGCLWEDDKDEL

SEQ ID NO: 1062 - protein, 17 aa, synthetic construct
MIRTLLLSTLVAGALSK

SEQ ID NO: 1063 - protein, 19 aa, synthetic construct
ILSSLTVTQLLRRLHQWIK

SEQ ID NO: 1064 - protein, 20 aa, synthetic construct
MIRTLLLSTLVAGALSKDEL

SEQ ID NO: 1065 - RNA, 19 nt, synthetic construct
  source 1..19: mol_type = other RNA
ttacggtatctacattcta

SEQ ID NO: 1066 - RNA, 21 nt, synthetic construct
  source 1..21: mol_type = other RNA
tagaatgtagataccgtaatt

SEQ ID NO: 1067 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4: mod_base = OTHER; note = 2'Fluoro ribonucleotide
  modified_base 5: mod_base = OTHER; note = 2'O-Methyl ribonucleotide
  modified_base 6: mod_base = OTHER; note = 2'Fluoro ribonucleotide
  modified_base 7: mod_base = OTHER; note = 2'O-Methyl ribonucleotide
  modified_base 8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11: mod_base = OTHER; note = 2'O-Methyl ribonucleotide
  modified_base 12: mod_base = OTHER; note = 2'Fluoro ribonucleotide
  modified_base 13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tttcattaaggccacgagatt

SEQ ID NO: 1068 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3..13: mod_base = OTHER; note = 2'O-Methyl ribonucleotide phosphorothioate
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttcgaattcatcctcccagtt

SEQ ID NO: 1069 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4: mod_base = OTHER; note = 2'Fluoro ribonucleotide
  modified_base 5: mod_base = OTHER; note = 2'O-Methyl ribonucleotide
  modified_base 6..8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11..13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19: mod_base = OTHER; note = 2'O-Methyl ribonucleotide phosphorothioate
  modified_base 20: mod_base = OTHER; note = 2'Fluoro ribonucleotide phosphorothioate
  modified_base 21: mod_base = OTHER; note = 2'O-Methyl ribonucleotide
ttagatgccacccaccttgtt

SEQ ID NO: 1070 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgaaattgttggtccgcgctt

SEQ ID NO: 1071 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tgcgttgcaaagatggctctt

SEQ ID NO: 1072 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
ttgcgttgcaaagatggcttt

SEQ ID NO: 1073 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tagaatgtagataccgtaatt

SEQ ID NO: 1074 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4..13: mod_base = OTHER; note = 2'O-Methyl ribonucleotide
  modified_base 14: mod_base = OTHER; note = 2'Fluoro ribonucleotide
  modified_base 15..18: mod_base = OTHER; note = 2'O-Methyl ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tcctcgaggtaaatgtgggtt

SEQ ID NO: 1075 - RNA, 21 nt, synthetic construct
  source 1..21: mol_type = other RNA
  modified_base 1: mod_base = OTHER; note = 2'O methyl vinyl phosphonate uridine
  modified_base 2: mod_base = OTHER; note = 2'Fluoro ribonucleotide phosphorothioate
  modified_base 3: mod_base = OTHER; note = 2'O-Methyl ribonucleotide phosphorothioate
  modified_base 4: mod_base = OTHER; note = 2'Fluoro ribonucleotide
  modified_base 5: mod_base = OTHER; note = 2'O-Methyl ribonucleotide
  modified_base 6: mod_base = OTHER; note = 2'Fluoro ribonucleotide
  modified_base 7: mod_base = OTHER; note = 2'O-Methyl ribonucleotide
  modified_base 8: mod_base = OTHER; note = 2'Fluoro ribonucleotide
  modified_base 9: mod_base = OTHER; note = 2'O-Methyl ribonucleotide
  modified_base 10: mod_base = OTHER; note = 2'Fluoro ribonucleotide
  modified_base 11: mod_base = OTHER; note = 2'O-Methyl ribonucleotide
  modified_base 12: mod_base = OTHER; note = 2'Fluoro ribonucleotide
  modified_base 13: mod_base = OTHER; note = 2'O-Methyl ribonucleotide
  modified_base 14: mod_base = OTHER; note = 2'Fluoro ribonucleotide
  modified_base 15: mod_base = OTHER; note = 2'O-Methyl ribonucleotide
  modified_base 16: mod_base = OTHER; note = 2'Fluoro ribonucleotide
  modified_base 17: mod_base = OTHER; note = 2'O-Methyl ribonucleotide
  modified_base 18: mod_base = OTHER; note = 2'Fluoro ribonucleotide
  modified_base 19..20: mod_base = OTHER; note = 2'O-Methyl ribonucleotide phosphorothioate
  modified_base 21: mod_base = OTHER; note = 2'O-Methyl ribonucleotide
tttcattaaggccacgagatt